(19) United States
(12) Patent Application Publication
(10) Pub. No.: US 2025/0066841 A1
(43) Pub. Date: Feb. 27, 2025

(54) COMPOSITIONS AND METHODS FOR DETECTING BINDING INTERACTIONS UNDER EQUILIBRIUM OR NON-EQUILIBRIUM CONDITIONS

(71) Applicant: Nautilus Subsidiary, Inc., Seattle, WA (US)

(72) Inventors: Robert GROTHE, San Jose, CA (US); Kara JUNEAU, San Carlos, CA (US); Michael Augusto DARCY, Fremont, CA (US); Parag MALLICK, San Mateo, CA (US); Jacinto VILLANUEVA, Pleasant Hill, CA (US); Vivekananda BUDAMAGUNTA, San Carlos, CA (US); Jonathan LEANO, Foster City, CA (US); Pengyu HAO, Belmont, CA (US); Terren CHANG, Redwood City, CA (US); Aimee SANFORD, San Mateo, CA (US); Maureen NEWMAN, Redwood City, CA (US); Filip BARTNICKI, Redwood City, CA (US); Rukshan PERERA, Newark, CA (US); Grant NAPIER, San Carlos, CA (US)

(21) Appl. No.: 18/813,886

(22) Filed: Aug. 23, 2024

Related U.S. Application Data
(60) Provisional application No. 63/578,624, filed on Aug. 24, 2023.
(60) Provisional application No. 63/640,664, filed on Apr. 30, 2024.

(51) Int. Cl. C12Q 1/6818; G01N 33/542

(52) U.S. Cl. CPC C12Q 1/6818; G01N 33/542

(57) ABSTRACT

Provided are methods of detecting analytes. In some configurations, the methods can employ analytes attached to a solid support or particle and affinity reagents that are attached to the solid support or particle via a flexible linker. In some configurations, the methods can employ analytes attached to a solid support or particle and solution-phase affinity reagents can be attracted to the analytes via application of a stimulus.

### CROSS-REFERENCE

This application claims priority to U.S. Provisional Application No. 63/578,624, filed on Aug. 24, 2023, and U.S. Provisional Application No. 63/640,664, filed on Apr. 30, 2024, each of which is incorporated herein by reference in its entirety.

### SEQUENCE LISTING

This application contains a Sequence Listing which has been submitted electronically in XML file format and is hereby incorporated by reference in its entirety. Said XML copy, created on Aug. 21, 2024, is named SL_50109_4023WO.xml and is 1,780 bytes in size.

### BACKGROUND

Affinity reagents include a broad class of reagents that form detectable interactions with analytes and other molecules. Affinity reagents can be configured to form reversible bound complexes with analytes. The observation of binding between an affinity reagent and analyte can provide useful information for characterizing the structure and properties of the analyte based on known recognition properties of the affinity reagent. Substantial resources and effort are typically invested into producing affinity reagents that bind to analytes of interest with sufficient specificity to distinguish the analytes in complex mixtures, such as biopsy samples, which typically contain a large number and variety of proteins, nucleic acids and other biochemical analytes. The specificity and strength of binding between an affinity reagent and analyte can be heavily influenced by small changes in conditions used for the assay such as changes in temperature, ionic strength, pH, and concentration of affinity reagent and analyte. This combination of factors can constrain the design of multiplex assays in which a large number of different analytes are to be evaluated, in parallel, for binding to a given affinity reagent. The present disclosure provides compositions and methods that improve binding assays and provide advantages that extend to multiplexed formats. Other advantages are provided as well.

### SUMMARY

The present disclosure provides a method of detecting analytes. The method can include steps of (a) providing a mixture of analytes with affinity reagents, wherein the analytes are immobilized on a surface and the affinity reagents are in fluid phase; (b) applying a stimulus to attract the affinity reagents to the surface; and (c) detecting binding of affinity reagents to analytes on the surface. For ease of explanation, several configurations of the method will be exemplified herein in the context of using immobilized analytes and fluid-phase affinity reagents. However, it will be understood that the method can be carried out using immobilized affinity reagents and fluid-phase analytes. Accordingly, the method can include steps of (a) providing a mixture of analytes with affinity reagents, wherein the affinity reagents are immobilized on a surface and the analytes are in fluid phase; (b) applying a stimulus to attract the analytes to the surface; and (c) detecting binding of analytes to affinity reagents on the surface.

In some configurations, a method of detecting analytes on an array can include steps of (a) contacting an array with affinity reagents in fluid phase, wherein analytes are attached at addresses in the array; (b) allowing affinity reagents in the fluid phase to bind to analytes attached at addresses in the array; (c) removing a plurality of the affinity reagents from contact with the array, thereby retaining a fraction of the affinity reagents in contact with the array; (d) applying a stimulus to attract the fraction of affinity reagents to the array; and (e) detecting binding of affinity reagents in the fraction to analytes attached at addresses in the array. Again, the method will be exemplified herein in the context of using immobilized analytes and fluid-phase affinity reagents. However, the method can be carried out using immobilized affinity reagents and fluid-phase analytes. Accordingly, the method can include steps of (a) contacting an array with analytes in fluid phase, wherein affinity reagents are attached at addresses in the array; (b) allowing analytes in the fluid phase to bind to affinity reagents attached at addresses in the array; (c) removing a plurality of the analytes from contact with the array, thereby retaining a fraction of the analytes in contact with the array; (d) applying a stimulus to attract the fraction of analytes to the array; and (e) detecting binding of analytes in the fraction to affinity reagents attached at addresses in the array.

The present disclosure further provides a method of detecting an analyte, including steps of (a) providing an analyte attached to a solid support or particle; (b) attaching an affinity reagent to the solid support or particle via a flexible linker; and (c) detecting binding between the affinity reagent and the analyte on the solid support or particle.

Also provided is a method of identifying an analyte in an array. The method can include steps of (a) providing an array of individually resolved analytes, wherein analytes are attached at addresses in the array, and wherein individual addresses in the array are each attached to a single analyte; (b) attaching affinity reagents to the addresses in the array via flexible linkers, whereby individual addresses in the array each have a single, attached affinity reagent and a single, attached analyte; (c) detecting a plurality of addresses in the array, thereby distinguishing a higher level of binding between an attached affinity reagent and an attached analyte at a first address in the array from a lower level of binding between an attached affinity reagent and an attached analyte at a second address in the array; and (d) identifying an analyte detected at an address in the array in step (c).

Also provided is a method of detecting a first reaction, comprising: (a) providing immobilized on a solid support: (i) an analyte; and (ii) a first reactant, the first reactant being immobilized on the support within a first distance from the analyte; (b) contacting the immobilized analyte with a probe, the probe comprising an affinity reagent and a second reactant, the affinity reagent having binding specificity for the analyte, and the second reactant being capable of a second reaction with the first reactant when within a second distance from the first reactant; (c) forming a first reaction between the analyte and the affinity reagent, thereby bringing the second reactant within the second distance of the first reactant; (d) after forming the first reaction, forming the second reaction between the first reactant and the second reactant; and (e) detecting the first reaction.

Also provided is a method, comprising: (a) contacting a plurality of affinity reagents to a plurality of analytes for an equilibration period, wherein the equilibration period is sufficient to form a binding equilibrium between affinity reagents of the plurality of affinity reagents and analytes of the plurality of analytes; and (b) after the equilibration period, detecting complexes comprising an affinity reagent bound to an analyte, wherein the quantity of detected complexes is greater than a steady-state quantity of complexes that exists at the binding equilibrium.

Also provided is a method, comprising: (a) contacting a plurality of affinity reagents to a plurality of analytes for an equilibration period, wherein each analyte of the plurality of analytes is individually paired to a unique identifier; (b) during the equilibration period, recording detection proxies at unique identifiers containing an affinity reagent bound to an analyte; and (c) detecting signals from detection proxies at a subset of the unique identifiers, thereby identifying analytes bound by affinity reagents during the equilibration period.

Also provided is a method, comprising: (a) contacting a plurality of affinity reagents to a plurality of analytes for an equilibration period, thereby coupling affinity reagents of the plurality of affinity reagents to analytes of the plurality of analytes, wherein each analyte of the plurality of analytes is paired to a unique identifier; (b) forming a detectable reaction for analytes bound by an affinity reagent during the equilibration period; and (c) detecting presence of the detectable reaction at a subset of unique identifiers, thereby detecting the subset of analytes of the plurality of analytes bound by an affinity reagent of the plurality of affinity reagents.

Also provided is a method, comprising: (a) contacting a plurality of affinity reagents to a plurality of analytes for an equilibration period, wherein the equilibration period is sufficient to form a binding equilibrium between affinity reagents of the plurality of affinity reagents and analytes of the plurality of analytes; and (b) detecting during the equilibration period complexes comprising an affinity reagent bound to an analyte, wherein the quantity of detected complexes is greater than an steady-state quantity of complexes that exists at the binding equilibrium.

Also provided is a method, comprising: (a) binding an affinity reagent to an analyte at a fixed spatial address; (b) coupling a kinetically-controlled detection agent to the fixed spatial address; and (c) after coupling the kinetically-controlled detection agent to the fixed spatial address, detecting a signal at the fixed spatial address.

Also provided is a system, comprising: (a) a solid support comprising a plurality of sites, wherein each site of the plurality of sites is optically resolvable from any other site of the plurality of sites; and (b) a plurality of analytes immobilized on the solid support, wherein each site of the plurality of sites is attached to one and only one analyte of the quantity of analytes; wherein, in a first configuration, the system further comprises a plurality of affinity reagents, wherein a fraction of the affinity reagents is bound to analytes of the plurality of analytes, wherein a quantity of the fraction of affinity reagents is determined by a binding equilibrium of the affinity reagents for the analytes; and wherein, in a second configuration, the system is substantially devoid of affinity reagents, wherein each individual site of a fraction of sites of the plurality of sites comprises a detectable label, wherein a quantity of the fraction of sites is greater than a quantity of the second fraction of affinity reagents.

Also provided is a system, comprising: (a) a solid support comprising a plurality of sites, wherein each site of the plurality of sites is optically resolvable from any other site of the plurality of sites; (b) a plurality of analytes immobilized on the solid support, wherein each site of the plurality of sites is attached to one and only one analyte of the quantity of analytes; and (c) a plurality of affinity reagents bound analytes of the plurality of analytes at sites of the plurality of sites, wherein the affinity reagents have a known binding equilibrium for binding to analytes of the plurality of analytes, and wherein a quantity of the sites of the plurality of sites is greater than a quantity of analytes bound to affinity reagents based upon the known binding equilibrium.

Also provided is a method of detecting a first reaction, comprising: (a) providing immobilized on a solid support: (i) an analyte; and (ii) a first reactant, the first reactant being immobilized on the support within a first distance from the analyte; (b) contacting the immobilized analyte with a probe, the probe comprising an affinity reagent and a second reactant, the affinity reagent having binding specificity for the analyte, and the second reactant being capable of a second reaction with the first reactant when within a second distance from the first reactant; (c) forming a first reaction between the analyte and the affinity reagent, and forming a second reaction between the first reactant and the second reactant; and (d) detecting the first reaction.

Also provided is a composition, comprising: (a) an affinity reagent having a binding specificity for at least a first analyte; (b) a first reactant, wherein the first reactant has reactivity with a second reactant; and (c) a linker, wherein the linker couples the affinity reagent to the first reactant, wherein the linker permits the first reactant to react with the second reactant when the affinity reagent is bound to the analyte.

### INCORPORATION BY REFERENCE

All publications, items of information available on the internet, patents, and patent applications cited in this specification are herein incorporated by reference to the same extent as if each individual publication, patent, or patent application was specifically and individually indicated to be incorporated by reference. To the extent publications, items of information available on the internet, patents, or patent applications incorporated by reference contradict the disclosure contained in the specification, the specification is intended to supersede and/or take precedence over any such contradictory material.

### BRIEF DESCRIPTION OF DRAWINGS

FIGS. 1A and 1B provide a diagrammatic representation of a binding assay method wherein an applied stimulus is used to attract affinity reagents to proteins in an array, in accordance with some embodiments.

FIG. 2 shows a cross section of a region of a flow cell having two protein analytes (white globules) immobilized on the lower surface and a fluid phase containing affinity reagents (Y shapes), wherein the affinity reagents are attached to a first luminophore (open circle) and the protein analytes are attached to a second luminophore (closed circle), in accordance with some embodiments.

FIG. 3A shows a diagram of a protein (white globule) attached to a surface, wherein the protein is attached to a first luminophore (closed circle) via a nucleic acid docker, and an affinity reagent (Y-shape) that is bound to the protein, wherein the affinity reagent is attached to a second luminophore (open circle) via a nucleic acid tether, and wherein the docker and tether hybridize to position the luminophores for proximity-based detection (e.g. FRET), in accordance with some embodiments.

FIG. 3B shows a diagram of a protein (white globule) attached to a surface, wherein the protein is attached to a first luminophore (closed circle) via a nucleic acid docker, and an affinity reagent (Y-shape) that is bound to the protein, wherein the affinity reagent is attached to a second luminophore (open circle) via a nucleic acid tether, and wherein the docker and tether hybridize to a splint oligonucleotide to position the luminophores for proximity-based detection (e.g. FRET), in accordance with some embodiments.

FIGS. 4A and 4B provide a diagrammatic representation of a binding assay method wherein analytes are attached to addresses on a surface and affinity reagents are linked to the addresses, in accordance with some embodiments.

FIG. 5A shows protein analytes (white globules) attached to addresses of an array and an affinity reagents (Y-shapes) attached to each address via a nucleic acid linker, wherein FRET occurs between luminophores (12 point stars) when the affinity reagent is bound to the protein (lefthand address) and FRET is not observed when the affinity reagent is dissociated from the protein (righthand address), in accordance with some embodiments.

FIG. 5B shows a configuration similar to FIG. 5A except the second fluorophore (closed 12 point star) is attached to the linker via a complementary oligonucleotide, in accordance with some embodiments.

FIG. 5C shows an exemplary configuration in which analytes (white globules) are attached to addresses of an array and an affinity reagent (Y-shape) is attached to each address via a nucleic acid linker. A first fluorophore (open 12 point star) is attached to the affinity reagent via a nucleic acid tether and a second fluorophore (closed 12 point star) is attached to the linker via a nucleic acid docker. At the address on the left (“+”), the affinity reagent is bound to the analyte and the tether is hybridized to the docker. A nucleotide sequence of the tether is complementary to a nucleotide sequence of the docker.

FIG. 6 shows protein analytes (white globules) attached to zero mode waveguides (ZMWs) and an affinity reagents (Y-shapes) attached to each ZMW via a nucleic acid linker, wherein emission from a luminophores (12 point star) is detectable when the affinity reagent is bound to the protein (lefthand ZMW) and emission is not observed when the affinity reagent is dissociated from the protein (righthand ZMW), in accordance with some embodiments.

FIGS. 7A and 7B show cross-sections of respective substrates that are capable of generating an electrophoretic force to attract affinity reagents, in accordance with some embodiments.

FIGS. 8A, 8B, and 8C illustrate methods of indirectly recording a binding interaction by transferring a transferrable moiety from an affinity reagent that is bound to an analyte to a unique identifier co-localized with the analyte, in accordance with some embodiments.

FIGS. 9A, 9B, and 9C illustrate a method of indirectly recording a binding interaction by catalytically modifying a unique identifier co-localized with an analyte, in accordance with some embodiments.

FIG. 10A depicts a method of directly recording a binding interaction between an analyte and an affinity reagent utilizing a bridging moiety that immobilizes the affinity reagent at the unique identifier containing the analyte, in accordance with some embodiments.

FIG. 10B depicts a method of dissociating an affinity reagent from an analyte after recording a binding interaction, in accordance with some embodiments.

FIG. 11 depicts oligonucleotide systems that utilize secondary structure of the oligonucleotides to control the kinetic on-rate of binding between the oligonucleotides, in accordance with some embodiments.

FIG. 12 illustrates a system for directly recording a binding interaction between an affinity reagent and an analyte that utilizes a luminescent enzyme, in accordance with some embodiments.

FIGS. 13A, 13B, 13C, and 13D display various systems for recording binding interactions that utilize a gap between docker and tether strands, in accordance with some embodiments.

FIGS. 14A, 14B, and 14C depict various configurations of an affinity reagent attached to a unique identifier by a linker, in which the configurations vary with respect to reaction for recording the binding interaction, in accordance with some embodiments.

FIG. 15 shows a diagram of a binding reaction in which a protein (cloud shape), which is attached to a first target nucleic acid (open, black line arrow) via a retaining component (black rectangle), binds to an affinity reagent (Y shape), which is attached to a second target nucleic acid (hatched, black line arrow). The target nucleic acids are hybridized with first hairpin oligonucleotides (labeled “1”) and second hairpin oligonucleotides (labeled “2”) to produce a nicked double helix. The arrow heads indicate 3′ ends of the nucleic acids and oligonucleotides, in accordance with some embodiments.

FIG. 16A shows a hairpin oligonucleotide hybridizing to a pair of target nucleic acids via annealing of the 3′ overhang (shown as the open section of the strand) of the oligonucleotide to a first target nucleic acid and annealing of a first of two hairpin sequences (shown as the grey hatched section of the strand) of the oligonucleotide to a second target nucleic acid, in accordance with some embodiments.

FIG. 16B shows a hairpin oligonucleotide hybridizing to a pair of target nucleic acids via annealing of the 3′ overhang (shown as the open section of the strand) of the oligonucleotide to a portion of the first target nucleic acid and annealing of a first of two hairpin sequences (shown as the grey hatched section of the strand) of the oligonucleotide to portions of both the first and second target nucleic acids, in accordance with some embodiments.

FIG. 17 shows a diagram of a reaction in which two species of oligonucleotides (black and grey arrows) hybridize to target nucleic acids of a protein-affinity reagent complex to form a nicked double helix. The oligonucleotides form hairpins and include labels (asterisks) which are in a quenched state in the hairpin conformation. The labels are competent for detection when hybridized in the nicked double helix, in accordance with some embodiments.

FIG. 18 illustrates differences in observation of binding equilibrium between a plurality of affinity reagents and a plurality of analytes when the binding interactions are not recorded (upper) or are recorded (lower), in accordance with some embodiments.

FIG. 19 displays a plot of effective affinity reagent concentration as a function of linker length when the linker co-localizes the affinity reagent with an analyte on a solid support, in accordance with some embodiments.

FIG. 20 displays a plot of equilibrium binding curves for an affinity reagent to a higher affinity target (superordinate) and a lower affinity target (subordinate), in accordance with some embodiments.

FIG. 21 displays detection rates for recording of binding interactions of affinity reagents with target peptides and non-target peptides via a biotin ligase-facilitated recording method.

### DETAILED DESCRIPTION

A fundamental challenge to the design and implementation of binding assays is to detect binding of an affinity reagent to an analyte of interest in the presence of an excess of non-bound affinity reagent and/or non-bound analyte. Typically, a binding assay includes an incubation period wherein at least one reactant (e.g., affinity reagent and/or analyte) is present at a relatively high concentration in solution. During this incubation period, an equilibrium is typically established between three species: non-bound affinity reagent, non-bound analyte and analyte:affinity reagent complex. For many assays, the complex is detected via a label that is attached to the affinity reagent or analyte. However, one or both of the non-bound species are typically present in a large excess compared to the complex, and this can result in a level of background signal from non-bound species that overwhelms detection of signal from the complexes. One approach to reducing unwanted background signal is to separate non-bound species from complexes, for example, by removal of the non-bound species from the assay, prior to detection. However, removal of the non-bound species results in a disequilibrium condition wherein, depending upon the kinetics of the binding reaction, the complexes may begin to dissociate. Substantial degrees of dissociation prior to or during the timeframe of detection create a risk of anomalous results.

Binding equilibrium represents a dynamic balance between association and dissociation of molecules. For example, in considering the binding equilibrium between a first species and a second species, observation of complexed molecules and unbound molecules will provide a steady-state measurement of the equilibrium, but over a long enough time, virtually all molecules of the first species and the second species will at least briefly form a complex with the other species. Observation of binding at equilibrium between molecules may provide incomplete information due to not observing all binding interactions that occurred.

The present disclosure provides binding assays that are used to form a complex between an affinity reagent and analyte, optionally with at least one of the affinity reagent and analyte labeled in the non-bound state, and detecting the complex via the detection of the label. Alternatively, some binding assay may introduce a third molecule that binds to the complex formed by the affinity reagent and analyte, in which the third molecule optionally contains a label. In some configurations, the binding assays allow detection of the labeled complex in the presence of non-bound species that also contain the label. In some cases, non-bound species are in substantial excess compared to the concentration or amount of complex present during a detection step. A binding assay of the present disclosure can be configured to detect complexes under an equilibrium condition, a disequilibrium condition, or in a simulated equilibrium condition.

The present disclosure provides a method of detecting a first reaction, comprising: (a) providing immobilized on a solid support: (i) an analyte, and (ii) a first reactant, the first reactant being immobilized on the support within a first distance from the analyte, (b) contacting the immobilized analyte with a probe, the probe comprising an affinity reagent and a second reactant, the affinity reagent having binding specificity for the analyte, and the second reactant being capable of a second reaction with the first reactant when within a second distance from the first reactant, (c) forming a first reaction between the analyte and the affinity reagent, and forming a second reaction between the first reactant and the second reactant, and (d) detecting the first reaction. The present disclosure further provides a method of detecting a first reaction, comprising: (a) providing immobilized on a solid support: (i) an analyte, and (ii) a first reactant, the first reactant being immobilized on the support within a first distance from the analyte, (b) contacting the immobilized analyte with a probe, the probe comprising an affinity reagent and a second reactant, the affinity reagent having binding specificity for the analyte, and the second reactant being capable of a second reaction with the first reactant when within a second distance from the first reactant, (c) forming a first reaction between the analyte and the affinity reagent, thereby bringing the second reactant within the second distance of the first reactant, (d) after forming the first reaction, forming the second reaction between the first reactant and the second reactant, and (e) detecting the first reaction.

Methods set forth herein may comprise a step of forming a reaction between a first reactant and a second reactant. The reaction may require bringing the first reactant into a sufficient proximity to the second reactant to form the reaction. Sufficient proximity between a first reactant and a second reactant can depend upon the nature of the interaction between the pair. A reaction may occur at a nanometer-scale separation distance between the first reactant and the second reactant (e.g., a photon transfer reaction between a dye pair), or may occur at an Angstrom-scale separation distance (e.g., covalent bond formation, certain electrostatic non-covalent interactions). A separation distance between a first reactant and a second reactant may be no more than about 50 nanometers (nm), 40 nm, 30 nm, 20 nm, 10 nm, 5 nm, 1 nm, 0.5 nm, 0.1 nm, 0.05 nm, 0.01 nm, or less than 0.01 nm. Alternatively or additionally, a separation distance between a first reactant and a second reactant may be at least about 0.01 nm, 0.05 nm, 0.1 nm, 0.5 nm, 1 nm, 5 nm, 10 nm, 20 nm, 30 nm, 40 nm, 50 nm, or more than 50 nm. In some cases, a separation distance between a first reactant and a second reactant may be less than a separation distance between an analyte and the first reactant, in which the analyte and first reactant are co-localized at a same address.

A first reactant that is configured to react with a second reactant may be co-located with an analyte at an address of a solid support. There may be a separation distance between the first reactant and the analyte at an address, for example as measured by the distance between their respective attachment points to the address. Alternatively, a reactive moiety of a first reactant may be attached by a linker to a solid support, thereby facilitating some diffusion of the reactive moiety adjacent to a co-located analyte. In such case, the separation distance between the first reactant and the analyte may be an average distance between the analyte and the reactive moiety of the first reactant. The separation distance between a first reactant and an analyte at an address may be chosen to balance between the selectivity of the reaction between the first reactant and the second reactant and the reactivity of the first reactant. For example, it may be preferable to attach a first reactant in close proximity to an analyte to decrease the likelihood that the first reactant binds to a second reactant when the affinity reagent attached to the second reactant is not bound to the analyte co-located with the first reactant. Further, if a first reactant is too close to an analyte, the first reactant may become partially-or fully-occluded (e.g., sterically, electrostatically, etc.) from reacting with a second reactant. Alternatively, partial occlusion of a first reactant may facilitate kinetic control of the reaction rate of the first reactant. Accordingly, a separation distance between a first reactant and an analyte at an address may be an optically non-resolvable distance, such as no more than about 500 nm, 400 nm, 300 nm, 200 nm, 100 nm, 50 nm, 40 nm, 30 nm, 20 nm, 15 nm, 10 nm, 5 nm, 1 nm, or less than 1 nm. Alternatively or additionally, a separation distance between a first reactant and an analyte at an address may be at least about 1 nanometer (nm), 5 nm, 10 nm, 15 nm, 20 nm, 30 nm, 40 nm, 50 nm, 100 nm, 200 nm, 300 nm, 400 nm, 500 nm, or more than 500 nm.

Further provided herein is a probe composition, comprising: (a) an affinity reagent having a binding specificity for at least a first analyte, (b) a first reactant, wherein the first reactant has reactivity with a second reactant, and (c) a linker, wherein the linker couples the affinity reagent to the first reactant, wherein the linker permits the first reactant to react with the second reactant when the affinity reagent is bound to the analyte. In some cases, the length of a linker coupling an affinity reagent to a first reactant may be greater than a separation distance between an analyte and a second reactant that is co-localized with the analyte. In other cases, the length of a linker coupling an affinity reagent to a first reactant may be less than a separation distance between an analyte and a second reactant that is co-localized with the analyte. The linker length may be a useful parameter for controlling binding equilibrium and binding kinetics according to the methods set forth herein.

The present disclosure further provides binding assays that record the presence of binding interactions, facilitating detection of such binding interactions even if the binding interaction becomes dissociated. In some configurations, a binding interaction may be directly recorded by binding together a molecular complex between bound species for a sufficiently long period of time to detect the complex. In other configurations, a binding interaction may be indirectly recorded by providing a detectable label at an address that can be subsequently detected at the address after a molecular complex between bound species has been disrupted.

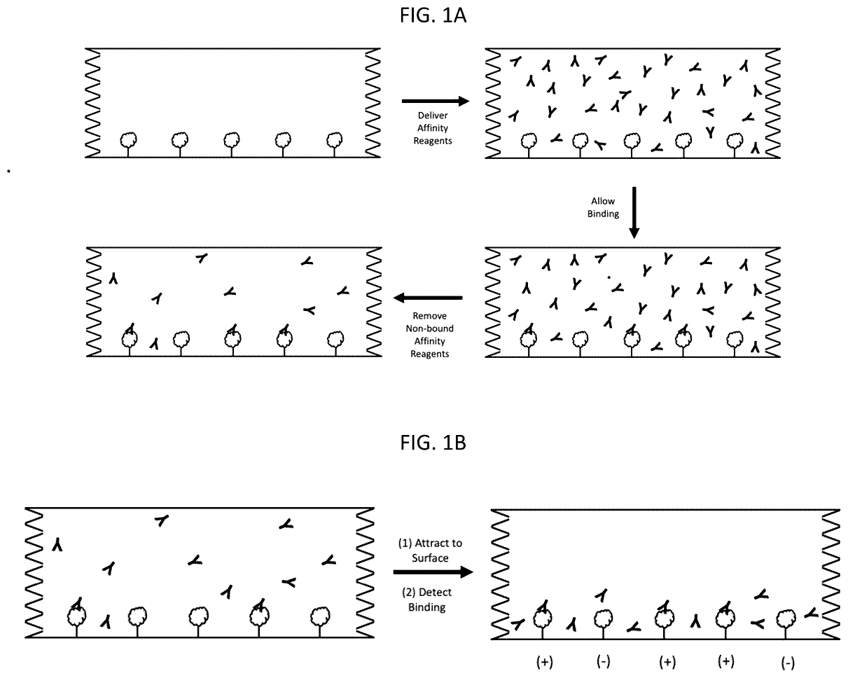

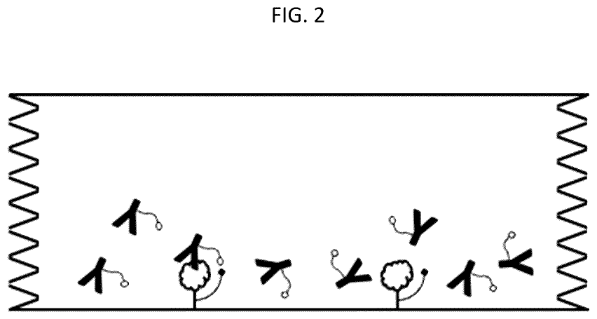

FIGS. 1A and 1B provides a diagrammatic representation of a first binding assay method. A section of a flow cell is shown in profile. Five proteins (white globules) are attached to respective addresses in an array that is formed on the bottom, inner surface of the flow cell. In the first step, a plurality of affinity reagents (Y-shapes) is delivered to the lumen of the flow cell. The fluid-phase affinity reagents are allowed to bind to proteins. In the example shown, the first, third and fourth proteins from the left are recognized by and bound to affinity reagents. Then at least some of the non-bound affinity reagents are removed from contact with the array, for example, being flowed out of the flow cell lumen. Moving to FIG. 1B, the subset of affinity reagents that are retained in the flow cell are subjected to an applied stimulus that attracts them to the proteins in the array. Any of a variety of stimuli can be applied as set forth in further detail below herein, including for example, magnetic attraction of affinity reagents having magnetic or paramagnetic moieties, electrophoretic attraction of affinity reagents having charged moieties, colloidal attraction in response to volume exclusion agents, or pH-dependent attraction of protein moieties to polyacidic moieties. The result is an increased concentration of affinity reagents in the volume that is localized to the array of proteins. However, the relatively low concentration of affinity reagents in the volume of the lumen that is distal from the array surface can prevent or substantially reduce background signal that is produced from non-bound species in the assay. Typically, an applied stimulus that is used to attract an affinity reagent (or analyte) to an array is variably applied in a method set forth herein.

The step of removing non-bound affinity reagents shown in FIG. 1A is optional. For example, an assay can be configured to deliver a relatively low concentration of affinity reagents in the first step followed by attraction of the affinity reagents to the surface via the applied stimulus and then detecting binding. This configuration can provide the advantage of reducing the amount of affinity reagent used, thereby reducing costs and resources required to make and/or use the affinity reagents. The application of a stimulus that transfers affinity reagents toward the analyte can create a higher effective concentration adjacent to the analytes than the bulk concentration in the lumen of the flow cell, thereby driving equilibrium toward complex formation.

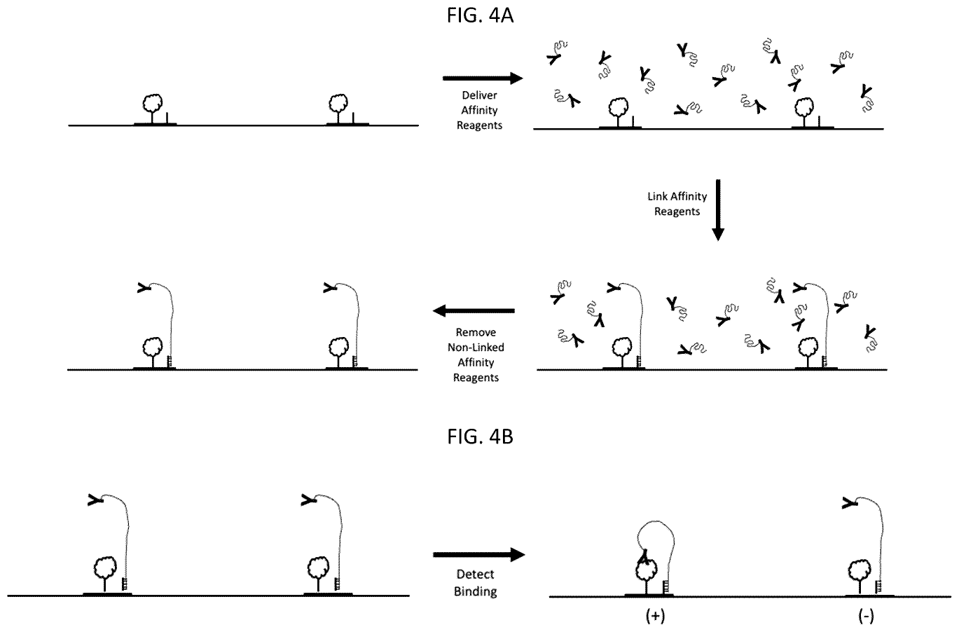

FIGS. 4A and 4B provide a diagrammatic representation of a second binding assay. Two addresses in an array are shown in FIG. 4A, each attached to a protein (white globule). In the first step, a plurality of affinity reagents (Y-shapes) is delivered to the array. Each of the affinity reagents is attached to a linker and the linker is reactive with an attachment moiety at each address of the array. In the example shown, an oligonucleotide serves as the attachment moiety at each of the addresses and the linkers include a nucleic acid sequence that is complementary to the oligonucleotide. Each address includes a single protein and a single linked affinity reagent. Non-linked affinity reagents are then removed from contact with the array. Turning to FIG. 4B, binding can then be allowed to occur, wherein the protein on the left bound to the affinity reagent thereby generating a signal (+) and the protein on the right is not bound by the affinity reagent and thus does not produce signal (−). The bound and unbound proteins can be distinguished using a detection technique that preferentially collects signal from a location at or near the protein relative to locations distal from the protein, or by collecting signals produced by energy transfer between the affinity reagent and protein. Other proximity-based detection methods can also be used. Exemplary methods for detecting complexes and distinguishing from non-bound components are set forth in further detail below.

The present disclosure further provides a method of detecting binding of an affinity reagent to an analyte by (a) contacting the analyte with the affinity reagent to form a complex, wherein the complex includes a first target nucleic acid that is co-localized with the affinity reagent and further includes a second target nucleic acid that is co-localized with the analyte; (b) contacting the complex with a set of oligonucleotides that hybridize to the first and second target nucleic acids, thereby forming a nicked double helix; and (c) detecting the nicked double helix, thereby detecting binding of the affinity reagent to the analyte.

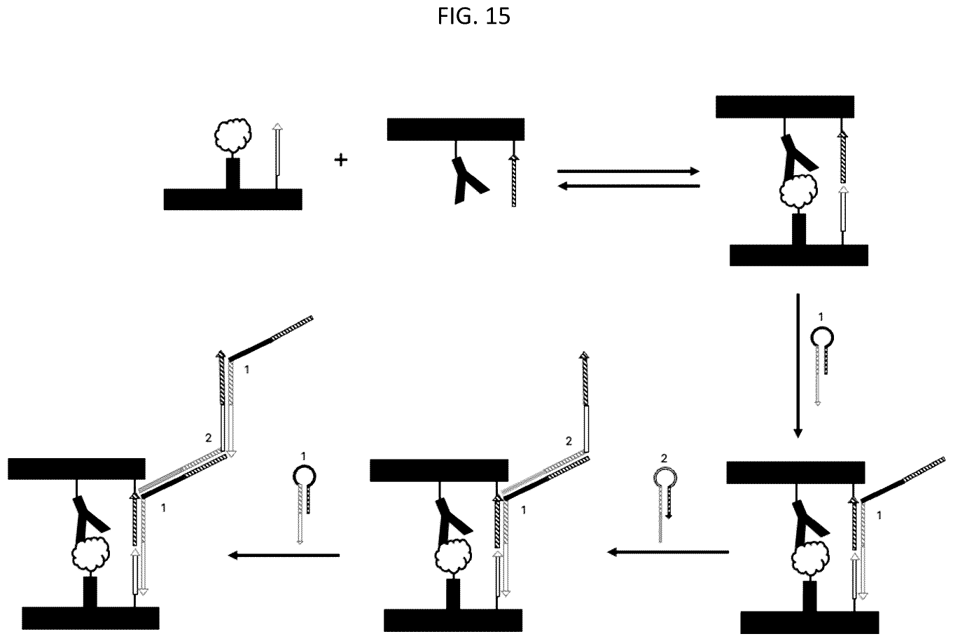

Optionally, the set of oligonucleotides can include oligonucleotide species that are in a self-annealed state, such as a stem-loop conformation (also referred to as a hairpin conformation). Hybridization of oligonucleotides of the set to the first and second target nucleic acids can initiate a hybridization chain reaction in which regions of the oligonucleotides that had been self-annealed instead anneal to each other to form the nicked double-helix. An exemplary configuration for the oligonucleotides and chain reaction for forming the nicked double helix is shown in FIG. 15, and described in further detail below.

The compositions, methods and apparatus set forth herein are particularly useful for single molecule-resolved binding assays because the nicked double helix can be grown to effectively provide signal amplification. For example, oligonucleotides that are incorporated into a nicked double helix of an analyte-affinity reagent complex can include labels and as the helix grows more signal-producing labels are added to the complex. Even though the complex includes only a single affinity reagent and a single analyte, the nucleotide sequence of the oligonucleotides, type of labels used and conditions provided for growth of the nicked double helix can be optimized to allow for adequate signal production for any of a variety of detection apparatus.

The compositions, methods and apparatus set forth herein provide advantages for use in equilibrium binding conditions. A common dilemma with binding assays is being able to maintain equilibrium conditions while distinguishing bound complexes from unbound components. For a typical binding reaction, equilibrium constitutes a relatively large concentration of unbound components and a relatively small concentration of bound complex. Furthermore, one of the components of the binding reaction is typically labeled to facilitate detection. As a result, detection of the relatively small amount of signal arising from the labeled complex will often be overwhelmed or at least obscured by the signal arising from the labeled unbound component. In contrast, the methods set forth herein can be configured to detect the mass of the nicked double helix, which is distinguishable from the individual oligonucleotides from which it is generated due to its substantially larger mass. Optionally, the oligonucleotides can include labels that are quenched or otherwise not-competent to generate signal when the oligonucleotides are in the unbound state, but become competent for signal production when bound to each other in the nicked double helix. For example, an oligonucleotide can include a luminescent label that is quenched by proximity to a quenching moiety when the oligonucleotide is in a self-annealed conformation (thereby decreasing luminescence intensity of the luminescent label), but competent for signal production when the oligonucleotide changes conformation to hybridize to the nicked double helix.

Detection schemes set forth herein are particularly useful for cyclic assay in which a given analyte is repeatedly contacted with affinity reagents and detected to observe any resulting complexes. This is because a nicked double helix can be configured to dissociate readily from a complex once the affinity reagent and analyte components of the complex dissociate. More specifically, a nicked double helix can be co-localized with a complex via binding to both a first target nucleic acid that is co-localized with the affinity reagent and a second target nucleic acid that is co-localized with the analyte. After detecting the complex, the affinity reagent can be dissociated from the analyte to effectively separate the first and second nucleic acids, thereby destabilizing the nicked double helix and causing it to dissociate from the complex and its components. A nicked double helix, as it is non-covalently assembled of individual oligonucleotides, can be cooperatively disassembled into component monomers by strand displacement reaction, heat, denaturing agents or other techniques common to the art for dehybridizing short oligonucleotides. The nucleic acid sequences of the target nucleic acids and oligonucleotides can be tailored to optimize hybridization energetics for formation and dissociation of the nicked double helix at the appropriate steps of the method.

Methods set forth herein may be particularly useful for characterizing pluralities of analytes, in which the plurality of analytes is characterized by a measure of heterogeneity. Heterogeneity of a plurality of analytes may be characterized by the presence of chemical or structural diversity amongst the analytes. For example, a plurality of proteins may contain two or more species of proteins, in which the species of proteins are distinguished from each other by differing primary structures (e.g., a first species having a different amino acid sequence than a second species). Additionally or alternatively, a plurality of proteins may contain two or more species of proteins, in which the species of proteins are distinguished from each other by differing proteoforms (e.g., a first species having a different complement of post-translational modifications than a second species). Additionally or alternatively, a plurality of proteins may contain two or more species of proteins, in which the species of proteins are distinguished from each other by differing isoforms (e.g., a first species having a different amino acid sequence splicing than a second species). A method set forth herein may be useful for characterizing a heterogeneous plurality of analytes that contains at least about 1%, 5%, 10%, 20%, 25%, 30%, 40%, 50%, 60%, 70%, 75%, 80%, 90%, 95%, 99%, or more than 99% of the species diversity of a genome, transcriptome, and/or proteome.

A plurality of analytes may comprise at least about 1, 2, 5, 10, 50, 100, 200, 500, 1000, 2000, 5000, 10000, 20000, 30000, 50000, 100000, 1000000, or more than 1000000 different species of analytes. Alternatively or additionally, a plurality of analytes may comprise no more than about 1000000, 100000, 50000, 30000, 20000, 10000, 5000, 2000, 1000, 500, 200, 100, 50, 10, 5, 2, or less than 5 different species of analytes.

Methods set forth herein may utilize one or more affinity reagents. It may be particularly useful to utilize an affinity reagent that binds to two or more differing species of analytes that are present in a plurality of analytes. In some cases, an affinity reagent may bind to an epitope that is present in two or more differing species of analytes that are present in a plurality of analytes. For example, an affinity agent may be configured to bind to an epitope (e.g., a trimer or tetramer amino acid sequence) that is common to two or more differing species of analytes.

An affinity reagent may bind to two or more differing binding targets with differing binding characteristics. For example, an affinity reagent may bind to a first binding target (e.g., a first analyte, a first epitope common to two or more differing analytes, etc.) with a first dissociation constant, and may further bind to a second binding target (e.g., a second analyte, a second epitope common to two or more differing analytes, etc.) with a second dissociation constant, in which the first dissociation constant differs from the second dissociation constant. Thus, the affinity reagent may dissociate, or separate, from one of the first binding target or the second binding target at a higher propensity than the other binding target. Useful distinguishing binding characteristics can also include the binding on-rate and/or the binding off-rate. Accordingly, for a plurality of analytes containing a first binding target and a second binding target for an affinity agent, there may exist two or more separate binding equilibria for the affinity agent. Some methods set forth herein may be useful for distinguishing between differing binding targets with a common affinity to an affinity reagent based upon the differences in binding equilibrium.

For affinity reagents described herein, a binding interaction with a binding target or plurality thereof having a highest characterized binding specificity (e.g., as determined by a lowest value of the affinity reagent's dissociation constant) may be referred to as a superordinate binding interaction. Likewise, for affinity reagents described herein, a binding interaction with a binding target or plurality thereof having a lower characterized binding specificity (e.g., as determined by a higher value of the affinity reagent's dissociation constant) relative to a superordinate binding interaction may be referred to as a subordinate binding interaction. An affinity reagent may have two or more superordinate binding interactions. An affinity reagent may have two or more subordinate binding interactions.

Binding equilibrium may depend in part on the relative concentrations of unbound affinity reagents and/or unbound analytes. For example, increasing the concentration of unbound affinity reagents may tend to increase the quantity of target analytes bound by the affinity reagents. Dissociation constant is a frequently used characterization of an affinity reagent for a binding target that can be utilized to estimate the steady-state fraction of binding targets that will be bound by an affinity reagent at binding equilibrium. A method set forth herein may comprise forming a binding equilibrium between affinity reagents and a plurality of binding targets (e.g., two or more species of analytes, analytes comprising a common epitope, etc.), in which at least about 10%, 20%, 30%, 40%, 50%, 60%, 70%, 80%, 90%, 95%, 99%, or more than 99% of binding targets are bound by an affinity reagent at equilibrium. Alternatively or additionally, a method set forth herein may comprise forming a binding equilibrium between affinity reagents and a plurality of binding targets, in which no more than about 99%, 95%, 90%, 80%, 70%, 60%, 50%, 40%, 30%, 20%, 10%, or less than 10% of binding targets are bound by an affinity reagent at equilibrium. Some methods set forth herein may comprise a step of recording a binding interaction between an affinity reagent and a binding target such that the binding interaction can be detected even after the affinity reagent has dissociated from the binding target. Accordingly, a method may comprise detecting a binding interaction for a percentage of binding targets (e.g., two or more species of analytes, analytes comprising a common epitope, etc.) that exceeds a percentage of binding targets bound by the affinity reagent at the steady-state binding equilibrium.

An affinity reagent may bind to at least about 1, 2, 5, 10, 50, 100, 200, 500, 1000, 2000, 5000, 10000, 20000, 30000, 50000, 100000, 1000000, or more than 1000000 different species of analytes present in a plurality of analytes. Alternatively or additionally, an affinity agent may bind to no more than about 1000000, 100000, 50000, 30000, 20000, 10000, 5000, 2000, 1000, 500, 200, 100, 50, 10, 5, or less than 5 different species of analytes present in a plurality of analytes.

Methods set forth herein may be particularly well-suited to array-based techniques. In some cases, a method may utilize a single-analyte array, in which each individual analyte of a plurality of analytes is co-localized with a unique identifier, such that each individual analyte is identifiable by its unique identifier. In some cases, a unique identifier may comprise an address on a solid support, in which the address is a spatially resolvable distance from any other address of the solid support that contains an analyte. In some cases, an address on a solid support may contain a site that is configured to bind an analyte. Accordingly, the site may comprise one or more moieties that facilitate attachment of the analyte and/or unique identifier co-localized with the analyte. In some cases, a unique identifier may comprise a barcode (e.g., a nucleic acid barcode, a peptide barcode, etc.) or a tag (e.g., a nucleic acid tag, a peptide tag) that is co-localized with an analyte. A tag may be especially useful for methods that comprise a step of recording a binding interaction of an affinity agent to an analyte via transfer of a detectable label to a tag co-localized with the analyte.

Terms used herein will be understood to take on their ordinary meaning in the relevant art unless specified otherwise. Several terms used herein and their meanings are set forth below.

In some of the implementations described herein, the term “address,” when used in reference to an array, can mean a location in an array occupied by, or configured to be occupied by, a particular molecule or analyte such as a protein, nucleic acid, structured nucleic acid particle or reactive moiety. An address can contain a single analyte molecule, or it can contain a population of several analyte molecules of the same species (i.e. an ensemble of the molecules). Alternatively, an address can include a population of molecules that are different species such as an attached analyte and a linked affinity reagent. Addresses of an array are typically discrete. The discrete sites can be contiguous, or they can have interstitial spaces between each other. An array useful herein can have, for example, addresses that are separated by less than 100 microns, 10 microns, 1 micron, 0.5 micron, 0.1 micron, 0.01 micron or less. Alternatively or additionally, an array can have addresses that are separated by at least 0.01 micron, 0.1 micron, 0.5 micron, 1 micron, 10 microns, 100 microns or more. The addresses can each have an area of less than 1 square millimeter, 500 square microns, 100 square microns, 25 square microns, 1 square micron or less. An array can include at least about 1×10⁴, 1×10⁵, 1×10⁶, 1×10⁷, 1×10⁸, 1×10⁹, 1×10¹⁰, 1×10¹¹, 1×10¹², or more addresses, some or all of which are occupied by analytes or molecules. An address that is configured to bind an analyte, for example by the presence of attachment moieties, is referred to herein as a “site.”

In some of the implementations described herein, the term “affinity reagent” can refer to a molecule or other substance that is capable of specifically or reproducibly binding to an analyte (e.g., protein) or moiety (e.g., post-translational modification of a protein). An affinity reagent can be larger than, smaller than or the same size as the analyte. An affinity reagent may form a reversible or irreversible bond with an analyte. An affinity reagent may bind with an analyte in a covalent or non-covalent manner. Affinity reagents may include chemically reactive affinity reagents, catalytic affinity reagents (e.g., kinases, proteases, etc.) or chemically non-reactive affinity reagents (e.g., antibodies or fragments thereof). An affinity reagent can be chemically non-reactive and non-catalytic, thereby not permanently altering the chemical structure of an analyte to which it binds. Affinity reagents that can be particularly useful for binding to proteins include, but are not limited to, antibodies such as full-length antibodies or functional fragments thereof (e.g., Fab′ fragments, F(ab′)₂ fragments, single-chain variable fragments (scFv), di-scFv, tri-scFv, or microantibodies), or aptamers, affibodies, affilins, affimers, affitins, alphabodies, anticalins, avimers, miniproteins, DARPins, monobodies, nanoCLAMPs, lectins, or functional fragments thereof. The term “affinity agent” is intended to be synonymous with the term “affinity reagent.”

In some of the implementations described herein, the term “antibody” can refer to a protein that binds to an antigen or epitope via at least one complementarity determining region (CDR). An antibody can include all elements of a full-length antibody. However, an antibody need not be full length and functional fragments can be particularly useful for many uses. The term “antibody” as used herein encompasses full length antibodies and functional fragments thereof.

In some of the implementations described herein, the term “array” can refer to a population of analytes (e.g., proteins) that are co-localized with unique identifiers such that the analytes can be distinguished from each other. A unique identifier can be a solid support (e.g., particle or bead), structured nucleic acid particle (SNAP), address on a solid support, tag, label (e.g., luminophore), or barcode (e.g., nucleic acid barcode) that is co-localized with an analyte and that is distinct from other identifiers in the array. Analytes can be co-localized with unique identifiers by attachment, for example, via covalent or non-covalent bonds. An array can include different analytes that are each attached to different unique identifiers. An array can include different unique identifiers that are attached to the same or similar types of analytes. An array can include separate solid supports, or separate sites on a given solid support, that each bear a respective analyte, wherein the respective analytes can be identified according to the locations of the solid supports or sites. Analytes that can be included in an array can be, for example, nucleic acids such as structured nucleic acid particles, proteins, enzymes, glycans, affinity reagents, ligands, or receptors.

In some of the implementations described herein, the term “artificial” when used in reference to a substance, can mean that the substance is made by human activity rather than occurring naturally. For example, a polymer that is made at least in part by human activity or that includes at least one artificial monomer unit is referred to as an “artificial polymer.” As used herein, the term “natural” when used in reference to a substance, means that the substance occurs naturally. A substance that is purified or refined from its naturally occurring state without further modification can be considered natural, but a substance that is chemically modified from its naturally occurring state can be considered artificial. For example, cellulose can be considered a natural polymer, but methyl cellulose can be considered an artificial polymer.

In some of the implementations described herein, the term “attached” can refer to the state of two things being joined, fastened, adhered, connected or bound to each other. Attachment can be covalent or non-covalent. For example, a particle can be attached to a protein by a covalent or non-covalent bond. A covalent bond is characterized by the sharing of pairs of electrons between atoms. A non-covalent bond is a chemical bond that does not involve the sharing of pairs of electrons and can include, for example, hydrogen bonds, ionic bonds, van der Waals forces, hydrophilic interactions, adhesion, adsorption, and hydrophobic interactions.

In some of the implementations described herein, the term “binding affinity” or “affinity” can refer to the strength or extent of binding between an affinity reagent and a binding partner. The binding affinity of an affinity reagent for a binding partner may be qualified as being “high affinity,” “medium affinity,” or “low affinity.” A binding affinity of an affinity reagent for a binding partner, affinity target, or target moiety may be quantified as being “high affinity” if the interaction has a dissociation constant of less than about 100 nM, “medium affinity” if the interaction has a dissociation constant between about 100 nM and 1 mM, and “low affinity” if the interaction has a dissociation constant of greater than about 1 mM. Binding affinity can be described in terms known in the art of biochemistry such as equilibrium dissociation constant (K_{D}), equilibrium association constant (K_{A}), association rate constant (kₒₙ), dissociation rate constant (k_{off}) and the like. See, for example, Segel, Enzyme Kinetics John Wiley and Sons, New York (1975), which is incorporated herein by reference in its entirety.

In some of the implementations described herein, the term “binding interaction” can refer to a reaction that associates an affinity reagent to an analyte. A binding reaction may be a covalent or non-covalent interaction. A binding interaction may associate an affinity reagent to an analyte for a sufficient length of time to detect a complex formed by the affinity reagent and analyte.

In some of the implementations described herein, the term “biomolecule” can refer to a molecule that is produced by a living organism or capable of being produced by a living system.

Biomolecules include, for example, proteins, amino acids, nucleic acids, nucleotides, nucleosides, polysaccharides, glycans, sugars, hormones, metabolites and the like. Biomolecules can encompass both natural and artificial biomolecules.

In some of the implementations described herein, the term “competent,” when used in reference to a label, can mean the label is in a state where it produces a detectable signal or is capable of producing a detectable signal. For example, a luminescent label is competent when it is able to produce a detectable photon when excited by light at an appropriate wavelength. A luminescent label that is quenched is considered to not be competent.

In some of the implementations described herein, the term “complex” can refer to two or more molecules held together by at least one non-covalent interaction. Two or more molecules of a complex can be dissociated from each other without necessarily breaking any covalent bonds. Rather, two or more molecules of a complex can be dissociated by breaking at least one non-covalent bond. It will be understood that in some cases covalent bonds can also be broken when dissociating two or more molecules of a complex.

The term “comprising” is intended herein to be open-ended, including not only the recited elements, but further encompassing any additional elements.

In some of the implementations described herein, the term “concatemer structure,” when used in reference to a nucleic acid, can mean the nucleic acid has at least two repeats of a nucleotide sequence. The repeated nucleotide sequence can include at least 5, 6, 8, 10, 12, 14, 16, 18, 20, 25 or more nucleotides. A concatemer structure can include at least two repeats of other structural elements. Exemplary structural elements include nicks or gaps in a double-stranded nucleic acid. Nicks or gaps can optionally separate repeated nucleotide sequence regions of a nicked double helix having a concatemeric structure. A concatemer can include at least 2, 3, 4, 5, 6, 8, 10, 15, 20, 25 or more repeats of a given sequence and/or other structural element.

In some of the implementations described herein, the term “covalent,” when used in reference to a bond between atoms or moieties of a molecule, can refer to bonding due to sharing of a pair of electrons between the two atoms or moieties. Covalent interaction can arise due to a chemical reaction between a first reactive moiety and a second reactive moiety, optionally in the presence of a third intermediary or catalytic moiety. Covalent binding interactions can form between two atoms or moieties due to various chemical mechanisms, including addition, substitution, elimination, oxidation, and reduction. In some cases, a covalent binding interaction may be formed by a Click-type reaction, as set forth herein (e.g., methyltetrazine (mTz)-tetracyclooctylene (TCO), azide-dibenzocyclooctene (DBCO), thiol-epoxy). In some cases, a ligand-receptor-type binding interaction can also form a covalent binding interaction. For example, SpyCatcher-SpyTag, SnoopCatcher-SnoopTag, and SdyCatcher-SdyTag are receptor-ligand binding pairs that can form covalent binding interactions due to isopeptide bond formation. Additional useful covalent interactions can include coordination bond formation, such as between a metal-containing substrate and a ligand. Exemplary coordination bonds can include silicon-silane, metal oxide-phosphate, and metal oxide-phosphonate. Useful reagents and mechanisms for forming covalent binding interactions, including bioorthogonal binding interactions, as set forth herein, are provided in U.S. Pat. Nos. 11,203,612 and 11,505,796, each of which is herein incorporated by reference in its entirety.

In some of the implementations described herein, the term “docker” can refer to a molecule or moiety that is configured to interact with a tether or that is interacting with a tether. A docker can be a moiety of a substance, object, molecule, solid support, address, particle, or bead. A docker can include a polymer, nucleic acid strand, nucleic acid duplex, nucleotide sequence, protein, affinity reagent, epitope, paratope, receptor, ligand or the like. A docker can interact with a tether via covalent or non-covalent bonding.

In some of the implementations described herein, the term “each,” when used in reference to a collection of items, can be intended to identify an individual item in the collection but does not necessarily refer to every item in the collection. Exceptions can occur if explicit disclosure or context clearly dictates otherwise.

In some of the implementations described herein, the term “epitope” can refer to a molecule or part of a molecule, which is recognized by or binds specifically to an affinity reagent or paratope. Epitopes may include amino acid sequences that are sequentially adjacent in the primary structure of a protein or amino acids that are structurally adjacent in the secondary, tertiary or quaternary structure of a protein. An epitope can optionally be recognized by or bound to an antibody. However, an epitope need not necessarily be recognized by any antibody, for example, instead being recognized by an aptamer, miniprotein or other affinity reagent. An epitope can optionally bind an antibody to elicit an immune response. However, an epitope need not necessarily participate in, nor be capable of, eliciting an immune response.

In some of the implementations described herein, the term “fluid-phase” or “fluid phase,” when used in reference to a molecule or particle participating in a binding interaction, can mean the molecule or particle is in a state wherein it is mobile in a fluid, for example, being capable of diffusing through the fluid to another molecule or particle that is complementary to the molecule or particle in the binding interaction. A fluid-phase molecule or particle may be attached to a solid support provided the moiety that attaches the molecule or particle to the solid support permits sufficient movement of the molecule or particle through the fluid to contact another molecule or particle that is complementary to the molecule or particle in a binding interaction. A fluid-phase molecule or particle may be attached to a solid support by a linker, such as a flexible linker. An immobilized analyte that is provided in a denatured or partially-denatured state may contain one or more fluid-phase portions of its molecular structure.

In some of the implementations described herein, the term “gap,” when used in reference to a double stranded nucleic acid, can refer to a region in one strand of the double helix which occurs between two double stranded regions and which lacks at least one nucleotide, whereby the gap is incapable of forming a Watson-Crick base pair with the other strand of the double helix. A gap in one strand of a double helix can span at least 1, 2, 3, 4, 5, 10, 15, 20, 25 or more nucleotide positions of the other strand of the double helix.

In some of the implementations described herein, the term “hairpin sequence” can mean a nucleotide sequence that includes a first region of the nucleotide sequence that is the reverse complement of a second region of the nucleotide sequence. The first region of the nucleotide sequence and second region of the nucleotide sequence are typically separated by an intermediate region that forms a single-stranded structure, that is referred to as a “loop,” when the nucleotide sequence self-anneals. The hairpin sequence can include a palindromic sequence of nucleotides or a gapped palindromic sequence of nucleotides. The gap in the palindrome can occur in the loop and/or portions of the palindromic sequence can occur in the loop. The reverse complementary regions of a hairpin sequence can anneal with the second region of the nucleotide sequence to form an intramolecular double helix that is referred to as a “stem” structure.

In some of the implementations described herein, the term “immobilized,” when used in reference to a molecule or particle that is in contact with a fluid phase, can refer to the molecule or particle or a portion thereof being prevented from diffusing in the fluid phase. For example, immobilization can occur due to confinement at, or attachment to, a solid phase. Immobilization can be temporary (e.g., for the duration of one or more steps of a method set forth herein) or permanent. Immobilization can be reversible or irreversible under conditions utilized for a method, system or composition set forth herein. A denatured molecule may be considered immobilized if the molecule as a whole cannot diffuse through the fluid phase, even if portions of the molecular structure have an ability to diffuse in regions of the fluid adjacent to the immobilization site of the molecule.

In some of the implementations described herein, the terms “label” and “detectable label” can refer synonymously to a molecule or moiety that provides a detectable characteristic. The detectable characteristic can be, for example, an optical signal such as absorbance of radiation, luminescence emission, luminescence lifetime, luminescence polarization, fluorescence emission, fluorescence lifetime, fluorescence polarization, or the like; Rayleigh and/or Mie scattering; binding affinity for a ligand or receptor; magnetic properties; electrical properties; charge; mass; radioactivity or the like. Exemplary labels include, without limitation, a luminophore (e.g., fluorophore), chromophore, nanoparticle (e.g., gold, silver, carbon nanotubes), heavy atoms, radioactive isotope, mass label, charge label, spin label, receptor, ligand, or the like. A label may produce a signal that is detectable in real-time (e.g., fluorescence, luminescence, radioactivity). A label may produce a signal that is detected off-line (e.g., sequencing of, or hybridization to, a nucleic acid barcode) or in a time-resolved manner (e.g., time-resolved fluorescence). A label may produce a signal with a characteristic frequency, intensity, polarity, duration, wavelength, sequence, or fingerprint.

In some of the implementations described herein, the terms “linker” and “linking moiety” can refer synonymously to a moiety that connects two objects to each other. One or both objects can be a molecule (e.g., affinity reagent or analyte), solid support, address, particle or bead. The term can also refer to an atom, moiety or molecule that is configured to react with two objects to form a moiety that connects the two objects. The connection of a linker to one or both objects can be a covalent bond or non-covalent bond. A linker may be configured to provide a chemical or mechanical property to the moiety connecting two objects, such as hydrophobicity, hydrophilicity, electrical charge, polarity, rigidity, or flexibility. A linker may comprise two or more functional groups that facilitate coupling of the linker to the first and second objects. A linker may include a polyfunctional linker such as a homobifunctional linker, heterobifunctional linker, homopolyfunctional linker, or heteropolyfunctional linker. Exemplary compositions for linkers can include, but are not limited to, a polyethylene glycol (PEG), polyethylene oxide (PEO), amino acid, polypeptide, nucleotide, nucleic acid, nucleic acid origami, dendrimer, peptide nucleic acid (PNA), polysaccharide, carbon, nitrogen, oxygen, ether, sulfur, or disulfide. A linker can be a bead or particle such as a structured nucleic acid particle.

In some of the implementations described herein, the term “moiety” can refer to a component or part of a molecule. The term does not necessarily denote the relative size of the component or part compared to the rest of the molecule, unless indicated otherwise. A moiety can include one or more atoms.

In some of the implementations described herein, the term “non-covalent,” when used in reference to a bond between atoms or moieties of a molecule, can refer to bonding due a mechanism other than electron pair-sharing between the two atoms or moieties. Non-covalent interaction can arise due to an electrostatic or magnetic interaction between moieties and/or atoms. Non-covalent binding interactions can include electrostatic interactions such as ionic bonding, hydrogen bonding, halogen bonding, Van der Waals interactions, Pi-Pi stacking, Pi-ion interactions, Pi-polar interactions, or magnetic interactions. In some cases, a non-covalent interaction may include hybridization of a first oligonucleotide to a complementary second oligonucleotide. In some cases, a non-covalent interaction may form between a receptor and ligand, such as streptavidin-biotin. Other useful non-covalent interactions can include affinity reagent-target interactions, such as antibody-epitope or aptamer-epitope interactions.

In some of the implementations described herein, the term “nucleic acid origami” can refer to a nucleic acid construct having an engineered tertiary or quaternary structure. A nucleic acid origami may include DNA, RNA, PNA, or modified or non-natural nucleic acids, or combinations thereof. A nucleic acid origami may include a plurality of oligonucleotides that hybridize via sequence complementarity to produce the engineered structuring of the origami. A nucleic acid origami may include sections of single-stranded or double-stranded nucleic acid, or combinations thereof. Exemplary nucleic acid origami structures may include nanotubes, nanowires, cages, tiles, nanospheres, blocks, and combinations thereof. A nucleic acid origami can optionally include a relatively long scaffold nucleic acid to which multiple smaller nucleic acids hybridize, thereby creating folds and bends in the scaffold that produce an engineered structure. The scaffold nucleic acid can be circular or linear. The scaffold nucleic acid can be single stranded but for hybridization to the smaller nucleic acids. A smaller nucleic acid (sometimes referred to as a “staple”) can hybridize to two regions of the scaffold, wherein the two regions of the scaffold are separated by an intervening region that does not hybridize to the smaller nucleic acid.

In some of the implementations described herein, the term “nick,” when used in reference to a double stranded nucleic acid, can refer to discontinuity in the sugar phosphate backbone of one strand of the double helix. In some cases, a nicked double stranded nucleic acid can further include a gap adjacent to the nick. Alternatively, nucleotide positions on either side of the discontinuity can occupied by nucleotides that are hybridized to nucleotides of the other strand in the double helix. As such, a nicked double stranded helix can include a gap adjacent to the site of the nick but need not necessarily include a gap adjacent to the site of the nick.

In some of the implementations described herein, the term “paratope” can refer to a molecule or part of an affinity reagent, which recognizes or binds to an epitope. A paratope may include an antigen binding site of an antibody. A paratope may include at least 1, 2, 3, or more complementarity-determining regions of an antibody. A paratope need not necessarily be present in nor derived from an antibody, for example, instead being present in a nucleic acid aptamer, lectin, streptavidin, miniprotein or other affinity reagent. A paratope need not necessarily participate in, nor be capable of, eliciting an immune response.

In some of the implementations described herein, the term “particle” can mean an object having a largest dimension between 10 nm and 1 mm. The object can be composed of a rigid or semi-rigid material. The particle can be insoluble in a fluid such as aqueous liquid. A particle can have a shape characterized, for example, as a sphere, ovoid, polyhedron, or other recognized shape whether having regular or irregular dimensions. Exemplary particles include, but are not limited to, structured nucleic acid particles (SNAPs) such as nucleic acid origami particles; optically detectable particles such as fluorescent nanoparticles, FluoSpheres™, and quantum dots; organic particles; inorganic particles; viral particles, such as phage particles having analytes displayed on their surfaces; gel particles; or particles made from solid support materials set forth herein or known in the art.

In some of the implementations described herein, the term “polyacid” can refer to a polymer having a plurality of repeating monomer subunits connected via a network of covalent bonds, wherein monomer subunits in the polymer each include a proton donating moiety. The network can include one or more chain(s) of the monomer subunits. The polymer can be linear or branched. A linear polymer includes only one chain in the network of covalent bonds. A branched polymer includes at least two chains in the network of covalent bonds. The polymer can include a single type of monomer subunit or multiple different types of monomer subunits. Accordingly, a polymer can include at least 1, 2, 3, 4, 5 or more different types of monomer subunits. Alternatively or additionally, a polymer can include at most 5, 4, 3, 2 or 1 different types of monomer subunits. A polymer having only one type of subunit in the network of covalent bonds is referred to as a “homopolymer.” In contrast, a “copolymer” includes two or more different types of subunits in the network of covalent bonds. The proton donating moiety can be, for example, a hydroxyl, carboxylic acid, sulfonic acid, or phosphoric acid. Exemplary polyacids include, but are not limited to, poly(styrene sulfonic acid) (PSS), polymethacrylic acid (PMAA) or poly(acrylic acid) (PAA).

In some of the implementations described herein, the term “polyacidic brush” can refer to a plurality of polyacids grafted to a surface. The polyacids can be grafted by one end of their polymer chains. The graft density on the surface can be at least 0.1 chains/nm², 0.2 chains/nm², 0.3 chains/nm², 0.4 chains/nm², 0.5 chains/nm², 0.6 chains/nm², 0.7 chains/nm², 0.8 chains/nm², 0.9 chains/nm², 1 chain/nm², 2 chains/nm² or higher. Polymer brushes are capable of transitioning from individually stable polymers to a volume-excluded or otherwise interacting system. Exemplary polyacidic brushes are set forth in Ritsema et al., ACS Appl. Polym. Mater. 4:3062-3087 (2022), which is incorporated herein by reference.

In some of the implementations described herein, the term “polymer” can refer to a molecule having a plurality of monomer subunits connected via a network of covalent bonds. The network may contain a single type of monomer subunit, or two or more types of monomer subunits. A polymer network may have a pattern of subunits or a random network of subunits (e.g., a protein). The network can include one or more chain(s) of the monomer subunits. A polymer can be linear or branched. A linear polymer includes only one chain in the network of covalent bonds. A branched polymer includes at least two chains in the network of covalent bonds. For example, a branched polymer can include at least 2, 3, 4, 5, 6, 8, 10 or more chains in the network of covalent bonds. Alternatively or additionally, a branched polymer can include at most 10, 8, 6, 5, 4, 3 or 2 chains in the network of covalent bonds. A polymer can include a single type of monomer subunit or multiple different types of monomer subunits. Accordingly, a polymer can include at least 1, 2, 3, 4, 5 or more different types of monomer subunits. Alternatively or additionally, a polymer can include at most 5, 4, 3, 2 or 1 different types of monomer subunits. A polymer having only one type of subunit in the network of covalent bonds is referred to as a “homopolymer.” In contrast, a “copolymer” includes two or more different types of subunits in the network of covalent bonds.

In some of the implementations described herein, the term “pool,” when used in reference to a plurality of objects or molecules, can refer to the objects or molecules being in fluidic communication with each other. A pool can be in a fluid phase that is, in turn, in contact with a solid phase. For example, the solid phase can include immobilized objects or substances that are in communication with fluid-phase objects or substances.

In some of the implementations described herein, the term “protein” can refer to a molecule including three or more amino acids joined by peptide bonds. A protein may also be referred to as a polypeptide, oligopeptide or peptide. Although the terms “protein,” “polypeptide,” “oligopeptide” and “peptide” may optionally be used to refer to molecules having different characteristics, such as amino acid composition, amino acid sequence, amino acid length, molecular weight, origin of the molecule or the like, the terms are not intended to inherently include such distinctions in all contexts. A protein can be a naturally occurring molecule, or synthetic molecule. A protein may include one or more non-natural amino acids, modified amino acids, or non-amino acid linkers. A protein may contain D-amino acid enantiomers, L-amino acid enantiomers or both. Amino acids of a protein may be modified naturally or synthetically, such as by post-translational modifications.

In some of the implementations described herein, the term “recognize” can refer to the capability of two or more molecules to interact with each other through non-covalent bonding such as hydrogen bonding, metal coordination, hydrophobic forces, van der Waals forces, T-T interactions, halogen bonding, or resonant interaction effects.

In some of the implementations described herein, the term “reaction,” when used in reference to a first molecule, particle, or moiety and a second molecule, particle, or moiety, can refer to a chemical interaction that involves both molecules, particle, or moieties. A reaction can include reversible or irreversible binding of a first molecule, particle, or moiety to the second molecule, particle or moiety. A reaction can include transfer of matter or energy from the first molecule, particle, or moiety to the second molecule, particle or moiety, such as transfer of one or more atoms, or transfer of a photon. A reaction can include a catalyzed change to the second molecule, particle, or moiety when it is associated to the first molecule, particle, or moiety. A reaction can include interactions of biomolecules, such as ligand-receptor binding, nucleic acid hybridization, protein-protein interactions, protein-nucleic acid interactions, etc.

In some of the implementations described herein, the term “recording,” when used in reference to a binding interaction, can refer to altering the presence of a detectable label at a unique identifier associated with an analyte, thereby providing a characteristic signal at the unique identifier until a detection event has occurred. Recording can include providing the detectable label to the unique identifier, thereby providing a characteristic presence of a signal from the detectable label at the unique identifier when the binding interaction has occurred at the unique identifier. Recording can include removing the detectable label from the unique identifier, thereby providing a characteristic absence of a signal from the detectable label at the unique identifier when the binding interaction has occurred at the unique identifier. Recording can include providing a reaction that binds an affinity reagent to an analyte in the presence of a detectable label until a detection event has occurred. Recording can include transferring a detectable label between an affinity reagent and an analyte or unique identifier, thereby providing a characteristic signal at the unique identifier until a detection event has occurred. A characteristic signal provided by the recording of a binding interaction may persist for any number of intermediate method steps that occur between the altering of a detectable label and a detection event of the characteristic signal.

In some of the implementations described herein, the term “quenched,” when used in reference to a label, can mean the label is inhibited or prevented from producing a detectable signal. For example, a luminophore can be quenched by physical proximity to a quenching agent that inhibits the luminophore from producing a detectable photon when excited by an appropriate wavelength of light. In other examples, a fluorophore can be quenched by excitation with light at a wavelength that differs from an excitatory wavelength for the fluorophore.

In some of the implementations described herein, the term “retaining component” can refer to a particle, molecule or material to which one or more moieties of an affinity reagent or analyte are attached. Exemplary retaining components include, but are not limited to, structured nucleic acid particles, nucleic acid origami, particles made of solid support materials, or polymers such as branched polymers or dendrimers. Affinity reagent moieties that can be attached to a retaining component, directly or indirectly, include for example, one or more paratopes, one or more labels, one or more antibodies, one or more nucleic acid aptamers, one or more nucleic acid tags or the like.

In some of the implementations described herein, the term “single,” when used in reference to an object such as an analyte, can mean that the object is individually manipulated or distinguished from other objects. A single object can also be referred to as one, and only one, object. A single analyte can be a single molecule (e.g., single protein), a single complex of two or more molecules (e.g., a multimeric protein having two or more separable subunits, a single protein attached to a structured nucleic acid particle or a single protein attached to an affinity reagent), a single particle, or the like. Reference herein to a “single analyte” in the context of a composition, system or method herein does not necessarily exclude application of the composition, system or method to multiple single analytes that are manipulated or distinguished individually, unless indicated contextually or explicitly to the contrary.

In some of the implementations described herein, the term “single-analyte resolution” can refer to the detection of, or ability to detect, an analyte on an individual basis, for example, as distinguished from its nearest neighbor in an array. The term when used in reference to a single-analyte array, refers to detection of a single-analyte under the conditions that: 1) the single-analyte is detected by a signal with a magnitude that exceeds the magnitude of background signals for the detection system, and 2) the single-analyte is detected by a signal at a location that is spatially separated from the location of a signal corresponding to a different single-analyte (i.e., a spatial minimum of signal magnitude exists between a first single-analyte and a second single-analyte for the two single-analytes to be spatially resolved). In some cases, a signal corresponding to a first single-analyte may be considered spatially resolved from a signal corresponding to a second single-analyte if a signal minimum occurs between the locations of the two single-analytes with a magnitude that is substantially less than an average or peak signal maximum of one or both signal maxima corresponding to the first and second single analytes. For example, a signal minimum between two signal maxima corresponding respectively to a first single analyte and a second single analyte may have a magnitude that is no more than about 95%, 90%, 80%, 70%, 60%, 50%, 40%, 30%, 20%, 10%, 5%, 1%, or less than 1% of an average or peak signal maximum of the two signal maxima. In some cases, signals corresponding to two or more analytes may be considered spatially resolved if a spatial resolution criterion is achieved, such as the Rayleigh Criterion. A signal magnitude (peak or average) corresponding to a single-analyte may have a signal-to-noise ratio relative to an average background signal of at least about 1.1:1, 1.5:1, 2:1, 2.5:1, 3:1, 4:1, 5:1, 10:1, 20:1, 50:1, 100:1 or more than 100:1.

In some of the implementations described herein, the term “solid support” can refer to a rigid substrate that is insoluble in aqueous liquid. The substrate can be non-porous or porous. The substrate can optionally be capable of taking up a liquid (e.g., due to porosity) but will typically be sufficiently rigid that the substrate does not swell substantially when taking up the liquid and does not contract substantially when the liquid is removed by drying. A nonporous solid support is generally impermeable to liquids or gases. Exemplary solid supports include, but are not limited to, glass, modified or functionalized glass, plastics (including acrylics, polystyrene and copolymers of styrene and other materials, polypropylene, polyethylene, polybutylene, polyurethanes, Teflon™, cyclic olefins, polyimides etc.), nylon, ceramics, resins, Zeonor™, silica or silica-based materials including silicon, quartz, fused silica and modified silicon, carbon, metals, inorganic glasses, optical fiber bundles, and polymers.

In some of the implementations described herein, the term “structured nucleic acid particle” or “SNAP” can refer to a single- or multi-chain polynucleotide molecule having a compacted three-dimensional structure. The compacted three-dimensional structure can optionally be characterized in terms of hydrodynamic radius or Stoke's radius of the SNAP relative to a random coil or other non-structured state for a nucleic acid having the same sequence length as the SNAP. The compacted three-dimensional structure can optionally be characterized with regard to tertiary structure. For example, a SNAP can be configured to have an increased number of internal binding interactions between regions of a polynucleotide strand, less distance between the regions, increased number of bends in the strand, and/or more acute bends in the strand, as compared to a nucleic acid molecule of similar length in a random coil or other non-structured state. Alternatively or additionally, the compacted three-dimensional structure can optionally be characterized with regard to quaternary structure. For example, a SNAP can be configured to have an increased number of interactions between polynucleotide strands or less distance between the strands, as compared to a nucleic acid molecule of similar length in a random coil or other non-structured state. In some configurations, the secondary structure (i.e. the helical twist or direction of the polynucleotide strand) of a SNAP can be configured to be more dense than a nucleic acid molecule of similar length in a random coil or other non-structured state. A SNAP can optionally be modified to permit attachment of additional molecules to the SNAP. A SNAP may contain DNA, RNA, PNA, or modified or non-natural nucleic acids, or combinations thereof. A SNAP may include a plurality of oligonucleotides that hybridize to form the SNAP structure. The plurality of oligonucleotides in a SNAP may include oligonucleotides that are attached to other molecules (e.g., probes, analytes such as proteins, reactive moieties, or detectable labels) or are configured to be attached to other molecules (e.g., by functional groups). A SNAP may include engineered or rationally designed structures. Exemplary SNAPs include nucleic acid origami and nucleic acid nanoballs.

As used herein, the term “tether” refers to a molecule or moiety that is configured to interact with a docker or that is interacting with a docker. A tether can be a moiety of a substance, object, molecule (e.g., affinity reagent or analyte), solid support, address, particle, or bead. A tether can include a polymer, nucleic acid strand, nucleic acid duplex, nucleotide sequence, protein, affinity reagent, epitope, paratope, receptor, ligand or the like. A tether can interact with a docker via covalent or non-covalent bonding.

In some of the implementations described herein, the term “unique identifier” can refer to a moiety, object or substance that is co-localized with an analyte and that is distinct from other identifiers, throughout one or more steps of a process. The moiety, object or substance can be, for example, a solid support such as a particle or bead; a location on a solid support; a site in an array; a tag; a label such as a luminophore; a molecular barcode such as a nucleic acid having a unique nucleotide sequence or a protein having a unique amino acid sequence; or an encoded device such as a radiofrequency identification (RFID) chip, electronically encoded device, magnetically encoded device or optically encoded device. A unique identifier can be covalently or non-covalently attached to an analyte. A unique identifier can be exogenous to a co-localized analyte, for example, being synthetically attached to the co-localized analyte. Alternatively, a unique identifier can be endogenous to the analyte, for example, being attached or co-localized with the analyte in the native milieu of the analyte.

In some of the implementations described herein, the term “vessel” can refer to an enclosure that contains a substance. The enclosure can be permanent or temporary with respect to the timeframe of a method set forth herein or with respect to one or more steps of a method set forth herein. Exemplary vessels include, but are not limited to, a well (e.g., in a multiwell plate or array of wells), test tube, channel, tubing, pipe, flow cell, bottle, vesicle, droplet that is immiscible in a surrounding fluid, or the like. A vessel can be entirely sealed to prevent fluid communication from inside to outside, and vice versa. Alternatively, a vessel can include one or more ingress or egress to allow fluid communication between the inside and outside of the vessel.

The embodiments set forth below and recited in the claims can be understood in view of the above definitions.

### Methods of Detecting Binding Interactions

In an aspect, provided herein is a method, comprising: (a) contacting a plurality of affinity reagents to a plurality of analytes for an equilibration period, in which the equilibration period is sufficient to form a binding equilibrium between affinity reagents of the plurality of affinity reagents and analytes of the plurality of analytes, and (b) after the equilibration period, detecting complexes comprising an affinity reagent bound to an analyte, in which the quantity of detected complexes is greater than a steady-state quantity of complexes that exists at the binding equilibrium.

In another aspect, provided herein is a method, comprising: (a) contacting a plurality of affinity reagents to a plurality of analytes for an equilibration period, in which the equilibration period is sufficient to form a binding equilibrium between affinity reagents of the plurality of affinity reagents and analytes of the plurality of analytes, and (b) after the equilibration period, detecting which analytes of the plurality of analytes formed a binding interaction with an affinity reagent of the plurality of affinity reagents during the equilibration period, in which the quantity of binding interactions is greater than a steady-state quantity of binding interactions that exists at the binding equilibrium.

In another aspect, provided herein is a method, comprising: (a) contacting a plurality of affinity reagents to a plurality of analytes for an equilibration period, in which each analyte of the plurality of analytes is individually paired to a unique identifier, (b) during the equilibration period, recording binding interactions at unique identifiers containing an affinity reagent bound to an analyte, and (c) detecting presence of absence of signals at a subset of the unique identifiers, thereby identifying analytes bound by affinity reagents during the equilibration period at the subset of unique identifiers.

In another aspect, provided herein is a method, comprising: (a) contacting a plurality of affinity reagents to a plurality of analytes for an equilibration period, thereby coupling affinity reagents of the plurality of affinity reagents to analytes of the plurality of analytes, wherein each analyte of the plurality of analytes is paired to a unique identifier, (b) forming a detectable reaction for analytes bound by an affinity reagent during the equilibration period, and (c) detecting presence of the detectable reaction at a subset of unique identifiers, thereby detecting the subset of analytes of the plurality of analytes bound by an affinity reagent of the plurality of affinity reagents.

Methods set forth herein may contain the steps of: (i) forming a binding interaction between an affinity reagent and an analyte, and (ii) forming a reaction between a first moiety and a second moiety, in which the first moiety is co-localized with the affinity reagent and the second moiety is co-localized with the analyte. A moiety may be co-localized with an affinity reagent or analyte if the moiety is attached to the affinity reagent or analyte, or if the moiety and affinity reagent or analyte are each attached to a solid support, molecule, or particle at a separation distance such that the moiety and the affinity reagent or analyte are not optically resolvable from each other. Preferably, a moiety co-localized with a first affinity reagent or analyte may not be optically resolvable from the affinity reagent or analyte, but will be optically resolvable from a moiety co-localized with a second affinity reagent or analyte.

A moiety may be co-localized with an analyte if a separation distance between the moiety and the analyte is no more than about 100 nanometers (nm), 80 nm, 60 nm, 50 nm, 40 nm, 30 nm, 20 nm, 10 nm, 5 nm, 1 nm, or less than 1 nm. Alternatively or additionally, a moiety may be co-localized with an analyte if a separation distance between the moiety and the analyte is at least about 1 nm, 5 nm, 10 nm, 20 nm, 30 nm, 40 nm, 50 nm, 60 nm, 80 nm, 100 nm, or more than 100 nm.

A reaction may be any suitable interaction between a first moiety and a second moiety. A reaction may be characterized by at least one of: (i) inhibiting dissociation of an affinity reagent from an analyte due to the interaction of the first moiety with the second moiety, and (ii) facilitating transfer of a detectable label associated with a binding interaction between the affinity reagent and the analyte (e.g., transferring a detectable label to an analyte or a unique identifier co-localized with the analyte, transferring a detectable label from an analyte or a unique identifier co-localized with the analyte). Preferably, a reaction may co-localize a detectable label at an address containing an analyte or a unique identifier such that the detectable label remains detectable at the address in the presence of a condition that disrupts a binding equilibrium between an affinity reagent and the analyte (e.g., rinsing unbound affinity reagents from contact with the analyte), or in the presence of a condition that dissociates the binding interaction between the affinity reagent and the analyte (e.g., contacting with a denaturing species, altering a pH, altering an ionic strength, altering a temperature, etc.).

Affinity reagents may be contacted to analytes for an equilibration period, in which the equilibration period is sufficient for a binding equilibrium to occur between the affinity reagents and the analytes. A binding equilibrium can occur when the rate of affinity reagents associating to analytes is equal to the rate of affinity reagents dissociating from analytes. Accordingly, for fixed quantities of affinity reagents and analytes, the total quantity of complexes formed by affinity reagents associated to analytes will be substantially constant at binding equilibrium, although the specific associated or unassociated molecules may differ between two time points during the binding equilibrium. For affinity reagents contacted to immobilized analytes, binding equilibrium may be disrupted by removal of unbound affinity reagents. The decrease in concentration of affinity reagents around the analytes may facilitate dissociation of bound affinity reagents from analytes. Likewise, binding equilibrium may be altered by changes in the chemical environment surrounding the analytes and affinity reagents. Changes in pH, ionic strength, buffer composition, surfactant concentration, and temperature can increase or decrease the quantity of affinity reagent/analyte complexes occurring at steady state.

The amount of time necessary to achieve binding equilibrium can depend upon the binding characteristics of affinity reagents and analytes, as well as surrounding chemical environment. Binding on-rates and off-rates can vary depending upon the affinity reagent and its binding target. Methods of measuring binding characteristics of affinity reagents are known in the art, and the skilled person can readily determine a sufficient equilibration period to provide a binding equilibrium between affinity reagents and analytes. An equilibration period may occur for at least about 30 seconds(s), 1 minute (min), 2 mins, 3 mins, 4 mins, 5 mins, 10 mins, 15 mins, 30 mins, 60 mins, or more than 60 mins. Alternatively or additionally, an equilibration period may occur for no more than about 60 mins, 30 mins, 15 mins, 10 mins, 5 mins, 4 mins, 3 mins, 2 mins, 1 min, 30 s, or less than 30 s.

Some methods set forth herein may comprise a step of recording a binding interaction between an affinity reagent and an analyte. A binding interaction may be directly recorded if a reaction inhibits dissociation of the affinity reagent from the analyte, thereby facilitating detection of the complex comprising the affinity reagent and analyte (e.g., by detection of a detectable label co-localized with the affinity reagent or co-localized with a moiety that forms a reaction). A binding interaction may be indirectly recorded if a reaction transfers a detectable label between the affinity reagent and analyte, thereby providing a change in detectable signal that is detectable after the affinity reagent has dissociated from the analyte.

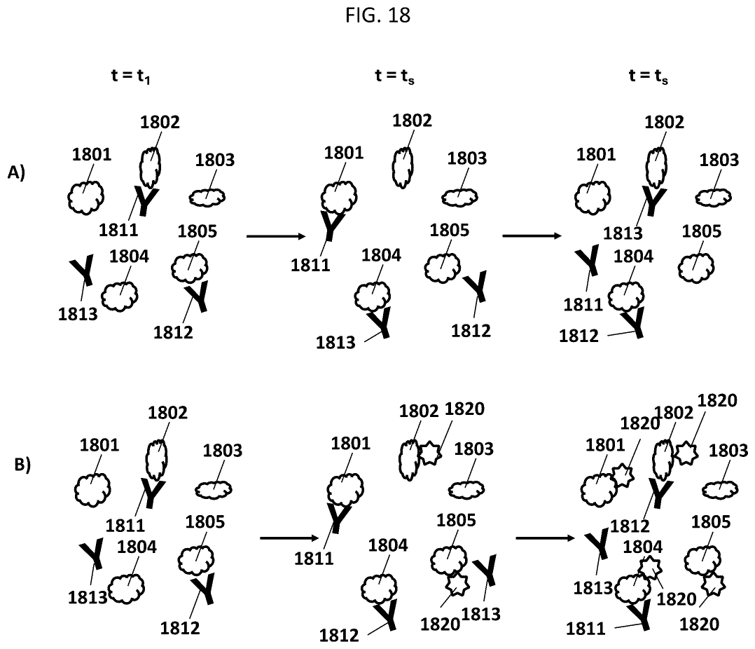

FIG. 18 illustrates a potential advantage of recording binding interactions by methods set forth herein. Steady-state binding between a plurality of analytes (1801, 1802, 1803, 1804, and 1805) and a plurality of affinity agents (1811, 1812, 1813) is shown at three time points, t₁, t₂, and t₃, respectively. At time point t₁, affinity agent 1811 is bound to analyte 1802, affinity agent 1812 is bound to analyte 1805, and affinity agent 1813 is unbound. At time point t₂, affinity agent 1811 is bound to analyte 1801, affinity agent 1813 is bound to analyte 1804, and affinity agent 1812 is unbound. At time point t₃, affinity agent 1812 is bound to analyte 1804, affinity agent 1813 is bound to analyte 1802, and affinity agent 1811 is unbound. Under a steady-state equilibrium condition, 40% of analytes are observed to be bound by an affinity reagent at any given time point. In scenario A (upper), detection of the system can provide the equilibrium bound fraction, but provides no information on which analytes formed binding interactions with affinity reagents at time points before or after the detection event. In scenario B (lower), each binding interaction between an analyte and an affinity reagent is recorded by attaching a detectable label 1820 to the analyte. In scenario B, at time point t₃, a greater fraction of analytes can be detected to have participated in a binding interaction with an affinity agent than a fraction of analytes bound to affinity reagents at any given time point in the steady-state equilibrium condition. If analytes are provided in a spatially-resolvable format (e.g., an array of analytes), it may be possible to detect individually whether each analyte formed a binding interaction with an affinity reagent during an equilibration period by recording the binding interactions at unique identifiers co-localized with each individual analyte.

Accordingly, for binding interactions between a plurality of analytes and a plurality of affinity reagents, binding interactions between analytes and affinity reagents may be detected for a fraction of analytes that exceeds the steady-state fraction of analytes that are bound by the affinity reagents at equilibrium. The fraction of analytes of a plurality of analytes with a detected binding interaction may exceed the steady-state fraction of analytes of the plurality of analytes that are bound by the affinity reagents at equilibrium by at least about 0.1%, 0.5%, 1%, 2%, 3%, 4%, 5%, 6%, 7%, 8%, 9%, 10%, 15%, 20%, 30%, 40%, 50%, or more than 50%. For example, if the fraction of analytes of a plurality of analytes with a detected binding interaction exceeds the steady-state fraction of analytes that are bound by the affinity reagents at equilibrium by 5%, and the steady-state fraction of analytes of a plurality of analytes that are bound by the affinity reagents at equilibrium is 10%, the fraction of analytes with a detected binding interaction may be about 15% of the plurality of analytes. Alternatively or additionally, the fraction of analytes of a plurality of analytes with a detected binding interaction may exceed the steady-state fraction of analytes of the plurality of analytes that are bound by the affinity reagents at equilibrium by no more than about 50%, 40%, 30%, 20%, 15%, 10%, 9%, 8%, 7%, 6%, 5%, 4%, 3%, 2%, 1%, 0.5%, 0.1%, or less than 0.1%. In some cases, binding interactions between analytes and affinity reagents may be detected for a fraction of analytes that is substantially the same as or less than the steady-state fraction of analytes that are bound by the affinity reagents at equilibrium.

A method may comprise a step of removing unbound affinity reagents from contact with a plurality of analytes before detecting which analytes have formed a binding interaction with an affinity reagent during an equilibration period. Such a step may be useful if the binding interaction has been directly recorded. Alternatively, a method may comprise a step of dissociating complexes, each complex comprising an analyte associated to an affinity reagent, after contacting affinity reagents to analytes and before detecting which analytes have formed a complex with an affinity reagent during an equilibration period. Such a step may be useful if the binding interaction has been indirectly recorded. Alternatively, a method set forth herein may not comprise a step of removing unbound affinity reagents before detecting which analytes have formed a binding interaction with an affinity reagent during an equilibration period. Accordingly, detection of the binding interaction between affinity reagents and analytes may occur in the presence of unbound affinity reagents.

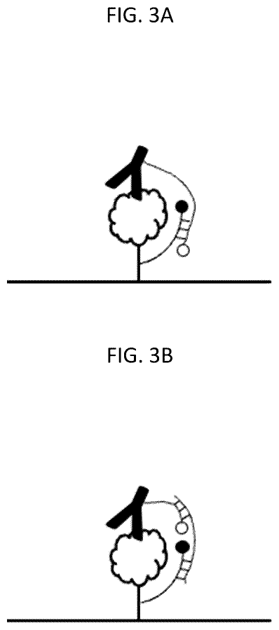

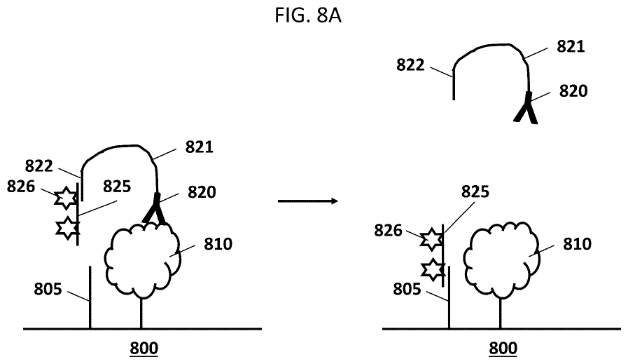

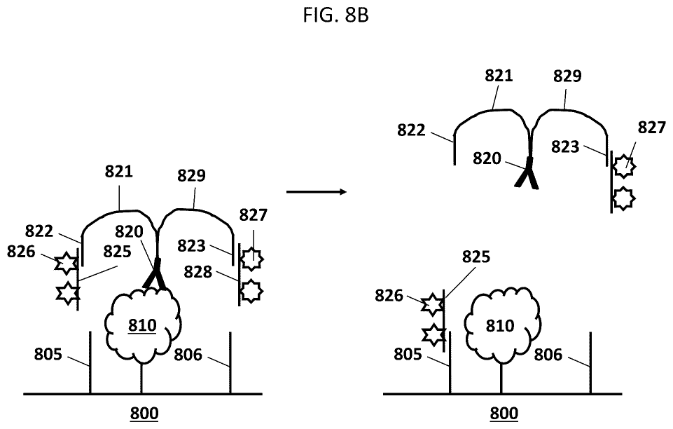

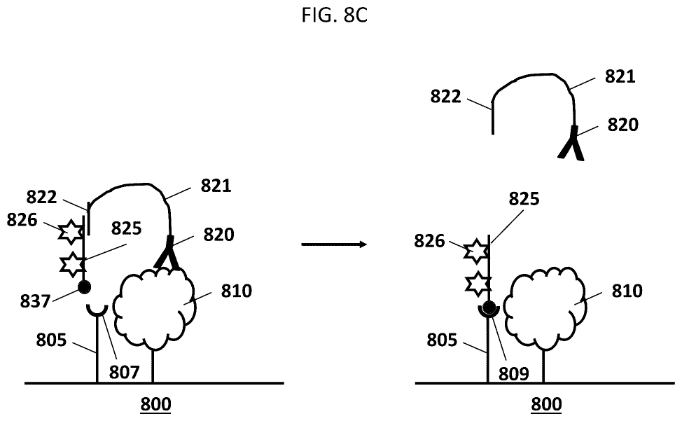

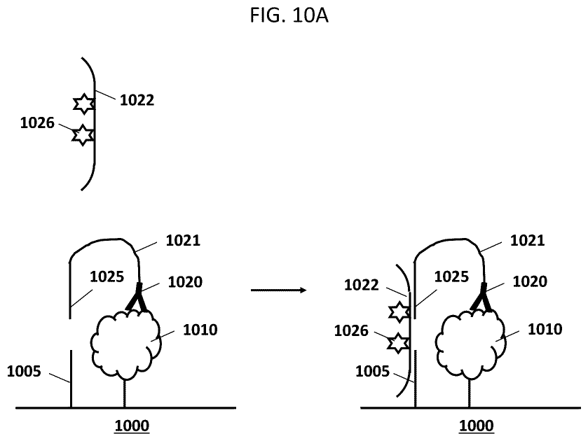

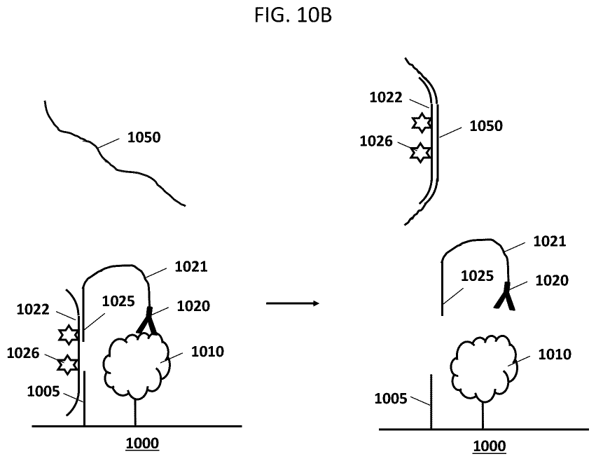

A method may comprise a step of recording a binding interaction between an affinity reagent and an analyte, in which recording the binding interaction between the affinity reagent and the analyte comprises forming a reaction between a first moiety co-localized with the affinity reagent and a second moiety co-localized with the analyte. In some cases, the reaction can inhibit dissociation of the affinity reagent from the complex. For example, methods of recording a binding interaction by inhibiting dissociation are shown in FIGS. 10A-10B, FIGS. 13C-13D, FIG. 14C, and FIGS. 15-17. In other cases, the reaction can produce a detectable signal. For example, methods of recording a binding interaction that produces a detectable signal are shown in FIGS. 3A-3C, FIGS. 5A-5C, FIGS. 8A-8C, FIGS. 9A-9C, FIGS. 10A-10B, FIG. 12, FIGS. 13A, 13B and 13D, FIGS. 14A-14C, and FIGS. 15-17. In yet other cases, the reaction can transfer a detectable label between an affinity agent and an analyte or a unique identifier co-localized with the analyte (e.g., transfer of the detectable label from the affinity reagent to the analyte or unique identifier, transfer of the detectable label from the analyte or unique identifier to the affinity reagent). For example, methods of recording a binding interaction by transferring a detectable label are shown in FIGS. 8A-8C, FIGS. 9A-9C, and FIG. 14C.

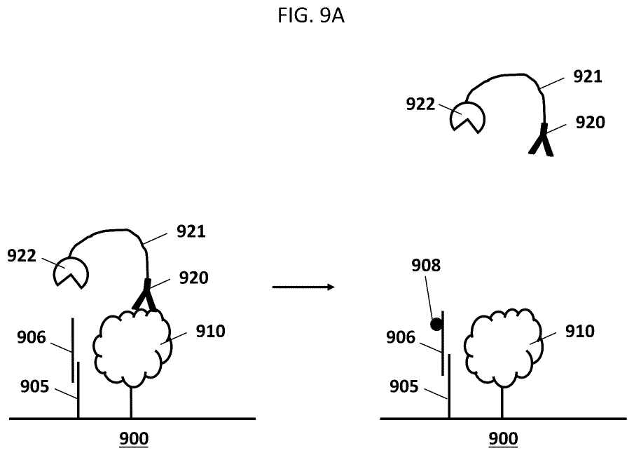

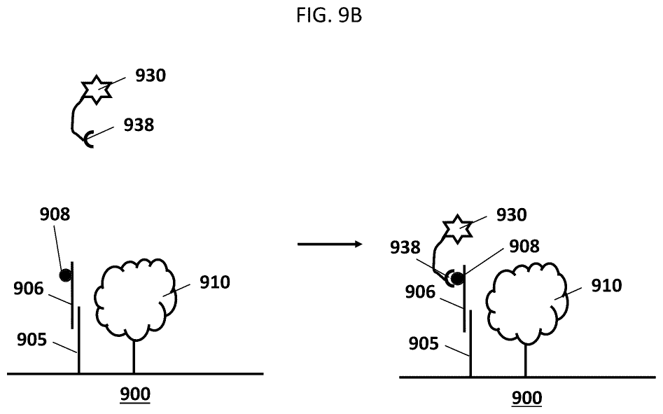

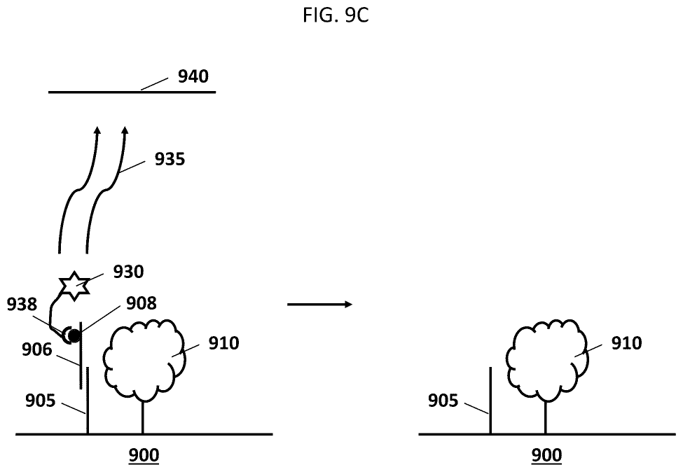

In another aspect, provided herein is a method, comprising: (a) binding an affinity reagent to an analyte at a fixed spatial address, (b) coupling a kinetically-controlled detection agent to the fixed spatial address, and (c) after coupling the kinetically-controlled detection agent to the fixed spatial address, detecting a signal at the fixed spatial address. It may be especially useful to incorporate a kinetically-controlled detection agent into methods that utilize an affinity reagent with characterized superordinate and subordinate binding interactions, as set forth herein. A kinetically-controlled detection reagent may facilitate detection of a superordinate or subordinate binding interaction between an affinity reagent and a binding target, thereby facilitating identification of the binding target. Examples of methods utilizing kinetically-controlled detection agents may be found, for example, in FIGS. 9A-9C, FIG. 11-12, FIG. 13C-13D, and FIGS. 14B-14C, and FIGS. 15-17.

In some methods, a binding interaction between an affinity reagent and an immobilized analyte may be directly recorded by forming a reaction that immobilizes the affinity reagent with the analyte. A kinetically-controlled binding reagent may be utilized to control the rate of formation of the reaction. Preferably, the on-rate or forward rate of the reaction is slower than the on-rate of the affinity reagent to the analyte, thereby reducing the likelihood of the reaction occurring before a binding interaction has occurred. In some cases, a reaction may bind a first moiety co-localized with the affinity reagent to a second moiety co-localized with the analyte. In such cases, a kinetically-controlled detection agent (e.g., an oligonucleotide comprising a secondary structure, a reactive functional group, a component of a ligand-receptor binding pair, etc.) may be incorporated into the first moiety, the second moiety, or both the first and second moiety. In other such cases, a kinetically-controlled detection agent (e.g., an oligonucleotide comprising a secondary structure, a moiety comprising a plurality of reactive functional groups, a moiety comprising a plurality of components of receptor-ligand binding pairs) may be incorporated into a third moiety that binds to the first moiety and the second moiety. Binding of a third moiety (e.g., a bridging oligonucleotide) may be kinetically controlled (e.g., via incorporation of secondary nucleic acid structures) with respect to its binding to the first moiety, the second moiety, or to both the first and second moiety.

In some methods, a binding interaction between an affinity reagent and an immobilized analyte may be directly recorded by forming a reaction that transfers a detectable label between the affinity reagent and the immobilized analyte or a unique identifier co-localized with the analyte). Kinetically-controlled indirect recording methods could include enzymatic labeling of nucleic acid or peptide tags, photo-catalyzed binding of detectable labels to analytes or unique identifiers co-localized therewith, or transfer of a transferrable moiety between an affinity reagent and an analyte or a unique identifier co-localized therewith. Oligonucleotide moieties comprising secondary structure may be useful transferrable moieties for controlling the rate of transfer of the transferrable moiety between an affinity reagent and an analyte or a unique identifier co-localized therewith.

In some cases, an affinity reagent may be contacted to an analyte in the presence of a kinetically-controlled detection agent. Such a configuration may be utilized if the on-rate or reaction rate of the kinetically-controlled detection agent is substantially slower than the on-rate of the affinity reagent to the analyte. Alternatively, a kinetically-controlled detection agent may be contacted to an analyte after contacting an affinity reagent to the analyte. Such a configuration may be utilized if the on-rate or reaction rate of the kinetically-controlled detection agent is substantially faster or equal to the on-rate of the affinity reagent to the analyte.

The on-rate or forward reaction rate of a kinetically-controlled detection agent may be chosen to distinguish superordinate binding interactions from subordinate binding interactions of an affinity reagent. For example, if a binding on-rate of a kinetically-controlled detection agent is slower than a binding off-rate of a subordinate binding interaction of the affinity agent, it is unlikely that the reaction facilitated by the kinetically-controlled detection agent will occur in the presence of the subordinate binding interaction. In another example, if a binding on-rate of a kinetically-controlled detection agent is faster than a binding off-rate of a superordinate binding interaction of the affinity agent, it is likely that the reaction facilitated by the kinetically-controlled detection agent will occur in the presence of the superordinate binding interaction. In some cases, a method may utilize two differing kinetically-controlled detection agents, in which the first kinetically-controlled detection agent has a slower on-rate than the off-rate of a subordinate binding interaction, in which the second kinetically-controlled detection agent has a faster on-rate than the off-rate of a subordinate binding interaction, and in which the first and second kinetically-controlled detection agents each have a faster on-rate than the off-rate of a superordinate binding interaction. In such cases, reactions facilitated by both kinetically-controlled binding reagents will only be detected for a superordinate binding interaction of an affinity reagent and an analyte, while only the reaction of the second kinetically-controlled detection agent will be detected for a subordinate binding interaction of the affinity reagent and an analyte. Many methods set forth herein may readily be modified to include two or more differing kinetically-controlled detection agents for identification of superordinate or subordinate binding interactions.

Binding kinetics of kinetically-controlled detection agents can readily be measured by various methods, including empirical measurement of binding or reaction rates, as well as in silico estimation of binding or reaction rates for certain types of kinetically-controlled detection agents (e.g., structured oligonucleotides, enzymes, etc.). Accordingly, measured binding kinetics of kinetically-controlled detection agents can be compared to binding kinetics of affinity reagents to identify suitable combinations for methods set forth herein.

In some cases, a kinetically-controlled detection agent can comprise a detectable label (e.g., a detectable label attached to or co-localized with the kinetically-controlled detection agent). For example, a bridging oligonucleotide may be provided with one or more detectable labels that facilitate detection of a signal from the one or more detectable labels when the bridging oligonucleotide is coupled to docker and tether strands, as set forth herein. Alternatively, a kinetically-controlled detection agent may not comprise a detectable label. For example, enzymatic detection agents may attach substrates to an analyte, an affinity agent, or a moiety co-localized therewith. In some cases, an affinity reagent, an analyte, or a unique identifier may comprises a detectable label.

The present disclosure further provides systems for measuring binding interactions by methods set forth herein. In another aspect, provided herein is a system, comprising: (a) a solid support comprising a plurality of sites, in which each site of the plurality of sites is optically resolvable from any other site of the plurality of sites, and (b) a plurality of analytes immobilized on the solid support, in which each site of the plurality of sites is attached to one and only one analyte of the quantity of analytes. in which, in a first configuration, the system further comprises a plurality of affinity reagents, in which a fraction of the affinity reagents is bound to analytes of the plurality of analytes, in which a quantity of the fraction of affinity reagents is determined by a binding equilibrium of the affinity reagents for the analytes, and in which, in a second configuration, the system is substantially devoid of affinity reagents, in which each individual site of a fraction of sites of the plurality of sites comprises a detectable label, in which a quantity of the fraction of sites is greater than a quantity of the second fraction of affinity reagents.

In another aspect, provided herein is a system, comprising: (a) a solid support comprising a plurality of sites, in which each site of the plurality of sites is optically resolvable from any other site of the plurality of sites, (b) a plurality of analytes immobilized on the solid support, in which each site of the plurality of sites is attached to one and only one analyte of the quantity of analytes, and (c) a plurality of affinity reagents bound analytes of the plurality of analytes at sites of the plurality of sites, in which the affinity reagents have a known binding equilibrium for binding to analytes of the plurality of analytes, and in which a quantity of the sites of the plurality of sites is greater than a quantity of analytes bound to affinity reagents based upon the known binding equilibrium.

### Attracting Affinity Reagents to Analytes Via Applied Stimuli

The present disclosure provides a method of detecting analytes. The method can include steps of (a) providing a mixture of analytes with affinity reagents, wherein the analytes are immobilized on a surface and the affinity reagents are in fluid phase; (b) applying a stimulus to attract the affinity reagents to the surface; and (c) detecting binding of affinity reagents to analytes on the surface. For ease of explanation, several configurations of the method will be exemplified herein in the context of using immobilized analytes and fluid-phase affinity reagents. However, it will be understood that the method can be carried out using immobilized affinity reagents and fluid-phase analytes. Accordingly, the method can include steps of (a) providing a mixture of analytes with affinity reagents, wherein the affinity reagents are immobilized on a surface and the analytes are in fluid phase; (b) applying a stimulus to attract the analytes to the surface; and (c) detecting binding of analytes to affinity reagents on the surface.

In some configurations, a method of detecting analytes on an array can include steps of (a) contacting an array with affinity reagents in fluid phase, wherein analytes are attached at addresses in the array; (b) allowing affinity reagents in the fluid phase to bind to analytes attached at addresses in the array; (c) removing a plurality of the affinity reagents from contact with the array, thereby retaining a fraction of the affinity reagents in contact with the array; (d) applying a stimulus to attract the fraction of affinity reagents to the array; and (e) detecting binding of affinity reagents in the fraction to analytes attached at addresses in the array. Again, the method will be exemplified herein in the context of using immobilized analytes and fluid-phase affinity reagents. However, the method can be carried out using immobilized affinity reagents and fluid-phase analytes. Accordingly, the method can include steps of (a) contacting an array with analytes in fluid phase, wherein affinity reagents are attached at addresses in the array; (b) allowing analytes in the fluid phase to bind to affinity reagents attached at addresses in the array; (c) removing a plurality of the analytes from contact with the array, thereby retaining a fraction of the analytes in contact with the array; (d) applying a stimulus to attract the fraction of analytes to the array; and (e) detecting binding of analytes in the fraction to affinity reagents attached at addresses in the array.

Any of a variety of analytes can be used in a method or composition set forth herein. For ease of explanation, various methods and compositions will be exemplified herein in the context of using proteins. It will be understood that the exemplified methods and compositions can be extended to other analytes. Exemplary analytes include, but are not limited to, a tissue, cell, organelle, virus, nucleic acid (e.g., DNA or RNA), carbohydrate (e.g., monosaccharide, oligosaccharide or polysaccharide), glycan, vitamin, enzyme cofactor, hormone, or small molecule such as a candidate therapeutic agent, metabolite, nucleotide, nucleoside, amino acid, sugar, lipid, or the like. In some configurations, a composition or method set forth herein can lack one or more of the analytes set forth herein.

A composition or method set forth herein can be configured for a single analyte or for a plurality of different analytes. A plurality of analytes can include, for example, a proteome, or substantial fraction thereof, including a variety of different proteins; a genome, or substantial fraction thereof, including a variety of different DNA sequences; a transcriptome, or substantial fraction thereof, including a variety of different RNA sequences; a metabolome, or substantial fraction thereof, including a variety of different metabolites; or a microbiome, or substantial fraction thereof, including a variety of different microbes. These and other analytes known in the art can be used in compositions and methods set forth herein.

A protein or other analyte provided to a method set forth herein can be derived from a natural or synthetic source. Exemplary sources include, but are not limited to a biological tissue, fluid, cell or subcellular compartment such as an organelle (e.g., nucleus, mitochondria, chloroplast, endoplasmic reticulum, vesicle, cytoskeleton, vacuole, lysosome, cell membrane, cytosol or Golgi apparatus). For example, a sample can be derived from a tissue biopsy, biological fluid (e.g., blood, plasma, extracellular fluid, urine, mucus, saliva, semen, vaginal fluid, sweat, synovial fluid, lymph, cerebrospinal fluid, peritoneal fluid, pleural fluid, amniotic fluid, intracellular fluid, extracellular fluid, etc.), fecal sample, hair sample, cultured cell, culture media, fixed tissue sample (e.g., fresh frozen or formalin-fixed paraffin-embedded) or protein synthesis reaction. A primary source for a cancer biomarker protein may be a tumor biopsy sample. Other sources include environmental samples or forensic samples.

Exemplary organisms from which a protein or other analyte can be derived include, but are not limited to, a mammal such as a rodent, mouse, rat, rabbit, guinea pig, ungulate, horse, sheep, pig, goat, cow, cat, dog, primate, non-human primate or human; a plant such as Arabidopsis thaliana, tobacco, corn, sorghum, oat, wheat, rice, canola, or soybean; an algae such as Chlamydomonas reinhardtii; a nematode such as Caenorhabditis elegans; an insect such as Drosophila melanogaster, mosquito, fruit fly, honey bee or spider; a fish such as zebrafish; a reptile; an amphibian such as a frog or Xenopus laevis; a dictyostelium discoideum; a fungi such as Pneumocystis carinii, Takifugu rubripes, yeast, Saccharamoyces cerevisiae or Schizosaccharomyces pombe; or a Plasmodium falciparum. A protein can also be derived from a prokaryote such as a bacterium, Escherichia coli, staphylococci or Mycoplasma pneumoniae; an archae; a virus such as Hepatitis C virus, influenza virus, coronavirus, or human immunodeficiency virus; or a viroid. A protein or other analyte can be derived from a homogeneous culture or population of the above organisms or alternatively from a collection of several different organisms, for example, in a community or ecosystem.

In some cases, a protein or other analyte can be derived from an organism that is collected from a host organism. A protein or other analyte may be derived from a parasitic, pathogenic, symbiotic, or latent organism collected from a host organism. A protein or other analyte can be derived from an organism, tissue, cell or biological fluid that is known or suspected of being associated with a disease state or disorder (e.g., an oncogenic virus). Alternatively, a protein or other analyte can be derived from an organism, tissue, cell or biological fluid that is known or suspected of not being associated with a particular disease state or disorder. For example, one or more proteins isolated from such a source can be used as a control for comparison to results acquired from a source that is known or suspected of being associated with the particular disease state or disorder. A sample may include a microbiome. A sample may include a plurality of proteins or other analytes of interest contributed by microbiome constituents. In some cases, one or more proteins (or other analytes) used in a method, composition or apparatus set forth herein may be obtained from a single organism (e.g., an individual human), single cell, single organelle, or single protein-containing particle (e.g., a viral particle).

In some cases, one or more proteins or other analytes of interest can be obtained from a single cell, protein-containing particle (e.g., a viral particle), or organelle. A single cell, protein-containing particle, or organelle may be collected by any known method in the art, such as fluorescence assisted cell sorting, magnetic-assisted cell sorting, and buoyancy-assisted cell sorting. In some cases, a single cell, protein-containing particle, or organelle may be collected by an emulsion technique such as liposome or micellar capture.

One or more analytes can optionally be separated or isolated from other components of the source for the analyte(s). For example, one or more proteins can be separated or isolated from lipids, nucleic acids, hormones, enzyme cofactors, vitamins, metabolites, microtubules, organelles (e.g., nucleus, mitochondria, chloroplast, endoplasmic reticulum, vesicle, cytoskeleton, vacuole, lysosome, cell membrane, cytosol or Golgi apparatus), other proteins or the like. Protein separation can be carried out using methods known in the art such as centrifugation (e.g., to separate membrane fractions from soluble fractions), density gradient centrifugation (e.g., to separate different types of organelles), precipitation, affinity capture, adsorption, liquid-liquid extraction, solid-phase extraction, chromatography (e.g., affinity chromatography, ion exchange chromatography, reverse phase chromatography, size exclusion chromatography, electrophoresis (e.g., polyacrylamide gel electrophoresis) or the like. Useful protein separation methods are set forth in Scopes, Protein Purification Principles and Practice, Springer; 3rd edition (1993).

A protein that is used in a composition or method set forth herein can be in a native or denatured conformation. For example, a protein can be in a native conformation, whereby it is capable of performing native function(s) such as catalysis of its natural substrate(s) or binding to its natural substrate(s). Alternatively, a protein can be in a denatured conformation whereby it is incapable of performing certain native function(s) such as catalysis of its natural substrate(s) or binding to its natural substrate(s). A protein can be in a native conformation for some manipulations set forth herein and in a denatured conformation for other manipulations set forth herein. A protein may be denatured at any stage during manipulation, including for example, upon removal from a native milieu or at a later stage of processing such as a stage where the protein is separated from other cellular components, fractionated from other proteins, functionalized to include a reactive moiety, attached to a particle or solid support, contacted with an affinity reagent, detected, or other manipulation. Any of a variety of denaturants can be used such as heat (e.g., temperatures greater than about 40° C., 60° C., 80° C. or higher), applied force such as magnetic force or fluidic force, excessive pH (e.g., pH lower than 4.0, 3.0 or 2.0; or pH greater than 10.0, 11.0 or 12.0); chaotropic agents (e.g., urea, guanidinium chloride, or sodium dodecyl sulfate), organic solvent (e.g., chloroform or ethanol), physical agitation (e.g., sonication) and/or radiation. A denatured protein may be refolded, for example, reverting to a native state for one or more steps of a process set forth herein.

An analyte, such as a protein, can be attached to a particle, solid support or other substance. An exemplary particle is a virus particle such as a phage. A particularly useful particle is a structured nucleic acid particle. Structured nucleic acid particles can optionally include nucleic acid origami. A nucleic acid origami can include one or more nucleic acids folded into a variety of overall shapes such as a disk, tile, cylinder, cone, sphere, cuboid, tubule, pyramid, polyhedron, or combination thereof. Examples of structures formed with DNA origami are set forth in Zhao et al. Nano Lett. 11, 2997-3002 (2011); Rothemund Nature 440:297-302 (2006); Sigle et al, Nature Materials 20:1281-1289 (2021); or U.S. Pat. Nos. 8,501,923 or 9,340,416, each of which is incorporated herein by reference. In some configurations, a structured nucleic acid particle can include a nucleic acid nanoball and the nucleic acid nanoball can include a concatemeric repeat of amplified nucleotide sequences. The concatemeric amplicons can include complements of a circular template amplified by rolling circle amplification. Exemplary nucleic acid nanoballs and methods for their manufacture are described, for example, in U.S. Pat. No. 8,445,194, which is incorporated herein by reference. Further examples of structured nucleic acid particles are set forth in U.S. Pat. Nos. 11,203,612 or 11,505,796; US Pat. App. Pub. No. 2022/0162684 A1, or U.S. patent application Ser. No. 18/058,000, each of which is incorporated herein by reference.

A particle, such as a structured nucleic acid particle, may have any of a variety of sizes and shapes to accommodate use in a desired application. For example, a particle can have a regular or symmetric shape or, alternatively, a particle can have an irregular or asymmetric shape. The shape can be rigid or pliable. The size or shape of a particle can be characterized with respect to length, area (i.e. footprint), or volume. The size or shape of a particle can be smaller than an address in an array to which it will associate or attach. Optionally, the size or shape of particles in a population are configured to preclude more than one of the particles from occupying an address in an array.

Optionally, a particle (e.g., a structured nucleic acid particle) or population of particles can have a minimum, maximum or average length of at least about 10 nm, 25 nm, 50 nm, 100 nm, 250 nm, 500 nm, 1 micron, 5 micron or more. Alternatively or additionally, a particle or population thereof can have a minimum, maximum or average length of no more than about 5 micron, 1 micron, 500 nm, 250 nm, 100 nm, 50 nm, 25 nm, 10 nm or less.

Optionally, a particle, such as a structured nucleic acid particle, or population thereof can have a minimum, maximum or average volume of at least about 1 micron³, 10 micron³, 100 micron³, 1 mm³ or more. Alternatively or additionally, a particle or population thereof can have a minimum, maximum or average volume of no more than about 1 mm³, 100 micron³, 10 micron³, 1 micron³ or less.

Optionally, the minimum, maximum or average area (i.e. footprint) for a particle, such as a structured nucleic acid particle, can be at least about 10 nm², 100 nm², 1 micron², 10 micron², 100 micron², 1 mm² or more. Alternatively or additionally, the minimum, maximum or average area for a particle footprint can be at most about 1 mm², 100 micron², 10 micron², 1 micron², 100 nm², 10 nm², or less. The footprint of a particle may have a regular shape or an approximately regular shape, such as triangular, square, rectangular, circular, ovoid, or polygonal shape.

A structured nucleic acid particle (e.g., having origami or nanoball structures) may include regions of single-stranded nucleic acid, regions of double-stranded nucleic acid, or combinations thereof. For example, a structured nucleic acid particle can have a nucleic acid origami structure which includes a scaffold strand and a plurality of staple strands. The scaffold strand can be configured as a single, continuous strand of nucleic acid, and the staples can be formed by nucleic acid strands that hybridize, in whole or in part, with the scaffold strand.

In some configurations, a nucleic acid origami includes a scaffold composed of a nucleic acid strand to which a plurality of oligonucleotides is hybridized. A nucleic acid origami may have a single scaffold molecule or multiple scaffold molecules. A scaffold strand can be linear (i.e. having a 3′ end and 5′ end) or circular (i.e. closed such that the scaffold lacks a 3′ end and 5′ end). A scaffold strand can be derived from a natural source, such as a viral genome or a bacterial plasmid. For example, a nucleic acid scaffold can include a single strand of an M13 viral genome. In other configurations, a scaffold strand may be synthetic, for example, having a non-naturally occurring nucleotide sequence in full or in part. A scaffold nucleic acid can be single stranded but for a plurality of oligonucleotides hybridized thereto or short regions of internal complementarity. The size of a scaffold strand may vary to accommodate different uses. For example, a scaffold strand may include at least about 100, 500, 1000, 2500, 5000 or more nucleotides. Alternatively or additionally, a scaffold strand may include at most about 5000, 2500, 1000, 500, 100 or fewer nucleotides.

A nucleic acid origami can include one or more oligonucleotides that are hybridized to a scaffold strand. An oligonucleotide can include two sequence regions that are hybridized to a scaffold strand, for example, to function as a ‘staple’ that restrains the structure of the scaffold. For example, a single oligonucleotide can hybridize to two regions of a scaffold strand that are separated from each other in the primary sequence of the scaffold strand. As such, the oligonucleotide can function to retain those two regions of the scaffold strand in proximity to each other or to otherwise constrain the scaffold strand to a desired conformation. Two sequence regions of an oligonucleotide staple that bind to a scaffold strand can be adjacent to each other in the nucleotide sequence of the oligonucleotide or separated by a spacer region that does not hybridize to the scaffold strand.

An oligonucleotide can include a first sequence region that is hybridized to a complementary sequence of a nucleic acid origami and a second region that provides a “handle” for attaching another moiety. For example, the moiety can include an analyte (e.g., protein), paratope, affinity moiety (e.g., antibody), organic linker, inorganic ion, chemical reactant such as a click chemistry reagent, docker or tether. Optionally, the moiety can be attached to an oligonucleotide that is complementary to the second region of the handle and the moiety can be attached to the nucleic acid origami via hybridization of the handle to the complementary oligonucleotide.

Oligonucleotides can be configured to hybridize with a nucleic acid scaffold, another oligonucleotide, a staple oligonucleotide, or a combination thereof. One or more regions of an oligonucleotide that hybridizes to another sequence of a nucleic acid origami or other structured nucleic acid particle can be located at or near the 5′ end of the oligonucleotide, at or near the 3′ end of the oligonucleotide, or in a region of the oligonucleotide that is between the end regions. The oligonucleotides can be linear (i.e. having a 3′ end and a 5′ end) or closed (i.e. circular, lacking both 3′ and 5′ ends). An oligonucleotide that is included in a nucleic acid origami or other structured nucleic acid particle can have any of a variety of lengths including, for example, at least about 10, 25, 50, 100, 250, 500, or more nucleotides. Alternatively or additionally, an oligonucleotide may have a length of no more than about 500, 250, 100, 50, 25, 10, or fewer nucleotides. An oligonucleotide may form a hybrid of at least about 5, 6, 7, 8, 9, 10, 15, 20, 25, 50 or more consecutive or total base pairs with another nucleotide sequence of a nucleic acid origami. Alternatively or additionally, an oligonucleotide may form a hybrid of no more than about 50, 25, 20, 15, 10, 9, 8, 7, 6, 5, or fewer consecutive or total base pairs with another nucleotide sequence.

A structured nucleic acid particle (e.g., nucleic acid origami, or nucleic acid nanoball) may be formed by an appropriate technique including, for example, those known in the art. Nucleic acid origami can be designed, for example, as described in Rothemund, Nature 440:297-302 (2006), or U.S. Pat. Nos. 8,501,923 or 9,340,416, each of which is incorporated herein by reference. Nucleic acid origami may be designed using a software package, such as CADNANO (cadnano.org), ATHENA (github.com/lcbb/athena), or DAEDALUS (daedalus-dna-origami.org).

An analyte can be attached to a label, for example, using compositions and methods set forth herein in the context of affinity reagents.

Optionally, an analyte of the present disclosure can be attached to a unique identifier in an array of unique identifiers. An array can include a number or variety of unique identifiers, for example, to accommodate a desired sample complexity. In some configurations, the array is configured for single-molecule resolution. For example, individual addresses of an array can each be attached to one, and only one, analyte. Alternatively, individual addresses of the array can each be attached to an ensemble of analytes. Several configurations for arrays and their methods of use will be exemplified below in the context of arrays having addresses as unique identifiers. It will be understood that the configurations can be extended to arrays having unique identifiers other than addresses.

The addresses of an array can optionally be optically observable and, in some configurations, adjacent addresses can be distinguishable when detected optically. Addresses of an array are typically discrete. The discrete addresses can be contiguous, or they can have interstitial spaces between each other. An array can have, for example, addresses that are separated by less than 100 microns, 10 microns, 1 micron, 500 nm, 100 nm, 10 nm or less. Alternatively or additionally, an array can have addresses that are separated by at least 10 nm, 100 nm, 500 nm, 1 micron, 5 microns, 10 microns, 50 microns, 100 microns or more. The addresses can each have an area of less than 1 square millimeter, 500 square microns, 100 square microns, 25 square microns, 1 square micron or less. An array can include at least about 1×10⁴, 1×10⁵, 1×10⁶, 1×10⁸, 1×10¹⁰, 1×10¹², or more addresses (e.g., addresses to which at least one analyte is attached).

Arrays can be made using methods known in the art such as those that deposit analytes at predefined addresses on a surface or those that contact an array surface with a plurality of analytes in fluid phase such that the analytes are randomly distributed to addresses in the array. Exemplary arrays and methods for making and using arrays are set forth, for example, in U.S. Pat. Nos. 11,203,612 or 11,505,796; or US Pat. App. Pub. No. US 2023/0167488 A1, each of which is incorporated herein by reference.

An array can have a size and complexity that is sufficient to accommodate a plurality of proteins such as those exemplified below. Arrays of other analytes or arrays of affinity reagents can also have size and complexity exemplified below for proteins. It will be understood that the pluralities of proteins set forth below need not be limited to array configurations.

A plurality of proteins, whether present in an array or other composition set forth herein, can be characterized in terms of total protein mass. The total mass of protein in a liter of plasma has been estimated to be 70 g and the total mass of protein in a human cell has been estimated to be between 100 μg and 500 pg depending upon cells type. See Wisniewski et al. Molecular & Cellular Proteomics 13:10.1074/mcp.M113.037309, 3497-3506 (2014), which is incorporated herein by reference. A plurality of proteins can include at least 1 μg, 10 pg, 100 μg, 1 ng, 10 ng, 100 ng, 1 ug, 10 ug, 100 ug, 1 mg, 10 mg, 100 mg or more protein by mass. Alternatively or additionally, a plurality of proteins may contain at most 100 mg, 10 mg, 1 mg, 100 ug, 10 ug, 1 ug, 100 ng, 10 ng, 1 ng, 100 pg, 10 pg, 1 pg or less protein by mass.

A plurality of proteins can include or be obtained from a proteomic sample. A proteomic sample can include substantially all proteins from a given source or a substantial fraction thereof. For example, a plurality of proteins may contain at least 60%, 75%, 90%, 95%, 99%, 99.9% or more of the total protein mass present in the source from which the sample was derived. Alternatively or additionally, a plurality of proteins may contain at most 99.9%, 99%, 95%, 90%, 75%, 60% or less of the total protein mass present in the source from which the sample was derived.

A plurality of proteins can be characterized in terms of total number of protein molecules. The total number of protein molecules in a Saccharomyces cerevisiae cell has been estimated to be about 42 million protein molecules. See Ho et al., Cell Systems (2018), DOI: 10.1016/j.cels.2017.12.004, which is incorporated herein by reference. A plurality of proteins used or included in a method, composition or apparatus set forth herein can include at least 1 protein molecule, 10 protein molecules, 100 protein molecules, 1×10⁴ protein molecules, 1×10⁶ protein molecules, 1×10⁸ protein molecules, 1×10¹⁰ protein molecules, 1 mole (6.02214076×10²³ molecules) of protein molecules, 10 moles of protein molecules, 100 moles of protein molecules or more. Alternatively or additionally, a plurality of proteins may contain at most 100 moles of protein molecules, 10 moles of protein molecules, 1 mole of protein molecules, 1×10¹⁰ protein molecules, 1×10⁸ protein molecules, 1×10⁶ protein molecules, 1×10⁴ protein molecules, 100 protein molecules, 10 protein molecules, 1 protein molecule or less.

A plurality of proteins can be characterized in terms of the variety of full-length amino acid sequences in the plurality. For example, the variety of full-length amino acid sequences in a plurality of proteins can be equated with the number of different protein-encoding genes in the source for the plurality of proteins. Whether or not the proteins are derived from a known genome or from any genome at all, the variety of full-length amino acid sequences can be counted independent of presence or absence of post translational modifications in the proteins. A human proteome is estimated to have about 20,000 different protein-encoding genes such that a plurality of proteins derived from a human can include up to about 20,000 different full-length amino acid sequences. See Aebersold et al., Nat. Chem. Biol. 14:206-214 (2018), which is incorporated herein by reference. Other genomes and proteomes in nature are known to be larger or smaller. A plurality of proteins used or included in a method, composition or apparatus set forth herein can have a complexity that includes substantially all different native-length amino acid sequences from a given source or a subfraction thereof. A proteome or subfraction can have a complexity of at least 2, 5, 10, 100, 1×10³, 1×10⁴, 2×10⁴, 3×10⁴ or more different native-length amino acid sequences. Alternatively or additionally, a proteome or subfraction can have a complexity that is at most 3×10⁴, 2×10⁴, 1×10⁴, 1×10³, 100, 10, 5, 2 or fewer different native-length amino acid sequences.

The diversity of a plurality of proteins can include at least one representative for substantially all proteins encoded by a source from which the plurality of proteins was derived or a substantial fraction thereof. For example, a plurality of proteins may contain at least one representative for at least 60%, 75%, 90%, 95%, 99%, 99.9% or more of the proteins encoded by a source from which the plurality of proteins was derived. Alternatively or additionally, a plurality of proteins may contain a representative for at most 99.9%, 99%, 95%, 90%, 75%, 60% or less of the proteins encoded by a source from which the plurality of proteins was derived.

A plurality of proteins can be characterized in terms of the variety of full-length amino acid sequences in the plurality including transcribed splice variants. The human proteome has been estimated to include about 70,000 different full-length amino acid sequences when splice variants are included. See Aebersold et al., Nat. Chem. Biol. 14:206-214 (2018), which is incorporated herein by reference. A plurality of proteins used or included in a method, composition or apparatus set forth herein can have a complexity of at least 2, 5, 10, 100, 1×10³, 1×10⁴, 7×10⁴, 1×10⁵, 1×10⁶ or more different full-length amino acid sequences. Alternatively or additionally, a plurality of proteins can have a complexity that is at most 1×10⁶, 1×10⁵, 7×10⁴, 1×10⁴, 1×10³, 100, 10, 5, 2 or fewer different full-length amino acid sequences.

A plurality of proteins can be characterized in terms of the variety of protein structures therein including, for example, different full-length amino acid sequences or different proteoforms among those sequences. Different molecular forms of proteins expressed from a given gene are considered to be different proteoforms. Proteoforms can differ, for example, due to differences in primary structure (e.g., shorter or longer amino acid sequences), different arrangement of domains (e.g., transcriptional splice variants), or different post translational modifications (e.g., presence or absence of phosphoryl, glycosyl, acetyl, or ubiquitin moieties). The human proteome is estimated to include hundreds of thousands of proteins when counting the different primary structures and proteoforms. See Aebersold et al., Nat. Chem. Biol. 14:206-214 (2018), which is incorporated herein by reference. A plurality of proteins used or included in a method, composition or apparatus set forth herein can have a complexity of at least 2, 5, 10, 100, 1×10³, 1×10⁴, 1×10⁵, 1×10⁶, 5×10⁶, 1×10⁷ or more different protein structures. Alternatively or additionally, a plurality of proteins can have a complexity that is at most 1×10⁷, 5×10⁶, 1×10⁶, 1×10⁵, 1×10⁴, 1×10³, 100, 10, 5, 2 or fewer different protein structures.

A plurality of proteins can be characterized in terms of the dynamic range for the different protein structures in the plurality. The dynamic range can be a measure of the range of abundance for all different protein structures in a plurality of proteins, the range of abundance for all different primary protein structures in a plurality of proteins, the range of abundance for all different full-length primary protein structures in a plurality of proteins, the range of abundance for all different full-length gene products in a plurality of proteins, the range of abundance for all different proteoforms expressed from a given gene, or the range of abundance for any other set of different proteins set forth herein. The dynamic range for all proteins in human plasma is estimated to span more than 10 orders of magnitude from albumin, the most abundant protein, to the rarest proteins that have been measured clinically. See Anderson and Anderson Mol Cell Proteomics 1:845-67 (2002), which is incorporated herein by reference. The dynamic range for plurality of proteins set forth herein can be a factor of at least 10, 100, 1×10³, 1×10⁴, 1×10⁶, 1×10⁸, 1×10¹⁰, or more. Alternatively or additionally, the dynamic range for plurality of proteins set forth herein can be a factor of at most 1×10¹⁰, 1×10⁸, 1×10⁶, 1×10⁴, 1×10³, 100, 10 or less.

A sample used herein, whether containing proteins or other analytes, need not be from a biological source and can instead be from an artificial source, such as a library from a combinatorial synthesis or a library from an in vitro synthesis that exploits biological components. An artificial sample can have a range of complexity similar to those set forth herein for proteomes. A method set forth herein can detect, identify or characterize some or all proteins in a proteome or other sample including, for example, at least about 1%, 5%, 10%, 25%, 50%, 75%, 90% or 99% of the proteins in the sample.

Any of a variety of affinity reagents can be used in a composition or method set forth herein. Particularly useful affinity reagents include, but are not limited to, antibodies whether full length or functional fragments thereof (e.g., Fab′ fragments, F(ab′) 2 fragments, single-chain variable fragments (scFv), di-scFv, tri-scFv, or microantibodies), or aptamers, affibodies, affilins, affimers, affitins, alphabodies, anticalins, avimers, miniproteins, DARPins, monobodies, nanoCLAMPs, lectins, or functional fragments thereof. The exemplified affinity reagents can be used individually. Alternatively, an affinity reagent set forth herein can be used as a paratope or moiety of an affinity reagent having a plurality of paratopes or moieties. For example, an affinity reagent set forth herein can provide one paratope (or a subset of paratopes) of an affinity reagent having a plurality of paratopes. In some configurations, a composition or method set forth herein can lack one or more of the affinity reagents set forth herein.

An antibody is a particularly useful affinity reagent for use in a composition or method set forth herein. The antibody can be any antigen-binding molecule or molecular complex having at least one complementarity determining region (CDR) that binds to a particular epitope with high affinity. An antibody can include four polypeptide chains: two heavy chains (HC1 and HC2) and two light chains (LC1 and LC2). HC1 and HC2 can be covalently connected by one, two or more disulfide bonds. HC1 can be covalently connected to LC1 by at least one disulfide bond. HC2 can be covalently connected to LC2 by at least one disulfide bond. Each heavy chain can include a heavy chain variable region (V_{H}) and a heavy chain constant region (C_{H}). The heavy chain constant region can include three domains, C_{H1}, C_{H2} and C_{H3}. Each light chain can include a light chain variable region (V_{L}) and a light chain constant region (C_{L}). The V_{H} and V_{L} regions can further include regions of hypervariability, termed complementarity determining regions (CDRs), interspersed with regions that are more conserved, termed framework regions (FR). Each V_{H} and V_{L} can include three CDRs and four FRs, arranged from amino-terminus to carboxy-terminus in the following order: FR1, CDR1, FR2, CDR2, FR3, CDR3, FR4.

An antibody can include all elements of a full-length antibody, such as those enumerated above. However, an antibody need not be full length and functional fragments can be particularly useful for many applications. The term “antibody” as used herein encompasses full length antibodies and functional fragments thereof. A functional fragment can be naturally occurring, enzymatically obtainable, synthetic, or genetically engineered. An antibody can be obtained using any suitable technique such as proteolytic digestion or recombinant genetic engineering techniques involving the manipulation and expression of DNA encoding one or more antibody domains. Such DNA is readily available, for example, from commercial sources, DNA libraries (e.g., phage-antibody libraries), or can be synthesized. The DNA may be manipulated chemically or by using molecular biology techniques, for example, to arrange one or more variable and/or constant domains into a suitable configuration, or to introduce codons, introduce cysteine residues, remove cysteine residues, modify, add or delete other amino acids, etc.

A functional fragment of an antibody can include any fragment that is capable of binding to an epitope with a detectable affinity, such as a Fab, Fab′, F(ab′)₂, Fd, Fv, dAb, single-chain variable (scFv), di-scFv, tri-scFv, microantibody, or minimal recognition unit consisting of the amino acid residues that mimic the hypervariable region of an antibody (e.g., an isolated complementarity determining region (CDR) such as a CDR1, CDR2 or CDR3 peptide). Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g., monovalent nanobodies, bivalent nanobodies, etc.), small modular immunopharmaceuticals (SMIPs), and shark variable IgNAR domains can also be useful.

A functional fragment of an antibody will typically include at least one variable domain. The variable domain may have any of a variety of sizes or amino acid compositions and will generally include at least one CDR which is adjacent to or in frame with one or more framework sequences. For antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, a functional fragment of an antibody may contain a monomeric V_{H} or V_{L} domain.

In particular configurations, a functional fragment of an antibody contains at least one variable domain covalently connected to at least one constant domain. Non-limiting, exemplary configurations of variable and constant domains that may be found within an antigen-binding fragment of an antibody of the present disclosure include: (i) V_{H}-C_{H1}; (ii) V_{H}-C_{H2}; (iii) V_{H}-C_{H3}; (iv) V_{H}—C_{H1}-C_{H2}; (v) V_{H}—C_{H1}-C_{H2}-C_{H3}; (vi) V_{H}-C_{H2}-C_{H3}; (vi) V_{H}-C_{L}; (viii) V_{L}-C_{H1}; (ix) V_{L}-C_{H2}; (x) V_{L}-C_{H3}; (xi) V_{L}-C_{H2}; (xii) V_{L}-C_{H1}-C_{H2}-C_{H3}; (xiii) V_{L}-C_{H2}-C_{H3}; and (xiv) V_{L}-C_{L}. In any configuration of variable and constant domains, including any of the exemplary configurations listed above, the variable and constant domains may be either directly connected to one another or may be connected by a full or partial hinge or linker region. A hinge region may consist of at least 2 (e.g., at least 5, 10, 15, 20, 40, 60 or more) amino acids which result in a flexible or semi-flexible linkage between adjacent variable and/or constant domains in a single polypeptide molecule. Moreover, an antigen-binding fragment of an antibody may include a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

An affinity reagent of the present disclosure can include one or more paratopes. For example, an affinity reagent can include at least 1, 2, 3, 4, 5, 10, 15, 20, 25 or more paratopes. Alternatively or additionally, an affinity reagent can include at most 25, 20, 15, 10, 5, 4, 3, 2, or 1 paratopes. Multiple paratopes that are present in an affinity reagent can have the same function or different functions compared to each other. For example, the multiple paratopes can each have affinity for the same epitope or same set of epitopes. In some cases, the strength and/or specificity of the affinity can be substantially the same, for example, in cases where the paratopes have the same structure. In other cases, the strength and/or specificity of two or more paratopes for a given epitope or set of epitopes can overlap despite some differences in the functional or structural characteristics of the two or more paratopes. In some configurations, multiple paratopes of a given affinity reagent can have affinity for different epitopes. This can be the case, whether the different epitopes are found in the same analyte (e.g., two different amino acid trimer epitopes present in a given protein analyte) or in different analytes (e.g., a first trimer epitope being found in a first protein that lacks a second trimer epitope, and a second trimer epitope being found in a second protein that lacks the first trimer epitope).

In some configurations of the methods, compositions or systems set forth herein, two or more affinity reagents can be present as moieties of a multimeric affinity reagent. For example, an affinity reagent can include two or more affinity moieties, wherein the affinity moieties are selected from an affinity reagent set forth herein or known in the art. Two or more affinity moieties can be combined via attachment to any of a variety of retaining components including, for example, a structured nucleic acid particle (SNAP), nucleic acid origami, artificial polymer or particle. Other particles (e.g., particles composed of solid support material set forth herein or known in the art) or substances, such as those set forth herein in the context of mediating attachment of a protein to a solid support, can be used as retaining components for affinity reagents. The presence of multiple affinity moieties in an affinity reagent can provide increased binding strength, for example, due to increased avidity as compared to any one of the affinity moieties when used as an individual affinity reagent. In some configurations an affinity reagent can include at least 2, 3, 4, 5, 6, 7, 8, 9, 10, 15, 20 or more affinity moieties. Alternatively or additionally, an affinity reagent can include at most 20, 15, 10, 9, 8, 7, 6, 5, 4, 3, 2 or fewer affinity moieties. It may be convenient to characterize an affinity reagent with respect to the number of paratopes it includes. For example, an affinity reagent can include at least 2, 3, 4, 5, 6, 7, 8, 9, 10, 15, 20 or more paratopes. Alternatively or additionally, an affinity reagent can include at most 20, 15, 10, 9, 8, 7, 6, 5, 4, 3, 2 or fewer paratopes. Typically, the affinity moieties or paratopes that are present in an affinity reagent will be structurally identical. For example, a plurality of antibodies in an affinity reagent can have identical amino acid sequences.

Whether or not a plurality of affinity moieties or a plurality of paratopes include structurally identical members, the members can recognize the same epitopes. In some cases, the members can recognize the same epitopes with substantially the same binding strength. It will be understood, however, that in some cases an affinity reagent can include two or more affinity moieties having different structures and different binding affinities compared to each other. Similarly, an affinity reagent can include two or more paratopes having different structures and different binding affinities compared to each other.

An affinity reagent of the present disclosure can include one or more labels. For example, an affinity reagent can include at least 1, 2, 3, 4, 5, 10, 15, 20 or more labels. Alternatively or additionally, an affinity reagent can include at most 20, 15, 10, 5, 4, 3, 2, or 1 labels. In some configurations, an affinity reagent can be attached to one or more labels. For example, an affinity reagent can include a particle (e.g., structured nucleic acid particle) that is attached to at least one paratope and further attached to at least one label. Methods and compositions set forth herein in the context of labels although exemplified for affinity reagents can be extended to other molecules such as analytes (e.g., proteins).

Multiple labels that are present in an affinity reagent can have the same structure as each other or they can differ structurally from each other. Optionally, multiple labels can have different detectable characteristics. For example, two or more optical labels can differ in terms of luminescence lifetime, luminescence polarity, extinction coefficient, quantum yield of luminescence, spectral region for absorbance, spectral region for excitation or spectral region for emission. Alternatively, two or more optical labels can have overlapping detectable characteristics, for example, in terms of luminescence lifetime, luminescence polarity, extinction coefficient, quantum yield of luminescence, spectral region for absorbance, spectral region for excitation or spectral region for emission. This can result from the two or more optical labels having the same structure, but in some cases two or more labels can have the same or overlapping detection properties despite having different structures.

A label of a molecule, such as an affinity reagent, can be exogenous or endogenous to the molecule. Exogenous labels can be attached to an affinity reagent or other molecule using methods and compositions known in the art such as artificial linkers or genetic fusions. A wide variety of labels can be used in a composition or method set forth herein including, for example, optically detectable labels, such as luminophores (e.g., fluorophores), enzymes (e.g., enzymes which catalyze reactions with colored reagents or products), electrochemical labels (e.g., highly charged moieties). Magnetic contrast imaging moieties can be used such as gadolinium-diethylenetriaminepentacetate (Gd-DTPA), gadolinium-dodecane tetraacetic acid (Gd-DOTA) or others used for magnetic resonance techniques. Moieties that are detected through subsequent processing, such as nucleic acid barcode labels, can also be useful. Nucleic acids can be detected or identified via nucleic acid amplification, sequencing or hybridization assays.

Any of a variety of luminophores may be used herein. Luminophores may include labels that emit in the ultraviolet, visible, or infrared region of the spectrum. In some cases, the luminophore may be selected from the group consisting of FITC, Alexa Fluor 350, Alexa Fluor 405, Alexa Fluor 488, Alexa Fluor 532, Alexa Fluor 546, Alexa Fluor 555, Alexa Fluor 568, Alexa Fluor 594, Alexa Fluor 647, Alexa Fluor 680, Alexa Fluor 750, Pacific Blue, Coumarin, BODIPY μL, Pacific Green, Oregon Green, Cy3, Cy5, Pacific Orange, TRITC, Texas Red, R-Phycoerythrin, Allophcocyanin (APC). In some cases, the label may be an Atto dye, for example Atto 390, Atto 425, Atto 430, Atto 465, Atto 488, Atto 490, Atto 495, Atto 514, Atto 520, Atto 532, Atto 540, Atto 550, Atto 565, Atto 580, Atto 590, Atto 594, Atto 610, Atto 611, Atto 612, Atto 620, Atto 633, Atto 635, Atto 647, Atto 655, Atto 680, Atto 700, Atto 725, Atto 740, Atto MB2, Atto Oxa12, Atto Rho101, Atto Rho12, Atto Rho13, Atto Rho14, Atto Rho3B, Atto Rho6G, or Atto Thio12. In some cases, the luminophore may be a fluorescent protein such as green fluorescent protein (GFP), cyan fluorescent protein (CFP), red fluorescent protein (RFP), blue fluorescent protein (BFP), orange fluorescent protein (OFP), and yellow fluorescent protein (YFP). A wide range of effective luminophores are commercially available, for example, from the Molecular Probes division of ThermoFisher Scientific and/or generally described in the Molecular Probes Handbook (11th Edition) which is hereby incorporated by reference. Label components may also include intercalation dyes, such as ethidium bromide, propidium bromide, crystal violet, 4′,6-diamidino-2-phenylindole (DAPI), 7-aminoactinomycin D (7-AAD), Hoescht 33258, Hoescht 33342, Hoescht 34580, YOYO-1, DiYO-1, TOTO-1, DiTO-1, or combinations thereof.

Optionally, an affinity reagent can be attached to a solid support or particle. A particularly useful particle is a structured nucleic acid particle (e.g., nucleic acid origami), for example, having structural or functional characteristics set forth herein in the context of attachment to proteins and other analytes. An analyte, affinity reagent, docker, tether, label or other moiety can be attached to a nucleic acid origami via a scaffold component or oligonucleotide component. For example, the scaffold or oligonucleotide can include a nucleotide analog that forms a covalent or non-covalent bond with the attached moiety.

Any of a variety of chemistries can be used to attach an analyte, affinity reagent, docker, tether or other moiety to a solid support or particle (e.g., structured nucleic acid particle). The attachment can be covalent. Exemplary covalent chemistries include, but are not limited to, click chemistries or chemistries set forth in U.S. Pat. Nos. 11,203,612 or 11,505,796; or US Pat. App. Pub. No. 2022/0162684 A1, each of which is incorporated herein by reference. Another example is the Spy Tag/SpyCatcher system (See, Zakeri et al. Proceedings Nat'l Acad. Sciences USA. 109 (12): E690-7 (2012)). In this system, a 13 amino acid tag polypeptide (Spy Tag) forms a first coupling handle, with a 12.3 kDa protein (Spy-Catcher) forming the other coupling handle. The SpyCatcher can function by irreversibly bonding to a SpyTag through an isopeptide bond. Any of a variety of non-covalent bonds can be used to attach an analyte, affinity reagent or other moiety to a solid support or particle (e.g., structured nucleic acid particle). Receptors and their ligands can be particularly useful. Examples include, but are not limited to, antibodies, antigens, (strept) avidin (or analogs thereof), biotin (or analogs thereof), affibodies, affilins, affimers, affitins, alphabodies, anticalins, avimers, DARPins, monobodies, nanoCLAMPs, nucleic acids, peptide nucleic acids, polypeptides, nucleic acid aptamers, protein aptamers, lectins (or analogs thereof), carbohydrates or functional fragments thereof. Complementary nucleic acids can be used to non-covalently attach a functional moiety to a solid support or particle (e.g., structured nucleic acid particle). Useful nucleic acids can have complementary sequences that are at least 5, 10, 15, 20, 25, 30, 40, 50, 60, 70, 80, 90 or more nucleotides in length. Alternatively or additionally, nucleic acids can have complementary sequences that are at most 90, 80, 70, 60, 50, 40, 30, 25, 20, 15, 10, 5 or fewer nucleotides in length. Reagents and techniques that can be used to non-covalently attach an affinity reagent or other moiety to a particle (e.g., structured nucleic acid particle) are set forth in U.S. Pat. Nos. 11,203,612 or 11,505,796; or US Pat. App. Pub. No. 2022/0162684 A1, each of which is incorporated herein by reference.

A structured nucleic acid particle that is made or used in a method set forth herein can be suspended in a fluid, immobilized on a solid support, or immobilized in another material such as a gel or solid support material. This can be the case before, during or after being attached to a moiety of interest, such as a tether, docker, linker, label, analyte or affinity moiety. For example, a population of structured nucleic acid particles can be colloidal for some, or all steps of a method set forth herein. Alternatively, a population of structured nucleic acid particles can be immobilized in, or on, a solid support for some, or all steps of a method set forth herein. For example, analytes or affinity reagents can be attached to addresses (or other unique identifiers) of an array via structured nucleic acid molecules.

A particle need not be composed primarily of nucleic acid and, in some cases, may be devoid of nucleic acids. For example, an analyte, affinity reagent, label, linker, docker or tether can be attached to an artificial polymer that is configured to form a particle. In other examples, a particle can be composed of a solid support material, such as those set forth herein, glass, silicon, silica, carbon, cellulose, polyethylene glycol (PEG), upconversion nanocrystal, or a quantum dot.

Delivery and removal of fluids will be set forth below in the context of fluid-phase affinity reagents and immobilized analytes (e.g., proteins attached to addresses of an array). This is done for ease of explanation. It will be understood that similar compositions and methods can be used for fluid-phase analytes and immobilized affinity reagents.

A method of the present disclosure can include a step of contacting an array of analytes with affinity reagents. Typically, the affinity reagents are in a fluid phase that is delivered to the array and the analytes are immobilized to addresses in the array. Any of a variety of fluidics techniques or apparatus can be employed for delivery of the fluid phase. For example, a fluid phase can be provided in a tube, pipette tip, syringe or the like and delivered via fluid displacement. In some cases, the array can be present in the lumen of a flow cell or other vessel having an inlet and outlet, and a fluid phase can enter the lumen via displacement of fluid through the inlet. The fluid phase can also exit the flow cell, at least in part, via displacement through the outlet. Other methods can be employed to deliver fluid to an array such as dipping the array into a vessel containing the fluid, flowing fluid through a nozzle to the array, or spin coating fluid on the surface of the array.

A fluid phase that is in contact with an array can contain affinity reagents in a desired quantity as measured, for example, by concentration, mass or number of affinity reagent molecules. For example, the concentration of affinity reagents in contact with an array can be at least 10 nM, 100 nM, 1 μM, 10 μM, 100 μM, 1 mM, 10 mM, 100 mM or higher. Alternatively or additionally, the concentration of affinity reagents in contact with an array can be at most 100 mM, 10 mM, 1 mM, 100 μM, 10 μM, 1 μM, 100 nM, 10 nM or lower. The quantity of affinity reagents in a fluid phase is typically greater than the quantity of analytes in an array to which the fluid is in contact. For example, a fluid phase can contain a number of affinity reagent molecules that is at least 1.5×, 2×, 5×, 10×, 50×, 100×, 1000×, 10000× or greater relative to the number of analytes in an array to which the fluid phase is in contact. However, in some cases the fluid can contain an equivalent or lower quantity of affinity reagents compared to the quantity of analytes in the array. For example, a fluid phase can contain a number of affinity reagent molecules that is at most 1×, 0.5×, 0.1×, 0.01× or less relative to the number of analytes in an array to which the fluid phase is in contact.

In particular configurations of the methods set forth herein, a fluid phase is contacted with an array and the affinity reagents are present at a first concentration in the fluid phase. A plurality of the affinity reagents can then be removed, thereby retaining a fraction of the affinity reagents in contact with the array. Thus, the quantity of affinity reagents in contact with the array is reduced. The fraction of affinity reagents can be contained in a fluid and can be present at a second concentration that is higher, equal or lower compared to the first concentration. For example, the concentration of affinity reagents can be reduced for the retained fraction if the fluid volume is equivalent before and after removal of the plurality of affinity reagents. Alternatively, a higher concentration of affinity reagents can result for the retained fraction if the volume of fluid for the retained fraction is substantially lower after removal of the plurality of affinity reagents.

Removal of fluid from an array can be carried out using any of a variety of fluidics techniques or apparatus including, but not limited to, those set forth above in the context of delivering fluids to an array. For example, at least a portion of the fluid in contact with an array can be removed by fluid displacement. The fluid can be displaced with an equivalent volume of liquid or gas. Displacement with gas can be particularly useful for reducing the volume of fluid in contact with the array. Liquid displacement can be performed to result in replacement of at least a portion of a first fluid phase with a second fluid phase. Displacement with a second fluid phase can be useful for adding or replacing reagents, cofactors, salts, or other fluid components. For example, the second fluid can contain affinity reagents of the same or different type as the first fluid that is displaced. Other methods that can be employed to remove affinity reagents from an array or vessel include, for example, precipitation of the affinity reagents, solid-phase extraction of the affinity reagents, liquid-liquid extraction of the affinity reagents, capture of the affinity reagents via a solid-phase receptor or ligand that binds to the affinity reagents, or dialysis of the affinity reagents.

Optionally, a step of removing a plurality of the affinity reagents from contact with an array can be carried out after allowing affinity reagents to bind to analytes in the array. For example, an array can be in contact with a fluid that contains affinity reagents and, after allowing affinity reagents in the fluid to bind to analytes in the array, a plurality of the affinity reagents can be removed. Binding can be allowed to occur under passive diffusion conditions. Alternatively, binding can be facilitated by active transport, for example, by application of a stimulus, such as a variably applied stimulus. For example, an affinity reagent can include charged moieties and an electric field or electrophoretic force can be applied to actively transport the affinity reagents to addresses of an array to which analytes are attached. The electric field or electrophoretic force can be reversed or otherwise varied to reduce or reverse attraction of the affinity reagent to the array. In another example, an affinity reagent can include magnetic or paramagnetic moieties and a magnetic field or magnetic force can be applied to actively transport the affinity reagents to addresses of an array to which analytes are attached. Again, the magnetic field or magnetic force can be variably applied. In some configurations of the methods set forth herein, active transport is not employed during one or more binding step. For example, a method can be configured to include steps of (a) contacting an array of analytes with affinity reagents in fluid phase; (b) allowing affinity reagents in the fluid phase to bind to analytes in the array without employing active transport (e.g., using passive diffusion of affinity reagents to the array); (c) removing a plurality of the affinity reagents from contact with the array, thereby retaining a fraction of the affinity reagents in contact with the array; (d) attracting the fraction of affinity reagents to the array via active transport or application of a force; and (e) detecting binding of affinity reagents in the fraction to analytes attached at addresses in the array.

Detection techniques and apparatus will be set forth below in the context of fluid-phase affinity reagents and immobilized analytes (e.g., proteins attached to addresses of an array). This is done for ease of explanation. It will be understood that similar compositions and methods can be used for fluid-phase analytes and immobilized affinity reagents.

A method of the present disclosure can include a step of detecting binding of affinity reagents to analytes in an array. Binding of affinity reagents with analytes can be detected using any of a variety of techniques that are appropriate to the assay components used. For example, an affinity reagent can be detected at a unique identifier (e.g., address) in an array by acquiring a signal from a label attached to the affinity reagent when bound to an analyte at the unique identifier. In some configurations, a complex between an affinity reagent and analyte need not be directly detected, for example, in formats where a nucleic acid tag or other moiety is created or modified as a result of binding. Optical detection techniques such as luminescent intensity detection, luminescence lifetime detection, luminescence polarization detection, or surface plasmon resonance detection can be useful. Other detection techniques include, but are not limited to, electronic detection such as techniques that utilize a field-effect transistor (FET), ion-sensitive FET, or chemically-sensitive FET. Exemplary detection techniques and apparatus are set forth in U.S. Pat. No. 10,473,654 or US Pat. App. Pub. No. 2022/0162684 A1, each of which is incorporated herein by reference. A detection technique used in a method set forth herein can be configured to resolve addresses (or other unique identifiers) of an array. For example, a detection technique can be configured for single-molecule resolution of analytes.

In some configurations of the methods set forth herein, affinity reagents that are not bound to analytes at addresses of an array are removed from contact with the array prior to detecting affinity reagents that are bound to the addresses. Removal of non-bound affinity reagents can provide the advantage of reducing unwanted background when detecting bound affinity reagents. This can be particularly helpful when affinity reagents have labels that produce detectable signals in both the bound and non-bound state. One option to avoid or reduce unwanted background signals is to remove non-bound affinity reagents. However, this approach may change the dynamics of binding between affinity reagent and analyte unless remediation is performed, such as detecting bound affinity reagents on a time scale that is faster than the rate at which binding equilibrium is substantially shifted. Another option for reducing unwanted signal from non-bound affinity reagents is to use a detection apparatus that is capable of spatially confined collection of signals. For example, total internal reflectance can be used to collect signals for affinity reagents bound at or near an array surface while rejecting signals produced further away from the surface of the array. Another example is the use of waveguides such as zero mode waveguides for spatially resolved excitation of labels and/or spatially resolved acquisition of emission signals from excited labels. Other examples of spatially resolved detection of labeled affinity reagents include, but are not limited to, detection of luminescent moieties using confocal fluorescence microscopy, detection of affinity reagents using surface plasmon resonance (SPR) or detection of charged moieties using field effect transistors.

Any of a variety of proximity-based detection techniques can be used to detect binding of affinity reagents to analytes. A particularly useful method is detection of energy transfer between a donor and acceptor. For example, Förster resonance energy transfer (FRET) can be detected between a first luminophore (i.e. donor) and second luminophore (i.e. acceptor). Energy transfer techniques utilize a donor that is excited by an energy source, such as a laser or other radiation source, and an acceptor that receives energy from the donor to, in turn, produce a detectable signal, such as a luminescence signal. The efficiency of energy transfer is inversely proportional to distance between donor and acceptor, making energy transfer sensitive to proximity on a scale that is comparable to the distances between affinity reagents and the analytes to which they bind. Alternatively, methods described below and shown in FIGS. 15-17 may be useful for providing an amplified signal without rinsing unbound affinity reagents from an array.

FIG. 2 shows a cross section of a region of a flow cell having two protein analytes (white globules) immobilized on the lower surface and a fluid phase containing affinity reagents (Y shapes), wherein the affinity reagents are attached to a first luminophore (open circle) and the protein analytes are attached to a second luminophore (closed circle). The first luminophore and second luminophore are capable of FRET when in proximity to each other, with either being the donor and the other being the acceptor. Using appropriate optics including, for example, a filter to selectively collect emission from the acceptor and to reject emission from the donor, binding between the affinity reagent and protein can be detected in the presence of unbound affinity reagent. Thus, detection can occur while equilibrium is maintained due to the concentration of free affinity reagent in solution phase; however, emission produced by free affinity reagent does not produce substantial background interference. Returning to FIG. 2, FRET can be detected due to binding of the affinity reagent to the protein on the left and absence of signal from the address for the protein on the right is indicative of the affinity reagent not recognizing the protein nor binding to the protein. It will be understood that FRET can be used similarly if the protein is in fluid phase and the affinity reagent is immobilized on the lower surface of the flow cell. The use of FRET in the context of FIG. 2 is exemplary and it will be understood that other proximity-based detection techniques can be used instead.

Other proximity-based detection techniques that can be used in a method set forth herein include those that employ various pairs of components that produce unique signals when in proximity to each other compared to when they are apart. For example, a luminophore component and quencher component can be used to detect proximity via quenching of signal that would otherwise be produced by the luminophore. A two component luminophore systems can also be useful. For example, fluorogen-activated-proteins utilize a protein component and fluorogen component to produce unique fluorescent signal when in proximity to each other (see, for example, Gallo, Bioconjugate Chem. 31:16-27 (2020). In another example, an intercalating dye component and nucleic acid component can produce a unique optical signal when in proximity to each other. Also useful are detection components used in split protein assays such as two parts of an enzyme that are uniquely functional when in proximity to each other. Exemplary enzymes that can be split into components for use in proximity-based detection include, but are not limited to beta lactamase, dihydrofolate reductase, focal adhesion kinase, Green Fluorescent Protein (and variants thereof), horseradish peroxidase, and luciferase.

An analyte or a unique identifier (e.g., address) to which an analyte is attached can be co-localized with a first component of a proximity-based detection pair (e.g., a FRET donor), for example, via covalent or non-covalent attachment; and an affinity reagent can be co-localized with a second component of the proximity-based detection pair (e.g., a FRET acceptor), for example, via covalent or non-covalent attachment. The opposite placement of donor and acceptor is also possible (i.e. the analyte or its unique identifier can be co-localized with the acceptor, and the affinity reagent can be co-localized with the donor). Using a pair of proximity-based detection components (e.g., donor and acceptor) allows interaction between affinity reagent and analyte to be distinguished even in the presence of unbound affinity reagent.

Proximity-based detection can be facilitated by dockers and tethers. An analyte used in a composition or method of the present disclosure can have a docker and the docker can be attached to a first component of a proximity-based detection pair (e.g., a FRET donor or acceptor used for energy transfer). An affinity reagent used in a composition or method of the present disclosure can have a tether and the tether can be attached to a second component of a proximity-based detection pair (e.g., a FRET donor or acceptor used for energy transfer). The docker and tether can be configured to position the components of the pair in proximity to each other when the affinity reagent is bound to the analyte. As such, the docker and tether can facilitate proximity-based detection (e.g. energy transfer) and, thus, facilitate detection of binding between the affinity reagent and analyte.

FIG. 3A shows a diagram of a protein (white globule) attached to a surface, wherein the protein is co-localized with a first luminophore (closed circle), and an affinity reagent (Y-shape) that is bound to the protein, wherein the affinity reagent is co-localized with a second luminophore (open circle). The first luminophore is co-localized with the protein via a nucleic acid docker that attaches the luminophore to the address where the protein resides. The second luminophore is co-localized with the affinity reagent via a nucleic acid tether that attaches the luminophore to the affinity reagent. In this example, short regions of the docker and tether are complementary to each other and function to position the first and second luminophores for FRET. It will be understood that FRET can be used similarly to FIG. 3A if the protein is in fluid phase and the affinity reagent is immobilized on the lower surface of the flow cell.

FIG. 3B shows a diagram of a protein (white globule) attached to a surface, wherein the protein is co-localized with a first luminophore (closed circle), and an affinity reagent (Y-shape) that is bound to the protein, wherein the affinity reagent is co-localized with a second luminophore (open circle). The first luminophore is co-localized with the protein via a nucleic acid docker that attaches the luminophore to the address where the protein resides. The second luminophore is co-localized with the affinity reagent via a nucleic acid tether that attaches the luminophore to the affinity reagent. In this example, the docker and tether are not complementary to each other, but are complementary to adjacent regions of a splint oligonucleotide. Hybridization of the docker and tether to the splint oligonucleotide positions the first and second luminophores for FRET. An advantage of using a splint oligonucleotide is that modifications can be made to the strength and timing of the interaction that brings the luminophores into proximity for FRET, for example, by adjusting the concentration of the splint oligonucleotide independently from the concentration of the affinity reagent. Thus, the concentration of affinity reagent can be maintained at a level that facilitates binding of the affinity reagent to the protein and changes can be made to the concentration of the splint oligonucleotide to reduce, or even minimize, the impact of docker-tether-splint oligonucleotide interactions on the apparent binding affinity of the affinity reagent for the protein. Similarly, the splint oligonucleotide can be added after formation of a binding complex between the affinity reagent and the protein and, optionally, after removal of unbound affinity reagents, thereby reducing the impact of the docker-tether-splint oligonucleotide interactions on the apparent binding affinity of the affinity reagent for the protein. It will be understood that FRET can be used similarly to the example of FIG. 3B if the protein is in fluid phase and the affinity reagent is immobilized on the lower surface of the flow cell.

Any of a variety of molecules or chemical moieties can be used as dockers or tethers. Polymers can be particularly useful and can be selected for use based on a variety of characteristics such as length, flexibility, chemical reactivity or inertness to various chemicals. A docker or tether can have a composition exemplified herein in the context of linkers. A particularly useful docker or tether is a nucleic acid. The nucleic acid can be single-stranded or double-stranded. Single-stranded nucleic acids are typically more flexible whereas double-stranded nucleic acids provide relative rigidity. Optionally, a docker strand can have a nucleotide sequence that complements a nucleotide sequences of a tether strand. Similarly, a tether strand can have a nucleotide sequence that complements a nucleotide sequences of a docker strand. The length of a complementary region formed between a docker and tether can be at least 2, 3, 4, 5, 6, 7, 8, 9, 10, 15 or more base pairs. Alternatively or additionally, the complementary region can be at most 15, 10, 9, 8, 7, 6, 5, 4, 3, or 2 base pairs. The complementary region can include A:T basepairs and G:C basepairs, only G:C basepairs, at most 90% G:C basepairs, at most 80% G:C basepairs; at most 70% G:C basepairs, at most 60% G:C basepairs, at most 50% G:C basepairs, at most 40% G:C basepairs, at most 30% G:C basepairs, at most 20% G:C basepairs, at most 10% G:C basepairs, only A:T basepairs, at most 90% A:T basepairs, at most 80% A:T basepairs; at most 70% A:T basepairs, at most 60% A:T basepairs, at most 50% A:T basepairs, at most 40% A:T basepairs, at most 30% A:T basepairs, at most 20% A:T basepairs, or at most 10% A:T basepairs. In some configurations of the methods and compositions set forth herein a docker and tether are not substantially complementary with each other.

A docker can be co-localized with an analyte via covalent and/or non-covalent attachment of the docker to the analyte. Exemplary attachment chemistries include those set forth herein in the context of attaching analytes and affinity reagents to solid supports and particles. In some configurations, a docker can be attached to a particle (e.g. structured nucleic acid particle) or solid support to which an analyte is attached. Optionally, one or more dockers can be co-localized with an analyte via attachment to an address or other unique identifier to which the analyte is attached.

An analyte can be co-localized with a single docker or, alternatively, with a plurality of dockers. For example, an analyte can be co-localized with at least 2, 3, 4, 5, 6, 7, 8, 9, 10 or more dockers. Alternatively or additionally, an analyte can be co-localized with at most 10, 9, 8, 7, 6, 5, 4, 3, 2 or fewer dockers. The dockers can be substantially identical to each other. Alternatively, a plurality of dockers can include dockers that differ from each other. In some cases, the different dockers will have different lengths or different nucleotide sequences. In some configurations, an analyte and the docker with which it is co-localized will have binding characteristics that are orthogonal to each other. As such, a paratope of an affinity reagent that recognizes or binds to the analyte will not recognize or bind to the docker, and a tether will not recognize or bind to the analyte.

An array of analytes can include a plurality of unique identifiers (e.g. addresses) each of which is co-localized with a different analyte. For example, the unique identifiers in an array can each be attached to a different analyte, such as a different protein. Each unique identifier in an array can also be attached to at least one docker. Optionally, the array utilizes universal dockers, whereby the dockers are substantially the same for some or all unique identifiers in the array. As such, each unique identifier in an array can be co-localized with an analyte that differs from analytes at other unique identifiers in the array and a universal docker that is the same as universal dockers at other unique identifiers in the array. In an exemplary configuration, addresses in an array are each attached to a different protein selected from a protein sample, and the universal docker that is attached to each of the addresses is the same. Accordingly, individual addresses can be distinguished based upon the unique structure of the attached analyte but the universal docker provides a structure that is universal across the addresses.

In some configurations, an array can include indexed dockers, wherein unique identifiers in one subset are distinguishable from unique identifiers in another subset based on detectable properties of the indexed dockers with which they are respectively co-localized. Accordingly, an array can include at least 1, 2, 3, 4, 5, 10 or more subsets of indexed dockers, wherein each subset of indexed dockers is co-localized with a respective subset of unique identifiers in the array. An array need not use universal nor indexed dockers. For example, individual addresses (or other unique identifiers) in an array can be attached to unique dockers, wherein the addresses are distinguishable based on the structure of the attached docker.

A tether can be co-localized with an affinity reagent via covalent and/or non-covalent attachment of the tether to the affinity reagent. Exemplary attachment chemistries include those set forth herein in the context of attaching analytes and affinity reagents to solid supports and particles. In some configurations, a tether can be attached to a particle (e.g. structured nucleic acid particle) or solid support to which an affinity reagent is attached. For example, one or more tethers can be co-localized with an affinity reagent via attachment to a structured nucleic acid particle to which the affinity reagent is attached.

An affinity reagent can be co-localized with a plurality of tethers. For example, an affinity reagent can be co-localized with at least 2, 3, 4, 5, 6, 7, 8, 9, 10, or more tethers. Alternatively or additionally, an affinity reagent can be co-localized with at most 10, 9, 8, 7, 6, 5, 4, 3, 2 or fewer tethers. The tethers can be substantially identical to each other. Alternatively, a plurality of tethers can include tethers that differ from each other. In some cases, the different tethers will have different lengths or different nucleotide sequences. In some configurations, an affinity reagent and the tether with which it is co-localized will have orthogonal binding recognition. As such, an analyte that recognizes or binds to a paratope of the affinity reagent will not recognize or bind to the tether, and a docker will not recognize or bind to the paratope.

Optionally, an analyte, affinity reagent, docker, tether, label or other moiety can be attached to a solid support or particle. A particularly useful particle is a structured nucleic acid particle (e.g. nucleic acid origami), for example, having structural or functional characteristics set forth herein in the context of attachment to proteins and other analytes. An analyte, affinity reagent, docker, tether, label or other moiety can be attached to a nucleic acid origami via a scaffold component or oligonucleotide component. For example, the scaffold or oligonucleotide can include a nucleotide analog that forms a covalent or non-covalent bond.

Any of a variety of chemistries can be used to attach an analyte, affinity reagent, docker, tether or other moiety to a solid support or particle (e.g. structured nucleic acid particle). The attachment can be covalent. Exemplary covalent chemistries include, but are not limited to, click chemistries or chemistries set forth in U.S. Pat. Nos. 11,203,612 or 11,505,796; or US Pat. App. Pub. No. 2022/0162684 A1, each of which is incorporated herein by reference. Another example is the Spy Tag/SpyCatcher system (See, Zakeri et al. Proceedings Nat'l Acad. Sciences USA. 109 (12): E690-7 (2012)). In this system, a 13 amino acid tag polypeptide (Spy Tag) forms a first coupling handle, with a 12.3 kDa protein (Spy-Catcher) forming the other coupling handle. The SpyCatcher can function by irreversibly bonding to a SpyTag through an isopeptide bond. Any of a variety of non-covalent bonds can be used to attach an analyte, affinity reagent, docker, tether or other moiety to a solid support or particle (e.g., structured nucleic acid particle). Receptors and their ligands can be particularly useful. Examples include, but are not limited to, antibodies, antigens, (strept) avidin (or analogs thereof), biotin (or analogs thereof), affibodies, affilins, affimers, affitins, alphabodies, anticalins, avimers, DARPins, monobodies, nanoCLAMPs, nucleic acids, peptide nucleic acids, polypeptides, nucleic acid aptamers, protein aptamers, lectins (or analogs thereof), carbohydrates or functional fragments thereof. Complementary nucleic acids can be used to non-covalently attach a functional moiety to a solid support or particle (e.g. structured nucleic acid particle). Useful nucleic acids can have complementary sequences that are at least 5, 10, 15, 20, 25, 30, 40, 50, 60, 70, 80, 90 or more nucleotides in length. Alternatively or additionally, nucleic acids can have complementary sequences that are at most 90, 80, 70, 60, 50, 40, 30, 25, 20, 15, 10, 5 or fewer nucleotides in length. Reagents and techniques that can be used to non-covalently attach an affinity reagent or other moiety to a particle (e.g., structured nucleic acid particle) are set forth in U.S. Pat. Nos. 11,203,612 or 11,505,796; or US Pat. App. Pub. No. 2022/0162684 A1, each of which is incorporated herein by reference.

A method of detecting analytes can include multiple cycles of binding the analytes with affinity reagents. In a first configuration, a multicycle method of detecting analytes can include steps of (a) providing a mixture of analytes with first affinity reagents, wherein the analytes are immobilized on a surface and the first affinity reagents are in fluid phase; (b) applying a stimulus to attract the first affinity reagents to the surface; (c) detecting binding of first affinity reagents to analytes on the surface; and (d) repeating steps (a) through (c) using second affinity reagents instead of the first affinity reagents.

In a second configuration, a multicycle method of detecting analytes in an array can include steps of (a) contacting an array with first affinity reagents in fluid phase, wherein analytes are attached at addresses in the array; (b) allowing first affinity reagents in the fluid phase to bind to analytes attached at addresses in the array; (c) removing a plurality of the first affinity reagents from contact with the array, thereby retaining a fraction of the first affinity reagents in contact with the array; (d) applying a stimulus to attract the fraction of first affinity reagents to the array; (e) detecting binding of first affinity reagents in the fraction to analytes attached at addresses in the array, and (f) repeating steps (a) through (e) using second affinity reagents instead of the first affinity reagents.

For a method that utilizes first and second affinity reagents, such as the multicycle configurations above, the second affinity reagents can recognize the same epitopes or molecules as the first affinity reagents. For example, the second affinity reagents can include the same paratope(s) as the first affinity reagents. Alternatively, the second affinity reagents, although recognizing the same epitopes or molecules as the first affinity reagents, can have a different composition compared to the first affinity reagents, for example, having different paratope(s) compared to the paratope(s) present in the first affinity reagents. In some configurations of the methods, the second affinity reagents can recognize different epitopes or molecules from the first affinity reagents. Optionally, the second affinity reagents and first affinity reagents can recognize the same epitope(s) or molecule(s) but with different affinities. For example, the second affinity reagents can differ from the first affinity reagents with regard to K_{D}, k_{off}, kₒₙ or binding probability for a given epitope or set of epitopes.

The first and second affinity reagents can have the same labels and/or tethers. This can be the case independent of the similarity or difference of the paratopes of the first and second affinity reagents. Alternatively, the first and second affinity reagents can differ with respect to their labels and/or tethers. Again, this can be the case independent of the similarity or difference of the paratopes of the first and second affinity reagents. Different labels or tethers, whether in number or kind, can allow tuning of the detectable properties of the affinity reagents, for example, to provide for more uniform detector settings or to accommodate a range of affinities for various types of affinity reagents.

A method of the present disclosure can be configured to be repeated using first analytes and second analytes. For example, a multicycle method can include steps of (a) providing a mixture of first analytes with affinity reagents, wherein the affinity reagents are immobilized on a surface and the first analytes are in fluid phase; (b) applying a stimulus to attract the first analytes to the surface; (c) detecting binding of first analytes to affinity reagents on the surface; and (d) repeating steps (a) through (c) using second analytes instead of the first analytes.

Optionally, a multicycle method can include steps of (a) contacting an array with first analytes in fluid phase, wherein affinity reagents are attached at addresses in the array; (b) allowing first analytes in the fluid phase to bind to affinity reagents attached at addresses in the array; (c) removing a plurality of the first analytes from contact with the array, thereby retaining a fraction of the first analytes in contact with the array; (d) applying a stimulus to attract the fraction of first analytes to the array; (e) detecting binding of first analytes in the fraction to affinity reagents attached at addresses in the array, and (f) repeating steps (a) through (e) using second analytes instead of the first analytes.

For a method that utilizes first and second analytes, such as the multicycle configurations above, the second analytes can recognize the same paratopes as the first analytes. For example, the second analytes can include the same epitope(s) as the first analytes. Alternatively, the second analytes, although recognizing the same paratopes as the first analytes, can have a different composition compared to the first analytes, for example, having different epitopes(s) compared to the epitopes(s) present in the first affinity reagents. In some configurations of the methods, the second analytes can recognize different paratopes from the first analytes. Optionally, the second analytes and first analytes can recognize the same paratopes(s) but with different affinities. For example, the second analytes can differ from the first analytes with regard to K_{D}, k_{off}, kₒₙ or binding probability for a given paratope or set of paratopes.

One or more steps of a method set forth herein can be repeated at least 1, 2, 3, 4, 5, 10, 50, 100, 200, 300, 400, 500 or more times. Alternatively or additionally, one or more steps of a method set forth herein can be repeated at most 500, 400, 300, 200, 100, 50, 10, 5, 4, 3, 2 or 1 times. Some or all of the repetitions can be carried out using different affinity reagents and/or different analytes from one cycle to the next. Some or all of the repetitions can be carried out using affinity reagents and/or analytes having the same function from one cycle to the next. Some or all of the repetitions can be carried out using affinity reagents and/or analytes having the same composition from one cycle to the next. Some or all of the repetitions can be carried out using different conditions from one cycle to the next. Exemplary conditions that can differ include, but are not limited to, the composition of labels attached to affinity reagents or analytes, concentration of affinity reagents in fluid phase, concentration of analytes in fluid phase, quantity of affinity reagents in fluid phase, quantity of analytes is fluid phase, duration for one or more step in the cycle, temperature for one or more step in the cycle, ionic strength for one or more step in the cycle, pH for one or more step in the cycle, detector gain or sensitivity for one or more step in the cycle, presence or concentration of denaturants for one or more step in the cycle, or presence or one or more reagent set forth herein for one or more step in the cycle.

A multicycle method can include a step of removing first affinity reagents and then delivering second affinity reagents. In some configurations, a multicycle method can include a step of removing first analytes and then delivering second analytes. Typically, removal of affinity reagents or analytes is performed after a detection step. However, in some cases it may be desirable to remove affinity reagents or analytes prior to detection. Optionally, a removal step can be followed by one or more wash steps, wherein a wash fluid is used to remove residual analytes or affinity reagents left behind by the removal step.

Methods set forth herein may comprise the steps of: (i) providing to an array a fluid comprising a plurality of fluid phase entities (e.g., affinity reagents, analytes, etc.), in which the fluid is provided at an initial concentration, and (ii) applying a stimulus to the fluid phase entities, thereby increasing a concentration of the fluid phase entities adjacent to a surface of the array. Increasing the concentration of the fluid phase entities adjacent to the surface of the array may facilitate formation of affinity reagent/analyte complexes in greater quantity than would be predicted based upon the equilibrium that would occur at the initial concentration of fluid phase entities. Increasing the concentration of the fluid phase entities adjacent to the surface of the array may facilitate a faster rate of affinity reagent/analyte complex formation.

In particular configurations of the methods set forth herein, a magnetic force is applied as a stimulus to attract affinity reagents to a surface or array to which analytes are attached. For example, individual affinity reagents can each include a magnetic moiety or paramagnetic moiety that is responsive to a magnetic field. Similarly, a magnetic force can be applied to attract analytes to a surface or array to which affinity reagents are attached. For example, individual analytes can each include a magnetic moiety or paramagnetic moiety that is responsive to a magnetic field. Optionally, the affinity reagents further include a linker and the magnetic force is applied to facilitate attachment of the linker to an address of an array. In some cases, the magnetic force is applied to an affinity reagent that is linked to a solid support, for example, at an address of an array. As such, a linked affinity reagent can be attracted to a solid support surface or address by a magnetic force.

A magnetic moiety or paramagnetic moiety can be attached directly to an affinity reagent or analyte. Alternatively, an affinity reagent or analyte can be attached to a particle, such as a structured nucleic acid particle, and the particle can in turn be attached to a magnetic moiety or paramagnetic moiety. A magnetic particle can be used such as any of a variety available form commercial sources. An affinity reagent or analyte can be attached to any of a variety of scaffolds whether or not the scaffold has a particle composition. For example, scaffolds having polymer structures such as nucleic acids or non-naturally occurring polymers can be useful. Particles and other scaffolds for analyte-scaffold composites and affinity reagent-scaffold composites, along with methods for making and using the composites are set forth, for example, in U.S. Pat. Nos. 11,505,796 or 11,692,217; US Pat. App. Pub. No. 2023/0167488 A1; or U.S. patent application Ser. No. 18/438,973, each of which is incorporated herein by reference.

Binding of affinity reagents to analytes can occur during application of a magnetic force or magnetic field. Optionally, the magnetic force or magnetic field can be pulsed or oscillated. The pulsing or oscillation can facilitate specificity of binding between binding partners by allowing them to interact multiple times. Also, the pulsing or oscillation can mitigate unwanted adhesion of fluid phase binding partners to surfaces that may otherwise occur is a constant force or field is applied. Detection of complexes formed between affinity reagents and analytes can be performed while applying a magnetic force or magnetic field. Alternatively, the magnetic force can be turned off during a detection step. It will be understood that the magnetic force or magnetic field can be applied before or after detecting the complexes.

A method of the present disclosure which includes a step of applying a magnetic field to attract fluid-phase affinity reagents to a surface or array to which analytes are attached, can further include a step of changing polarity of the magnetic field. In some cases, the force can be strong enough to remove affinity reagents from contact with the array. Similarly, a method which includes a step of applying a magnetic field to attract fluid-phase analytes to a surface or array to which affinity reagents are attached, can further include a step of changing polarity of the magnetic field. Again, the force can optionally be strong enough to remove analytes from contact with the array. After reversing field polarity, one or more steps of the method can then be repeated using the surface or the array, and using a fluid phase having second affinity reagents or second analytes (instead of the previously used affinity reagents or analytes). Typically, the second affinity reagents or second analytes will be co-localized with magnetic moieties or paramagnetic moieties. However, this is not necessary for all configurations of the methods.

In particular configurations of the methods set forth herein, an electrophoretic force or electric field is applied as a stimulus to attract affinity reagents to a surface or array to which analytes are attached. For example, individual affinity reagents can each include a charged moiety (e.g. cationic or anionic moiety) that is responsive to an electrophoretic force or electric field. Similarly, an electrophoretic force or electric field can be applied to attract analytes to a surface or array to which affinity reagents are attached. For example, individual analytes can each include a charged moiety that is responsive to an electrophoretic force or electric field.

A charged moiety can be attached directly to an affinity reagent or analyte. Optionally, the affinity reagents further include a linker and the electrophoretic force or electric field is applied to facilitate attachment of the linker to a solid support, for example, at an address of an array. In some cases, the electrophoretic force or electric field is applied to an affinity reagent that is linked to a solid support, for example, at an address of an array. As such, a linked affinity reagent can be attracted to a solid support surface or address by an electrophoretic force or electric field. Alternatively, an affinity reagent or analyte can be attached to a particle, such as a structured nucleic acid particle, and the particle can in turn be attached to a charged moiety. An electrically charged particle can be used such as a structured nucleic acid particle or any of a variety or particles available form commercial sources. An affinity reagent or analyte can be attached to any of a variety of scaffolds whether or not the scaffold has a particle composition. For example, scaffolds having polymer structures such as nucleic acids or non-naturally occurring polymers can be useful. Particles and other scaffolds for analyte-scaffold composites and affinity reagent-scaffold composites, along with methods for making and using the composites are set forth, for example, in U.S. Pat. Nos. 11,505,796 or 11,692,217; US Pat. App. Pub. No. 2023/0167488 A1; or U.S. patent application Ser. No. 18/438,973, each of which is incorporated herein by reference.

Binding of affinity reagents to analytes can occur during application of an electrophoretic force or electric field. Optionally, the electrophoretic force or electric field can be pulsed or oscillated. The pulsing or oscillation can facilitate specificity of binding between binding partners by allowing them to interact multiple times. Also, the pulsing or oscillation can mitigate unwanted adhesion of fluid phase binding partners to surfaces that may otherwise occur is a constant force or field is applied. Detection of complexes formed between affinity reagents and analytes can be performed while applying an electrophoretic force or electric field. Alternatively, the electrophoretic force or electric field can be turned off during a detection step. It will be understood that the electrophoretic force or electric field can be applied before or after detecting the complexes.

A method of the present disclosure which includes a step of applying an electrophoretic force or electric field to attract fluid-phase affinity reagents to a surface or array to which analytes are attached, can further include a step of changing polarity of the electrophoretic force or electric field. In some cases, the force can be strong enough to remove affinity reagents from contact with the array. Similarly, a method which includes a step of applying an electrophoretic force or electric field to attract fluid-phase analytes to a surface or array to which affinity reagents are attached, can further include a step of changing polarity of the electrophoretic force or electric field. Again, the force can optionally be strong enough to remove analytes from contact with the array. After reversing field polarity, one or more steps of the method can then be repeated using the surface or the array, and using a fluid phase having second affinity reagents or second analytes (instead of the previously used affinity reagents or analytes). Typically, the second affinity reagents or second analytes will be co-localized with charged moieties. However, this is not necessary for all configurations of the methods.

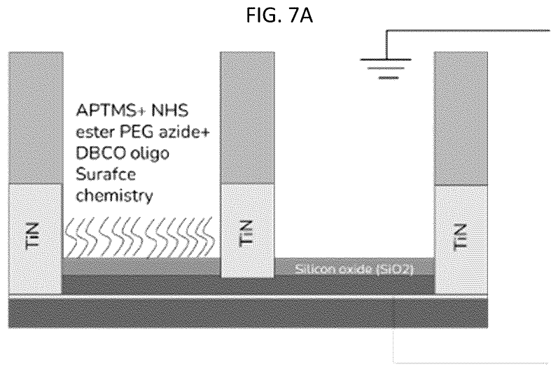

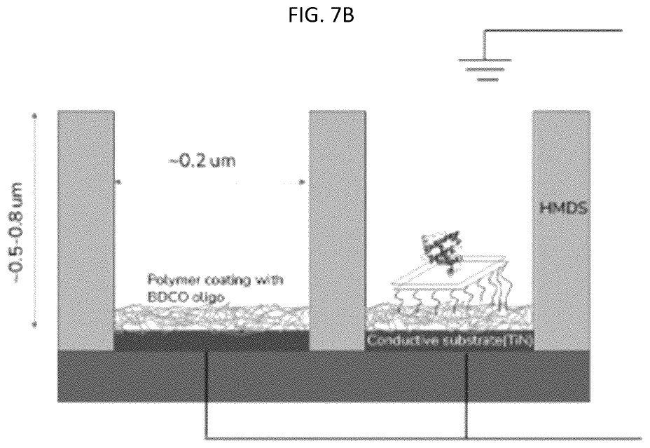

An exemplary substrate that is capable of generating an electrophoretic force to attract affinity reagents, analytes or other molecules of interest is shown in FIG. 7A. The figure shows two wells in cross-section. Trans electrodes (e.g. TiN) are embedded in the well walls and a cis electrode is in contact with fluid phase surrounding the wells. As shown for the well on the left, the silicon oxide layer at the bottom of the well can be coated with oligonucleotides. The oligonucleotides can bind to complementary sequences of a structured nucleic acid particle to which an analyte is attached, thereby attaching the analyte to the well. The voltage can be applied (or ground compared to cis electrode) alternatively or constantly to attract affinity reagents to the wells. FIG. 7B shows a microwell design for attracting reagents or analytes via electrophoretic force. Here a trans electrode (e.g., TiN) is deposited at the bottom surface of the well. The bottom surface can be coated with oligonucleotides that interact with complementary strands on structured nucleic acid particles to which an analyte is attached as shown in the well on the right.

A surface of a well shown in FIG. 7A or 7B can be attached to any of a variety of moieties for attachment of analytes or moieties for attachment of particles including, for example, nucleic acids (as shown) or click reactive moieties, receptors, ligand or other moieties used for attachment chemistries set forth herein or known in the art. A hydrogel such as an acrylamide based polymer can provide a useful coating.

In particular configurations of the methods set forth herein, analytes are co-localized with polyacids (e.g., polyacidic brushes), affinity reagents include protein moieties, and a stimulus is applied to attract the polyacids (or polyacidic brushes) to the protein moieties, wherein the applied stimulus includes a reduced pH. In another configuration, affinity reagents are co-localized with polyacids (e.g., polyacidic brushes), analytes include protein moieties, and a stimulus is applied to attract the polyacids (or polyacidic brushes) to the protein moieties, wherein the applied stimulus includes a reduced of pH. Although not wishing to be limited to mechanism, low pH can facilitate hydrogen bonding between protein moieties and polyacids (e.g., polyacidic brushes). See, for example, Ferrand-Drake del Castillo et al., Chem. Commun. 56:5889-5892 (2020), which is incorporated herein by reference. Optionally, the pH can be less than or equal to 7.0, 6.5, 6.0, 5.5, 5.0, 4.5, 4.0, 3.5, 3.0, 2.0 or lower. In some configurations a reduction in pH can facilitate attraction between polyacids (e.g., polyacidic brushes) and protein moieties. For example, the magnitude of the reduction can be at least 0.1, 0.5, 1.0, 1.5, 2.0, 2.5, 3.0 or more pH units.

A protein moiety that interacts with a polyacid (e.g. polyacidic brush) at low pH can be an endogenous component of an affinity reagent or analyte. For example, the protein moiety can be an antibody that has affinity for an analyte of interest. Alternatively, the protein moiety can be an exogenous moiety that is attached to a particle, such as a structured nucleic acid particle, and the particle can in turn be attached to the affinity reagent or analyte. Optionally, a protein moiety can be attached to a surface, such as an address in an array, and the surface can in turn be attached to the affinity reagent or analyte. A polyacid (or polyacidic brush) that interacts with a protein moiety at low pH can be an endogenous component of an affinity reagent or analyte. Alternatively, a polyacid (or polyacidic brush) can be an exogenous moiety that is attached to a particle, such as a structured nucleic acid particle, and the particle can in turn be attached to the affinity reagent or analyte. Optionally, a polyacid (or polyacidic brush) can be attached to a surface, such as an address in an array, and the surface can in turn be attached to the affinity reagent or analyte. An affinity reagent or analyte can be attached to any of a variety of scaffolds whether or not the scaffold has a particle composition. Exemplary scaffolds and particles include, but are not limited to, those set forth herein in the context of magnetic moieties, paramagnetic moieties and charged moieties.

Binding of affinity reagents to analytes can occur under low pH. Optionally, detection of complexes formed between affinity reagents and analytes is performed under low pH. Alternatively or additionally, the pH can be low before or after detecting the complexes.

A method of the present disclosure which includes a step of applying low pH to attract fluid-phase affinity reagents to a surface or array to which analytes are attached, can further include a step of applying high pH or increasing the pH, thereby removing affinity reagents from contact with the array. Similarly, a method which includes a step of applying low pH to attract fluid-phase analytes to a surface or array to which affinity reagents are attached, can further include a step of applying high pH or increasing the pH, thereby removing analytes from contact with the array. One or more steps of the method can then be repeated using the surface or the array, and using a fluid phase having second affinity reagents or second analytes (instead of the previously used affinity reagents or analytes). High pH can be at least 7.5, 8.0, 8.5, 9.0, 9.5, 10.0 or higher. An increase in pH can be at least 0.1, 0.5, 1.0, 1.5, 2.0, 2.5, 3.0 or more pH units. Typically, the second affinity reagents or second analytes will be co-localized with polyacids (e.g. polyacidic brushes) or protein moieties. However, this is not necessary for all configurations of the methods.

In particular configurations of the methods set forth herein, analytes are attached to first particles, affinity reagents are attached to second particles, and a stimulus is applied to attract the first particles to the second particles, wherein the applied stimulus includes volume exclusion, for example, via presence of a volume exclusion agent. Although not wishing to be limited to mechanism, the presence of volume exclusion agents can facilitate colloidal attraction between the particles. See, for example, Hudoba et al., ACS Nano 11:6566-6573 (2017), which is incorporated herein by reference.

Volume exclusion agents useful in the present methods may include any of a variety of polar water soluble or swellable agents including, for example, glycerol, poly(methacrylic acid), poly(acrylic) acid, polyethylene glycol (PEG), anionic polymers of polyacrylate or polymethylacrylate, or charged saccharidic polymers (e.g. anionic saccharidic polymers, such as dextran sulfate). In particular configurations, the polymer weight for a volume exclusion agent is at least 10,000, 100,000, 500,000, 1,000,000 or 2,000,000 Daltons. Alternatively or additionally, the polymer weight for a volume exclusion agent can be at most 2,000,000, 1,000,000, 500,000, 100,00 or 10,000 Daltons. Macromolecules such as proteins, nucleic acids and polysaccharides can also be useful as volume exclusion agents. Exemplary volume exclusion agents are set forth in U.S. Pat. No. 4,886,741, which is incorporated herein by reference. In some configurations of the compositions and methods set forth herein, it may be desirable to omit one or more of the volume exclusion agents set forth herein. For example, a method that is configured to detect or manipulate protein analytes can be configured to omit proteins or peptides as volume exclusion agents.

Optionally, a particle to which an analyte or affinity reagent is attached can, in turn, be attached to a surface of a solid support. For example, the particle can be attached to the surface via a flexible linker. The linker can have a composition set forth herein for linkers used to attach affinity reagents to addresses of an array. Other compositions for the linker include those set forth herein for dockers or tethers. Exemplary particles include, but are not limited to structured nucleic acid particles such as those having nucleic acid origami structures.

Binding of affinity reagents to analytes can occur in the presence of volume exclusion agents. Optionally, detection of complexes formed between affinity reagents and analytes is performed in the presence of volume exclusion agents. Alternatively or additionally, volume exclusion agents can be present before or after detecting the complexes.

A method of the present disclosure which includes a step of attracting analytes to affinity reagents in the presence of volume exclusion reagents, can further include a step of removing volume exclusion reagents, thereby separating affinity reagents from analytes. One or more steps of the method can then be repeated using second affinity reagents or second analytes (instead of the previously used affinity reagents or analytes). In other configurations, the structure of a particle to which an affinity reagent or analyte is attached can be changed to facilitate colloidal attraction or to inhibit colloidal attraction. For example, one or more nucleic acid strands can be added or removed from a structured nucleic acid particle to change the composition of the particle. A nucleic acid strand can be added to hybridize to a structured nucleic acid particle, for example, the nucleic acid strand hybridizing to a scaffold, staple or other component of a nucleic acid origami. Optionally, a nucleic acid strand can be removed from a structured nucleic acid particle, for example, a scaffold, staple or other component being removed from a nucleic acid origami. The resulting change of the structured nucleic acid particle can cause increased colloidal attraction or inhibition of colloidal attraction between the changed particle and another particle.

Colloidal attraction can be achieved if the space (or smallest dimension) between two structured nucleic acid particles is smaller than the excluded volume (or smallest dimension) of the volume exclusion agent. For example, high molecular weight PEG can cause colloidal attraction of nucleic acid origami particles described in U.S. Pat. Nos. 11,692,217 or 11,505,796, or U.S. patent application Ser. No. 18/744,286, each of which is incorporated herein by reference. Colloidal attraction can be achieved by screening charges (e.g. salt) and other properties of the solution and solute that impact solubility. This can be achieved in accordance with the DLVO theory (named after Boris Derjaguin, Lev Landau, Evert Verwey and Theodoor Overbeek)). See Gooch Encyclopedic Dictionary of Polymers. pp. 318 (2007) ISBN 978-1-4419-6246-1, which is incorporated herein by reference.

### Linked Binding Components

The present disclosure provides a method of detecting an analyte, including steps of (a) providing an analyte attached to a solid support or particle; (b) attaching an affinity reagent to the solid support or particle via a flexible linker; and (c) detecting binding between the affinity reagent and the analyte on the solid support or particle.

Also provided is a method of identifying an analyte in an array. The method can include steps of (a) providing an array of individually resolved analytes, wherein analytes are attached at addresses in the array, and wherein individual addresses in the array are each attached to a single analyte; (b) attaching affinity reagents to the addresses in the array via flexible linkers, whereby individual addresses in the array each have a single, attached affinity reagent and a single, attached analyte; (c) detecting a plurality of addresses in the array, thereby distinguishing a higher level of binding between an attached affinity reagent and an attached analyte at a first address in the array from a lower level of binding between an attached affinity reagent and an attached analyte at a second address in the array; and (d) identifying an analyte detected at an address in the array in step (c).

For ease of explanation the use of linked binding components will be exemplified herein for configurations in which an analyte is attached to a solid support (e.g. address of an array) and an affinity reagent is attached at or near the analyte via a flexible linker. It will be understood that the positions of the analyte and affinity reagent can be switched (i.e. the affinity reagent can be attached to the solid support or particle, and the analyte can be attached at or near the analyte via a flexible linker).

The above method can utilize any of a variety of analytes and arrays including, for example, those set forth previously herein in the context of applying various stimuli to attract fluid-phase affinity reagents toward array surfaces. Proteins are particularly well suited to the method and can be arrayed on a surface using techniques set forth herein or known in the art. For example, individual proteins can each be attached to a particle, such as a structured nucleic acid particle, and the protein-attached particles can be distributed to addresses on the surface of a solid support. The protein-attached particles can be delivered to a solid support, thereby providing a fluid-phase pool, and the particles in the pool can randomly land at addresses on the solid support to form an array. The particles can further include flexible linkers to which affinity reagents can be subsequently attached or the particles can contain an attachment moiety that is competent for attachment to a flexible linker. However, the particles need not necessarily include flexible linkers nor attachment moieties. Instead, flexible linkers or attachment moieties can be attached to a solid support at or near addresses of an array on the solid support. In some configurations, a first particle can be attached to an analyte and a second particle can be attached to a flexible linker or to an attachment moiety for a flexible linker. The first and second particle can be collocated at an address of an array.

A method of the present disclosure can include a step of attaching affinity reagents to addresses in an array via flexible linkers, whereby individual addresses in the array each have a single, attached affinity reagent and a single, attached analyte. Optionally, attachment is carried out by (i) contacting the array with a fluid phase including a plurality of affinity reagents, thereby attaching at least a subset of the affinity reagents to the addresses in the array via flexible linkers, whereby individual addresses in the array each comprise a single, attached affinity reagent and a single, attached analyte. The process can further include (ii) removing from contact with the array a second subset of affinity reagents that are not attached to the individual addresses in the array. Optionally, the affinity reagents that are in fluid phase during the contacting of step (i) can include flexible linkers that become attached to the addresses. For example, a flexible linker that is attached to an affinity reagent can react with an attachment moiety at a site in an array, thereby attaching the affinity reagent to the address via the flexible linker. Alternatively, individual addresses in the array can each be attached to a flexible linker and the immobilized flexible linker can react with a fluid-phase affinity moiety to attach the affinity reagent to the address via the flexible linker. In another configuration, a linker can be formed by the combination of linker regions present in the affinity reagent and in the address. For example, a first flexible linker region that is attached to an affinity reagent can react with a second flexible linker region that is attached at a site in an array, thereby forming a flexible linker that attached the affinity reagent to the address via a flexible linker that includes the first and second regions.

Optionally, attachment of affinity reagents to linkers at addresses or attachment of linker-bearing affinity reagents to addresses can be carried out while applying a magnetic force, magnetic field, electrophoretic force or electric field. The force or field can be applied consistently for some or all of the duration of the attachment step. Alternatively, the force or field can be pulsed or oscillated for some or all of the duration of the attachment step.

In some configurations, a flexible linker can be attached to a solid support, address of an array, particle (e.g., structured nucleic acid particle), affinity reagent, analyte or other substance using a chemical reaction that produces a covalent linkage. Click reactions such as those exemplified herein can be particularly useful. As such, a flexible linker can include a first reactive moiety for a click reaction and the attachment moiety with which it is to react can include a second reactive moiety for the click reaction. Alternatively, a flexible linker can be attached to a solid support, address of an array, particle (e.g., structured nucleic acid particle), affinity reagent, analyte or other substance using a non-covalent linkage. For example, a flexible linker can include a receptor (or a ligand), and the attachment moiety with which it is to bind can include a ligand for the receptor (or a receptor for the ligand). Exemplary pairs of receptors and ligands that can be used include, but are not limited to, antibodies and their epitopes; (strept) avidin (or analogs thereof) and biotin (or analogs thereof), complementary nucleic acid strands, or lectins (or analogs thereof) and carbohydrates. Complementary nucleic acids can be used to non-covalently attach a functional moiety to a solid support or particle (e.g., structured nucleic acid particle). The nucleic acids can form a complementary region that is at least 5 base pairs (bp), 10 bp, 15 bp, 20 bp, 25 bp, 30 bp, 40 bp, 50 bp, 60 bp, 70 bp or longer. Alternatively or additionally, the nucleic acids can form a complementary region that is at most 70 bp, 60 bp, 50 bp, 40 bp, 30 bp, 25 bp, 20 bp, 15 bp, 10 bp, 5 bp or shorter. Other moieties that can be used for covalent or non-covalent attachment are set forth in U.S. Pat. Nos. 11,203,612, 11,505,796 or 11,692,217; US Pat. App. Pub. No. 2022/0050049 A1, 2023/0167488 A1, or 2024/0053333 A1, each of which is incorporated herein by reference.

A linker can be composed of any of a variety of substances that provide a flexible attachment. For example, a linker can be composed of a polyethylene glycol (PEG), polyethylene oxide (PEO), protein, non-protein polymer, nucleic acid (e.g., single-stranded nucleic acid or double-stranded nucleic acid), dendrimer, peptide nucleic acid (PNA), polysaccharide, or the like.

A linker can demonstrate differential flexibility in response to environmental conditions such as temperature, pH or light. For example, the flexibility of poly(N-isopropylacrylamide) (PNIPA) and various co-polymers thereof are temperature or pH responsive. See, for example, Schild Progress in Polymer Science. 17 (2): 163-249 (1992) and Okano et al., Journal of Controlled Release. 11 (1-3): 255-265 (1990), each of which is incorporated herein by reference. Exemplary light responsive polymers include, but are not limited to, those having azobenzene, stilbene, cyanostilbene, stiff-stilbene, diarylethene, spiropyrans, hydrozones, orthonitrobenzyl moieties, triarylamine or cinnamic acid derivatives. See, for example, Xu and Feringa Adv. Mater. 35:2204413 (2023), which is incorporated herein by reference. A light-sensitive moiety, azobenzene, can be incorporated into polyacrylamide to yield copolymers that are light responsive. See, for example, Zhao ACS Appl. Polym. Mater. 2:256-262 (2020), which is incorporated herein by reference. Differential flexibility can be employed in the course of a method set forth herein. For example, a polymer can be in a flexible state during binding and detection steps. This can facilitate interaction between an affinity reagent and analyte. Optionally, the polymer can be in a rigid or reduced flexibility state during steps used to (1) attach the polymer to a solid support, (2) attach an affinity reagent to the polymer, (3) remove the polymer from the solid support or particle, or (4) remove an affinity reagent from the solid support or particle.

A linker for methods set forth herein can be characterized in terms of its effective length. The effective length can be measured for a given conformation of a linker as the shortest distance between two moieties to which the linker is attached when the liker is in the conformation. For example, the effective length can be measured between an affinity reagent to which the linker is attached and a particle or solid support surface to which the linker is also attached. A linker can be characterized in terms of its maximum effective length, which is the effective length when the linker is in its most extended conformation. A linker that is used in a method or composition set forth herein can have a maximum effective length of at least 50 nm, 75 nm, 100 nm, 200 nm, 300 nm, 400 nm, 500 nm, 600 nm, 700 nm, 800 nm, 900 nm, 1 micron or longer. Alternatively or additionally, a linker that is used in a method of composition set forth herein can have a maximum effective length of at most 1 micron, 900 nm, 800 nm, 700 nm, 600 nm, 500 nm, 400 nm, 300 nm, 200 nm, 100 nm, 75 nm, 50 nm, or shorter.

A linker can be characterized in terms of its average effective length, for example under a particular condition of use. A linker that is used in a method or composition set forth herein can have an average effective length of at least 50 nm, 100 nm, 250 nm, 500 nm, 750 nm, 1 micron or longer. Alternatively or additionally, a linker that is used in a method of composition set forth herein can have an average effective length of at most 1 micron, 750 nm, 500 nm, 250 nm, 100 nm, 50 nm or shorter.

The length of a linker used for a particular pair of affinity reagent and analyte can be selected based on the known or expected affinity between the pair. The affinity can be considered in terms of an equilibrium binding constant (e.g., K_{D} or K_{A}), half maximal effective concentration (EC50), kinetic binding constant (e.g., kₒₙ or k_{off}) or probability of binding. An equilibrium binding constant or EC50 for a given pair of affinity reagent and analyte can be expressed in terms of the average molecular distance between analytes at equilibrium. For example, an EC50 of 10 nM for an affinity reagent and analyte pair corresponds to an average distance between analytes of 550 nm. Accordingly, a linker that attaches the affinity reagent to an address where the analyte resides can have an average or maximum effective length of 550 nm to simulate an equilibrium in which the affinity reagent has a 50% probability of being bound to the analyte. The average or maximum linker length can be increased to simulate a lower concentration or the average or maximum linker length can be decreased to simulate a higher concentration. Thus, linker length can be increased as (1) K_{A} decreases, (2) EC50 decreases or (3) K_{D} increases. Linker length can be decreased as (1) K_{A} increases, (2) EC50 increases or (3) K_{D} decreases. As exemplified above, linker length can correspond to the average molecular distance between analytes at EC50, K_{A} or K_{D}. However, it may be beneficial in some configurations of the methods set forth herein to select a linker length that corresponds to an average distance that is at least 0.1 fold (x), 0.25×, 0.5×, 0.75×, 0.9×, 1×, 2×, 3× or more higher than the length corresponding to the EC50, K_{A} or K_{D} for the linked affinity reagent. Alternatively or additionally, it may be beneficial in some configurations of the methods set forth herein to select a linker length that corresponds to an average distance that is at least 0.1 fold (×), 0.25×, 0.5×, 0.75×, 0.9×, 1×, 2×, 3× or more lower than the length corresponding to the EC50, K_{A} or K_{D} for the linked affinity reagent.

An array can include a plurality of addresses, each of which is attached to an affinity reagent via a flexible linker. In such configurations, the effective length of the linkers can be shorter than the distance between nearest neighbor addresses. As such, an affinity reagent that is attached to a given address is prevented from binding to an analyte at the neighboring address. It may also be desirable for the sum of the lengths of the linkers attached to nearest neighbor addresses to be shorter than the distance between nearest neighbor addresses. For example, when the nearest neighbor addresses are attached to linkers having the same length, the length of the linkers can be shorter than half the distance between the addresses. This can be beneficial to prevent interactions between affinity reagents linked to neighboring addresses.

A method of the present disclosure can include a step of binding a flexibly linked affinity reagent to a surface immobilized analyte. Binding can optionally proceed via passive diffusion of the linked affinity reagent in the vicinity of the immobilized analyte. For example, the conditions can be similar to those employed when binding non-linked affinity reagents to surface immobilized analytes. Alternatively, binding of a flexibly linked affinity reagent to a surface immobilized analyte can proceed via active transport. For example, a linked affinity reagent can be transported toward the surface immobilized analyte under an applied force such as an electrophoretic force, electric field, magnetic force or magnetic field. A linked affinity reagent can include a paramagnetic moiety, magnetic moiety or charged moiety to facilitate active transport under such a force or field. The force or field can be consistently applied, oscillated or pulsed during binding.

A method of the present disclosure can include a step of detecting binding between a surface immobilized analyte and a flexibly linked affinity reagent. For example, a method of the present disclosure can include a step of detecting binding between an analyte that is attached to an address of an array and an affinity reagent that is attached to the address via a flexible linker. A plurality of addresses can be detected to measure the level of binding between analytes and linked affinity reagents at individual addresses. For example, a method can include a step of detecting a plurality of addresses in an array, thereby distinguishing a higher level of binding between an attached affinity reagent and an attached analyte at a first address in the array from a lower level of binding between an attached affinity reagent and an attached analyte at a second address in the array. An electrophoretic force, electric field, magnetic force or magnetic field can be applied during a detection step. Alternatively, a force or field that is applied during a binding step can be turned off or reversed during a detection step.

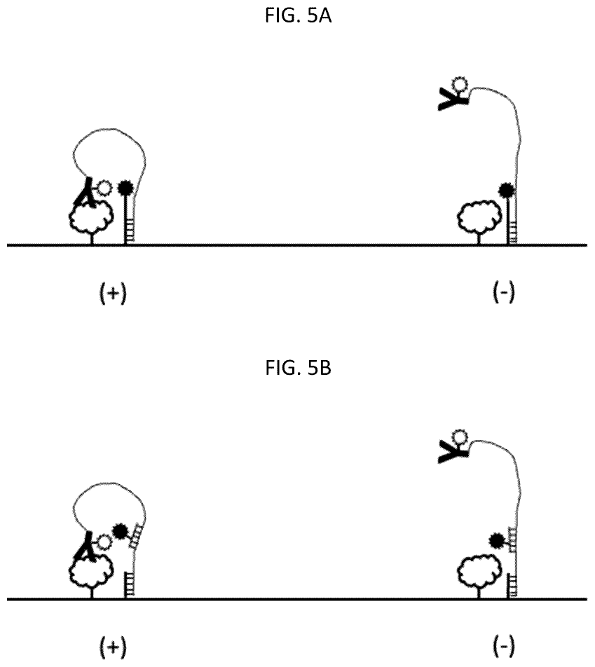

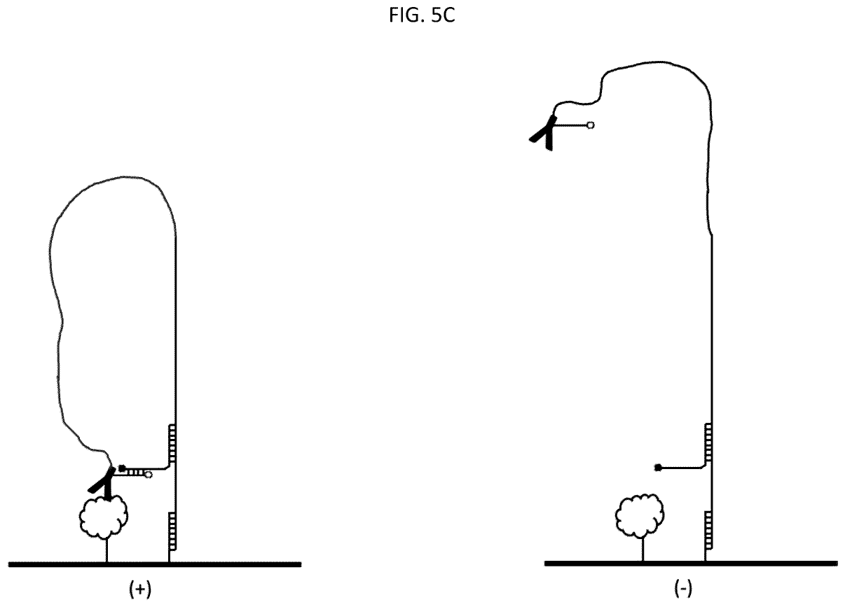

In some configurations, detection of binding between an analyte and flexibly linked affinity reagent can utilize components of a proximity-based detection technique such as energy transfer between a donor and acceptor, or other technique set forth herein. For example, Förster resonance energy transfer (FRET) between a first luminophore attached proximal to the affinity reagent and a second luminophore attached proximal to the analyte. FIG. 5A shows an exemplary configuration in which analytes (white globules) are attached to addresses of an array and an affinity reagent (Y-shape) is attached to each address via a nucleic acid linker. A first fluorophore (open 12 point star) is attached proximal to the affinity reagent and a second fluorophore (closed 12 point star) is attached proximal to the protein. In the configuration shown, the first fluorophore is attached directly to the affinity reagent and the second fluorophore is attached to an attachment moiety to which the linker hybridizes. The affinity reagent is bound to the protein in the lefthand address and thus FRET can be detected when the address is excited. In contrast, the affinity reagent in the righthand address is dissociated from the protein and FRET is insubstantial or undetectable when the address is excited. A FRET donor can be attached in proximity to either of the analyte or linked affinity reagent and a FRET acceptor can be attached in proximity to the other member of the analyte and linked affinity reagent pair.

The positions for attachments of luminophores shown in FIG. 5A is exemplary. Proximity-based detection components can be attached to other locations as well. FIG. 5B shows a configuration in which the first fluorophore (open 12 point star) is attached directly to the affinity reagent and the second fluorophore (closed 12 point star) is attached to the linker via a complementary oligonucleotide. Alternatively, the second fluorophore can be directly attached to the linker. For example, when using a nucleic acid linker, the second fluorophore can be attached to a nucleotide in the sequence of the linker. Attaching the second fluorophore to the linker allows the second fluorophore to be easily removed and replaced after use. This can provide a benefit for situations in which radiation used during FRET causes unwanted photobleaching of the luminophores. Other proximity-based detection components can be used in place of the FRET components. Exemplary configurations for associating or attaching the components to surfaces, analytes and affinity reagents are set forth herein.

Proximity-based detection can be facilitated by dockers and tethers. An analyte used in a composition or method of the present disclosure can have a docker and the docker can be attached to a first component of a proximity-based detection pair (e.g., a FRET donor or acceptor used for energy transfer). An affinity reagent used in a composition or method of the present disclosure can have a tether and the tether can be attached to a second component of a proximity-based detection pair (e.g., a FRET donor or acceptor used for energy transfer). The docker and tether can be configured to position the components of the pair in proximity to each other when the linked affinity reagent is bound to the analyte. As such, the docker and tether can facilitate proximity-based detection (e.g. energy transfer) and, thus, facilitate detection of binding between the linked affinity reagent and analyte.

FIG. 5C shows an exemplary configuration in which analytes (white globules) are attached to addresses of an array and an affinity reagent (Y-shape) is attached to each address via a nucleic acid linker. A first fluorophore (open 12 point star) is attached to the affinity reagent via a nucleic acid tether and a second fluorophore (closed 12 point star) is attached to the linker via a nucleic acid docker. At the address on the left (“+”), the affinity reagent is bound to the analyte and the tether is hybridized to the docker. A nucleotide sequence of the tether is complementary to a nucleotide sequence of the docker. Hybridization of the tether and docker positions the first and second fluorophore into proximity for FRET, thereby being capable of producing a signal indicative of binding between affinity reagent and analyte. At the address on the right (“−”), the affinity reagent is dissociated from the analyte and the tether is dissociated from the docker. Thus, a FRET signal is not produced, indicating the affinity reagent is not bound to the analyte.

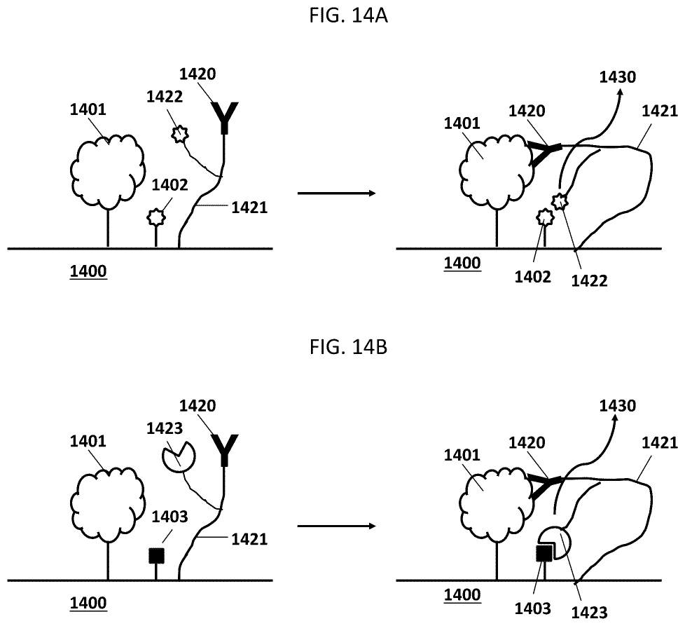

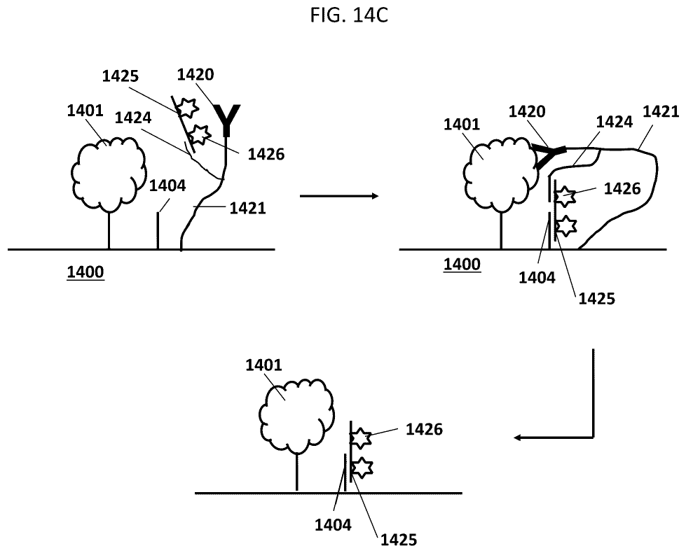

FIGS. 14A-14C depict additional configurations of methods and systems utilizing an affinity reagent attached to a linker. The left-hand configuration of FIG. 14A depicts an analyte 1401 that is immobilized to a fixed address of a solid support 1400. A first FRET dye 1402 is co-localized at the fixed address of the solid support 1400, optionally by a linking moiety. An affinity reagent 1420 is also co-localized at the fixed address by a linker 1421 that is attached to the solid support 1400. A second FRET dye 1422 is attached to the linker 1421, optionally by a branched linking moiety. In the right-hand configuration of FIG. 14A, binding of the affinity reagent 1420 to the analyte 1401 brings the first FRET dye 1402 in sufficient proximity to the second FRET dye 1422 to provide a detectable fluorescent signal 1430. FIG. 14B depicts a system in which the FRET dyes have been replaced by a luminescent protein 1423 (e.g., a luciferase) that provides a detectable signal 1430 when the luciferase protein 1423 binds to a cofactor 1403 (e.g., coelenterazine). The configurations of FIGS. 14A and 14B could also be substituted with a split fluorescent (e.g., split green fluorescent protein) or luminescent protein (e.g., split luciferase) that provides a detectable signal when two components of the split protein are brought into close enough proximity to bind to each other by the binding interaction of the affinity reagent and the analyte.

FIG. 14C depicts a system that is configured to indirectly record a binding interaction between an affinity reagent attached to a linker and an immobilized analyte. The upper left-hand configuration of FIG. 14C depicts an analyte 1401 that is immobilized to a fixed address of a solid support 1400. A first transferring moiety 1404 is co-localized at the fixed address of the solid support 1400. An affinity reagent 1420 is also co-localized at the fixed address by a linker 1421 that is attached to the solid support 1400. A second transferring moiety 1424 is attached to the linker 1421. A transferrable moiety 1425 comprising one or more detectable labels 1426 is attached to the second transferring moiety 1424. In the upper right-hand configuration of FIG. 14C, binding of the affinity reagent 1420 to the analyte 1401 facilitates binding of the transferrable moiety 1425 to the first transferring moiety 1404 due to the close proximity of the transferrable moiety 1425 to the first transferring moiety 1404. In the bottom center configuration, the affinity reagent 1420 and linker 1421 may be released, leaving the transferrable moiety 1425 attached to the first transferring moiety 1404. A signal may from the one or more detectable labels 1426 may be detected at the fixed address of the solid support 1400, indicating the prior presence of a binding interaction between the affinity reagent 1420 and analyte 1401. Additional methods and system for forming reactions that record the presence of a binding interaction between an affinity reagent and analyte are set forth below in the section titled “Additional Methods of Proximity-Based Detection.” The skilled person can readily envision additional combinations of components that can be used to directly or indirectly identify presence of binding interactions between an affinity reagent and an analyte.

When using a linker-attached affinity reagent in combination with a docker and tether, the lengths for docker and tether can be substantially shorter than the length of the linker. As set forth above, the length of the linker can be selected based on the known or suspected strength of binding between the affinity reagent and analyte. Typically, the docker and tether are configured to position proximity-based detection components without substantially contributing to the binding of the affinity reagent to the analyte. As such, the length of the docker and tether can be selected to facilitate hybridization when the docker and tether are in close proximity without necessarily driving interactions from a larger distance. The relative lengths of the linker, docker and tether can be selected to balance binding interactions between affinity reagent and analyte with binding interactions between docker and tether. The relative lengths can be selected to correspond to the relative binding affinities. For example, the length for the docker and/or tether can be at most 50%, 25%, 20%, 10%, 5%, 1%, 0.1% or less of the effective length of the linker. The length of the complementary region formed by hybridization of a nucleic acid tether and nucleic acid docker can also be selected to balance binding interactions between affinity reagent and analyte with binding interactions between docker and tether. For example, the length of a complementary region formed between a docker and tether can be at least 2, 3, 4, 5, 6, 7, 8, 9, 10, 15 or more base pairs. Alternatively or additionally, the complementary region can be at most 15, 10, 9, 8, 7, 6, 5, 4, 3, or 2 base pairs. The strength of binding between a tether and docker can also be selected to position proximity-based detection components without substantially contributing to the binding of the affinity reagent to the analyte. For example, a nucleic acid tether and docker can be configured to have a relatively low melting temperature (Tₘ). Melting temperature can be influenced by the GC and AT composition of the hybrid region formed between a docker and tether. The complementary region can include A:T basepairs and G:C basepairs, only A:T basepairs, only G:C basepairs, or various percentages of A:T basepairs and G:C basepairs such as those exemplified above herein. In some configurations, a nucleic acid docker and nucleic acid tether are not substantially complementary with each other

In some configurations, detection of binding between an analyte and linked affinity reagent can utilize a spatially selective detection technique. Any of a variety of spatially selective optical detection techniques can be used to detect binding of an analyte, at an address of an array, to an optically detectable affinity reagent that is linked at the address. The spatially selective optical detection technique can be configured to selectively filter, or even reject, signals produced by the affinity reagent when it is dissociated from the analyte and thus outside of a detection zone. For example, confocal fluorescence microscopy can be used to collect fluorescent signals from a spatially resolved region where an analyte binds to a fluorescently detectable affinity reagent while rejecting signals from the affinity reagent when it is outside of the region. Total internal reflection fluorescence can also be used to collect fluorescent signals proximal to a surface where an analyte binds to a fluorescently detectable affinity reagent while rejecting signals from the affinity reagent when it is located distal from the surface. Waveguides can be useful for exciting optically detectable affinity reagents to provide spatially selective optical detection. For example, a zero mode waveguide can be used.

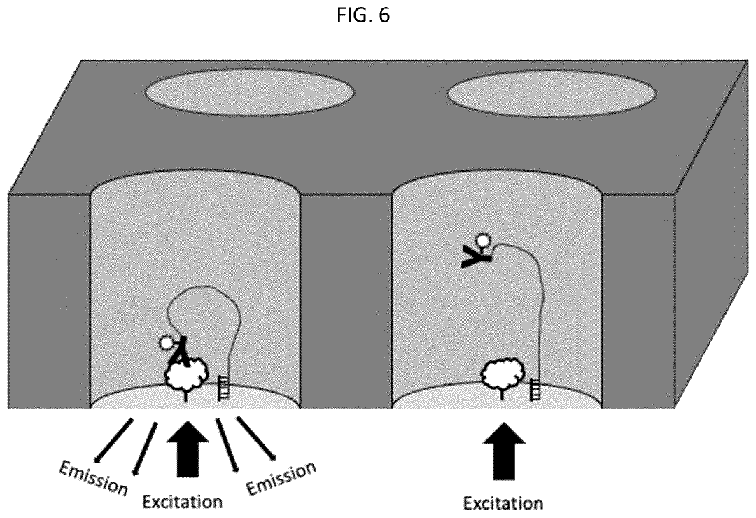

FIG. 6 shows a section of an array of zero mode waveguides (ZMW) in which two ZMWs are shown in cross section. The array is configured to selectively deliver excitation radiation to a region of each ZMW that is near the bottom where a protein analyte (white globule) is attached. The bottom of the ZMW provides the highest detection sensitivity and the sensitivity decays with distance from the bottom. A detection zone can be delineated based on the parameters selected for detection, such as a threshold for signal intensity, threshold for signal-to-noise ratio, duration for signal acquisition etc. Both ZMWs have a protein analyte in a detection zone and both also have an affinity reagent (Y-shape) to which a fluorophore (12 point star) is attached. The affinity reagents are attached to the ZMW via a nucleic acid linker. The affinity reagent is bound to the protein in the lefthand ZMW and thus produces a detectable emission when the ZMW is excited. In contrast, the affinity reagent in the righthand ZMW is dissociated from the protein and no emission is produced when the ZMW is excited. ZMWs can be made and used as set forth, for example, in lizuka et al., Biophysics and Physicobiology 19, e190032 (2002); Eid et. Al., Science 323:133-138 (2009); U.S. Pat. Nos. 7,313,308, or U.S. Pat. App. Pub. Nos. 2008/0128627 A1, 2012/0085894 A1, 2016/0334334 A1, 2016/0363728 A1, 2016/0273034 A1, 2016/0061740 A1, or 2017/0145498 A1, each of which is incorporated herein by reference.

Analytes can be distinguished in a method set forth herein according to the level of binding between the analytes and a given type of affinity reagent. In some cases, analytes can be distinguished according to the level of binding between the analytes and a plurality of different affinity reagents. For example, a method can include a step of distinguishing a higher level of binding between an attached affinity reagent and an attached analyte at a first address in an array from a lower level of binding between an attached affinity reagent and an attached analyte at a second address in the array. The level of binding can be determined or reported as the intensity of signal collected from a measurement of binding between affinity reagent and analyte (e.g., higher intensity is correlated with higher level of binding), the duration of signal production while measuring binding between affinity reagent and analyte (e.g., longer duration is correlated with higher level of binding), relative proportion of acceptor emission to donor emission in a FRET detection system (e.g., higher FRET is correlated directly with higher level of binding), or the like.

A method of detecting an analyte using a linked affinity reagent can include multiple cycles of binding the analyte with affinity reagents. A multicycle method can include steps of (a) providing an analyte attached to a solid support or particle; (b) attaching an affinity reagent to the solid support or particle via a flexible linker; (c) detecting binding between the affinity reagent and the analyte on the solid support or particle; (d) detaching the affinity reagent from the solid support or particle after step (c), and (e) repeating steps (b) and (c) using second affinity reagents instead of the affinity reagents. It will be understood that the positions of the analyte and affinity reagent can be reversed (i.e. the affinity reagent can be attached to the solid support or particle, and the analyte can be replaced when repeating steps (b) and (c))

A multicycle method can include steps of (a) providing an array of individually resolved analytes, wherein analytes are attached at addresses in the array, and wherein individual addresses in the array are each attached to a single analyte; (b) attaching affinity reagents to the addresses in the array via flexible linkers, whereby individual addresses in the array each have a single, attached affinity reagent and a single, attached analyte; (c) detecting a plurality of addresses in the array, thereby distinguishing a higher level of binding between an attached affinity reagent and an attached analyte at a first address in the array from a lower level of binding between an attached affinity reagent and an attached analyte at a second address in the array; (d) identifying an analyte detected at an address in the array in step (c); (e) detaching affinity reagents from the addresses in the array after step (c), and (f) repeating steps (b) and (c) using second affinity reagents instead of the affinity reagents. It will be understood that step (d) can be performed before or after steps (e) and (f). Again, the positions of the analyte and affinity reagent can be reversed (i.e. the affinity reagent can be attached to the solid support or particle, and the analyte can be replaced when repeating steps (b) and (c)).

For a method that utilizes first and second affinity reagents, such as the multicycle configurations above, the second affinity reagents that are linked to the solid support, address or particle can recognize the same epitopes or molecules as the first affinity reagents. For example, the second affinity reagents can include the same paratope(s) as the first affinity reagents. Alternatively, the second affinity reagents, although recognizing the same epitopes or molecules as the first affinity reagents, can have a different composition compared to the first affinity reagents, for example, having different paratope(s) compared to the paratope(s) present in the first affinity reagents. In some configurations of the methods, the second affinity reagents can recognize different epitopes or molecules from the first affinity reagents. Optionally, the second affinity reagents and first affinity reagents can recognize the same epitope(s) or molecules(s) but with different affinities. For example, the second affinity reagents can differ from the first affinity reagents with regard to K_{D}, k_{off}, kₒₙ or binding probability for a given epitope or set of epitopes.

For a method that utilizes first and second analytes, such as the multicycle configurations above, the second analytes can recognize the same paratopes as the first analytes. For example, the second analytes can include the same epitope(s) as the first analytes. Alternatively, the second analytes, although recognizing the same paratopes as the first analytes, can have a different composition compared to the first analytes, for example, having different epitopes(s) compared to the epitopes(s) present in the first affinity reagents. In some configurations of the methods, the second analytes can recognize different paratopes from the first analytes. Optionally, the second analytes and first analytes can recognize the same paratopes(s) but with different affinities. For example, the second analytes can differ from the first analytes with regard to K_{D}, k_{off}, kₒₙ or binding probability for a given paratope or set of paratopes.

One or more steps of a method set forth herein can be repeated at least 1, 2, 3, 4, 5, 10, 50, 100, 200, 300, 400, 500 or more times. Alternatively or additionally, one or more steps of a method set forth herein can be repeated at most 500, 400, 300, 200, 100, 50, 10, 5, 4, 3, 2 or 1 times. Some or all of the repetitions can be carried out using different affinity reagents and/or different analytes from one cycle to the next. Some or all of the repetitions can be carried out using affinity reagents and/or analytes having the same function from one cycle to the next. Some or all of the repetitions can be carried out using affinity reagents and/or analytes having the same composition from one cycle to the next. Some or all of the repetitions can be carried out using different conditions from one cycle to the next. Exemplary conditions that can differ include, but are not limited to, the composition of labels attached to affinity reagents or analytes, effective length of the flexible linker attached to each affinity reagent or analyte, composition of the flexible linker attached to each affinity reagent or analyte, flexibility of the flexible linker attached to each affinity reagent or analyte, length of dockers and/or tethers, length of complementary sequences for a docker-tether pair, duration for one or more steps in the cycle, temperature for one or more steps in the cycle, ionic strength for one or more steps in the cycle, pH for one or more steps in the cycle, detector gain or sensitivity during detection, presence or concentration of denaturants for one or more step in the cycle, or presence or one or more reagent set forth herein for one or more step in the cycle.

### Additional Methods of Proximity-Based Detection

Methods set forth herein may comprise a step of recording a binding interaction between an affinity reagent and an analyte. Recording of a binding interaction may occur during an equilibration period between a plurality of affinity reagents and a plurality of analytes. Preferably, the equilibration period may occur for a sufficient time to establish a binding equilibrium between the affinity reagents and the analytes. A method of recording a binding interaction between an affinity reagent and an analyte may comprise forming a reaction, in which the reaction facilitates detection of a signal associated with a complex containing an analyte and an affinity reagent after an equilibration period has ended. Preferably, binding interactions may be recorded in a spatially resolved manner such that a binding interaction between a first analyte and a first affinity reagent can be resolved from a binding interaction between a second analyte and a second affinity reagent. Array-based methods set forth herein may be especially useful for resolving binding interactions between pluralities of analytes and pluralities of affinity reagents.

While numerous methods are exemplified by a signal becoming detectable when a binding interaction occurs, many described methods can easily be reversed to have a signal become undetectable when a binding interaction occurs (e.g., via removal or dissociation of a detectable label, etc.).

A method may comprise direct recording of a binding interaction between an affinity reagent and an analyte if a reaction of a first moiety co-localized with the affinity reagent with a second moiety co-localized with the analyte inhibits dissociation of the affinity reagent from the analyte, thereby facilitating detection of a signal associated with a complex containing the affinity reagent and the analyte. A method may comprise indirect recording of a binding interaction if a detectable label is retained with the affinity reagent or analyte after the affinity reagent has dissociated from the analyte, thereby facilitating detection of a signal associated with a complex containing the affinity reagent and analyte after the complex has dissociated.

FIG. 8A depicts a method of transferring a detectable label 826 to a site containing an immobilized analyte 810. Initially (left-hand side), the analyte 810 is immobilized at a fixed address of a solid support 800. An immobilized first transferring moiety 805 (e.g., an oligonucleotide, a member of a receptor-ligand binding pair, a covalently-reactive moiety, etc.) is immobilized at the fixed address of the solid support 800 containing the immobilized analyte 810. Preferably, the first transferring moiety 805 is co-localized at a distance from the immobilized analyte 810 that is not optically-resolvable. An affinity reagent 820 is bound to the immobilized analyte 810, thereby immobilizing the affinity reagent 820 at the fixed address of the solid support. The affinity reagent 820 is attached to a linking moiety 821 that contains a second transferring moiety 822 (e.g., an oligonucleotide). A transferrable moiety 825 is bound to the second transferring moiety 822. The transferrable moiety 825 comprises one or more detectable labels 826. In a second configuration (right-hand side of FIG. 8A), the transferrable moiety 825 has been dissociated from the second transferring moiety 822 and has become associated to the first transferring moiety 805 when the transferrable moiety 825 has been positioned in a close enough proximity to the first transferring moiety 805 to facilitate transfer. The transferrable moiety 825 and transferring moieties (805, 822) may be designed to have a greater stability when the transferrable moiety 825 is attached to the second transferring moiety 822. For example, if the transferrable moiety 825 is a nucleic acid, a larger quantity of nucleotides of the transferrable moiety 825 may hybridize to the second transferring moiety 822 than hybridize to the first transferring moiety 805. Accordingly, the binding interaction of the affinity reagent 820 with the analyte 810 is recorded at the fixed address by the transfer of the transferrable moiety 825 from the second transferring moiety 822 of the affinity reagent 820 to the first transferring moiety 805 that is co-localized with the analyte 810. A detectable signal from the one or more detectable labels 826 can be detected at the fixed address after the affinity reagent 820 has dissociated from the analyte 810.

FIG. 8B depicts a similar method to the method of FIG. 8A, with the addition of a second transferring moiety 823 to the affinity reagent 820. The affinity reagent 820 is provided an optional second linking moiety 829 containing the second transferring moiety 823. In another configuration, the linking moiety 821 could contain the second transferring moiety 823. In an initial configuration (left-hand side), a second transferrable moiety 828 is bound to the second transferring moiety 823. The second transferrable moiety 828 contains one or more second detectable labels 827. Preferably, signals from the second detectable labels 827 are distinguishable from the detectable labels 826 (e.g., by emission wavelength, by excitation wavelength, by luminescence lifetime, etc.). The fixed address of the solid support 800 further comprises an immobilized fourth transferring moiety 806. Preferably, the fourth transferring moiety 806 is co-localized at a distance from the immobilized analyte 810 that is not optically-resolvable. Preferably, the kinetics of transfer of the transferrable moiety 825 to the first transferring moiety 805 is faster than the kinetics of transfer of the second transferrable moiety 828 to the fourth transferring moiety 806. Accordingly, a time length of binding or binding strength of the affinity reagent 820 to the analyte 810 can be estimated by whether neither, one or both transferring moieties were transferred to the fixed address. In the second configuration (right-hand side), only the transferrable moiety 825 will be detectable at the fixed address of the solid support 800 due to insufficient binding time of the affinity reagent 820 to the analyte 810 to facilitate transfer of the second transferrable moiety 828 to the fourth transferring moiety 806.

For configurations of transferrable moieties comprising oligonucleotide transferrable moieties, such as those depicted in FIGS. 8A-8B, it may be preferable to control the directionality of transfer of a transferrable moiety. For example, a transferrable moiety is likely to remain associated to a transferring moiety if it has greater number of nucleotides of sequence complementarity with the transferring moiety. Accordingly, a transferrable moiety may have at least about 1, 2, 3, 4, 5, 6, 7, 8, 9, 10, or more than 10 nucleotides of additional sequence complementarity with a receiving transferring moiety with respect to its complementarity to a delivering transferring moiety.

Alternatively, directionality of transfer of a transferrable moiety can be controlled by attaching the transferrable moiety to a receiving transferring moiety by a non-dissociable binding interaction (e.g., a covalent bond, a non-dissociable receptor-ligand binding interaction). A non-dissociable binding interaction may comprise any binding interaction that is unlikely to spontaneously dissociate before a detection event has occurred. FIG. 8C depicts a similar configuration to FIG. 8A, with a non-dissociable attachment moiety 807 attached to the transferring moiety 805. The transferrable moiety 825 further comprises a complementary non-dissociable attachment moiety 827. In a second configuration (right-hand side of FIG. 8C), the transferrable moiety 825 has been dissociated from the second transferring moiety 822 and has become associated to the first transferrable moiety 805 when the complementary non-dissociable attachment moiety 827 binds to the non-dissociable attachment moiety 807, thereby forming a non-dissociable interaction 809 between the transferrable moiety 825 and the transferring moiety 805.

In some cases, a transferrable moiety may be attached to a transferring moiety by an irreversible non-dissociable interaction. Exemplary irreversible non-dissociable binding interactions can include covalent binding interactions (e.g., Click-type reactions) and receptor-ligand binding interactions (e.g., streptavidin-biotin, SpyCatcher-SpyTag, SnoopCatcher-SnoopTag, SdyCatcher-SdyTag, etc.). For methods having multiple possible detection events, it may be preferable to have a receiving transferring moiety be configured to be releasable so that the signal associated with transfer of the transferrable moiety can be erased after a detection event by release of the transferrable moiety-transferring moiety complex. For example, a non-dissociable attachment moiety may be attached to a first oligonucleotide, in which the first oligonucleotide is hybridized to a second oligonucleotide, and in which the second oligonucleotide is attached to a solid support. Accordingly, the first oligonucleotide can be released (e.g., by a toehold-mediated strand displacement reaction, by contacting with a chaotrope, by heating, etc.), thereby releasing any transferrable moiety attached to the first oligonucleotide.

Alternatively, a transferrable moiety may be attached to a transferring moiety by a reversible non-dissociable interaction. A reversible non-dissociable interaction can include certain photoreactive covalent bonds. Accordingly, a method may comprise contacting a complex comprising an analyte and an affinity reagent with light of a wavelength that activates a photoreactive reaction. Another useful reversible non-dissociable interaction can include use of immobilized metal affinity chromatography (IMAC)-type binding interactions. An IMAC-type binding interaction may be formed by attaching a histidine tag to a transferring moiety or a transferrable moiety, and attaching a metal-chelating complex (e.g., nitrilotriacetic acid, iminodiacetic acid, N-(carboxymethyl) aspartate, N,N,N′-tris(carboxymethyl)ethylenediamine, etc.) to the complement. The metal-chelating complex can strongly bind the histidine tag in the presence of metal ions (e.g., Cu²⁺, Ni²⁺, Zn²⁺, Co²⁺). The binding of the histidine tag to the metal-chelating complex can be reversed by use of a low-pH solution (e.g., a pH of less than about 6.5, 6.0, 5.5, 5.0, 4.5, or 4.0), a solution comprising EDTA, a solution comprising imidazole, a solution comprising histidine or polyhistidine, or combinations thereof. An IMAC-type binding interaction may also be utilized for directly recording a binding interaction by attaching the histidine tag or metal-chelating complex to an affinity reagent, and attaching the complement to an analyte or a fixed address containing the analyte.

FIGS. 9A-9C depict a method of recording a binding interaction via a catalyzed labeling reaction. FIG. 9A depicts in an initial configuration an analyte 910 that is immobilized at a fixed address of a solid support 900. A modifiable moiety 906 (e.g., an oligonucleotide, a peptide, a polymer chain, etc.) is immobilized at the fixed address of the solid support 900 containing the immobilized analyte 910. In some cases, the modifiable moiety may be releasably attached to the solid support 900. For example, the modifiable moiety 906 may comprise an oligonucleotide that is coupled to a complementary oligonucleotide 905, in which the complementary oligonucleotide is coupled to the solid support. Preferably, the modifiable moiety 906 is co-localized at a distance from the immobilized analyte 910 that is not optically-resolvable. An affinity reagent 920 is bound to the immobilized analyte 910, thereby immobilizing the affinity reagent 920 at the fixed address of the solid support 900. The affinity reagent 920 is attached to a linking moiety 921 that is also attached to a catalyzing moiety 922 (e.g., an enzyme, a catalyst particle). In a second configuration (right-hand side of FIG. 9A), the modifiable moiety 906 may be modified by the catalyzed formation of a detectable label 908 on the modifiable moiety. Exemplary methods of adding a detectable label can include polymerase extension of an oligonucleotide (e.g., by incorporation of modified or fluorescently-labeled nucleotides), ligation of a labeled nucleic acid to the modifiable moiety 906, or addition of ligands to the modifiable moiety (e.g., BirA-catalyzed biotinylation of a modifiable moiety comprising an avi-tag, addition or modification of amino-acid sidechains by kinases, etc.). Accordingly, the detectable label remains co-located at the fixed address containing the analyte 910 after the affinity reagent 920 has dissociated.

FIG. 9B depicts a method of secondary labeling to detect the detectable label 908 added to a modifiable moiety 906. In the left-hand configuration, a secondary detectable label 930 (e.g., a fluorophore, a luminophore, etc.) is contacted to the modifiable moiety 906. The secondary detectable label 930 is attached to a moiety 938 with a binding specificity for the detectable label 908. For example, a biotinylated avi-tag would be bound by a fluorescently-labeled streptavidin or avidin molecule. Similarly, an enzymatically-modified peptide (e.g., phosphorylated, ubiquinated, methylated, etc.) could be bound by an affinity agent that is specific to the modified peptide. Certain covalent modifications could be bound by complementary covalent compounds. The right-hand side configuration of FIG. 9B depicts the secondary detectable label 930 bound to the modifiable moiety 906 by binding of the moiety 938 to the detectable label 908. The left-hand side of FIG. 9C depicts detection of a signal 935 from the secondary detectable label 930 at the fixed address of the solid support 900 with an optical sensor 940. After detecting a signal from the modifiable moiety 906, the modifiable moiety may be optionally removed (e.g., by a toehold-mediated strand displacement reaction, by contacting with a chaotrope, etc.). After removing the modifiable moiety 906, an unmodified modifiable moiety 906 may be immobilized at the fixed address before another affinity reagent 920 (e.g., an affinity reagent with a different binding specificity) is bound to the analyte 910.

Methods and systems of the present disclosure set forth systems of dockers and tethers that may be useful for inhibiting dissociation of an affinity reagent from an analyte after the affinity reagent has become bound to the analyte. Oligonucleotide-based docker/tether systems may be particularly useful for methods and systems set forth herein. In some cases, it may be advantageous to provide an oligonucleotide in a fluid phase that is configured to bind to a docker nucleic acid strand and a tether nucleic acid strand.

FIGS. 10A-10B illustrate a method of directly recording a binding interaction between an analyte 1010 and an affinity reagent 1020 utilizing a bridging nucleic acid strand. In an initial configuration (left-hand side), FIG. 10A depicts an analyte 1010 immobilized at a fixed address of a solid support 1000. A docker nucleic acid strand 1005 is immobilized at the fixed address of the solid support 1000 containing the immobilized analyte 1010. Preferably, the docker nucleic acid strand 1005 is co-localized at a distance from the immobilized analyte 1010 that is not optically-resolvable. An affinity reagent 1020 is bound to the immobilized analyte 1010, thereby immobilizing the affinity reagent 1020 at the fixed address of the solid support 1000. The affinity reagent 1020 is attached to a linking moiety 1021 that is also attached to a tether nucleic acid strand 1025. The complex comprising the affinity reagent 1020 bound to the analyte 1010 is contacted with a fluid phase bridging nucleic acid strand 1022. Optionally, the bridging nucleic acid strand 1022 is attached to one or more detectable labels 1026. Alternatively, the one or more detectable labels 1026 may be attached to the affinity reagent 1020 or the linking moiety 1021. In a subsequent configuration (right-hand side of FIG. 10A), the bridge nucleic acid strand 1022 is hybridized to the docker nucleic acid strand 1005 and the tether nucleic acid strand 1025, thereby inhibiting dissociation of the affinity reagent 1020 from the fixed address of the solid support 1000. Preferably, the bridging nucleic acid strand 1022 may be provided with one or more nucleotide sequences that do not hybridize to the docker nucleic acid strand 1005 and/or tether nucleic acid strand 1025. For example, as depicted in the right side of FIG. 10A, a portion of the bridge nucleic acid strand 1022 between the portions that hybridize to the tether nucleic acid strand 1025 and to the docker nucleic acid strand 1005 does not bind to either of the tether nucleic acid strand 1025 or the docker nucleic acid strand 1005. Such pendant nucleotide sequences may facilitate dissociation of the bridging nucleic acid strand 1022 (e.g., via toehold mediated strand displacement) after a signal from a detectable label 1026 has been detected at an address containing the bridging nucleic acid strand 1022. FIG. 10B depicts displacement of the bridging nucleic acid strand 1022 by contacting the complex containing the affinity reagent 1020 bound to the analyte 1010 with a fluid-phase displacing nucleic acid strand 1050 (left-hand side configuration). In a second figuration of FIG. 10B, the displacing nucleic acid strand 1050 has hybridized to the bridging nucleic acid strand 1022, thereby dissociating the bridging nucleic acid strand 1022 from the docker nucleic acid strand 1005 and/or the tether nucleic acid strand 1025. Once the bridging interaction has been dissociated, the affinity reagent 1020 may dissociate from the analyte 1010, optionally in the presence of a dissociation medium (e.g., a buffer comprising a chaotrope or surfactant).

A bridging nucleic acid strand may be configured to join a docker nucleic acid strand to a tether nucleic acid strand when the docker and tether are in proximity to each other. A bridging nucleic acid strand may comprise a first nucleotide sequence that is configured to hybridize to a nucleic acid sequence of a docker nucleic acid strand, a second nucleotide sequence that is configured to hybridize to a nucleic acid sequence of a tether nucleic acid strand, and optionally a linker (e.g., a linking nucleic acid, a linking non-nucleic acid) of a chosen length. A longer linker will facilitate bridging of a docker and a tether when there is a greater separation between the two strands (i.e., the docket nucleic strand and the tether nucleic strand). Accordingly, the length of a linker of a bridging nucleic acid strand can be chosen to provide a desired separation gap between a docker and tether when both nucleic acid strands are hybridized to the bridging nucleic acid strand. For nucleic acid linkers, a separation distance can be estimated as 0.6-0.7 nanometers per nucleotide.

A bridging nucleic acid strand may have a linker containing at least about 1, 2, 3, 4, 5, 6, 7, 8, 9, 10, 15, 20, 25, 30, 35, 40, 50, 100, or more than 100 nucleotides. Alternatively or additionally, a bridging nucleic acid strand may have a linker containing no more than about 100, 50, 40, 35, 30, 25, 20, 15, 10, 9, 8, 7, 6, 5, 4, 3, 2, 1 or less than 1 nucleotides. In some cases, a bridging nucleic acid strand may not have a linker.

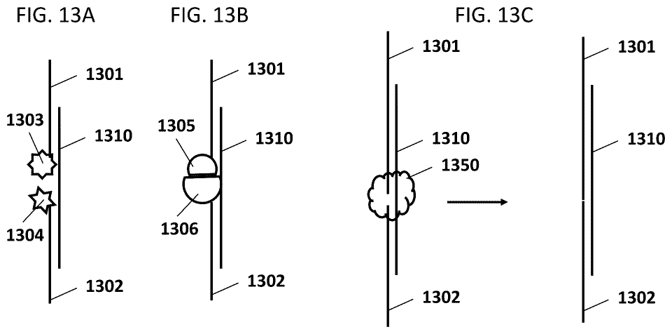

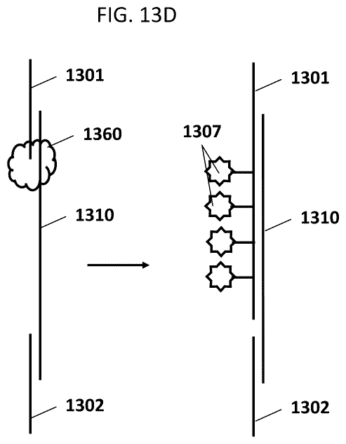

FIGS. 13A-13D depict different configurations that may affect a chosen length of a linker in a bridging nucleic acid strand. In some cases, a linker of a bridging nucleic acid strand may be chosen to facilitate formation of an interaction between a first moiety and a second moiety. FIG. 13A depicts a bridging nucleic acid strand 1310 that can be utilized to control the separation distance between a pair of fluorescent FRET dyes. A docker strand 1301 attached to a first FRET dye 1303 may be held in proximity to a tether strand 1302 attached to a second FRET dye 1304 such that the FRET dye pair are sufficiently close (e.g., within about 5 nanometers of each other) to undergo a signal-producing FRET interaction in the presence of a stimuli (e.g., an exciting photon). FIG. 13B depicts a bridging nucleic acid strand 1310 that can be utilized to bring together two halves of a split protein system (e.g., a split fluorescent protein, a split luminescent protein). A docker strand 1301 attached to a first half of a split protein system 1305 may be held in proximity to a tether strand 1302 attached to a second half of a split protein system 1306 such that the split protein components are sufficiently close to bind, thereby facilitating emission of a signal from the joined split protein components. FIG. 13C depicts a bridging nucleic acid strand 1310 with a zero-length linker moiety that joins a docker nucleic acid strand 1301 to a tether nucleic acid strand 1302. In the left-hand configuration, the nucleic acid complex may be bound by a ligase enzyme 1350, thereby producing a linkage between the docker strand and tether strand. Such a system may be useful for retaining co-localization of an affinity reagent and an analyte even after a bridging nucleic acid strand has dissociated.

Alternatively, it may be preferable to provide a larger separation gap between a docker strand and a tether strand with a bridging nucleic acid strand. In the left-hand configuration, FIG. 13D depicts a bridging nucleic acid strand 1310 hybridized to a docker nucleic acid strand 1301 and a tether nucleic acid strand 1302. The complex containing the bridged nucleic acid strands is coupled to a polymerase enzyme 1360. In the presence of modified nucleotides, the polymerase 1360 may extend the docker nucleic acid strand 1301 or tether nucleic acid strand 1302, thereby incorporating modified nucleotides into the extended nucleic acid strand. The right-hand configuration of FIG. 13D depicts an extended nucleic acid strand that has had incorporated detectable labels 1307 attached to the modified nucleotides. The size of the separation between the docker and tether and the concentration of modified nucleotides may be utilized to control the extent of incorporation of the modified nucleotides, thereby controlling the strength of a signal from the extended nucleic acid strand. In some cases, a polymerase may be utilized to incorporated fluorescently-labeled modified nucleotides. In other cases, a polymerase may be utilized to incorporate modified nucleotides that are subsequently labeled via the covalent attachment of fluorescent dye molecules.

A separation gap between a docker nucleic acid strand and a tether nucleic acid strand, when coupled by a bridging nucleic acid strand, may be at least about 0.1 nanometers (nm), 0.5 nm, 1 nm, 2 nm, 3 nm, 4 nm, 5 nm, 10 nm, 20 nm, 30 nm, 40 nm, 50 nm, or more than 50 nm. Alternatively or additionally, a separation gap between a docker nucleic acid strand and a tether nucleic acid strand, when coupled by a bridging nucleic acid strand, may be no more than about 50 nm, 40 nm, 30 nm, 20 nm, 10 nm, 5 nm, 4 nm, 3 nm, 2 nm, 1 nm, 0.5 nm, 0.1 nm, or less than 0.1 nm. For docker and tether systems containing FRET-based dye pairs, it may be preferable to provide a separation gap of 5 nanometers or less. A separation gap between a docker nucleic acid strand and a tether nucleic acid strand, when coupled by a bridging nucleic acid strand, may include a single-stranded region of the bridging nucleic acid strand of at least about 1, 2, 3, 4, 5, 10, 15, 20, 25, 30, 35, 40, 50, or more than 50 nucleotides. Alternatively or additionally, a separation gap between a docker nucleic acid strand and a tether nucleic acid strand, when coupled by a bridging nucleic acid strand, may include a single-stranded region of the bridging nucleic acid strand of no more than about 50, 40, 35, 30, 25, 20, 15, 10, 5, 4, 3, 2, 1, or 0 nucleotide.

Although some reactions between a first reactant and a second reactant have been exemplified with respect to a bridging nucleic acid strand, it will be recognized that other types of bridging molecules may be utilized. A bridging molecule may be configured to form a non-covalent binding interaction with a first reactant, a second reactant, or both the first reactant and the second reactant. For example, a bridging molecule may comprise a first affinity reagent attached to a second affinity reagent (optionally by a linking moiety), in which the first affinity reagent binds to a ligand (e.g., an affinity tag) of the first reactant and the second affinity reagent binds to a ligand of the second reactant, thereby coupling the first reactant to the second reactant. A bridging molecule may be configured to form a covalent binding interaction with a first reactant, a second reactant, or both the first reactant and the second reactant. For example, a bridging molecule may comprise covalent cross-linking moiety comprising a first reactive moiety and a second reactive moiety, in which the first reactive moiety bonds to a reactive moiety of the first reactant and the second reactive moiety bonds to a reactive moiety of the second reactant, thereby coupling the first reactant to the second reactant.

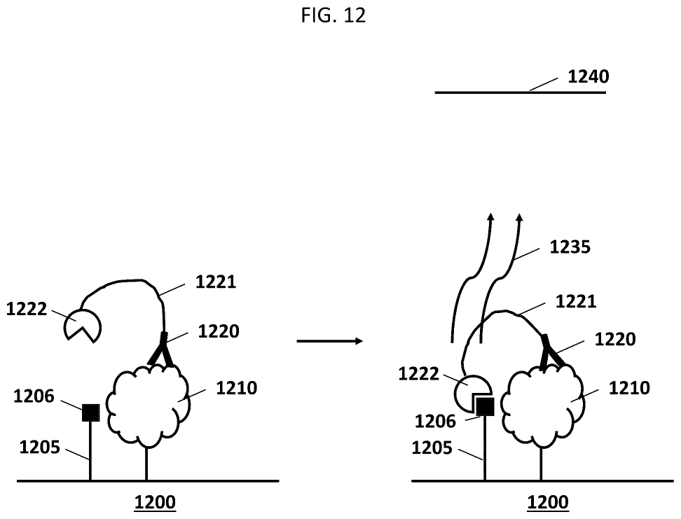

FIG. 12 depicts another method of directly recording a binding interaction between an affinity reagent and an analyte. In an initial configuration (left-hand side), FIG. 12 depicts an analyte 1210 immobilized at a fixed address of a solid support 1200. A linking moiety 1205 is immobilized at the fixed address of the solid support 1200 containing the immobilized analyte 1210. The linking moiety 1205 is attached to a cofactor 1206 or a plurality thereof for a luminescent protein 1222. Preferably, the linking moiety 1205 is co-localized at a distance from the immobilized analyte 1210 that is not optically-resolvable. An affinity reagent 1220 is bound to the immobilized analyte 1210, thereby immobilizing the affinity reagent 1220 at the fixed address of the solid support 1200. The affinity reagent 1220 is attached to a linking moiety 1221 that is also attached to the luminescent protein 1222. In the second configuration (right-hand side) of FIG. 12, the luminescent protein 1222 binds to the cofactor 1206, thereby inducing emission of photons 1235 from the luminescent protein 1222. The luminescent signal may be detected at the fixed address of the solid support 1200 by a detector 1240.

Certain methods set forth herein for recording a binding interaction between an affinity reagent and an analyte may be amenable to a method of kinetic control. Kinetic control may comprise any suitable method for controlling the rate of a secondary binding reaction that records the binding interaction between the affinity reagent and the analyte. Preferably, the binding on-rate for the reaction is slower than the binding on-rate for the affinity reagent with the analyte, thereby limiting the likelihood of a false detection event. It will be recognized that an affinity reagent may have a known and/or characterized tendency to bind to certain epitopes or analytes other than its primary target (i.e., off-target binding). Off-target binding may be characterized by a faster off-rate of the affinity reagent from an analyte having a secondary binding target relative to its off-rate from an analyte having a primary binding target for the affinity reagent. In such cases, it may be preferable for a reaction to have a binding on-rate that is slower than the binding off-rate for an affinity reagent with a secondary binding target. Accordingly, the reaction will be more likely to record binding interactions between an affinity reagent and its primary binding target.

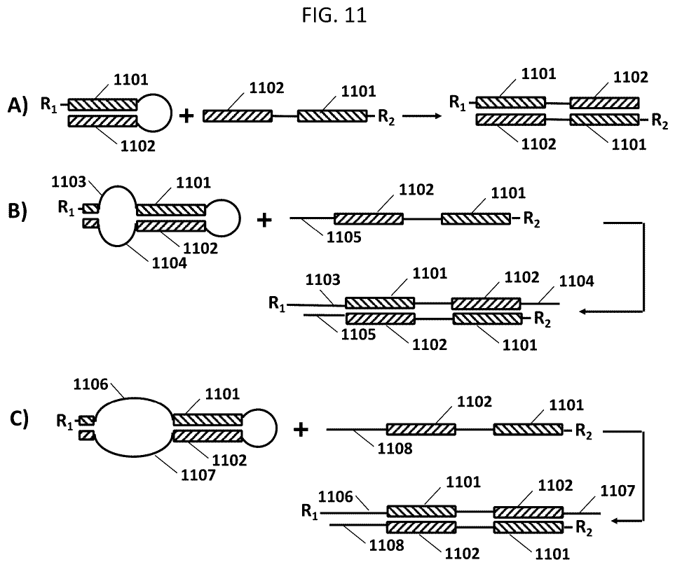

Oligonucleotide systems may be especially useful for providing kinetic control to reactions. Oligonucleotides having internal complementarity of their nucleotide sequences can fold into secondary structures in which a rate of unfolding is governed by the secondary structure morphology and extent of internal complementarity. FIG. 11 illustrates oligonucleotide systems that may have different kinetic rates of hybridization due to differences in oligonucleotide structure. In case A, a first oligonucleotide attached to moiety R1 (e.g., an analyte, an affinity reagent, a linking moiety, a docker strand, a tether strand, etc.) has a loop structure formed by internal complementarity of nucleotide sequence 1101 with nucleotide sequence 1102. The first oligonucleotide is contacted with a second oligonucleotide attached to moiety R2 (e.g., an analyte, an affinity reagent, a linking moiety, a docker strand, a tether strand, etc.) also contains nucleotide sequences 1101 and 1102. The kinetic rate of unfolding the first oligonucleotide and hybridizing it to the second oligonucleotide may be very slow due to the absence of a toehold sequence for the second oligonucleotide to bind to, thereby initiating dehybridization of the internally complementary nucleotide sequences. In case B, the first oligonucleotide further comprises nucleotide sequences 1103 and 1104, whose lack of complementarity introduces a smaller loop structure into the secondary structure of the first oligonucleotide. The second oligonucleotide comprises a toehold nucleotide sequence 1105 that can hybridize to nucleotide sequence 1103 of the first oligonucleotide, thereby initiating dehybridization of the secondary structure of the first oligonucleotide. Case C depicts a first oligonucleotide with a larger loop structure due to the non-complementarity of nucleotide sequences 1106 and 1107. The second oligonucleotide comprises a larger toehold nucleotide sequence 1108 that can hybridize to nucleotide sequence 1106 of the first oligonucleotide, thereby initiating dehybridization of the secondary structure of the first oligonucleotide. The rate of binding of the first oligonucleotide to the second oligonucleotide in FIG. 11 from slowest to fastest can be ordered as A<B<C. Incorporation of secondary structures into one or more oligonucleotides similar to those depicted in FIG. 11 may be utilized to control the binding on-rates of oligonucleotides in any system or method depicted herein.

Other methods and systems may be designed to introduce kinetic control in a secondary binding reaction. For example, binding reactions or chemical reactions can be controlled by the concentration of enzymes, reactants, or substrates, as well as the use of modified enzymes, catalysts or substrate derivatives that can increase or decrease the binding on-rate of a secondary binding reaction. For example, use of a biotin ligase enzyme to label an affinity tag for the indirect recording of a binding interaction between an affinity reagent and an analyte may be kinetically throttled by the length of the linker moiety attaching the biotin ligase to the affinity reagent or affinity tag, as well as the concentration of free biotin in solution. Likewise, the biotin ligase enzyme may be modified to have a faster or slower rate of biotin attachment, thereby affecting the enzyme's processivity. Further, the affinity tag can be modified by amino acid sequence or presence of modified or unnatural amino acids to control the rate of biotin attachment.

It will be understood that methods and systems exemplified herein via a single analyte and a single affinity reagent can readily be expanded into methods and system containing pluralities of analytes and/or affinity reagents. Array-based methods may be especially useful for observing binding interactions between pluralities of analytes and pluralities of affinity reagents at single-analyte resolution. For some array-based methods, particles (e.g., nucleic acid nanoparticles) may be useful for co-localizing certain components at a single fixed address of the array. For example, a single analyte and a docker strand, as set forth herein, may be attached to a single nucleic acid nanoparticle, in which the nucleic acid nanoparticle is attached to a fixed address of a solid support. Accordingly, each individual address of a plurality of addresses of a single-analyte array may comprise a single particle, in which the single particle is attached to one and only one analyte, and at least one other moiety that is configured to facilitate formation of a reaction, as set forth herein.

It will be further understood that methods and systems exemplified by a single set of moieties configured to form a reaction may further comprise additional moieties configured to form reactions. For example, an analyte may be co-localized with a plurality of docker strands, and/or an affinity reagent may be attached to a plurality of tether strands. In another example, a linking moiety may comprise a plurality of FRET dyes that can be brought into proximity of a plurality of complementary FRET dyes.

### Hybridization Chain Reactions

The present disclosure further provides a method of detecting binding of an affinity reagent to an analyte. The method can include steps of (a) contacting an analyte with an affinity reagent, thereby forming a complex, wherein the affinity reagent includes a paratope and a first target nucleic acid, wherein the analyte includes an epitope and a second target nucleic acid, and wherein the complex includes the analyte bound to the affinity reagent via interaction between the paratope and the epitope; (b) contacting the complex with a set of first oligonucleotides and a set of second oligonucleotides, thereby forming a nicked double helix, wherein the first oligonucleotides include a first hairpin nucleotide sequence, wherein the second oligonucleotides include a second hairpin nucleotide sequence, and wherein the nicked double helix includes (i) a first oligonucleotide of the set of first oligonucleotides hybridized to the first target nucleic acid and further hybridized to the second target nucleic acid, and (ii) a plurality of second oligonucleotides of the set of second oligonucleotides hybridized to a plurality of first oligonucleotides of the set of first oligonucleotides; and (c) detecting the nicked double helix, thereby detecting binding of the affinity reagent to the analyte.

The present disclosure also provides a molecular complex, including (a) an affinity reagent, wherein the affinity reagent includes a paratope and a first target nucleic acid; (b) an analyte, wherein the analyte includes an epitope and a second target nucleic acid, and wherein the affinity reagent is bound to the analyte via interaction between the paratope and the epitope; and (c) a nicked double helix, wherein the nicked double helix includes: (i) a plurality of first oligonucleotides, each of the first oligonucleotides having a first hairpin nucleotide sequence, and (ii) a plurality of second oligonucleotides, each of the second oligonucleotides having a second hairpin nucleotide sequence, wherein a first oligonucleotide of the nicked double helix is hybridized to the first target nucleic acid and further hybridized to the second target nucleic acid, and wherein a plurality of second oligonucleotides of the nicked double helix is hybridized to a plurality of first oligonucleotides of the nicked double helix.

It will be understood that embodiments and configurations of the invention exemplified herein for proteins can be extended to other analytes. Sources for proteins and techniques for manipulating proteins are exemplified below. The exemplified sources can provide other analytes using techniques similar to those set forth herein for protein isolation or other techniques known by those skilled in the art. Moreover, an analyte that is used in a method or composition set forth herein can include any of a variety of epitopes. For example, an epitope can include one or more amino acids, nucleotides, or sugars. Conversely, an epitope that is utilized in a method or composition of the present disclosure can lack amino acids, nucleotides or sugars.

An analyte can be attached to a target nucleic acid. Several methods and reagents will be exemplified herein for attachment of an analyte to a target nucleic acid. It will be understood that similar methods and reagents can be used to attach an analyte to other entities such as retaining components, structured nucleic acid particles, nucleic acid origami or artificial polymers. Moreover, similar methods and reagents can be used to attach affinity reagents to a variety of entities such as target nucleic acids, retaining components, structured nucleic acid particles, nucleic acid origami or artificial polymers.

A protein that is to be attached to a target nucleic acid can include at least one amino acid that is reactive with an attachment moiety on the moiety of interest. Optionally, attachment can exploit a reactive moiety that is present in a natural amino acid. For example, attachment can occur between an attachment moiety and (A) an amine that is present at the amino terminus of a protein or in the side chain of a lysine, histidine or arginine; (B) a sulfur that is present in the side chain of a cysteine or methionine; (C) a carboxylate that is present at the carboxy terminus of a protein or in the side chain of an aspartic acid or glutamic acid; (D) an oxygen that is present in the side chain of a serine, threonine or tyrosine; or (E) an amide that is present in the side chain of a glutamine or asparagine.

Optionally, a protein can be modified to incorporate an exogenous moiety that is reactive with an attachment moiety on a target nucleic acid or other entity. For example, one or more amino acids of a protein can be modified to include an exogenous moiety that forms a covalent bond with a chemically reactive attachment moiety on a target nucleic acid. Optionally, one or more amino acids of a protein can be modified to include an exogenous moiety that participates in a binding reaction to form a non-covalent bond with a target nucleic acid or other entity. An amino acid can be functionalized with an exogenous moiety by exploiting reactivities of amines, sulfurs, carboxylates, oxygens, amides or other reactive moieties found in native amino acids. Exemplary reactive moieties (e.g., native or exogenous to proteins) and attachment moieties with which they react are set forth below or in WO 2019/195633 A1; US Pat. App. Pub. Nos. 2021/0101930 A1, 2022/0162684 A1 or 2022/0227890 A1; or U.S. Pat. Nos. 11,203,612 or 11,505,796, each of which is incorporated herein by reference.

A protein and an attachment moiety with which the protein will react can include components of a SpyTag/SpyCatcher system (See, Zakeri et al. Proceedings Nat'l Acad. Sciences USA. 109 (12): E690-7 (2012)). In this system, a 13 amino acid tag protein (Spy Tag) forms a first coupling handle, with a 12.3 kDa protein (Spy-Catcher) forming the partner to the first coupling handle. Optionally, the SpyCatcher can be attached to a protein. The SpyCatcher can irreversibly bond to a SpyTag on another entity through an isopeptide bond. As will be appreciated, either the SpyTag or SpyCatcher can be on a protein and the entity to which the protein is to be attached can be functionalized with the other.

In some configurations, an attachment moiety on a protein can be reactive in a click reaction. Attachment can be accomplished in part by chemical reaction of a click moiety with a reactive moiety on another entity. The chemical conjugation may proceed via an amide formation reaction, reductive amination reaction, N-terminal modification, thiol Michael addition reaction, disulfide formation reaction, copper (I)-catalyzed alkyne-azide cycloaddition (CuAAC) reaction, strain-promoted alkyne-azide cycloaddtion reaction (SPAAC), Strain-promoted alkyne-nitrone cycloaddition (SPANC), inverse electron-demand Diels-Alder (IEDDA) reaction, oxime/hydrazone formation reaction, free-radical polymerization reaction, or a combination thereof.

Moieties that participate in cycloaddition reactions may be utilized to attach a protein to another entity such as a target nucleic acid. In cycloaddition reactions, two or more unsaturated moieties form a cyclic product with a reduction in the degree of unsaturation, these reaction partners are typically absent from natural systems, and so the use of cycloadditions for conjugation utilizes the introduction of unnatural functionality within a coupling partner. Exemplary moieties and their attachment reactions include:

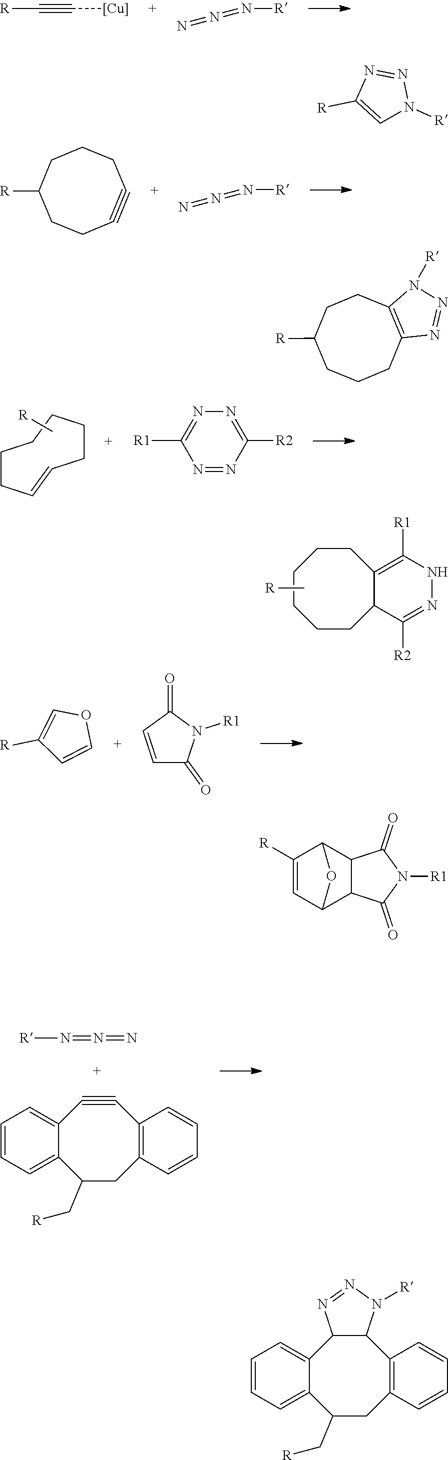

In some cases, moieties that participate in Copper-Catalyzed Azide-Alkyne Cycloadditions (CuAAC) may be utilized to attach a protein to a target nucleic acid or other entity. Optionally, moieties that participate in Strain-Promoted Azide-Alkyne Cycloadditions (SPAAC) may be utilized. One of an azide or alkyne can be connected to a protein and reacted with an entity connected to the other. A CuAAC or SPAAC reaction can be performed to produce a triazole attachment of a protein to a target nucleic acid or other entity.

Moieties that participate in inverse-electron demand Diels-Alder (IEDDA) reactions may be utilized to attach a protein to a target nucleic acid or other entity. One of a 1,2,4,5-tetrazine moiety, strained alkene moiety or strained alkyne can be connected to protein and, optionally, subjected to an IEDDA reaction. Exemplary moieties include, but are not limited to, trans-cyclooctenes, functionalized norbornene derivatives, triazines, or spirohexene. In some cases, a maleimide or furan can be used as an attachment moiety and, optionally, used in a hetero-Diels-Alder cycloaddition between a maleimide and furan. In some cases, a Diels-Alder reaction can achieve covalent coupling of a diene moiety with an alkene moiety to form a six-membered ring complex for attachment.

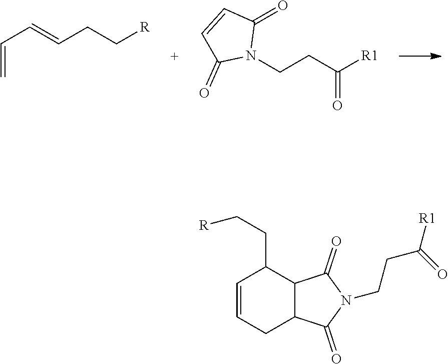

A protein can be attached to a target nucleic acid or other entity via binding moieties having affinity for each other. For example, a protein can include a first binding moiety that binds to a second binding moiety on a target nucleic acid or other entity. A first binding moiety may bind with a second binding moiety in a non-covalent manner. Some binding moieties can also be chemically reactive or catalytic (e.g., kinases, proteases, etc.). A binding moiety can be chemically non-reactive and non-catalytic, thereby not permanently altering the chemical structure of another binding moiety to which it binds. Exemplary pairs of binding moieties include, but are not limited to, an antibody, such as a full-length antibody or functional fragment thereof which bind to epitopes. Other useful binding moiety pairs include, for example, (strept) avidin (or analogs thereof) and biotin (or analogs thereof), complementary nucleic acids, nucleic acid aptamers and their ligands, lectins and carbohydrates or the like.

A protein or other analyte may be attached to a retaining components, such as a structured nucleic acid particle, as set forth herein, or an artificial polymer. In some cases the artificial polymers are configured as dendrons. Dendrons include at least one branched chain polymer. A branched chain polymer can include at least 1, 2, 3, 4, 5, 6, 8 or 10 branch points. Alternatively or additionally, a branched chain can include at most 10, 8, 6, 5, 4, 3, 2 or 1 branch points. A branch point is a covalent intersection between at least two chains. For example, at least 2, 3, 4, 5 or more chains can intersect at a branch point of a branched chain. Alternatively or additionally, at most 5, 4, 3 or 2 chains can intersect at a branch point of a branched chain. A polymer, whether branched or not, can include a single type of monomer subunit or multiple different types of monomer subunits. Accordingly, a polymer can include at least 1, 2, 3, 4, 5 or more different types of monomer subunits. Alternatively or additionally, a polymer can include at most 5, 4, 3, 2 or 1 different types of monomer subunits. A polymer having only one type of subunit in the network of covalent bonds is referred to as a “homopolymer.” In contrast, a “copolymer” includes two or more different types of subunits in the network of covalent bonds.

A retaining component that includes an artificial polymer can have a volume or footprint in a range set forth above for SNAPs. A retaining component can be further characterized in terms of molecular weight (or molecular weight distribution) in a desired size range. For example, the molecular weight, average molecular weight distribution, minimum molecular weight distribution or maximum molecular weight distribution can be at least 1 kDa, 2 kDa, 5 kDa, 10 kDa, 25 kDa, 50 kDa or more. Alternatively or additionally, the molecular weight, average molecular weight distribution, minimum molecular weight distribution or maximum molecular weight distribution can be at most 50 kDa, 25 kDa, 10 kDa, 5 kDa, 2 kDa, 1 kDa or less. An artificial polymer can be characterized in term of degree of polymerization (i.e. number of monomer subunits) present. For example, an artificial polymer can include at least 2, 10, 20, 30, 40, 50, 100, 200, 300 or more monomers. Alternatively or additionally, an artificial polymer can include at most 300, 200, 100, 50, 40, 30, 20, 10, or 2 monomers.

An artificial polymer can lack natural polymers or monomers found in natural polymers. For example, the skeletal structure of the artificial polymer can lack natural polymers or monomers. This can be the case whether or not the artificial polymer has attached moieties that include natural polymers or monomers. Examples of natural moieties that can be absent from an artificial polymer, for example in the skeletal structure include, but are not limited to, nucleic acids (e.g. DNA or RNA), nucleotides (e.g. deoxyribonucleotides or ribonucleotides), nucleosides (e.g. deoxyribonucleosides or ribonucleosides), proteins, amino acids, or sugars (e.g. saccharide monomers, monosaccharides, oligosaccharides, polysaccharides or glycans). An artificial polymer can optionally lack any polymer or monomer that is synthesized in vivo or that is capable of being synthesized in vivo. Alternatively, an artificial polymer can include natural moieties that are combined to form a non-naturally occurring molecule. For example, an artificial polymer can be composed of nucleic acid monomers or nucleic acid strands that form a non-naturally occurring nucleic acid dendrimer structure.

Particularly useful artificial polymers include, for example, poly(amidoamine) (PAMAM) dendrimer, poly(amidoamine) dendron, hyperbranched polymers such as linear and branched polyethyleneimine (PEI) and polypropyleneimine (PPI), star polymers, grafted polymers, peptide-based linear or branched dendrimers such as branched poly-L-lysine (PLL) and silane-cored dendrimer. Other useful artificial polymers include dendrimer nucleic acids having branching structures. See, for example, Liu et al., J. Mater. Chem. B 9:4991-5007 (2021) and Meng et al., ACS Nano 8:6171-6181 (2014), each of which is incorporated herein by reference. Examples of useful polymers are set forth in Tomalia, et al. J Polym Sci Part A: Polym Chem 40:2719-2728 (2002); Higashihara, et al. Polym J 44, 14-29 (2012); Gupta, et al. J. Phys. Chem. B 124, 20, 4193-4202 (2020); Ren, et al. Chem. Rev. 116, 12, 6743-6836 (2016); Chis, et al. Molecules 25 (17): 3982 (2020); Zheng, et al. Chem. Soc. Rev. 44, 4091-4130 (2015), or U.S. patent application Ser. No. 18/438,973, each of which is incorporated herein by reference.

A further example of a useful retaining component is a bead or particle made from a solid support material such as those exemplified herein in the context of arrays. For example, a retaining component can include one or more of glass and modified or functionalized glass, plastics (including acrylics, polystyrene and copolymers of styrene and other materials, polypropylene, polyethylene, polybutylene, polyurethanes, Teflon™, cyclic olefins, polyimides etc.), nylon, ceramics, resins, Zeonor™, silica or silica-based materials including silicon and modified silicon, carbon, metals, inorganic glasses, optical fiber bundles, gels, or polymers. A retaining component that includes a solid support material can have a volume or footprint in a range set forth above for SNAPs.

A method of the present disclosure can include a step of coupling one or more analytes (e.g. proteins) to a solid support or a surface thereof, for example, prior to performing a binding assay set forth herein. The coupling of one or more analytes to a solid support may include covalent and/or non-covalent coupling. Covalent coupling of an analyte to a solid support can include direct covalent coupling of the analyte to the solid support (e.g., formation of coordination bonds) or indirect covalent coupling between a functional moiety that is coupled to a retaining component of the analyte and a functional moiety that is coupled to the solid support (e.g., a CLICK-type reaction). Non-covalent coupling can occur between a solid support and an analyte or between a solid support and a retaining component of the analyte. Exemplary non-covalent interactions include electrostatic or magnetic interactions, or non-covalent bonding interactions (e.g., ionic bonds, van der Waals interactions, hydrogen bonding, etc.).

An array may be provided with a dynamic range of analytes, as exemplified below for proteins. Dynamic range can refer to the ratio of abundance between a more populous protein species and a less populous protein species. A dynamic range can be a comprehensive measure (ratio of most populous protein species to least populous protein species) or a limited measure (ratio of a first protein species to a second protein species). An array of proteins may be provided with a dynamic range of at least about 10, 10², 10³, 10⁴, 10⁵, 10⁶, 10⁷, 10⁸, 10⁹, 10¹⁰, 10¹¹, 10¹², or more. Alternatively or additionally, an array of analytes may be provided with a dynamic range of no more than about 10¹², 10¹¹, 10¹⁰, 10⁹, 10⁸, 10⁷, 10⁶, 10⁵, 10⁴, 10³, 10², 10, or less.

In some configurations of the methods, compositions or systems set forth herein, an affinity reagent can include a plurality of labels. The labels can produce substantially identical signals, for example, due to the labels having identical structures. In some cases, the labels need not be structurally identical but may nevertheless produce signals that are indistinguishable using a given detector. For example, two luminophores may have different structure but may produce overlapping emission signals at a wavelength that is used for detection in a method set forth herein. The presence of multiple labels can be beneficial for increasing signal to noise compared to affinity reagents having only a single label. This can be especially helpful for use in an assay that is configured for single-protein resolution.

Optionally, an affinity reagent can include at least 1, 2, 3, 4, 5, 6, 7, 8, 9, 10, 15, 20, 25 or more labels. Alternatively or additionally, an affinity reagent can include at most 25, 20, 15, 10, 9, 8, 7, 6, 5, 4, 3, 2 or 1 label. Again, the labels can be the same or different with regard to structure or function. One or more labels can be attached to an affinity reagent via a retaining component, such as a retaining component that is also attached to a plurality of affinity moieties. One or more labels can be attached to other moieties of an affinity reagent. For example, label(s) can be attached to one or more affinity moieties of an affinity reagent. The presence of multiple labels on an affinity reagent can provide increased signal to noise and can thus increase sensitivity of detection to accommodate single-molecule resolved detection in a method set forth herein.

Exemplary labels and detectable signals they produce include, without limitation, optical labels such as luminophores (e.g. fluorophores) which emit photons at particular wavelengths when excited by radiation and which can be distinguished due to luminescence lifetime or polarization. Other useful optical labels include chromophores which absorb radiation at particular wavelengths and nanoparticles which can interact with light to produce signals such as photon emissions or light scatter. Other labels include heavy atoms, radioactive isotopes, mass labels, charge labels, spin labels, nucleic acids having particular sequence, receptors, ligands, or the like. Signals that can be detected from labels in methods set forth herein include, for example, optical signals such as absorbance of radiation, luminescence emission, luminescence lifetime, luminescence polarization, fluorescence emission, fluorescence lifetime, fluorescence polarization, or the like; Rayleigh and/or Mie scattering; binding affinity for a ligand or receptor; magnetic properties; electrical properties; charge; mass; radioactivity, nucleotide sequences detected via nucleic acid sequencing platforms, sequence specific hybridization of nucleic acid labels to complementary probes or the like.

In some configurations of the methods, compositions or systems set forth herein, an affinity reagent can include a target nucleic acid or can be attached to a target nucleic acid. A target nucleic acid can be attached directly to an affinity reagent, for example, via modification of the affinity reagent and target nucleic acid with moieties that are capable of binding to each other non-covalently or with moieties that are capable of reacting with each other to form a covalent bond. Alternatively, a target nucleic acid can be attached to a retaining component that is also attached to at least one affinity reagent or paratope. Useful retaining components and methods for attaching target nucleic acids to the retaining components include those set forth above in the context of attaching retaining components to analytes and target nucleic acids. The retaining components can have sizes, dimensions, compositions and other characteristics as set forth herein in the context of retaining components for analytes.

One or more steps of a method set forth herein can be carried out in a vessel. The reactions set forth herein, such as binding between affinity reagent and analyte or producing a nicked double stranded nucleic acid, can occur in solution phase or on solid phase. In the case of a solution phase reaction, affinity reagents, analytes, and nucleic acid components used for detection can interact in solution. In the case of a solid phase reaction, at least one of the complex-forming components can be immobilized and another complex-forming component can be present in solution. A reaction set forth herein can be initiated by delivering a fluid to a vessel, wherein the vessel includes at least one reactant and the fluid contains at least one other reactant. For example, an analyte can be immobilized in a vessel and a fluid that contains an affinity reagent can be delivered to the vessel. The opposite configuration is also possible, whereby an affinity reagent is immobilized in a vessel and a fluid that contains an analyte is delivered to the vessel. In some configurations of the methods, a fluid that contains an affinity reagent or analyte can also contain oligonucleotides that are used to produce a nicked double helix. Thus, the oligonucleotides can be delivered to the vessel simultaneously with the affinity reagents or analytes. For example, the oligonucleotides can be delivered prior to formation of a complex between an affinity reagent and an analyte. Alternatively, the oligonucleotides need not be delivered simultaneously with either the affinity reagents or the analytes. For example, the oligonucleotides can be delivered after formation of a complex between an affinity reagent and an analyte.

A method that is used in accordance with the present disclosure can be configured for single molecule-resolved detection. This can be achieved for example by attaching one of the complex-forming components to a solid support and contacting the solid support with a fluid containing the other component for forming the complex. For example, an analyte (e.g. protein) can be immobilized on a solid support and then contacted with a fluid phase containing affinity reagents. Aspects of the methods are exemplified herein in the context of immobilized analytes and fluid phase affinity reagents. However, those skilled in the art will recognize that the teachings herein can be extended to a format in which affinity reagents are immobilized and contacted with fluid phase analytes.

A plurality of analytes can be provided in an array format, and each of the analytes in the array can be individually resolved from every other analyte in the array. As such, binding of each analyte to an affinity reagent can be measured, thereby providing single molecule-resolved detection. However, population dynamics can be determined from a combination of single molecule-resolved measurements. For example, the same species of proteins can be attached to each of a plurality of addresses and the addresses can be contacted with a fluid containing a plurality of affinity reagents that form complexes with the proteins. By counting the number of addresses that form a complex, a quantification of proteins of a given species can be determined. An advantage of monitoring single molecule-resolved proteins is that subpopulations of proteins can be identified based on different apparent binding characteristics. The observations can be used to identify population dynamics that would otherwise be averaged out in assays that detect ensemble-based addresses in arrays or that detect bulk solutions.

Methods are exemplified herein in the context of using fluid phase affinity reagents having optical labels (e.g., luminescent labels) and detecting optical signals (e.g., luminescence) at array addresses where the labeled affinity reagents have bound to a resident analyte (e.g., protein). For configurations in which the affinity reagents reside at the addresses and the analytes are in solution phase, the labelling scheme can be reversed (i.e. the analytes can bear the labels). Moreover, it will be understood that any of a variety of labels and detectors of their signals can be used as will be evident to those skilled in the art based on the teachings herein.

A set of affinity reagents can be contacted with an array using a fluidic technique that is appropriate to the hardware used. For example, fluid phase affinity reagents can be delivered by dipping the array in the fluid, dispensing the fluid onto the array surface (e.g., via a pipette), or flowing the fluid across the array surface. In particular embodiments, the array is contained in a flow cell having an ingress through which fluid is delivered and an egress through which fluid is removed.

A set of affinity reagents that is delivered to an array can include a quantity (e.g., concentration) of affinity reagents that is known or suspected to facilitate binding to the analytes in the array. Typically, the set of affinity reagents will include a single species of affinity reagent. However, in some cases a mixed pool of affinity reagent species can be present in the set. Optionally, the different species of affinity reagent can be distinguishably labeled. As such, addresses that bind to different species can be distinguished to allow characterization of binding properties for each respective species of affinity reagent in the set.

Binding of an affinity reagent to an analyte (e.g., protein) at a given address can be detected as signal emanating from the address. Absence of the signal at other addresses indicates that labeled affinity reagent has not bound at those other addresses. Detection can be carried out as set forth in further detail below.

Following detection, affinity reagents can be dissociated and optionally separated from analytes. For example, affinity reagents can be removed from contact with an array of proteins after detection of binding between the affinity reagents and proteins. An assay of the present disclosure can be carried out in multiple cycles, each cycle including two or more steps. An assay of the present disclosure can include at least 2, 5, 10, 25, 50, 100, 250 or more cycles. Alternatively or additionally, an assay can include at most 250, 100, 50, 25, 10, 5 or 2 cycles. Any number or combination of steps set forth herein can be included in the cycles. For example, each cycle can include steps of contacting an array with a set of affinity reagents, forming a nicked double helix at addresses where the affinity reagent binds, and detecting the nicked double helix at addresses of the array. As such, an assay of the present disclosure can include a cycle in which a first set of affinity reagents is contacted with an array and a subsequent cycle in which a second set of affinity reagents is contacted with the array. Two or more sets of affinity reagents that are used in respective cycles of an assay can contain the same species of affinity reagent. Alternatively, two or more sets of affinity reagents that are used in respective cycles of an assay can differ with regard to affinity reagent compositions. For example, affinity reagents can differ with regard to the epitopes recognized by affinity moieties that are present in the affinity reagents used for respective cycles.

A method of the present disclosure can include a step of hybridizing target nucleic acids of an analyte-affinity reagent complex with oligonucleotides to form a nicked double helix. The oligonucleotides can be configured to initiate formation of the nicked double helix when both first and second target nucleic acids are present in the complex. The target nucleic acids and oligonucleotides can be configured to require both the first and second target nucleic acids to produce the nicked double helix. For example, an affinity reagent, although having a first target nucleic acid, can be inhibited from initiating formation of the nicked double helix unless it is also in the presence of a second target nucleic acid from an analyte. Continuing with the example, the analyte, although having the second target nucleic acid, can be insufficient for initiating formation of the nicked double helix unless it is in the presence of a first target nucleic acid from the affinity reagent. In this example, the first and second target nucleic acids can be brought into proximity with each other when the affinity reagent binds to the analyte, and this can in turn allow formation of the nicked double helix in the presence of the oligonucleotides. Accordingly, detection of the nicked double helix can indicate binding between the affinity reagent and analyte even in the presence of unbound affinity reagent or unbound analyte.

FIG. 15 shows an exemplary configuration for detecting a complex between an analyte and affinity reagent. A first retaining component (black rectangle) is attached to an affinity reagent (Y shape) and to a first target nucleic acid (hatched arrow). A second retaining component (black rectangle with orthogonal post) is attached to a protein (cloud shape) and to a second target nucleic acid (open arrow). The arrow heads indicate 3′ ends of the nucleic acids and oligonucleotides. In the first reaction step, the affinity reagent binds to the protein to form the complex. The binding is reversible as indicated by the double arrows. Formation of the complex brings the first and second target nucleic acids into proximity with each other. In the second reaction step, a first oligonucleotide species (labeled “1”) hybridizes to the pair of target nucleic acids. The first oligonucleotide species is initially in a stem-loop conformation (also referred to as a hairpin conformation), wherein the stem is formed by reverse complementary sequences (indicated by black hatched and grey hatched portions of the strand) which self-anneals to form a double-stranded region, wherein the single stranded portion between the reverse complementary sequences forms the loop region (solid black portion of the strand), and wherein a single-stranded overhang occurs at the 3′ end of the first oligonucleotide species (grey bordered open section of the strand). The hybridization of the first oligonucleotide species to the target nucleic acids involves annealing of the 3′ overhang to the second target nucleic acid and annealing of a first of the two reverse complementary sequences (grey hatched section) to the first target nucleic acid. As a result of the first oligonucleotide species hybridizing to the target nucleic acids, the portion of the first oligonucleotide species that had formed a single stranded loop (solid black section of the strand) and the second of the two reverse complementary sequences are in a single stranded conformation and available to hybridize with a second oligonucleotide species (labeled “2”). The second oligonucleotide species is initially in a stem-loop conformation, wherein the stem is formed by reverse complementary sequences (indicated by black hatched and grey hatched portions of the strand) which form a double-stranded region, wherein the single stranded portion between the reverse complementary sequences form the loop region (black bordered open portion of the strand), and wherein a single-stranded overhang occurs at the 5′ end of the second oligonucleotide species (grey section of the strand). The hybridization of the second oligonucleotide species to the first oligonucleotide species involves annealing of the 5′ overhang of the second oligonucleotide species to the portion of the first oligonucleotide species that had formed a single stranded loop (solid black section of the strand) and annealing of a first of the two reverse complementary sequences (grey hatched section) of the second oligonucleotide species to the second of the two reverse complementary sequences of the first oligonucleotide species (black hatched section). As a result of the second oligonucleotide species hybridizing to the first oligonucleotide species, the portion of the second oligonucleotide species that had formed a single stranded loop (black bordered open portion of the strand) and the second of the two reverse complementary sequences are in a single stranded conformation and available to hybridize with another first oligonucleotide species as shown in the fourth reaction step. The nicked double helix can continue to grow by alternating hybridization of the first and second oligonucleotides in a chain reaction.

Generally, the rate of unwanted product formation, such as hybridization between the first oligonucleotide species (labeled “1” in FIG. 15) and the second oligonucleotide species (labeled “2” in FIG. 15) be low. For example, oligonucleotides can be designed or their sequences to preclude base-pair interaction. This could include, for example, the addition of loops, mismatches, modified bases, or other assay mechanisms to favor intended product formation such as the addition of ammonium sulfate, DMSO, DMF, PEG, or other additives known to improve the specificity of oligo interaction.

As demonstrated by the reaction diagrammed in FIG. 15, the juxtaposition of two target nucleic acids initiates a chain reaction in which a plurality of oligonucleotides are each converted from a self-annealed conformation (exemplified by the stem-loop structures) to a component of a nicked double helix containing multiple oligonucleotides. As each oligonucleotide is added to the double helix, a region of the oligonucleotide that was self-annealed becomes available to hybridize with another oligonucleotide. This reaction can potentially proceed until all oligonucleotides have been hybridized to the nicked double helix. In the reaction exemplified in FIG. 1, the plurality of oligonucleotides includes two oligonucleotide species which alternately hybridize to each other to form a single nicked double helix. It will be understood that more than two oligonucleotide species can be used. For example, at least 2, 3, 4, 5, 6 or more oligonucleotide species can be used. Moreover, the oligonucleotides need not form a single nicked double helix. For example, a branching double helix can be formed (e.g. a nucleic acid dendrimer) by hybridization of a plurality of oligonucleotides. Furthermore, the sequence regions of the oligonucleotide species that hybridize to each other need not be adjacent. For example, at least one of the oligonucleotide species can include gaps between the sequence regions that hybridize to each other. In some cases, at least one of the oligonucleotide species can include tails at the 3′ or 5′ end that do not anneal to another oligonucleotide in the assay. Gaps and tails can provide toeholds for hybridization of disruptor oligonucleotides which can disassemble the nicked double helix via strand displacement initiated at the toeholds. Thus, delivery of disruptor oligonucleotides can be used to remove nicked double helices, for example, to prepare for subsequent rounds of complex formation and detection. Exemplary, oligonucleotides and methods for producing nucleic acids in various conformations are set forth in Dirks and Pierce Proc. Nat'l. Acad. Sci. USA 101:15275-15278 (2004), Ang and Yung Chem. Comm. 52:4219-4222 (2016), or Liu et al. Sci. Adv. 8 eabk0133 (2022), each of which is incorporated herein by reference.

The formation of a nicked double helix can provide a detectable product that is indicative of binding between an affinity reagent and an analyte. As exemplified above, the affinity reagent and analyte can each include target nucleic acids that when both present in a complex provide a template to initiate hybridization of oligonucleotides to form a nicked double helix. Target nucleic acids and oligonucleotides can be designed to distinguish a formed complex having two target nucleic acids from the non-complexed components which only contain one of the target nucleic acids. This can be achieved, for example, by balancing the energetics of self-annealed oligonucleotides (e.g., stem-loop conformations) with the energetics of the oligonucleotides annealed to target nucleic acids. Balancing of the energetics provides specificity in distinguishing a complex, which has both target nucleic acids, from its non-complexed components, which each have only one of the target nucleic acids. Balancing the energetics of self-annealed oligonucleotides (e.g., stem-loop conformations) with the energetics of the oligonucleotides when annealed to each other to form a nicked double helix can be used to influence the rate at which the nicked double helix is produced and how many oligonucleotides get incorporated into the nicked double helix. The energetics can be balanced by choice of nucleotide sequences for the target nucleic acids and oligonucleotides. For example, the lengths of the nucleotide sequences, positioning of various sequence regions and percent content of G and C bases can be varied to alter the energetics of self-annealing and annealing with each other.

The lengths of nucleotide sequences in the oligonucleotides and target nucleic acids can be manipulated to achieve a desired balance of energetics. For example, the overall length of each target sequence which hybridizes to an oligonucleotide can be adjusted to at least 3, 4, 5, 6, 7, 8, 9, 10, 11, 12, 13, 14, 15 or more nucleotides. Alternatively or additionally, the overall length of each target sequence which hybridizes to an oligonucleotide can be adjusted to at most 15, 14, 13, 12, 11, 10, 9, 8, 7, 6, 5, 4, 3 or fewer nucleotides. The entire length of a pair of combined target nucleic acids which hybridizes to an oligonucleotide can be, for example, at least 4, 6, 8, 10, 12, 14, 16, 18, 20, 22, 24, 26, 28, 30 or more nucleotides. Alternatively or additionally, the entire length of a pair of combined target nucleic acids which hybridizes to an oligonucleotide can be for example, at most 30, 28, 26, 24, 22, 20, 18, 16, 14, 12, 10, 8, 6, 4 or fewer nucleotides. The length of sequence in an oligonucleotide which hybridizes to a pair of target nucleic acids can be, for example, at least 4, 6, 8, 10, 12, 14, 16, 18, 20, 22, 24, 26, 28, 30 or more nucleotides. Alternatively or additionally, length of sequence in an oligonucleotide which hybridizes to a pair of target nucleic acids can be, for example, at most 30, 28, 26, 24, 22, 20, 18, 16, 14, 12, 10, 8, 6, 4 or fewer nucleotides.

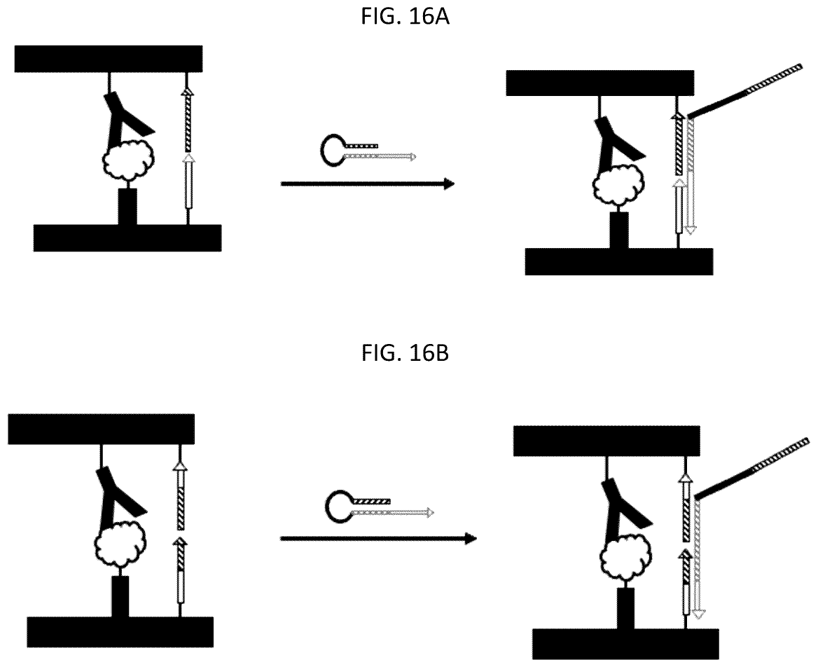

Moreover, the portion of an oligonucleotide that binds to a pair of target nucleic acids can be adjusted. FIG. 16A shows a first example, wherein a hairpin oligonucleotide hybridizes to a pair of target nucleic acids via annealing of the 3′ overhang (shown as the open section of the strand) of the oligonucleotide to a first target nucleic acid and annealing of a first of two reverse complementary sequences (shown as the grey hatched section of the strand) of the oligonucleotide to a second target nucleic acid. FIG. 16B shows a second example, wherein a hairpin oligonucleotide hybridizes to a pair of target nucleic acids via annealing of the 3′ overhang (shown as the open section of the strand) of the oligonucleotide to a portion of the first target nucleic acid and annealing of a first of two reverse complementary sequences (shown as the grey hatched section of the strand) of the oligonucleotide to portions of both the first and second target nucleic acids. A configuration in which both target nucleic acids are required to anneal to the stem region of a hairpin oligonucleotide can provide a differing route to specificity of the oligonucleotides for distinguishing a complex (i.e. having both target nucleic acids) from its unbound components (i.e. each having only one of the target nucleic acids). A reverse complementary sequence of a hairpin oligonucleotide can be configured to hybridize to two target nucleic acids via at least 2 nucleotides of both targets (i.e. at least 4 nucleotides total), via at least 3 nucleotides of both targets (i.e. at least 6 nucleotides total), via at least 4 nucleotides of both targets (i.e. at least 8 nucleotides total), via at least 5 nucleotides of both targets (i.e. at least 10 nucleotides total), via at least 6 nucleotides of both targets (i.e. at least 12 nucleotides total), via at least 7 nucleotides of both targets (i.e. at least 14 nucleotides total), or more. It will be understood that the reverse complementary region need not bind to an equal number of nucleotides in both target nucleic acids.

Another structural characteristic that can be manipulated to achieve a desired balance of energetics is the length of the hairpin sequences in an oligonucleotide that self-hybridize to from a stem. For example, an oligonucleotide can include a stem region or palindromic sequences capable of forming a self-hybridized region of at least 3, 4, 5, 6, 7, 8, 9, 10, 11, 12, 13, 14, 15 or more base pairs. Alternatively or additionally, the stem region or hairpin sequences capable of forming a self-hybridized region can include at most 15, 14, 13, 12, 11, 10, 9, 8, 7, 6, 5, 4, 3 or fewer base pairs. The length of a loop region or sequence capable of forming a loop when a stem-loop is formed can be at least 3, 4, 5, 6, 7, 8, 9, 10, 11, 12, 13, 14, 15 or more nucleotides. Alternatively or additionally, the loop region or sequence capable of forming a loop when a stem-loop is formed can be at most 15, 14, 13, 12, 11, 10, 9, 8, 7, 6, 5, 4, 3 or fewer nucleotides.

The GC content (i.e. percent of G and C bases) for a target nucleic acid or the portion of the target nucleic acid that hybridizes to an oligonucleotide can be at least 30%, 35%, 40%, 45%, 50%, 55%, 60%, 65%, 70% or more. Alternatively or additionally, the GC content for a target nucleic acid or the portion of the target nucleic acid that hybridizes to an oligonucleotide can be at most 70%, 65%, 60%, 55%, 50%, 45%, 40%, 35%, 30% or less. As such, the portion of an oligonucleotide that hybridizes to a pair of target nucleic acids can have a GC content in one of the aforementioned ranges. The GC content for a region of an oligonucleotide that is self-annealed or capable of self-annealing can be at least 30%, 35%, 40%, 45%, 50%, 55%, 60%, 65%, 70% or more. Alternatively or additionally, the GC content for a region of an oligonucleotide that is self-annealed or capable of self-annealing can be at most 70%, 65%, 60%, 55%, 50%, 45%, 40%, 35%, 30% or less.

Temperature is known to be a determinant of the stability of duplex DNA complexes. However, temperature also may affect globally the detection process between an affinity reagent and epitope. It therefore may be desirable for a region of an oligonucleotide that is self-annealed or capable of self-annealing to have typical melting temperature (Tₘ) between 4° C. and 50° C. under conditions used. For example, the Tₘ can be at least 4° C., 5° C., 10° C., 15° C., 20° C., 25° C., 30° C., 35° C., 40° C., 45° C., or more. Alternatively or additionally, the Tₘ can be at most 50° C., 45° C., 40° C., 35° C., 30° C., 25° C., 20° C., 15° C., 10° C., 5° C., or less. Oligonucleotide complexes which are not intended to exhibit binding may have non-measurable or insubstantial melting temperature (i.e. less than 0° C.) under conditions of use. Additionally, the melting temperatures for subdomains of oligonucleotides used in a method set forth herein, including their stem regions, loop regions or target recognition sites, may have melting temperatures to their binding partners as described in the sections above, also ranging from 4 to 50° C. Optionally, this is in addition or separate from the entire oligonucleotide melting temperature between two partners being at some other temperature from 4 to 50° C. As a further option, oligonucleotides can have intrastrand melting temperatures at a Tₘ from 4 to 50° C.

A method of the present disclosure can be performed to produce a nicked double helix that includes a plurality of oligonucleotide including, for example, at least 2, 5, 10, 15, 20, 25, 50, 100, 250, 500, 1000 or more oligonucleotides. Alternatively or additionally, a nicked double helix can include at most 1000, 500, 250, 100, 50, 25, 20, 15, 10, 5 or 2 oligonucleotides. A nicked double helix can optionally include at least 10, 25, 50, 100, 250, 500, 1000, 5000 or more base pairs. Alternatively or additionally, a nicked double helix can include at most 5000, 1000, 500, 250, 100, 75, 50, 40, 30, 20, 10 or fewer base pairs. A nicked double helix can include at least 1, 2, 5, 10, 15, 20, 25, 50, 100, 250, 500, 1000 or more nicks. Alternatively or additionally, a nicked double helix can include at most 1000, 500, 250, 100, 50, 25, 20, 15, 10, 5, 2 or 1 nick. In some cases, one or more nicks in a nicked double helix can each be adjacent to a gap of at least 1, 2, 3, 4, 5, 10 or more nucleotide positions. Alternatively or additionally, one or more gaps can span at most 10, 5, 4, 3, 2 or 1 nucleotide positions. Typically, gaps in a nicked double helix will have a uniform length with respect to each other. Similarly, a nicked double helix can include nicks and no gaps.

A nicked double helix can be produced by annealing of a plurality of labeled oligonucleotides. Individual oligonucleotides can each include at least 1, 2, 3, 4, 5, 10 or more labels. Alternatively or additionally, individual oligonucleotides can each include at most 10, 5, 4, 3, 2 or 1 label. A nicked double helix that is formed from a plurality of oligonucleotides can include at least 1, 2, 5, 10, 15, 20, 25, 50, 100, 250, 500, 1000 or more labels. Alternatively or additionally, a nicked double helix that is formed from a plurality of oligonucleotides can include at most 1000, 500, 250, 100, 50, 25, 20, 15, 10, 5, 2 or 1 label.

A nicked double helix can be produced by annealing a plurality of oligonucleotides selected from at least 1, 2, 3, 4, 5, 6 or more different species. Oligonucleotides of a given species will be understood to have identical nucleotide sequences, whereas different species will have different nucleotide sequences. Optionally, a nicked double helix can include a series of the first oligonucleotide species hybridized to a series of second oligonucleotide species. As such, the first species of oligonucleotides can include nucleotide sequences that are complementary to nucleotide sequences of the second species of oligonucleotides. Each of the strands in a nicked double helix can constitute a concatemer of one or more oligonucleotide species. Each of the strands in a nicked double helix can constitute a concatemer, wherein each unit of the concatemer is a sequence of one or more oligonucleotide species. In cases, where the oligonucleotides include hairpin sequences, a nicked double helix can include a concatemer, wherein the units of the concatemer include hairpin nucleotide sequences.

One or both strands of a nicked double helix can be nicked. The nicks can be a component of the concatemeric structure of a nicked double helix. For example, a nicked double helix can have a concatemeric structure, wherein the unit of the concatemer includes a sequence of an oligonucleotide and a nick. In another example, a nicked double helix can have a concatemeric structure, wherein the unit of the concatemer includes a sequence of an oligonucleotide and a gap of a particular length. Nicks or gaps can separate repeats of a nucleotide sequence in a nicked double helix.

In some configurations of the present methods one or more oligonucleotide species that are incorporated into a nicked double helix can include a label. Optionally, the label can be capable of being quenched. For example, the label can be in a quenched state when the oligonucleotide to which it is attached is in a self-annealed conformation, and the label can be in a competent state when the oligonucleotide is incorporated into a nicked double helix. By way of more specific example, a luminescent label can be attached to an oligonucleotide at a position that places the label in proximity to a quenching moiety when the oligonucleotide is in a self-annealed conformation. As such, the label will not produce substantial emission signal when the oligonucleotide is self-annealed. However, the label can be positioned such that it moves away from the quenching moiety when the oligonucleotide is annealed to another oligonucleotide. Exemplary positions for a label and quencher are at or near a reverse complementary sequence or other sequence that toggles between being self-annealed and being annealed to another oligonucleotide in a nicked double helix.

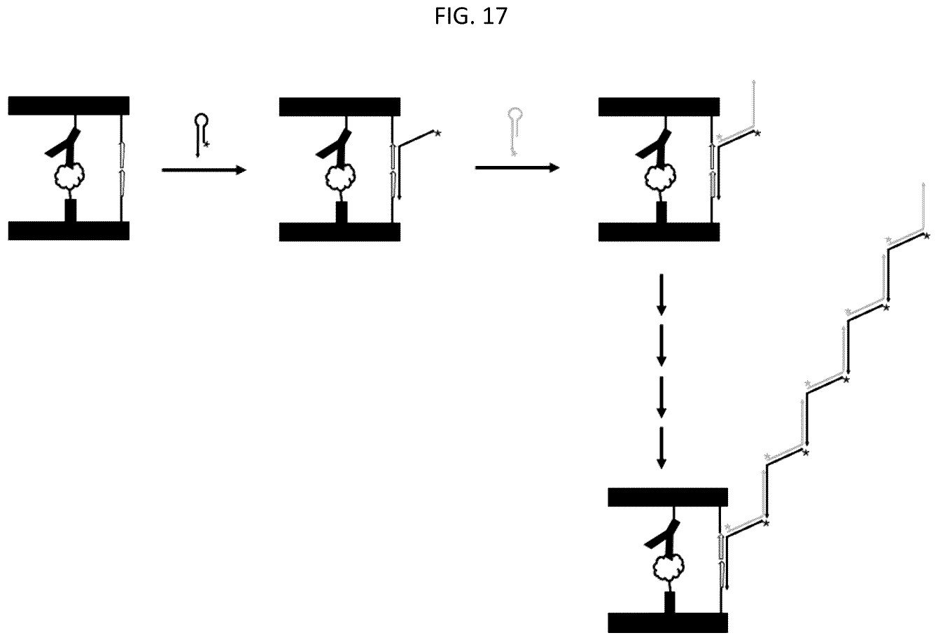

FIG. 17 shows an exemplary method for detecting a complex between an affinity reagent and protein. The affinity reagent, protein and related components including the target nucleic acids and retaining components are represented as set forth in the context of FIG. 15. In the first step, a first oligonucleotide species (black hairpin shape) is converted from a stem-loop (i.e. hairpin) conformation to a linearized shape when hybridized to the target nucleic acids. The first oligonucleotide species includes a label (shown as a black asterisk) that is attached to a strand adjacent to the self-annealed region of the stem-loop structure. In this conformation the label is quenched due to proximity to a quenching moiety on the opposite strand. When the first oligonucleotide species is linearized by hybridization to the target nucleic acids, the label is distant from the quenching moiety and thus competent to produce signal. In the second step, a second oligonucleotide species (grey hairpin shape) hybridizes to the first oligonucleotide species, thereby being converted from a stem-loop conformation to a linearized conformation. The second oligonucleotide species also includes a label (grey asterisk) which toggles from a quenched state in the stem-loop conformation to a competent state in the nicked double helix. Although it is possible to deliver the two species of oligonucleotide alternately to produce the nicked double helix, generally both oligonucleotide species can be present initially and simultaneously. The respective oligonucleotide species can spontaneously add to the growing nicked double helix in an alternating fashion. Multiple arrows in the last steps of FIG. 17 indicate further steps in the hybridization chain reaction whereby first and second oligonucleotide species are alternately added to the nicked double helix. In the example shown, ten labeled oligonucleotides (i.e. 5 of each of the two oligonucleotide species) have been added to the nicked double helix, thus providing ten labels and a high degree of signal amplification for detection of the complex. The skilled person will readily recognize that use of self-quenching may be useful in other methods set forth herein, such as those depicted in FIGS. 8A-8B, or FIGS. 14A-14C.

A nicked double helix that is hybridized to a complex via first and second target nucleic acids can be dissociated by separating the first target nucleic acid from the second nucleic acid. The first target nucleic acid, if attached to an affinity reagent, can be separated from the second target nucleic acid, if attached to an analyte, by dissociation of the affinity reagent from the analyte. For example, the complex can form at an address of an array where the analyte resides due to binding of the affinity reagent to the analyte and due to formation of the nicked double helix at the address. Dissociation of the affinity reagent from the protein will result in physical separation of the second target nucleic acid from the address which will, in turn, result in dissociation of the nicked double helix from the address. In an equilibrium binding condition, dynamic binding between the affinity reagent and analyte can cause affinity reagents to repeatedly associate and dissociate from the address. The dwell time of the affinity reagent at the address can provide a measure of strength or avidity of binding between the analyte at the address and the affinity reagent. Accordingly, detection of the dwell time for a nicked double helix at the address can provide a measure of binding strength or avidity of the affinity reagent for the analyte at the address. Moreover, the intensity of signal from a given address after a given period of time can provide a measure of binding strength or avidity in cases where the nicked double helix grows at a predictable rate. For example, as labeled oligonucleotides are added to the growing nicked double helix at an array address, signal will increase as a measure of the persistence of the complex at the address.

Detection can be carried out using hardware that resolves individual addresses in an array set forth herein. As such, a detection technique used in a method set forth herein can resolve any number of addresses up to an including all addresses in an array set forth herein. In some cases, optical labels can be detected using an optical detector. For example, luminophores (e.g. fluorophores) can be detected using a luminescence detector (e.g., fluorometer). A particularly useful configuration for detecting an array is an epiluminescence configuration. However, other configurations such as total internal reflection (TIR) can also be used.

Exemplary labels and detectable signals they produce include, without limitation, optical labels such as luminophores (e.g., fluorophores) which emit photons at particular wavelengths when excited by radiation and which can be distinguished due to luminescence lifetime or polarization. Other useful optical labels include chromophores which absorb radiation at particular wavelengths and nanoparticles which can interact with light to produce signals such as photon emissions or light scatter. Other labels include heavy atoms, radioactive isotopes, mass labels, charge labels, spin labels, nucleic acids having particular sequences, receptors, ligands, or the like. Signals that can be detected from labels in methods set forth herein include, for example, optical signals such as absorbance of radiation, luminescence emission, luminescence lifetime, luminescence polarization, fluorescence emission, fluorescence lifetime, fluorescence polarization, or the like; Rayleigh and/or Mie scattering; binding affinity for a ligand or receptor; magnetic properties; electrical properties; charge; mass; radioactivity, nucleotide sequences detected via nucleic acid sequencing platforms, sequence specific hybridization of nucleic acid labels to complementary probes or the like.

In some configurations of a method set forth herein, detection can include quantifying signals produced by a nicked double helix. Signals can optionally be quantified after a given period of time. For example, signal can be analyzed as a single acquisition that is optionally compared to a background signal. In other configurations, signal can be acquired over a period of time and a change in the signal can be analyzed. As such, signal can be acquired as the mass of a nicked double helix increases. This will typically result in an increase in signal intensity over time. However, in some configurations a method can measure a loss of signal intensity over time, for example, as a nicked double helix grows.

Any of a variety of assay formats can be used to detect an analyte. Several methods will be exemplified below in the context of detecting proteins but can readily be extended to other analytes by modifications that will be apparent to those skilled in the art. A protein can be detected using one or more affinity reagents having binding affinity for the protein. The affinity reagent and the protein can bind each other to form a complex. The affinity reagent and protein can each be co-localized with a target nucleic acid, for example, via direct attachment or attachment mediated by a retaining component. The complex can be detected due to formation of a nicked double helix. Exemplary assay formats that can utilize the binding and detection steps set forth herein are provided below. Others will be known or otherwise accessible to those skilled in the art.

In some configurations, a plurality of different affinity reagents can be detected in parallel and distinguished from each other. For example, each of the different affinity reagents can be distinguished by the target nucleic acid to which it is attached. Hybridization chain reactions using different pairs of oligonucleotides can be initiated by respective target nucleic acids to form unique nicked double helices. Each pair of oligonucleotides can optionally be attached to a unique label. Looking to the example of an array of proteins, a variety of different proteins can be attached to the array, with each address of the array being attached to a single species of protein. Each address can be attached to a universal target nucleic acid (i.e. the target nucleic acid at each address is the same as all addresses in the array). The array can be contacted with two different affinity reagents, wherein the first affinity reagent is attached to a first target nucleic acid and the second affinity reagent is attached to a second target nucleic acid. The nucleic acid sequence of the first target nucleic acid differs from the second target nucleic acid. For sake of this example, assume that the first affinity reagent binds to a protein that is present at a first address to form a first complex and the second affinity reagent binds to a protein that is present at a second address to form a second complex. In this configuration, the first complex includes the universal target nucleic acid and the first target nucleic acid, and the second complex includes the universal target nucleic acid and the second target nucleic acid. The array can be in contact with a first set of hairpin oligonucleotides that is initiated by the combination of the universal target nucleic acid and the first target nucleic acid to form a first nicked double helix. The array can also be in contact with a second set of hairpin oligonucleotides that is initiated by the combination of the universal target nucleic acid and the second target nucleic acid to form a second nicked double helix. The first set of hairpin oligonucleotides can include a first label that is distinguishable from a second label that is present in the second set of hairpin oligonucleotides. As such, each of the addresses that binds to a respective affinity reagent can be distinguished based on the label that is detected there. By appropriate choice of target nucleic acid sequences and oligonucleotide probe sets, any desired number of affinity reagent species can be multiplexed in a given assay or a given cycle of an assay including, for example, at least 2, 3, 4, 5, 10 or more species.

### Assaying Proteins

Any of a variety of assay formats can be used to detect an analyte. Several methods will be exemplified below in the context of detecting proteins but can readily be extended to other analytes by modifications that will be apparent to those skilled in the art. Assay methods and formats set forth below can be modified to include linked affinity reagents (or linked analytes) or to apply various stimuli to attract affinity reagents (or analytes) as set forth above.

A protein can be detected using one or more affinity reagents having binding affinity for the protein. The affinity reagent and the protein can bind each other to form a complex and the complex can be detected during or after formation. The complex can be detected directly, for example, due to a label that is present on the affinity reagent or protein. In some configurations, the complex need not be directly detected, for example, in formats where the complex is formed and then the affinity reagent, protein, or a label component that was present in the complex is subsequently detected.

Many protein assays, such as enzyme linked immunosorbent assay (ELISA), achieve high-confidence characterization of one or more proteins in a sample by exploiting high specificity binding of affinity reagents to the protein(s) and detecting the binding event while ignoring all other proteins in the sample. Binding assays can be carried out by detecting affinity reagents and/or proteins that are immobilized in multiwell plates, on arrays, or on particles in microfluidic devices. Exemplary plate-based methods include, for example, the MULTI-ARRAY technology commercialized by MesoScale Diagnostics (Rockville, Maryland) or Simple Plex technology commercialized by Protein Simple (San Jose, CA). Exemplary, array-based methods include, but are not limited to those utilizing Simoa® Planar Array Technology or Simoa® Bead Technology, commercialized by Quanterix (Billerica, MA). Further exemplary array-based methods are set forth in U.S. Pat. Nos. 9,678,068; 9,395,359; 8,415,171; 8,236,574; or 8,222,047, each of which is incorporated herein by reference. Exemplary microfluidic detection methods include those commercialized by Luminex (Austin, Texas) under the trade name xMAP® technology or used on platforms identified as MAGPIX®, LUMINEX® 100/200 or FLEXMAP 3D®. These and other formats can be modified to include linked affinity reagents (or linked analytes) or to apply various stimuli to attract affinity reagents (or analytes) as set forth herein.

Exemplary assay formats that can be performed at a variety of plexity scales up to and including proteome scale are set forth in U.S. Pat. No. 10,473,654 or US Pat. App. Pub. Nos. 2020/0318101 A1 or 2020/0286584 A1; U.S. patent application Ser. No. 18/045,036, or Egertson et al., BioRxiv (2021), DOI: 10.1101/2021.10.11.463967, each of which is incorporated herein by reference. A plurality of proteins can be assayed for binding to affinity reagents, for example, on single-molecule resolved protein arrays. Proteins can be in a denatured state or native state when manipulated or detected in a method set forth herein.

Turning to the example of an array-based assay configuration, the identity of an extant protein at any given address is typically not known prior to performing the assay. The assay can be used to identify extant proteins at one or more addresses in the array. A plurality of affinity reagents, optionally labeled (e.g. with fluorophores), can bind to proteins in the array, and the addresses can be detected to determine binding outcomes. A plurality of different affinity reagents can be used serially, such that each cycle detects binding outcomes for a given type of affinity reagent (e.g. a type of affinity reagent having affinity for a particular epitope) at each address. The assay can include a step of removing affinity reagents from the array after detecting the binding outcomes, such that the next affinity reagent can be delivered to the flow cell and detected. In some configurations, a plurality of different affinity reagents can be detected in parallel, for example, when different affinity reagents are distinguishably labeled.

In particular configurations, a method set forth herein can be used to identify a number of different extant proteins that exceeds the number of affinity reagents used. For example, the number of different protein species identified can be at least 5×, 10×, 25×, 50×, 100× or more than the number of affinity reagents used. This can be achieved, for example, by (1) using promiscuous affinity reagents that bind to multiple different candidate proteins suspected of being present in a given sample, and (2) subjecting the extant proteins to a set of promiscuous affinity reagents that, taken as a whole, are expected to bind each candidate protein in a different combination, such that each candidate protein is expected to generate a unique profile of binding and non-binding events. Promiscuity of an affinity reagent can arise due to the affinity reagent recognizing an epitope that is known to be present in a plurality of different candidate proteins. For example, epitopes having relatively short amino acid lengths such as dimers, trimers, tetramers or pentamers can be expected to occur in a substantial number of different proteins in a typical proteome. Alternatively or additionally, a given promiscuous affinity reagent may recognize multiple different epitopes (e.g., epitopes differing from each other with regard to amino acid composition or sequence). For example, a promiscuous affinity reagent that is designed or selected for its affinity toward a first trimer epitope may also have affinity for a second epitope that has a different sequence of amino acids compared to the first epitope.

The present disclosure provides compositions, apparatus and methods that can be useful for characterizing analytes, such as proteins, by obtaining multiple separate and non-identical measurements of the analytes. In particular configurations, the individual measurements may not, by themselves, be sufficiently accurate or specific to make the characterization, but in combination the multiple non-identical measurements can allow the characterization to be made with a high degree of accuracy, specificity and confidence. For example, the multiple separate measurements can include subjecting a sample to reagents that are promiscuous with regard to recognizing a variety of different analytes that are present in the sample. Accordingly, a first measurement carried out using a first promiscuous reagent may perceive a first subset of the analytes without distinguishing different analytes within the subset. A second measurement carried out using a second promiscuous reagent may perceive a second subset of analytes, again, without distinguishing one analyte in the second subset from other analytes in the second subset. However, a comparison of the first and second measurements can distinguish: (i) an analyte that is uniquely present in the first subset but not the second; (ii) an analyte that is uniquely present in the second subset but not the first; (iii) an analyte that is uniquely present in both the first and second subsets; or (iv) an analyte that is uniquely absent in the first and second subsets. The number of promiscuous reagents used, the number of separate measurements acquired, and degree of reagent promiscuity (e.g. the diversity of components recognized by the reagent) can be adjusted to suit the diversity of analytes expected for a particular sample.

The present disclosure provides assays that are useful for detecting one or more analytes. Exemplary assays are set forth herein in the context of detecting proteins. Those skilled in the art will recognize that methods, compositions and apparatus set forth herein can be adapted for use with other analytes such as cells, organelles, nucleic acids, polysaccharides, metabolites, vitamins, hormones, enzyme co-factors, therapeutic agents, candidate therapeutic agents and others set forth herein or known in the art. Particular configurations of the methods, apparatus and compositions set forth herein can be made and used, for example, as set forth in U.S. Pat. Nos. 10,473,654 or 11,282,585; US Pat. App. Pub. Nos. 2020/0082914A1 or 2023/0114905A1; or Egertson et al., BioRxiv (2021), DOI: 10.1101/2021.10.11.463967, each of which is incorporated herein by reference. Exemplary methods, systems and compositions are set forth in further detail below.

A composition, apparatus or method set forth herein can be used to characterize an analyte, or moiety thereof, with respect to any of a variety of characteristics or features including, for example, presence, absence, quantity (e.g., amount or concentration), chemical reactivity, molecular structure, structural integrity (e.g., full length or fragmented), maturation state (e.g., presence or absence of pre- or pro-sequence in a protein), location (e.g., in an analytical system, subcellular compartment, cell or natural environment), association with another analyte or moiety, binding affinity for another analyte or moiety, biological activity, chemical activity or the like. An analyte can be characterized with regard to a relatively generic characteristic such as the presence or absence of a common structural feature (e.g, amino acid sequence length, overall charge or overall pKa for a protein) or common moiety (e.g., a short primary sequence motif or post-translational modification for a protein). An analyte can be characterized with regard to a relatively specific characteristic such as a unique amino acid sequence (e.g., for the full length of the protein or a motif), an RNA or DNA sequence that encodes a protein (e.g., for the full length of the protein or a motif), or an enzymatic or other activity that identifies a protein. A characterization can be sufficiently specific to identify an analyte, for example, at a level that is considered adequate or unambiguous by those skilled in the art.

In some detection assays, a protein can be cyclically modified and the modified products from individual cycles can be detected. For example, a protein can be sequenced by a sequential process in which each cycle includes steps of detecting the protein and removing one or more terminal amino acids from the protein to produce a shortened protein. The shortened protein is then subjected to subsequent cycles. Optionally, a protein sequencing method can include steps of adding a label to the protein, for example, at the amino terminal amino acid or at the carboxy terminal amino acid. In particular configurations, a method a protein sequencing method can include steps of (i) removing a terminal amino acid from the protein, thereby forming a truncated protein; (ii) detecting a change in signal from the truncated protein, for example, in comparison to the protein prior to truncation; and (iii) identifying the type of amino acid that was removed in step (i) based on the change detected in step (ii). The terminal amino acid can be removed, for example, by removal of one or more amino acids from the amino terminus or carboxyl terminus of the protein. Steps (i) through (iii) can be repeated to produce a series of signal changes that is indicative of the sequence for the protein.

In a first configuration of a protein sequencing method, one or more types of amino acids in the protein can be attached to a label that uniquely identifies the type of amino acid. In this configuration, the change in signal that identifies the amino acid can be loss of signal from the respective label. For example, lysines can be attached to a distinguishable label such that loss of the label indicates removal of a lysine. Alternatively or additionally, other amino acid types can be attached to other labels that are mutually distinguishable from lysine and from each other. For example, lysines can be attached to a first label and cysteines can be attached to a second label, the first and second labels being distinguishable from each other. Exemplary compositions and techniques that can be used to remove amino acids from a protein and detect signal changes are those set forth in Swaminathan et al., Nature Biotech. 36:1076-1082 (2018); or U.S. Pat. Nos. 9,625,469 or 10,545,153, each of which is incorporated herein by reference. Methods and apparatus under development by Erisyon, Inc. (Austin, TX) may also be useful for sequencing, or otherwise detecting, proteins.

In a second configuration of a cyclical protein detection method, a terminal amino acid of a protein can be recognized by an affinity agent that is specific for the terminal amino acid, specific for a labeled terminal amino acid (e.g., the affinity agent can recognize the label alone or in combination with the side chain of a particular type of amino acid). The affinity agent can be detected on the array, for example, due to a label on the affinity agent. Optionally, the label is a nucleic acid barcode sequence that is added to a primer nucleic acid upon formation of a complex. For example, a barcode can be added to the primer via ligation of an oligonucleotide having the barcode sequence or polymerase extension directed by a template that encodes the barcode sequence. The formation of the complex and identity of the terminal amino acid can be determined by decoding the barcode sequence. Multiple cycles can produce a series of barcodes that can be detected, for example, using a nucleic acid sequencing technique. Exemplary affinity agents and detection methods are set forth in US Pat. App. Pub. No. 2019/0145982 A1; 2020/0348308 A1; or 2020/0348307 A1, each of which is incorporated herein by reference. Methods and apparatus under development by Encodia, Inc. (San Diego, CA) or Standard BioTools (e.g., technology developed by SomaLogic or Palamaedrix) may also be useful for detecting proteins.

Cyclical removal of terminal amino acids from a protein can be carried out using an Edman-type sequencing reaction. In some configurations, an Edman-type sequencing reaction can involve reaction of a phenyl isothiocyanate with an N-terminal amino group of a protein under mildly alkaline conditions (e.g., about pH 8) to form a cyclical phenylthiocarbamoyl Edman complex derivative. The phenyl isothiocyanate may be substituted or unsubstituted with one or more functional groups, linker groups, or linker groups containing functional groups. An Edman-type sequencing reaction can include variations to reagents and conditions that yield detectable removal of amino acids from a protein terminus, thereby facilitating determination of the amino acid sequence for a protein or portion thereof. For example, the phenyl group can be replaced with at least one aromatic, heteroaromatic or aliphatic group which may participate in an Edman-type sequencing reaction, non-limiting examples including: pyridine, pyrimidine, pyrazine, pyridazoline, fused aromatic groups such as naphthalene and quinoline), methyl or other alkyl groups or alkyl group derivatives (e.g., alkenyl, alkynyl, cyclo-alkyl). Under certain conditions, for example, acidic conditions of about pH 2, derivatized terminal amino acids may be cleaved, for example, as a thiazolinone derivative. The thiazolinone amino acid derivative under acidic conditions may form a more stable phenylthiohydantoin (PTH) or similar amino acid derivative which can be detected. This procedure can be repeated iteratively for residual protein to identify the subsequent N-terminal amino acid. Many variations of Edman-type degradation have been described and may be used including, for example, a one-step removal of an N-terminal amino acid using alkaline conditions (Chang, J. Y., FEBS LETTS., 1978, 91 (1), 63-68). In some cases, Edman-type reactions may be thwarted by N-terminal modifications which may be selectively removed, for example, N-terminal acetylation or formylation (e.g., see Gheorghe M. T., Bergman T. (1995) in Methods in Protein Structure Analysis, Chapter 8: Deacetylation and internal cleavage of Proteins for N-terminal Sequence Analysis. Springer, Boston, MA. https://doi.org/10.1007/978-1-4899-1031-8_8).

Non-limiting examples of functional groups for substituted phenyl isothiocyanate may include ligands (e.g. biotin and biotin analogs) for known receptors, labels such as luminophores, or reactive groups such as click functionalities (e.g. compositions having an azide or acetylene moiety). The functional group may be a DNA, RNA, peptide or small molecule barcode or other tag which may be further processed and/or detected.

Edman-type processes can be carried out in a multiplex format to detect, characterize or identify a plurality of proteins. A method of detecting a protein can include steps of (i) exposing a terminal amino acid on a protein at an address of an array; (ii) binding an affinity agent to the terminal amino acid, where the affinity agent includes a nucleic acid tag, and where a primer nucleic acid is present at the address; (iii) extending the primer nucleic acid in the presence of the nucleic acid tag, thereby producing an extended primer having a copy of the tag; and (iv) detecting the tag of the extended primer. The terminal amino acid can be exposed, for example, by removal of one or more amino acids from the amino terminus or carboxyl terminus of the protein. Steps (i) through (iv) can be repeated to produce a series of tags that is indicative of the sequence for the protein. The method can be applied to a plurality of proteins on the array and in parallel. The extending of a primer can be carried out, for example, by polymerase-based extension of the primer, using the nucleic acid tag as a template. Alternatively, the extending of a primer can be carried out, for example, by ligase-or chemical-based ligation of the primer to a nucleic acid that is hybridized to the nucleic acid tag. The nucleic acid tag can be detected via hybridization to nucleic acid probes (e.g., in an array), amplification-based detections (e.g. PCR-based detection, or rolling circle amplification-based detection) or nuclei acid sequencing (e.g. cyclical reversible terminator methods, nanopore methods, or single molecule, real time detection methods). Exemplary methods that can be used for detecting proteins using nucleic acid tags are set forth in US Pat. App. Pub. No. 2019/0145982 A1; 2020/0348308 A1; or 2020/0348307 A1, each of which is incorporated herein by reference.

A protein can optionally be detected based on its enzymatic or biological activity. For example, a protein can be contacted with a reactant that is converted to a detectable product by an enzymatic activity of the protein. In other assay formats, a first protein having a known enzymatic function can be contacted with a second protein to determine if the second protein changes the enzymatic function of the first protein. As such, the first protein serves as a reporter system for detection of the second protein. Exemplary changes that can be observed include, but are not limited to, activation of the enzymatic function, inhibition of the enzymatic function, attenuation of the enzymatic function, degradation of the first protein or competition for a reactant or cofactor used by the first protein. Proteins can also be detected based on their binding interactions with other molecules such as other proteins, nucleic acids, nucleotides, metabolites, hormones, vitamins, small molecules that participate in biological signal transduction pathways, biological receptors or the like. For example, a protein that participates in a signal transduction pathway can be identified as a particular candidate protein by detecting binding to a second protein that is known to be a binding partner for the candidate protein in the pathway.

In some configurations of the apparatus and methods set forth herein, one or more proteins can be detected on a solid support. For example, protein(s) can be attached to a solid support, the solid support can be contacted with detection agents (e.g. affinity agents) in solution, the agents can interact with the protein(s), thereby producing a detectable signal, and then the signal can be detected to determine the presence of the protein(s). In multiplexed versions of this approach, different proteins can be attached to different addresses in an array, and the probing and detection steps can occur in parallel. In another example, affinity agents can be attached to a solid support, the support can be contacted with proteins in solution, the proteins can interact with the affinity agents, thereby producing a detectable signal, and then the signal can be detected to determine presence, quantity or characteristics of the proteins. This approach can also be multiplexed by attaching different affinity agents to different addresses of an array.

Proteins, affinity agents or other objects of interest can be attached to a solid support via covalent or non-covalent bonds. For example, a linker can be used to covalently attach a protein or other object of interest to an array. A particularly useful linker is a structured nucleic acid particle such as a nucleic acid nanoball (e.g. a concatemeric amplicon produced by rolling circle replication of a circular nucleic acid template) or a nucleic acid origami. For example, a plurality of proteins can be conjugated to a plurality of structured nucleic acid particles, such that each protein-conjugated particle forms a respective address in the array. Exemplary linkers for attaching proteins, or other objects of interest, to an array or other solid support are set forth in U.S. Pat. Nos. 11,203,612 or 11,505,796 or US Pat. App. Pub. No. 2023/0167488 A1, each of which is incorporated herein by reference.

A protein can be detected based on proximity of two or more affinity agents. For example, the two affinity agents can include two components each: a receptor component and a nucleic acid component. When the affinity agents bind in proximity to each other, for example, due to ligands for the respective receptors being at the same address in an array, the nucleic acids can interact to cause a modification that is indicative of the two ligands being in proximity. Optionally, the modification can be polymerase catalyzed extension of one of the nucleic acids using the other nucleic acid as a template. As another option, one of the nucleic acids can form a template that acts as splint to position other nucleic acids for ligation to an oligonucleotide. Exemplary methods are commercialized by Olink Proteomics AB (Uppsala Sweden) or set forth in U.S. Pat. Nos. 7,306,904; 7,351,528; 8,013,134; 8,268,554 or 9,777,315, each of which is incorporated herein by reference.

In some configurations of the compositions, apparatus and methods set forth herein, one or more proteins can be present on a solid support, where the proteins can optionally be detected. For example, a protein can be attached to a solid support, the solid support can be contacted with a detection agent (e.g., affinity agent) in solution, the affinity agent can interact with the protein, thereby producing a detectable signal, and then the signal can be detected to determine the presence, absence, quantity, a characteristic or identity of the protein. In multiplexed versions of this approach, different proteins can be attached to different addresses in an array, and the detection steps can occur in parallel, such that proteins at each address are detected, quantified, characterized or identified. In another example, detection agents can be attached to a solid support, the support can be contacted with proteins in solution, the proteins can interact with the detection agents, thereby producing a detectable signal, and then the signal can be detected to determine the presence of the proteins. This approach can also be multiplexed by attaching different probes to different addresses of an array.

In multiplexed configurations, different proteins can be attached to different unique identifiers (e.g., addresses in an array), and the proteins can be manipulated and detected in parallel. For example, a fluid containing one or more different affinity agents can be delivered to an array such that the proteins of the array are in simultaneous contact with the affinity agent(s). Moreover, a plurality of addresses can be observed in parallel allowing for rapid detection of binding events. A plurality of different proteins can have a complexity of at least 5, 10, 100, 1×10³, 1×10⁴, 1×10⁵ or more different native-length protein primary sequences. Alternatively or additionally, a proteome, proteome subfraction or other protein sample that is analyzed in a method set forth herein can have a complexity that is at most 1×10⁵, 1×10⁴, 1×10³, 100, 10, 5 or fewer different native-length protein primary sequences. The total number of proteins of a sample that is detected, characterized or identified can differ from the number of different primary sequences in the sample, for example, due to the presence of multiple copies of at least some protein species. Moreover, the total number of proteins of a sample that is detected, characterized or identified can differ from the number of candidate proteins suspected of being in the sample, for example, due to the presence of multiple copies of at least some protein species, absence of some proteins in a source for the sample, or loss of some proteins prior to analysis.

A protein can be attached to a unique identifier using any of a variety of means. The attachment can be covalent or non-covalent. Exemplary covalent attachments include chemical linkers such as those achieved using click chemistry or other linkages known in the art or described in U.S. patent application Ser. No. 17/062,405, which is incorporated herein by reference. Non-covalent attachment can be mediated by receptor-ligand interactions (e.g., (strept) avidin-biotin, antibody-antigen, or complementary nucleic acid strands), for example, wherein the receptor is attached to the unique identifier and the ligand is attached to the protein or vice versa. In particular configurations, a protein is attached to a solid support (e.g. an address in an array) via a structured nucleic acid particle (SNAP). A protein can be attached to a SNAP and the SNAP can interact with a solid support, for example, by non-covalent interactions of the DNA with the support and/or via covalent linkage of the SNAP to the support. Nucleic acid origami or nucleic acid nanoballs are particularly useful. The use of SNAPs and other moieties to attach proteins to unique identifiers such as tags or addresses in an array are set forth in U.S. patents application Ser. Nos. 17/062,405 and 63/159,500, each of which is incorporated herein by reference.

A method set forth herein can be carried out in a fluid phase or on a solid phase. For fluid phase configurations, a fluid containing one or more proteins can be mixed with another fluid containing one or more affinity agents. For solid phase configurations one or more proteins or affinity agents can be attached to a solid support. One or more components that will participate in a binding event can be contained in a fluid and the fluid can be delivered to a solid support, the solid support being attached to one or more other component that will participate in the binding event. A solid support can be composed of a substrate that is insoluble in aqueous liquid. The substrate can have any of a variety of other characteristics such as being rigid, non-porous or porous. Exemplary solid supports include, but are not limited to, glass and modified or functionalized glass, plastics (including acrylics, polystyrene and copolymers of styrene and other materials, polypropylene, polyethylene, polybutylene, polyurethanes, Teflon™, cyclic olefins, polyimides etc.), nylon, ceramics, resins, Zeonor™, silica or silica-based materials including silicon and modified silicon, carbon, metals, inorganic glasses, optical fiber bundles, gels, and polymers. In some cases, a solid support may comprise silicon, fused silica, quartz, mica, or borosilicate glass. In particular configurations a flow cell contains the solid support such that fluids introduced to the flow cell can interact with a surface of the solid support to which one or more components of a binding event (or other reaction) is attached.

A method of the present disclosure can be carried out at single analyte resolution. As such, a single analyte (i.e. one and only one analyte), such as a single protein, can be individually manipulated or distinguished using a method set forth herein. A single analyte can be a single molecule (e.g. single protein), a single complex of two or more molecules (e.g. a single protein attached to a structured nucleic acid particle or a single protein attached to an affinity agent), a single particle, or the like. A single analyte may be resolved from other analytes based on, for example, spatial or temporal separation from the other analytes. Reference herein to a ‘single analyte’ in the context of a composition, apparatus or method does not necessarily exclude application of the composition, apparatus or method to multiple single analytes that are manipulated or distinguished individually, unless indicated to the contrary.

Alternatively to single-analyte resolution, a method can be carried out at ensemble-resolution or bulk-resolution. Bulk-resolution configurations acquire a composite signal from a plurality of different analytes or affinity agents in a vessel or on a surface. For example, a composite signal can be acquired from a population of different protein-affinity agent complexes in a well or cuvette, or on a solid support surface, such that individual complexes are not resolved from each other. Ensemble-resolution configurations acquire a composite signal from a first collection of proteins or affinity agents in a sample, such that the composite signal is distinguishable from signals generated by a second collection of proteins or affinity agents in the sample. For example, the ensembles can be located at different addresses in an array. Accordingly, the composite signal obtained from each address will be an average of signals from the ensemble, yet signals from different addresses can be distinguished from each other.

A composition, apparatus or method set forth herein can be configured to contact one or more analytes (e.g., an array of different proteins) with a plurality of different affinity agents. For example, a plurality of affinity agents (whether configured separately or as a pool) may include at least 2, 5, 10, 25, 50, 100, 250, 500 or more types of affinity agents, each type of affinity agent differing from the other types with respect to the epitope(s) recognized. Alternatively or additionally, a plurality of affinity agents may include at most 500, 250, 100, 50, 25, 10, 5, or 2 types of affinity agents, each type of affinity agent differing from the other types with respect to the epitope(s) recognized. Different types of affinity agents in a pool can be uniquely labeled such that the different types can be distinguished from each other. In some configurations, at least two, and up to all, of the different types of affinity agents in a pool may be indistinguishably labeled with respect to each other. Alternatively or additionally to the use of unique labels, different types of affinity agents can be delivered and detected serially when evaluating one or more proteins (e.g., in an array).

A method of the present disclosure can be performed in a multiplex format. In multiplexed configurations, different analytes can be attached to different unique identifiers (e.g. proteins can be attached to different addresses in an array). Multiplexed analytes can be manipulated and detected in parallel. For example, a fluid containing one or more different affinity agents can be delivered to a protein array such that the proteins of the array are in simultaneous contact with the affinity agent(s). Moreover, a plurality of addresses can be observed in parallel allowing for rapid detection of binding events.

A particularly useful multiplex format uses an array of analytes (e.g., proteins) and/or affinity agents. The analytes and/or affinity agents can be attached to unique identifiers (e.g., addresses of the array) such that the analytes can be distinguished from each other. An array can be used in any of a variety of processes such as an analytical process used for detecting, identifying, characterizing or quantifying an analyte. Analytes can be attached to unique identifiers via covalent or non-covalent (e.g., ionic bond, hydrogen bond, van der Waals forces etc.) bonds. An array can include different analyte species that are each attached to different unique identifiers. An array can include different unique identifiers that are attached to the same or similar analyte species. An array can include separate solid supports or separate addresses that each bear a different analyte, in which the different analytes can be identified according to the locations of the solid supports or addresses.

An address of an array can contain a single analyte, or it can contain a population of several analytes of the same species (i.e. an ensemble of the analytes). Alternatively, an address can include a population of different analytes.

A detection apparatus can include a light sensing device that is appropriate for detecting a characteristic set forth herein or known in the art. Particularly useful components of a light sensing device can include, but are not limited to, optical sub-systems or components used in nucleic acid sequencing systems. Examples of useful sub systems and components thereof are set forth in US Pat. App. Pub. No. 2010/0111768 A1 or U.S. Pat. Nos. 7,329,860; 8,951,781 or 9,193,996, each of which is incorporated herein by reference. Other useful light sensing devices and components thereof are described in U.S. Pat. Nos. 5,888,737; 6,175,002; 5,695,934; 6,140,489; or 5,863,722; or US Pat. Pub. Nos. 2007/007991 A1, 2009/0247414 A1, or 2010/0111768; or WO2007/123744, each of which is incorporated herein by reference. Light sensing devices and components that can be used to detect luminophores based on luminescence lifetime are described, for example, in U.S. Pat. Nos. 9,678,012; 9,921,157; 10,605,730; 10,712,274; 10,775,305; or 10,895,534, each of which is incorporated herein by reference.

For configurations that use optical detection (e.g., luminescent detection), one or more analytes (e.g., proteins) may be immobilized on a surface, and this surface may be observed by a microscope to detect any signal from the immobilized analytes. The microscope itself may include a digital camera or other luminescence detector configured to record, store, and analyze the data collected during the scan. A luminescence detector can further include an excitation source that is capable of irradiating analytes, for example, proteins at addresses on an array, at an appropriate wavelength. A luminescence detector of the present disclosure can be configured for epiluminescent detection, total internal reflection (TIR) detection, waveguide assisted excitation, or the like. Optical filters or other optical components can be present to tune the wavelength, polarization or other optical properties of excitation and/or emission radiation used by a luminescence detector.

A light sensing device may be based upon any suitable technology, and may be, for example, a charged coupled device (CCD) sensor that generates pixilated image data based upon photons impacting locations in the device. It will be understood that any of a variety of other light sensing devices may also be used including, but not limited to, a detector array configured for time delay integration (TDI) operation, a complementary metal oxide semiconductor (CMOS) detector, an avalanche photodiode (APD) detector, a Geiger-mode photon counter, a photomultiplier tube (PMT), charge injection device (CID) sensors, JOT image sensor (Quanta), or any other suitable detector. Light sensing devices can optionally be coupled with one or more excitation sources, for example, lasers, light emitting diodes (LEDs), arc lamps or other energy sources known in the art.

A light sensing device can be configured for single molecule resolution. For example, waveguides or optical confinements can be used to deliver excitation radiation to locations of a solid support where analytes are located. Zero-mode waveguides can be particularly useful, examples of which are set forth in U.S. Pat. Nos. 7,181,122, 7,302,146, or 7,313,308, each of which is incorporated herein by reference. Analytes can be confined to surface features that function as addresses and facilitate single molecule resolution. For example, analytes can be distributed into wells having nanometer dimensions such as those set forth in U.S. Pat. Nos. 7,122,482 or 8,765,359, or US Pat. App. Pub. No 2013/0116153 A1, each of which is incorporated herein by reference. The wells can be configured for selective excitation, for example, as set forth in U.S. Pat. No. 8,798,414 or 9,347,829, each of which is incorporated herein by reference. Analytes can be distributed to nanometer-scale posts, such as high aspect ratio posts which can optionally be dielectric pillars that extend through a metallic layer to improve detection of an analyte attached to the pillar. See, for example, U.S. Pat. Nos. 8,148,264, 9,410,887 or 9,987,609, each of which is incorporated herein by reference. Further examples of nanostructures that can be used to detect analytes are those that change state in response to the concentration of analytes such that the analytes can be quantitated as set forth in WO 2020/176793 A1, which is incorporated herein by reference.

A detection apparatus need not be configured for optical detection. For example, an electronic detector can be used for detection of protons or charged labels (see, for example, US Pat. App. Pub. Nos. 2009/0026082 A1; 2009/0127589 A1; 2010/0137143 A1; or 2010/0282617 A1, each of which is incorporated herein by reference in its entirety). A field effect transistor (FET) can be used to detect analytes or other entities, for example, based on proximity of a field disrupting moiety to the FET. The field disrupting moiety can be due to an extrinsic label attached to an analyte or affinity reagent, or the moiety can be intrinsic to the analyte or affinity agent being used. Surface plasmon resonance can be used to detect binding of analytes or affinity agents at or near a surface. Exemplary sensors and methods for attaching molecules to sensors are set forth in US Pat. App. Pub. Nos. 2017/0240962 A1; 2018/0051316 A1; 2018/0112265 A1; 2018/0155773 A1 or 2018/0305727 A1; or U.S. Pat. Nos. 9,164,053; 9,829,456; 10,036,064, each of which is incorporated herein by reference.

Luminescence lifetime can be detected using an integrated circuit having a photodetection region configured to receive incident photons and produce a plurality of charge carriers in response to the incident photons. The integrated circuit can include at least one charge carrier storage region and a charge carrier segregation structure configured to selectively direct charge carriers of the plurality of charge carriers directly into the charge carrier storage region based upon times at which the charge carriers are produced. See, for example, U.S. Pat. Nos. 9,606,058, 10,775,305, and 10,845,308, each of which is incorporated herein by reference. Optical sources that produce short optical pulses can be used for luminescence lifetime measurements. For example, a light source, such as a semiconductor laser or LED, can be driven with a bipolar waveform to generate optical pulses with FWHM durations as short as approximately 85 ps having suppressed tail emission. See, for example, in U.S. Pat. No. 10,605,730, which is incorporated herein by reference.

A detection apparatus can include a fluidics system, for example, configured for fluidic communication with a vessel, such as a flow cell. In some configurations, a detection apparatus can include one or more reservoirs containing affinity reagents or analytes that are delivered to a vessel. Optionally, a detection apparatus can be configured to include a waste receptacle to which waste from the vessel is collected. For example, a composition set forth herein can be delivered from the apparatus through an ingress of a flow cell and waste can be removed through an egress of the flow cell to the apparatus.

A solid support or a surface thereof may be configured to display an analyte or a plurality of analytes. A solid support may contain one or more addresses in formed or prepared surfaces. Multiple addresses can be configured to form a pattern. In some cases, a solid support may contain one or more patterned, formed, or prepared surfaces that contain a plurality of addresses, with each address configured to display one or more analytes. Accordingly, an array as set forth herein may comprise a plurality of analytes coupled to a solid support or a surface thereof. In some configurations, a solid support or a surface thereof may be patterned or formed to produce an ordered or repeating pattern of addresses. The deposition of analytes on the repeating pattern of addresses may be controlled by interactions between the solid support and the analytes such as, for example, electrostatic interactions, magnetic interactions, hydrophobic interactions, hydrophilic interactions, covalent interactions, or non-covalent interactions. Accordingly, the coupling of an analyte at each address of an array may produce an array of analytes whose average spacing between analytes is relatively uniform, for example, being determined based upon the tolerance of the ordering or patterning of the solid support and the size of an analyte-binding region for each address. An ordered or patterned array of analytes may be characterized as having a regular geometry, such as a rectangular, triangular, polygonal, or annular grid. In other configurations, a solid support or a surface thereof may have a random or non-repeating pattern of addresses. The deposition of analytes on the random or non-repeating pattern may be controlled by interactions between the solid support and the analytes, or inter-analyte interactions such as, for example, steric repulsion, electrostatic repulsion, electrostatic attraction, magnetic repulsion, magnetic attraction, covalent interactions, or non-covalent interactions.

A solid support or a surface thereof may contain one or more structures or features. A structure or feature may comprise an elevation, profile, shape, geometry, or configuration that deviates from an average elevation, profile, shape, geometry, or configuration of a solid support or surface thereof. A structure or feature may be a raised structure or feature, such as a ridge, post, pillar, or pad, if the structure or feature extends above the average elevation of a surface of a solid support. A structure or feature may be a depressed structure, such as a channel, well, pore, or hole, if the structure or feature extends below the average elevation of a surface of a solid support. A structure or feature may be an intrinsic structure or feature of a substrate (i.e., arising due to the physical or chemical properties of the substrate, or a physical or chemical mechanism of formation), such as surface roughness structures, crystal structures, or porosity. A structure or feature may be formed by a method of processing a solid support. In some configurations, a solid support or a surface may be processed by a lithographic method to form one or more structures or features. A solid support or a surface thereof may be formed by a suitable lithographic method, including, but not limited to photolithography, Dip-Pen nanolithography, nanoimprint lithography, nanosphere lithography, nanoball lithography, nanopillar arrays, nanowire lithography, immersion lithography, neutral particle lithography, plasmonic lithography, scanning probe lithography, thermochemical lithography, thermal scanning probe lithography, local oxidation nanolithography, molecular self-assembly, stencil lithography, laser interference lithography, soft lithography, magnetolithography, stereolithography, deep ultraviolet lithography, x-ray lithography, ion projection lithography, proton-beam lithography, or electron-beam lithography.

A solid support or surface may comprise a plurality of structures or features. Structures or features may be provided as analyte-binding sites for the coupling of analytes or other moieties (e.g., anchoring moieties). A plurality of structures or features may comprise a repeating pattern of structures or features. A plurality of structures or features may comprise a non-ordered, non-repeating, or random distribution of structures or features. A structure or feature may have an average characteristic dimension (e.g., length, width, height, diameter, circumference, etc.) of at least about 1 nanometer (nm), 5 nm, 10 nm, 20 nm, 30 nm, 40 nm, 50 nm, 75 nm, 100 nm, 150 nm, 200 nm, 250 nm, 300 nm, 400 nm, 500 nm, 750 nm, 1000 nm, or more than 1000 nm. Alternatively or additionally, a structure or feature may have an average characteristic dimension of no more than about 1000 nm, 750 nm, 500 nm, 400 nm, 300 nm, 250 nm, 200 nm, 150 nm, 100 nm, 75 nm, 50 nm, 40 nm, 30 nm, 20 nm, 10 nm, 5 nm, 1 nm, or less than 1 nm. An array of structures or features may have an average pitch, in which the pitch is measured as the average separation between respective centerpoints of adjacent structures or features. An array may have an average pitch of at least about 1 nm, 5 nm, 10 nm, 20 nm, 30 nm, 40 nm, 50 nm, 75 nm, 100 nm, 150 nm, 200 nm, 250 nm, 300 nm, 400 nm, 500 nm, 750 nm, 1 micron (μm), 2 μm, 5 μm, 10 μm, 50 μm, 100 μm, or more than 100 μm. Alternatively or additionally, an array may have an average pitch of no more than about 100 μm, 50 μm, 10 μm, 5 μm, 1 μm, 750 nm, 500 nm, 400 nm, 300 nm, 250 nm, 200 nm, 150 nm, 100 nm, 75 nm, 50 nm, 40 nm, 30 nm, 20 nm, 10 nm, 5 nm, 1 nm, or less than 1 nm.

A structure or feature of an array may have a characteristic dimension (e.g., a width, length, or diameter) that is smaller than a characteristic dimension of an analyte or other object (e.g., a nanoparticle) that is attached to the structure or feature. It may be preferable to provide structures or features that are smaller than analytes or other objects attached to the structure or feature to occlude the attachment of additional analytes or other objects to the structure or feature. Alternatively, a structure or feature may have a characteristic dimension that is larger than a characteristic dimension of an analyte or other object (e.g., a nanoparticle) that is attached to the structure or feature.

A solid support or a surface thereof may include a base substrate material and, optionally, one or more additional materials that are contacted or adhered with the substrate material. A solid support may comprise one or more additional materials that are deposited, coated, or inlayed onto the substrate material. Additional materials may be added to the substrate material to alter the properties of the substrate material. For example, materials may be added to alter the surface chemistry (e.g., hydrophobicity, hydrophilicity, non-specific binding, electrostatic properties), alter the optical properties (e.g., reflective properties, refractive properties), alter the electrical or magnetic properties (e.g., dielectric materials, conducting materials, electrically-insulating materials), or alter the heat transfer characteristics of the substrate material. Additional materials contacted or adhered with a substrate material may be ordered or patterned onto the substrate material to, for example, locate the additional material at addresses or locate the additional material at interstitial regions between addresses. Exemplary additional materials may include metals (e.g., gold, silver, copper, etc.), metal oxides (e.g., titanium oxide, silicon dioxide, alumina, iron oxides, etc.), metal nitrides (e.g., silicon nitride, aluminum nitride, boron nitride, gallium nitride, etc.), metal carbides (e.g., tungsten carbide, titanium carbide, iron carbide, etc.), metal sulfides (e.g., iron sulfide, silver sulfide, etc.), and organic moieties (e.g., polyethylene glycol (PEG), dextrans, chemically-reactive functional groups, etc.).

A method of the present disclosure can include the step of coupling one or more analytes to a solid support or a surface thereof, for example, prior to performing a detection step set forth herein. The coupling of one or more analytes to a solid support surface may include covalent or non-covalent coupling of the one or more analytes to the solid support. Covalent coupling of an analyte to a solid support can include direct covalent coupling of an analyte to a solid support (e.g., formation of coordination bonds) or indirect covalent coupling between a reactive functional group of the analyte and a reactive functional group that is coupled to the solid support (e.g., a CLICK-type reaction). Non-covalent coupling can include the formation of any non-covalent interaction between an analyte and a solid support, including electrostatic or magnetic interactions, or non-covalent bonding interactions (e.g., ionic bonds, van der Waals interactions, hydrogen bonding, etc.). The skilled person will readily recognize that the particular analyte and the choice of solid support can affect the selection of a coupling chemistry for the compositions and methods set forth herein.

Accordingly, a coupling chemistry may be selected based upon the criterium that it provides a sufficiently stable coupling of an analyte to a solid support for a time scale that meets or exceeds the time scale of a method as set forth herein. For example, a polypeptide identification method can require a coupling of the analyte to the solid support for a sufficient amount of time to permit a series of empirical measurements of the analyte to occur. An analyte may be continuously coupled to a solid support for an observable length of time such as, for example, at least about 1 minute, 1 hour (hr), 3 hrs, 6 hrs, 12 hrs, 1 day, 1.5 days, 2 days, 3 days, 1 week (wk), 2 wks, 3 wks, 1 month, or more. The coupling of an analyte to a solid support can occur with a solution-phase chemistry that promotes the deposition of the analyte on the solid support. Coupling of an analyte to a solid support may occur under solution conditions that are optimized for any conceivable solution property, including solution composition, species concentrations, pH, ionic strength, solution temperature, etc. Solution composition can be varied by chemical species, such as buffer type, salts, acids, bases, and surfactants. In some configurations, species such as salts and surfactants may be selected to facilitate the formation of interactions between an analyte and a solid support. Covalent coupling methods for coupling an analyte to a solid support may include species such as catalyst, initiators, and promoters to facilitate particular reactive chemistries.

An array of analytes may be provided for a method, composition, system, or apparatus set forth in the present disclosure. Although analytes are exemplified as proteins throughout the present disclosure, it will be understood that other analytes may be provided in a similar array format. Exemplary analytes include, but are not limited to, cells, organelles, biomolecules, polysaccharides, nucleic acids, lipids, metabolites, hormones, vitamins, enzyme cofactors, therapeutic agents, candidate therapeutic agents, or combinations thereof. An analyte can be a non-biological atom or molecule, such as a synthetic polymer, metal, metal oxide, ceramic, semiconductor, mineral, or a combination thereof.

An array of analytes may be provided on a solid support containing a plurality of discrete analyte-binding sites. The analyte-binding sites may be present at addresses. Each analyte-binding site may be separated from each other analyte-binding site by one or more interstitial regions. For example, each analyte-binding site may be located at a respective address, wherein the addresses are separated from each other by one or more interstitial regions. An array interstitial region may be configured to inhibit binding of analytes or other moieties to the interstitial region, for example by containing a surface coating or layer. Exemplary interstitial region surface layers or coatings can include hydrophobic moieties (e.g., hexmethyldisilazane, alkyl moieties) or hydrophilic moieties (e.g., polyethylene glycol moieties). Surface layers or coatings provided at an interstitial region can comprise linear, branched, or dendrimeric moieties. A surface layer or coating provided at an interstitial region may be a self-assembled monolayer. An address can include a single analyte-binding site (i.e. one and only one analyte-binding site or, alternatively, a plurality of analyte-binding sites can be present at a given address.

Array analyte-binding sites can comprise one or more moieties that are coupled or otherwise bound to a solid support at the analyte-binding site. Moieties may be bound to a solid support at an analyte-binding site for facilitating coupling of an analyte to the analyte-binding site, or to inhibit unwanted binding of moieties to the analyte-binding site. Moieties may be covalently or non-covalently bound to a solid support at an analyte-binding site.

An analyte-binding site may be provided with one or more moieties that couple an analyte to the analyte-binding site. Coupling moieties can include non-covalent coupling moieties (e.g., oligonucleotides, receptor-ligand binding pairs, electrically-charged moieties, magnetic moieties, etc.), or covalent coupling moieties (e.g., Click-type reactive groups, etc.). An analyte-binding site may be provided with one or more passivating moieties that inhibit unwanted or unexpected binding of moieties to the analyte-binding site. Exemplary passivating moieties can include polymeric molecules such as polyethylene glycol (PEG), bovine serum albumin, pluronic F-127, polyvinylpyrrolidone, and Teflon, or hydrophobic materials such as hexamethyldisilazane. A passivating moiety may be covalently or non-covalently bound to a solid support at an analyte-binding site. An analyte-binding site may contain a covalently bound passivating moiety and a non-covalently bound passivating moiety. For example, an analyte-binding site may contain a PEG moiety that is covalently attached to the solid support at the analyte-binding site and a bovine serum albumin moiety that is electrostatically bound to the analyte-binding site.

An analyte-binding site may comprise a plurality of moieties coupled to a solid support. The plurality of moieties can include a coupling moiety and an optional plurality of passivating moieties. Preferably, a moiety containing a coupling moiety may further comprise a passivating moiety. For example, an oligonucleotide coupling moiety may further comprise a PEG passivating moiety. In some configurations, each individual moiety of a plurality of moieties coupled to an analyte-binding site can contain a coupling moiety. Alternatively, in some configurations, only a fraction of moieties of a plurality of moieties coupled to an analyte-binding site may contain a coupling moiety. Coupling moieties and passivating moieties may be provided at an analyte-binding site in a ratio of at least about 1000:1, 100:1, 10:1, 5:1, 2:1, 1:1, 1:2, 1:5, 1:10, 1:100, or 1:1000 coupling-to-passivating moieties. Alternatively or additionally, coupling moieties and passivating moieties may be provided at an analyte-binding site in a ratio of no more than about 1:1000, 1:100, 1:10, 1:5, 1:2, 1:1, 2:1, 5:1, 10:1, 100:1, or 1000:1 coupling-to-passivating moieties.

Analyte-binding sites may have an average characteristic dimension of at least about 10 nm, 25 nm, 50 nm, 100 nm, 150 nm, 200 nm, 250 nm, 300 nm, 500 nm, 1 micron, or more than 1 micron. Alternatively or additionally, analyte-binding sites may have an average characteristic dimension of no more than about 1 micron, 500 nm, 300 nm, 250 nm, 200 nm, 150 nm, 100 nm, 50 nm, 25 nm, 10 nm, or less than 10 nm.

Analytes may be attached directly to analyte-binding sites, for example, by coupling of a moiety attached to an analyte to a moiety attached to an analyte-binding site. Alternatively, analytes may be attached to analyte-binding sites by an anchoring moiety. An anchoring moiety may attach an analyte to an analyte-binding site, and optionally orient the analyte and/or occlude additional analytes from attaching to the analyte-binding site. An anchoring moiety may comprise a nanoparticle, such as a metal nanoparticle, a metal oxide nanoparticle, a semiconductor nanoparticle, a carbon nanoparticle, or a polymeric nanoparticle. Preferably, an anchoring moiety may comprise a nucleic acid nanoparticle. A nucleic acid nanoparticle of an anchoring moiety may comprise a first face containing one or more coupling moieties, and a second face containing an analyte-coupling site. The first face and the second face of the anchoring moiety may be substantially opposed. The anchoring moiety may further comprise a linking moiety that attaches the analyte to the anchoring moiety. The linking moiety may spatially separate the analyte from the surface of the array, for example by a distance of at least about 5 nm, 10 nm, 15 nm, 20 nm, 25 nm, 30 nm, 40 nm, 50 nm, or more than 50 nm. The linking moiety may comprise a flexible linker (e.g., a PEG or alkyl moiety) or a rigid linker (e.g., a double-stranded nucleic acid linker). An anchoring moiety may be attached to one and only one analyte. An anchoring moiety may be attached to more than one analyte. Additional aspects of anchoring moieties are described in U.S. Pat. Nos. 11,203,612, and 11,505,796, each of which is incorporated herein by reference in its entirety.

An array of analytes may be provided with a characterized or characterizable analyte-binding site occupancy. The analyte-binding site occupancy can be measured as the fraction or percentage of analyte-binding sites of a plurality of analyte-binding sites containing an attached analyte. An array of analytes may be provided with an analyte-binding site occupancy of at least about 10%, 20%, 25%, 30%, 35%, 40%, 45%, 50%, 60%, 70%, 80%, 90%, 95%, 99%, 99.9%, or more than 99.9%. Alternatively or additionally, an array of analytes may be provided with an analyte-binding site occupancy of no more than about 99.9%, 99%, 95%, 90%, 80%, 70%, 60%, 50%, 45%, 40%, 35%, 30%, 25%, 20%, 10%, or less than 10%.

An array of analytes may be provided with a fraction or percentage of individual sites that each contain one and only one analyte. The fraction or percentage may be calculated relative to all other sites in the array including, but not limited to, those containing no analyte and those containing multiple analytes. Preferably, an array of analytes may be provided with super Poisson loading of single analytes (i.e., a fraction or percentage of attachments sites containing one and only one analyte exceeding 37%). An array of analytes may be provided with at least about 10%, 20%, 25%, 30%, 35%, 37%, 40%, 45%, 50%, 60%, 70%, 80%, 90%, 95%, 99%, 99.9%, or more than 99.9% of analyte-binding sites containing one and only one analyte. Alternatively or additionally, an array of analytes may be provided with no more than about 99.9%, 99%, 95%, 90%, 80%, 70%, 60%, 50%, 45%, 40%, 35%, 30%, 25%, 20%, 10%, or less than 10% of analyte-binding sites containing one and only one analyte.

It may be especially useful to provide an array of analytes with a diversity of polypeptide species. The diversity of polypeptide species may be measured with respect to a proteome, sub-proteome (e.g., a tissue proteome, a cell proteome, an organelle proteome, a metabolome, a signalome, an albuminome, etc.), or a microbiome. An array of analytes may be provided with a diversity of polypeptide species as measured by total number of polypeptide species, percentage of species of a proteome, subproteome, or microbiome, number of proteoforms of a polypeptide species, or polypeptide dynamic range.

An array of analytes may be provided with a fraction or percentage of species of a proteome, subproteome, or microbiome. An array of analytes may be provided with at least about 0.1%, 1%, 5%, 10%, 15%, 20%, 25%, 30%, 35%, 40%, 45%, 50%, 55%, 60%, 65%, 70%, 75%, 80%, 85%, 90%, 95%, 99%, 99.9%, or more than 99.9% of polypeptide species of a proteome, subproteome, or microbiome. Alternatively or additionally, an array of analytes may be provided with no more than about 99.9%, 99%, 95%, 90%, 85%, 80%, 75%, 70%, 65%, 60%, 55%, 50%, 45%, 40%, 35%, 30%, 25%, 20%, 15%, 10%, 5%, 1%, 0.1%, or less than 0.1% of polypeptide species of a proteome, subproteome, or microbiome.

An array of analytes may be provided with more than one proteoform of a polypeptide species. An array of analytes may be provided with more than one proteoform for two or more unique polypeptide species. Types of proteoforms of a polypeptide species can include coding variation proteoforms, translational variation proteoforms, post-translational modification proteoforms, splice variants, and combinations thereof. An array of analytes may be provided with at least about 2, 3, 4, 5, 10, 20, 50, 100, 200, 500, 1000, or more than 1000 proteoforms of a polypeptide species. Alternatively or additionally, an array of analytes may be provided with no more than about 1000, 500, 200, 100, 50, 20, 10, 5, 4, 3, or less than 3 proteoforms of a polypeptide species.

In some methods, providing an array of analytes may further comprise forming the array of analytes. An array of analytes may be formed by a process that includes a step of coupling analytes to analyte-binding sites of the array. An analyte may be coupled to an analyte-binding site by coupling of a coupling moiety attached to the analyte to a compatible coupling moiety attached to the analyte-binding site. In some cases where an analyte is attached to an anchoring moiety, a step of coupling the analyte to the analyte-binding site may comprise coupling the anchoring moiety to the analyte-binding site. In particular cases, an analyte may be coupled to an analyte-binding site by coupling of a coupling moiety attached to an anchoring moiety to a compatible coupling moiety attached to the analyte-binding site.

When forming an array of analytes, a plurality of analytes may be provided in a fluidic medium. A fluidic medium containing a plurality of analytes may be contacted to a solid support comprising a plurality of analyte-binding sites. After contacting the fluidic medium comprising the analytes to the solid support, analytes may couple to analyte-binding sites, thereby forming the array of analytes. In some cases, after contacting a fluidic medium containing analytes to a solid support containing analyte-binding sites, a mass transfer process may occur to facilitate coupling of the analytes to the analyte-binding sites. A mass transfer process can include chemical or mechanical processes that increase a rate of mass transfer of analytes to the surface of the solid support containing the analyte-binding sites. Chemical methods can include altering a pH (e.g., increasing the pH, decreasing the pH), ionic strength (e.g., increasing the ionic strength, decreasing the ionic strength), or temperature (e.g., increasing the temperature, decreasing the temperature) of a fluidic medium containing analytes. A chemical method of increasing mass transfer of analytes may depend upon the chemical composition of the analytes or moieties attached thereto (e.g., anchoring moieties). For example, an analyte attached to a nucleic acid nanoparticle (or any other particle having a net negative electrical surface charge) may transfer toward a hydrophobic surface more readily if the ionic strength of the fluidic medium is decreased. Mechanical methods of increasing mass transfer can include any suitable method of imparting a force on an analyte or a moiety attached thereto, such as centrifugation, electrophoresis, or magnetic attraction. Accordingly, it may be useful to provide an analyte attached to an electrically-charged particle, a magnetic particle, a particle that is denser than a fluidic medium, or a combination thereof.

A method of forming an array of analytes may include repeating one or more steps of attaching analytes to analyte-binding sites of the array. It may be preferable to repeat certain analyte-coupling steps to increase the analyte-binding site occupancy of an array of analytes. Fluidic media containing analytes may be repetitively or sequentially contacted to a solid support. A method of forming an array of analytes may further include a rinsing step (e.g., after contacting a fluidic medium to a solid support), thereby removing unbound or weakly-bound analytes or other moieties (e.g., anchoring moieties) from contact with the solid support.

Compositions set forth herein can interact with each other via covalent bonds. Molecules, moieties thereof or atoms thereof can form covalent bonds with other molecules, moieties or atoms. Covalent interactions can be reversible or irreversible in the context of a method set forth herein. A covalent bond can arise due to a chemical reaction between a first reactive moiety and a second reactive moiety, optionally in the presence of a third intermediary or catalytic moiety. Covalent bonds can be formed via various chemical mechanisms, including addition, substitution, elimination, oxidation, and reduction. In some cases, a covalent binding interaction may be formed by a Click-type reaction, as set forth herein (e.g., methyltetrazine (mTz)-tetracyclooctylene (TCO), azide-dibenzocyclooctene (DBCO), thiol-epoxy). In some cases, a ligand-receptor-type binding interaction can form a covalent binding interaction. For example, SpyCatcher-SpyTag, SnoopCatcher-SnoopTag, and SdyCatcher-SdyTag are receptor-ligand binding pairs that can form covalent binding interactions due to isopeptide bond formation. Additional useful covalent binding interactions can include coordination bond formation, such as between a metal-containing substrate and a ligand. Exemplary coordination bonds can include silicon-silane, metal oxide-phosphate, and metal oxide-phosphonate. Useful reagents and mechanisms for forming covalent binding interactions, including bioorthogonal binding interactions, as set forth herein, are provided in U.S. Pat. Nos. 11,203,612 or 11,505,796, each of which is herein incorporated by reference in its entirety

Compositions set forth herein can interact with each other via non-covalent bonds. A non-covalent bond can include an electrostatic or magnetic interaction between a first moiety and a second moiety. A non-covalent bond can include electrostatic interactions such as ionic bonding, hydrogen bonding, halogen bonding, Van der Waals interactions, Pi-Pi stacking, Pi-ion interactions, Pi-polar interactions, or magnetic interactions. In some cases, a non-covalent bond may be formed by hybridization of a first oligonucleotide to a complementary second oligonucleotide. Such bonding is also known as Watson-Crick base-pairing. In some cases, a non-covalent interaction may be formed by a receptor-ligand binding pair, such as streptavidin-biotin. Other useful non-covalent interactions can include affinity reagent-target interactions, such as antibody-epitope or aptamer-epitope interactions.

Systems and methods for forming and utilizing arrays, such as those set forth herein, may contain multiple types of covalent and/or non-covalent interactions. For example, a useful array site configuration may comprise an analyte (e.g., a polypeptide) that is covalently bonded to an oligonucleotide, in which the oligonucleotide is hybridized to a nucleic acid nanoparticle, in which the nucleic acid nanoparticle is hybridized to a surface-coupled oligonucleotide, and in which the surface-coupled oligonucleotide is covalently bonded to a surface of a solid support. This example may be extended to further include an affinity reagent that is non-covalently bound to the analyte. The affinity reagent bound to the analyte, in turn, may be covalently bonded to a nanoparticle or a moiety thereof (e.g., an oligonucleotide). The skilled person will recognize that the various covalent and non-covalent interactions occurring in the system and methods set forth herein may vary with respect to both time-scale and reversibility (or lack thereof) for association and/or dissociation of the binding interactions. Accordingly, it will be recognized that certain binding interactions (e.g., covalent binding of an analyte to an oligonucleotide) will be selected to inhibit or minimize a likelihood of association or dissociation over the duration of a method, or a step thereof, as set forth herein, and other binding interactions (e.g., non-covalent binding of an affinity reagent to an analyte) will be selected to facilitate or increase a likelihood of association or dissociation within the duration of a method or a step thereof, as set forth herein.

Entities, such as affinity reagents and their binding targets, can be associated with each other and dissociated form each other in a method set forth herein. Association of a first entity to a second entity can involve a contacting step, in which the first entity is brought into proximity of the second entity, and an association step in which a first coupling moiety of the first entity forms a binding interaction with a second coupling moiety of the second entity. Dissociation of a first entity and a second entity need not be construed as a reversal of an association process between the first entity and the second entity. For example, a first entity comprising a first oligonucleotide coupled to a second entity comprising a second oligonucleotide by hybridization of the first oligonucleotide to the second oligonucleotide could be dissociated by dehybridization of the nucleic acids (thereby returning the first entity and the second entity as originally provided before association), or dissociated by enzymatic cleavage of the hybridized nucleic acids (thereby providing the first and the second entities with each individually further comprising an at least partially double-stranded cleavage product).

Systems or methods set forth herein may utilize one or more fluidic media to implement a process or step thereof. For array-based processes and systems, fluidic media may be provided for various process steps, including preparing arrays, attaching analytes to arrays, associating affinity agents to analytes, dissociating affinity agents from analytes, rinsing unbound moieties from array surfaces, performing detection processes on arrays, displacing a fluidic medium from contact with an array or other system components, and various other chemical and/or physical alterations of analytes or array components. A fluidic medium may be formulated to deliver a plurality of macromolecules (e.g., analytes, affinity agents) to an array as set forth herein. A fluidic medium may be formulated to mediate an interaction between macromolecules (e.g., an interaction between an analyte and an affinity agent).

A fluidic medium may be a single-phase or multi-phase fluidic medium. A multi-phase fluidic medium can include a gas phase and a liquid phase or at least two immiscible liquids. A multi-phase fluidic medium may comprise an interface between a first phase and a second phase. An interface between two fluidic phases may be laminar (e.g., an oil phase floating on an aqueous phase) or dispersed (e.g., bubbles, vesicles or droplets). A dispersed interface may be formed by a process such as emulsification. A divided interface may be stable (e.g., an emulsion) or unstable (e.g., a flocculating suspension). A multi-phase fluidic medium may comprise a colloidal agent that mediates an interface between a first phase and a second phase.

A fluidic medium can further contain solids, including particles (e.g., microparticles, nanoparticles). A fluidic medium comprising solids may be provided as a mixture, a suspension, or a slurry. It may be advantageous to provide a fluidic medium comprising a mixture or suspension of macromolecules. In some cases, solubility or suspendability of solids, such as particles or macromolecules, within a fluidic medium can be modulated by the composition of the fluidic medium. For example, alteration of fluidic properties such as solvent composition, ionic strength, and/or pH can induce precipitation, sedimentation, or flocculation of solvated or suspended solids.

A fluidic medium may be formulated with any one of numerous components depending upon its intended application. A fluidic medium can comprise one or more solvents. A single-phase fluidic medium can comprise two or more miscible solvents. In a mixture of miscible solvents, a solvent may be considered a base solvent if it comprises a greater than 50% fraction on a mass, molar, or volumetric basis. A miscible solvent may be mixed into a base solvent to alter a physical property of the base solvent, such as polarity, density, pH, viscosity, or surface tension. A fluidic medium can comprise a polar solvent or a non-polar solvent. A fluidic medium can comprise a protic or aprotic solvent. A fluidic medium can comprise an aqueous medium. A fluidic medium can comprise an organic solvent, such as acetic acid, acetone, acetonitrile, benzene, a butanol, 2-butanone, carbon tetrachloride, chlorobenzene, chloroform, cyclohexane, 1,2-dichloroethane, diethylene glycol, diethyl ether, diglyme, 1,2-dimethoxy-ethane, dimethylformamide, dimethyl sulfoxide, 1,4-dioxane, ethanol, ethyl acetate, ethylene glycol, glycerin, heptane, hexamethylphosphoramide, hexamethylphophorus triamide, hexanes, methanol, methyl t-butyl ether, methylene chloride, N-methyl-pyrrolidinone, nitromethane, pentane, petroleum ether, 1-proponal, 2-propanol, pyridine, tetrahydrofuran, toluene, triethyl amine, xylene, or a combination thereof. A fluidic medium can comprise a polar solvent, such as N-methyl pyrrolidone, tetrahydrofuran, ethyl acetate, acetone, dimethylfuran, acetonitrile, dimethyl sulfoxide, propylene carbonate, N-butanol, isopropyl alcohol, nitromethane, ethanol, methanol, acetic acid, or a combination thereof. A fluidic medium can comprise a non-polar solvent, such as benzene, carbon tetrachloride, chloroform, cyclohexane, dichloromethane, dimethoxyethane, ethyl ether, heptane, hexachloroethane, hexane, limonene, naphtha, pentane, tetrachloroethylene, tetrahydrofuran, toluene, xylenes, and combinations thereof. In some cases, a fluidic medium may comprise an aprotic solvent, such as N-methyl pyrrolidone, tetrahydrofuran, ethyl acetate, acetone, dimethylfuran, acetonitrile, dimethyl sulfoxide, propylene carbonate, or a combination thereof.

A fluidic medium may further comprise one or more components, including: 1) an ionic species, 2) a buffering agent, 3) a surfactant or detergent, 4) a chelating agent, 5) a denaturing agent or a chaotrope, 6) a cosmotropic or crowding agent, 7) a clouding agent, 8) a reactive scavenger, and 9) a blocking agent.

A fluidic medium may comprise one or more ionic species. An ionic species may be provided to a fluidic medium as a salt, thereby providing an anionic species and a cationic species to the fluidic medium. An ionic species can include a zwitterionic species. A fluidic medium may comprise a cationic species such as Na⁺, K⁺, Ag⁺, Cu⁺, NH⁴⁺, Mg²⁺, Ca²⁺, Cu²⁺, Cd²⁺, Zn²⁺, Fe²⁺, Co²⁺, Ni²⁺, Cr²⁺, Mn²⁺, Ge²⁺, Sn²⁺, Al³⁺, Cr³⁺, Fe³⁺, Co³⁺, Ni³⁺, Ti³⁺, Mn³⁺, Si⁴⁺, V⁴⁺, Ti⁴⁺, Mn⁴⁺, Ge⁴⁺, Se⁴⁺, V⁵⁺, Mn⁵⁺, Mn⁶⁺, Se⁶⁺, and combinations thereof. A fluidic medium may comprise an anionic species such as F⁻, Cl⁻, Br⁻, ClO₃⁻, H₂PO₄⁻, HCO₃⁻, HSO₄⁻, OH⁻, I⁻, NO₃⁻, NO₂⁻, MnO₄⁻, SCN⁻, CO₃²⁻, CrO₄²⁻, Cr₂O₇²⁻, HPO₄²⁻, SO₄²⁻, SO₃²⁻, PO₄³⁻, and combinations thereof. A fluidic medium may comprise a chelating agent, such as ethylenediaminetetraacetic acid (EDTA), nitrilotriacetic acid, n-hydroxyethylenediaminetetraacetic acid (HEDTA), oxalic acid, malic, acid, rubeanic acid, citric acid, or combinations thereof.

A fluidic medium may include a buffering species including, but not limited to, MES, Tris, Bis-tris, Bis-tris propane, ADA, ACES, PIPES, MOPSO, MOPS, BES, TES, HEPES, HEPBS, HEPPSO, DIPSO, MOBS, TAPSO, TAPS, TABS, POPSO, TEA, EPPS, Tricine, Gly-Gly, Bicine, AMPD, AMPSO, AMP, CHES, CAPSO, CAPS, PBS, and CABS.

A fluidic medium may comprise a surfactant or detergent. A surfactant or detergent may comprise a cationic surfactant or detergent, an anionic surfactant or detergent, a zwitterionic surfactant or detergent, an amphoteric surfactant or detergent, or a non-ionic surfactant or detergent. A fluidic medium may include a surfactant species including, but not limited to, stearic acid, lauric acid, oleic acid, sodium dodecyl sulfate, sodium dodecyl benzene sulfonate, dodecylamine hydrochloride, hexadecyltrimethylammonium bromide, polyethylene oxide, nonylphenyl ethoxylates, Triton X, pentapropylene glycol monododecyl ether, octapropylene glycol monododecyl ether, pentaethylene glycol monododecyl ether, octaethylene glycol monododecyl ether, lauramide monoethylamine, lauramide diethylamine, octyl glucoside, decyl glucoside, lauryl glucoside, Tween 20, Tween 80, n-dodecyl-β-D-maltoside, nonoxynol 9, glycerol monolaurate, polyethoxylated tallow amine, poloxamer, digitonin, zonyl FSO, 2,5-dimethyl-3-hexyne-2,5-diol, Igepal CA630, Aerosol-OT, triethylamine hydrochloride, cetrimonium bromide, benzethonium chloride, octenidine dihydrochloride, cetylpyridinium chloride, adogen, dimethyldioctadecylammonium chloride, CHAPS, CHAPSO, cocamidopropyl betaine, amidosulfobetaine-16, lauryl-N,N-(dimethylammonio) butyrate, lauryl-N,N-(dimethyl)-glycinebetaine, hexadecyl phosphocholine, lauryldimethylamine N-oxide, lauryl-N,N-(dimethyl)-propanesulfonate, 3-(1-pyridinio)-1-propanesulfonate, 3-(4-tert-butyl-1-pyridinio)-1-propanesulfonate, N-laurylsarcosine, and combinations thereof.

A fluidic medium may comprise a denaturing or chaotropic species, such as acetic acid, trichloroacetic acid, sulfosalicylic acid, sodium bicarbonate, ethanol, ethylenediamine tetraacetic acid (EDTA), urea, guanidinium chloride, lithium perchlorate, sodium dodecyl sulfate, 2-mercaptoethanol, dithiothreitol, tris(2-carboxyethyl) phosphine (TCEP), or a combination thereof. A denaturing or chaotropic species may be provided to alter a conformational state of an array component (e.g., causing denaturation of a polypeptide), or may be provided to maintain a conformational state of an array component (e.g., maintaining a polypeptide in a denatured or partially-denatured state).

A fluidic medium may comprise a cosmotropic species, such as carbonate ion, sulfate ion, phosphate ion, magnesium ion, lithium ion, zinc ion, aluminum ion, trehalose, glucose, proline, tert-butanol, or a combination thereof. A fluidic medium may comprise a clouding agent such as sodium chloride, potassium chloride, sodium bromide, potassium bromide, sodium nitrate, sodium sulfate, sodium phosphate, or a combination thereof. A cosmotropic species may be provided to decrease a separation distance between molecules and array components (e.g., causing smaller separation between an affinity agent and an analyte).

A fluidic medium may comprise a reactive scavenger species. A reactive scavenger may be provided to reduce solution-phase concentrations of reactive species (e.g., oxidizing or reducing species). A reactive scavenger may be provided during a photon-mediated process (e.g., fluorescent imaging) to reduce photodamage or other deleterious photon-related processes (e.g., singlet oxygen generation, free radical generation). Exemplary reactive scavenger species can include ascorbic acid, 9,10-anthracenediyl-bis(methylene) dimalonic acid (ABDA), epigallocatechin gallate (EPGG), N-acetyl-L-cysteine, caffeic acid, reseveratrol, 4-hydroxy-2,2,6,6-tetramethylpiperidin-1-oxyl (TEMPOL), sodium sulfite, 1,4-diazabicyclo[2.2.2]octane (DABCO), sodium pyruvate, N,N′-dimethylthiourea (DMTU), mannitol, dimethyl sulfoxide (DMSO), 6-hydroxy-2,5,7,8-tetramethylchroman-2-carboxylic acid (Trolox), 2-phenyl-1,2-benzisoselenazol-3 (2H)-one (Ebselen), α-tocopherol, uric acid, sodium azide, manganese (III)-tetrakis (4-benzoic acid) porphyrin, 4,5-dihydroxybenzene-1,3-disulfonate, or a combination thereof. Other useful reactive scavengers and methods for their use in reducing photodamage or other deleterious photon-related processes are set forth in U.S. Pat. No. 10,106,851, which is incorporated herein by reference.

A fluidic medium may comprise a blocking agent. A blocking agent may include any species that inhibits orthogonal binding phenomena between assay agents and array components, such as polyethylene glycol, dextrans, albumin, or synthetic polymers such as PF-127 or polyvinylpyrrolidone.

A method set forth herein may involve a step of delivering a fluidic medium to a vessel (e.g., a flow cell, a fluidic cartridge, a reactor or microreactor, etc.) containing an array, as set forth herein. In some cases, after delivering a fluidic medium to a vessel, the fluidic medium may be incubated with an array within the vessel. Incubation of a fluidic medium with an array may be substantially quiescent. Alternatively, incubation of a fluidic medium with an array may be non-quiescent due to mixing, agitation, or circulation of the fluidic medium within or through the vessel.

A method set forth herein may involve a step of altering a fluidic medium with respect to one or more properties of the fluidic medium. Altered properties can include temperature, pH, ionic strength, and composition of the fluidic medium. In some cases, altering a fluidic medium may comprise displacing a first fluidic medium having a first property (e.g., temperature, pH, ionic strength, composition) with a second fluidic medium having a second property, in which the first property differs from the second property. In other cases, altering a fluidic medium may comprise mixing a second fluidic medium or chemical component (e.g., a solute) into a first fluidic medium. For example, a pH of a fluidic medium may be altered by adding an acid or base species to a fluidic medium in a vessel. In another example, a fluidic medium may be diluted or condensed with respect to ionic strength or concentration of a component by addition of a second fluidic medium to the vessel.

The present disclosure provides compositions, apparatus and methods that are useful for detecting, characterizing and identifying proteoforms. For example, the presence or absence of a particular post-translational modification or a particular post-translationally modified amino acid can be determined. In some embodiments, a proteoform can be characterized with respect to the location(s) of one or more post-translational modifications in the amino acid sequence of the proteoform. Locations can be identified, for example, at a specific position of the amino acid sequence for the proteoform. However, in some cases, the location of a post-translational modification in a proteoform can be determined relative to a particular structural motif of the proteoform. For example, a post-translational moiety of a proteoform can be located relative to a short sequence of amino acids in the proteoform or relative to another post-translational moiety in the proteoform.

Methods of the present disclosure are particularly well suited for manipulating and detecting proteoforms. The presence or absence of post-translational modifications (PTM) can be detected using a composition, apparatus or method set forth herein. A PTM can be detected using an affinity agent that recognizes the PTM or based on a chemical property of the PTM. In some configurations, methods set forth herein can be used to differentially manipulate proteoforms based on unique molecular properties or to distinguish one proteoform from another.

A post-translational modification may be one or more of myristoylation, palmitoylation, isoprenylation, prenylation, farnesylation, geranylgeranylation, lipoylation, flavin moiety attachment, Heme C attachment, phosphopantetheinylation, retinylidene Schiff base formation, dipthamide formation, ethanolamine phosphoglycerol attachment, hypusine, beta-Lysine addition, acylation, acetylation, deacetylation, formylation, alkylation, methylation, C-terminal amidation, arginylation, polyglutamylation, polyglyclyation, butyrylation, gamma-carboxylation, glycosylation, glycation, polysialylation, malonylation, hydroxylation, iodination, nucleotide addition, phosphoate ester formation, phosphoramidate formation, phosphorylation, adenylylation, uridylylation, propionylation, pyrolglutamate formation, S-glutathionylation, S-nitrosylation, S-sulfenylation, S-sulfinylation, S-sulfonylation, succinylation, sulfation, glycation, carbamylation, carbonylation, isopeptide bond formation, biotinylation, carbamylation, oxidation, reduction, pegylation, ISGylation, SUMOylation, ubiquitination, neddylation, pupylation, citrullination, deamidation, elminylation, disulfide bridge formation, isoaspartate formation, and racemization. Proteoforms can differ with regard to presence or absence of a post-translational modification, type of post-translational modification present, location of a post-translational modification, number of post-translational modifications present or combination thereof.

A post-translational modification may occur at a particular type of amino acid residue in a protein. For example, the phosphate moiety of a particular proteoform can be present on a serine, threonine, tyrosine, histidine, cysteine, lysine, aspartate or glutamate residue. In another example, an acetyl moiety of a particular proteoform can be present on the N-terminus or on a lysine of a protein. In another example, a serine or threonine residue of a proteoform can have an O-linked glycosyl moiety, or an asparagine residue of a proteoform can have an N-linked glycosyl moiety. In another example, a proline, lysine, asparagine, aspartate or histidine amino acid of a proteoform can be hydroxylated. In another example, a proteoform can be methylated at an arginine or lysine amino acid. In another example, a proteoform can be ubiquitinated at the N-terminal methionine or at a lysine amino acid. A method set forth herein can include a step of adding a post-translational modification to a protein by contacting the protein with an enzyme (e.g., a kinase) or other modifiying agent.

A post-translationally modified version of a given amino acid can include a post-translational moiety at a side chain position that is unmodified in a standard version of the amino acid. Post-translationally modified lysines can include epsilon amines attached to post-translational moieties, whereas standard lysines have epsilon amines lacking the post-translational moieties. Post-translationally modified histidines can include side-chain tertiary amines attached to post-translational moieties, whereas in standard histidines the side-chain amines are secondary amines lacking the post-translational moieties. Post-translationally modified versions of aspartates or glutamates can include side-chain carbonyls, esters or amides attached to post-translational moieties, whereas in standard versions of aspartates or glutamates the side-chains have carboxyls lacking the post-translational moieties. Post-translationally modified versions of arginines can include side-chain amines attached to post-translational moieties, whereas in standard versions of arginines the side-chain amines lack the post-translational moieties. Post-translationally modified versions of cysteines can include thioethers attached to post-translational moieties, whereas standard versions of cysteines have sulfurs lacking the post-translational moieties. Post-translationally modified versions of serines, threonines or tyrosines can include ethers or esters attached to post-translational moieties, whereas standard versions of serines, threonines or tyrosines have hydroxyls lacking the post-translational moieties.

A method of the present disclosure can include a step of removing post-translational moieties from post-translationally modified amino acids, thereby forming standard amino acids. In some cases, an enzyme can be used to remove a post-translational moiety from an amino acid. An enzyme that removes a post-translational moiety independently of amino acid sequence context surrounding the post-translationally modified amino acid can be used. In other cases, a sequence-specific enzyme can be used to remove a post-translational moiety.

A phosphatase enzyme can be used to remove a phosphate moiety from an amino acid. A broadscale (e.g. sequence agnostic) phosphatase such as alkaline phosphatase can be useful. Protein phosphatases are available for removing phosphate moieties from various types of amino acids. Exemplary protein phosphatases include, but are not limited to, tyrosine-specific kinases such as PTPIB; serine/threonine-specific phosphatases such as PP2C and PPP2CA; dual specificity phosphatases such as lambda protein phosphatase or VHR, both of which can remove phosphate moieties from serine, threonine or tyrosine residues; or histidine phosphatase such as PHP. Phosphatases or kinases that are specific to particular signal transduction pathways can be used to remove phosphates in a sequence specific manner if desired.

Several enzymes are available for removing post-translational moieties from lysines. Examples are set forth in Wang and Cole, Cell Chemical Biology 27:953-969 (2020) (which is incorporated herein by reference) and below. Lysine deacetylases can be used to remove acetyl moieties from lysines. For example, at least eighteen different protein lysine deacetylases (e.g. histone deacetylases) are known to remove acetyl moieties from lysines in human proteins. Lysine demethylases can be used to remove methyl moieties from lysines. Deubiquitinases (DUBs) are isopeptidases that sever the amide bond between a lysine side chain of a protein and the ubiquitin (Ub) C terminus. Many DUBs can cleave Ub-Ub amide linkages whereas others show selectivity for particular ubiquitinated proteins.

Optionally, glycan moieties can be released from proteins in a method of the present disclosure. For example, N-glycans or O-glycans can be released from glycoproteins using glycosidases. Any of a variety of enzymes can be used to remove glycans from proteins. For example, α-2-3,6,8,9-Neuraminidase can be used to cleave non-reducing terminal branched and unbranched sialic acids; β-1,4-galactosidase can be used to remove β-1,4-linked nonreducing terminal galactose from proteins; β-N-acetylglucosaminidase can be used to cleave non-reducing terminal β-linked N-acetylglucosamine from proteins; endo-a-N-acetylgalactosaminidase can be used to remove O-glycosylation, for example, removing serine- or threonine-linked unsubstituted Galb1,3GalNac; and PNGase F can be used to cleave oligosaccharides from asparagines. Exemplary reagents and methods for releasing glycans from proteins are set forth in Zhang et al. Frontiers in Chemistry, vol 8, Article 508 (2020) doi: 10.3389/fchem.2020.00508, which is incorporated herein by reference.

A plurality of extant proteins may contain two or more proteoforms of a single species of protein (e.g., at least 2, 3, 4, 5, 10, 20, 50, 100, or more than 100 proteoforms). Alternatively, a plurality of extant proteins may contain only a single proteoform of a single species. A plurality of extant proteins may contain at least one species of protein having two or more proteoforms (e.g., at least 2, 10, 50, 100, 500, 1000, 5000, 10000, or more than 10000 species of protein having two or more proteoforms). Alternatively, a plurality of extant proteins may contain at least one species of protein having only one proteoform (e.g., at least 2, 10, 50, 100, 500, 1000, 5000, 10000, or more than 10000 species of protein having only one proteoform).

A method of identifying extant proteins may further include identifying proteoforms of extant proteins. Accordingly, a method of identifying a proteoform of an individual protein can include the steps of: i) identifying a primary amino acid sequence of the protein based upon a binding profile of the protein, thereby identifying the protein, and ii) identifying a proteoform of the protein. Proteoform-specific affinity agents may be useful for identifying the proteoform of an extant protein. A proteoform-specific affinity agent can be a promiscuous affinity agent, for example binding to post-translational modifications (e.g., methylations, phosphorylations, glycosylations, etc.) of a plurality of protein species and/or proteoforms. A proteoform-specific affinity agent can be highly specific to a single proteoform of one or more protein species (e.g., only binding to a single post-translationally modified amino acid of a single protein species). A proteoform may be identified in part by detecting presence of binding of one or more affinity agents to an extant protein. Alternatively, a proteoform may be identified in part by an absence of detectable binding of one or more affinity agents to an extant protein (e.g., due to absence of a post-translational modification at an amino acid residue of the extant protein, due to absence of a bindable epitope due to splice variation of the extant protein, etc.).

In some cases, it may be preferable to contact extant proteins with a proteoform-specific affinity agent before contacting the extant proteins with other promiscuous or non-proteoform affinity agents. Presence of certain post-translational modification may inhibit binding of affinity agents to epitopes where said post-translational modification are present. Accordingly, a method may further comprise a step of removing post-translation modification (e.g., chemically or enzymatically) from extant proteins. After detecting binding of proteoform-specific affinity agents to extant proteins, and optionally removing one or more post-translational modification from the extant proteins, the extant proteins may be subsequently contacted with a series of promiscuous affinity agents, thereby providing binding profiles for each individual extant protein.

Although performing a single binding reaction between a promiscuous affinity reagent and a complex protein sample may yield ambiguous results regarding the identity of the different extant proteins to which it binds, the ambiguity can be resolved by decoding the binding profiles for each extant protein using machine learning or artificial intelligence algorithms that are based on probabilities for the affinity reagents binding to candidate proteins. For example, a plurality of different promiscuous affinity reagents can be contacted with a complex population of extant proteins, wherein the plurality is configured to produce a different binding profile for each candidate protein suspected of being present in the population. The plurality of promiscuous affinity reagents can produce a binding profile for each extant protein that can be decoded to identify a unique combination of positive outcomes (i.e. observed binding events) and/or negative binding outcomes (i.e. observed non-binding events), and this can in turn be used to identify the extant protein as a particular candidate protein having a high likelihood of exhibiting a similar binding profile.

Binding profiles can be obtained for extant proteins and decoded. In many cases one or more binding events produces inconclusive or even aberrant results and this, in turn, can yield ambiguous binding profiles. For example, observation of binding outcome at single-molecule resolution can be particularly prone to ambiguities due to stochasticity in the behavior of single molecules when observed using certain detection hardware. As set forth above, ambiguity can also arise from affinity reagent promiscuity. Decoding can utilize a binding model that evaluates the likelihood or probability that one or more candidate proteins that are suspected of being present in an assay will have produced an empirically observed binding profile. The binding model can include information regarding expected binding outcomes (e.g. positive binding outcomes and/or negative binding outcomes) for one or more affinity reagents with respect to one or more candidate proteins. A binding model can include a measure of the probability or likelihood of a given candidate protein generating a false positive or false negative binding result in the presence of a particular affinity reagent, and such information can optionally be included for a plurality of affinity reagents.

Decoding can be configured to evaluate the degree of compatibility of one or more empirical binding profiles with results computed for various candidate proteins using a binding model. For example, to identify an extant protein in a sample, an empirical binding profile for the extant protein can be compared to results computed by the binding model for many or all candidate proteins suspected to be in the sample. A machine learning or artificial intelligence algorithm can be used. An algorithm used for decoding can utilize Bayesian inference. In some configurations, identity for an extant protein is determined based on a likelihood of the extant protein being a particular candidate protein given the empirical binding pattern or based on the probability of a particular candidate protein generating the empirical binding pattern. Particularly useful decoding methods are set forth, for example, in U.S. Pat. No. 10,473,654; US Pat. App. Pub. Nos. 2020/0318101 A1 or 2023/0114905 A1, or Egertson et al., BioRxiv (2021), DOI: 10.1101/2021.10.11.463967, each of which is incorporated herein by reference.

One or more compositions set forth herein can be present in an apparatus or vessel. For example, a linker-attached affinity reagent and/or linker-attached analyte of the present disclosure can be present in a vessel, such as a flow cell. In some cases, the linker is attached to the affinity reagent or the analyte via a particle (e.g. structured nucleic acid particle). As a further option, the vessel can be engaged with a detection apparatus. The vessel can be permanently or temporarily engaged with the detection apparatus. A detection apparatus can be configured to detect contents of a vessel, for example, by acquiring signals arising from the vessel. For example, a detection apparatus can be configured to acquire optical signals through an optically transparent window of the vessel. Optionally, the detection apparatus can be configured for luminescence detection, for example, having an optical train that delivers radiation from an excitation source (e.g., a laser or lamp) then through a window of the vessel. The detection apparatus can further include a camera or other detector that acquires signals transmitted through the window of the vessel and through an optical train. Optionally excitation and emission can be transmitted through the same optical train; however, separate optical trains can also be useful.

A detection apparatus can include a fluidics system, for example, configured for fluidic communication with a vessel, such as a flow cell. In some configurations, a detection apparatus can include one or more reservoirs containing affinity reagents or analytes that are delivered to a vessel. The one or more reservoirs can contain linker-associated affinity reagents that recognize one or more analytes in the vessel. Alternatively, the one or more reservoirs can contain linker-associated analytes that recognize one or more affinity reagents in the vessel. Optionally, a detection apparatus can be configured to include a waste receptacle to which waste from the vessel is collected. For example, affinity reagents can be delivered from the apparatus through an ingress of a flow cell and waste can be removed through an egress of the flow cell to the apparatus.

One or more compositions set forth herein can be provided in kit form including, if desired, a suitable packaging material. Optionally, one or more compositions can be provided as a solid, such as crystals or a lyophilized pellet. Accordingly, any combination of reagents or components that is useful in a method set forth herein can be included in a kit.

The packaging material included in a kit can include one or more physical structures used to house the contents of the kit. The packaging material can be constructed by well-known methods, preferably to provide a sterile, contaminant-free environment. The packaging materials employed herein can include, for example, those customarily utilized in affinity reagent systems. Exemplary packaging materials include, without limitation, glass, plastic, paper, foil, and the like, capable of holding within fixed limits a component useful in the methods of the present disclosure.

Packaging material or other components of a kit can include a kit label which identifies or describes a particular method set forth herein. For example, a kit label can indicate that the kit is useful for detecting a particular protein or proteome. In another example, a kit label can indicate that the kit is useful for a therapeutic or diagnostic purpose, or alternatively that it is for research use only.

Instructions for use of the packaged reagents or components are also typically included in a kit. The instructions for use can include a tangible expression describing the reagent or component concentration or at least one assay method parameter, such as the relative amounts of kit components and sample to be admixed, maintenance time periods for reagent/sample admixtures, temperature, buffer conditions, and the like.

In some cases, a kit can be configured as a cartridge or component of a cartridge. The cartridge can in turn be configured to be engaged with a detection apparatus. For example, the cartridge can be engaged with a detection apparatus such that contents of the cartridge are in fluidic communication with the detection apparatus or with a flow cell engaged with the detection apparatus. A cartridge can be engaged with a detection apparatus such that contents of the cartridge can be observed by the detection apparatus, for example, using an assay set forth herein.

### EXAMPLES

### Example 1. Length of a Linker

An affinity reagent linked to solid support by a nucleic acid linker can move freely within a volume approximately bounded on one side by the solid support surface and a hemisphere with a radial dimension of L, in which L is the length of the nucleic acid linker at linear extension. Accordingly, the affinity reagent would be able to diffuse freely in a volume proportional to L³. If the nucleic acid linker length was halved to L/2, the effective concentration of the affinity reagent with respect to a co-localized analyte would be increased by a factor of 8.

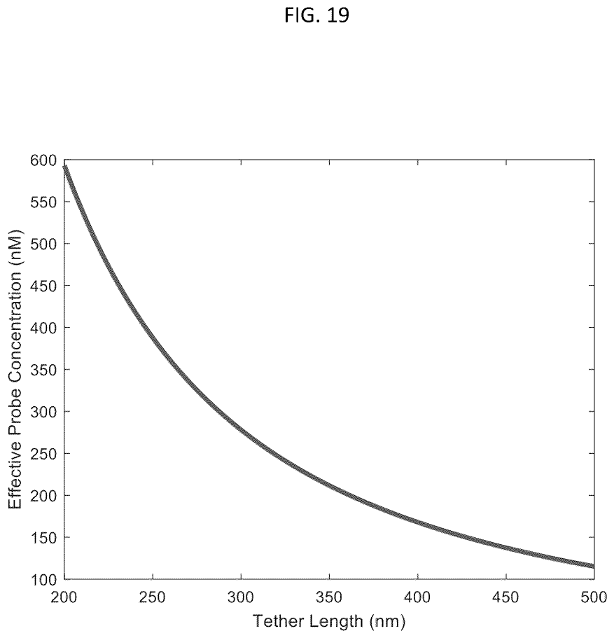

An affinity reagent attached to a double-stranded nucleic acid linker was simulated via a computational model to assess the effect of tether length on the effective probe concentration. FIG. 19 displays the simulation results, with the x-axis plotting the tether length and the y-axis plotting the effective probe concentration. For example, if a probe concentration of 400 nanomolar (nM) is desired to achieve a desired level of binding at equilibrium, a double-stranded nucleic acid linker length of approximately 250 nanometers (nm) would be sufficient.

Accordingly, if an affinity reagent has been characterized with respect to its dissociation constant, K_{D}), relative to a particular binding target, an excess concentration factor, f, can be chosen (e.g., 1.5-fold, 2-fold, 3-fold, 4-fold, 5-fold, 10-fold, etc.) to drive binding toward a high concentration of affinity reagent-analyte complexes. The effective probe concentration can then be determined as f*K_{D}. FIG. 19 can be utilized to determine a maximum linker length to achieve the desired effective probe concentration. Each affinity reagent utilized in an assay may be paired with a linker of a differing length based upon its measured binding affinity.

### Example 2. Optimal Affinity Reagent Concentration

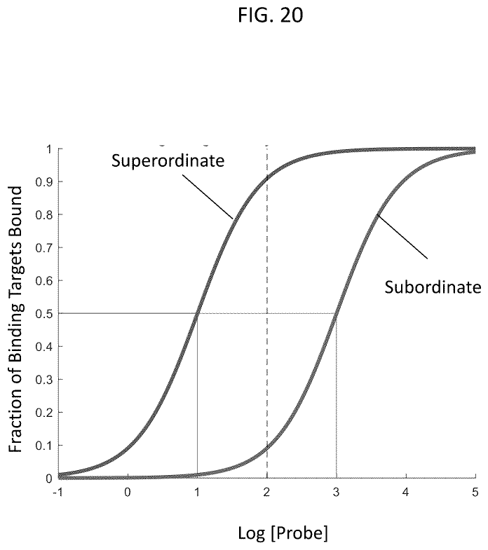

Equilibrium binding characteristics are measured for an affinity reagent with respect to binding to trimer targets. The affinity reagent is determined to have a superordinate binding interaction with the amino acid trimer sequence DSP with a dissociation constant of 10 nanomolar (nM). The affinity reagent is also determined to have a subordinate binding interaction with the amino acid trimer sequence DTP with a dissociation constant of 1000 nM. FIG. 20 displays equilibrium binding curves for the superordinate and subordinate binding interactions of the affinity reagent with respect to proteins containing the trimer binding targets.

An optimal concentration for assaying with the affinity reagent is chosen. It is preferred to maximize the likelihood of forming the superordinate binding interaction and reduce, or even minimize, the chances of forming the subordinate binding interaction. The dashed line in FIG. 20 shows the affinity reagent concentration at which the separation in binding likelihood is maximized. About 90% of proteins containing the epitope DSP will be bound at the optimal concentration, while about 10% of proteins containing the epitope DTP will be bound. The optimal concentration for the affinity reagent can be calculated as:

$$[ Affinity Reagent ] = \sqrt{{K}_{D , super} * {K}_{D , sub}} \qquad ( 1 )$$

The optimal concentration for the evaluated affinity reagent is 100 nM. For assays utilizing fluid phase affinity reagents that are not immobilized to a solid support, the affinity reagent can be provided at the 100 nM concentration. For assay utilizing immobilized affinity reagents, an optimal linker length can be calculated as described in Example 1.

### Example 3. Recording of Binding Interactions

Binding interactions between affinity reagents and peptide targets were recorded utilizing a biotin ligase enzyme. Arrays of peptide targets were prepared according to methods set forth in U.S. Pat. Nos. 11,203,612 and 11,505,796, and U.S. patent application. Ser. No. 18/613,879, each of which is herein incorporated by reference in its entirety. Two types of arrays were formed: 1) single-molecule arrays of peptide targets containing the amino acid sequence HSP (target peptide); and 2) single-molecule arrays of peptide targets containing the amino acid sequence DTR (off-target peptide). Peptide targets were attached to array sites by nucleic acid nanoparticles as described in U.S. patent application Ser. No. 18/744,286, which is herein incorporated by reference in its entirety. Each nucleic acid nanoparticle contained multiple pendant oligonucleotide adjacent to the attached peptide target, each pendant oligonucleotide configured to attach an oligonucleotide attached to an avi-tag (amino acid sequence GLNDIFEAQKIEWHE; SEQ ID NO: 1). Peptide targets were deposited on arrays at a 300 picoMolar concentration for 45 minutes of incubation time. After deposition of peptide targets on each array, the arrays were incubated with oligonucleotides attached to avi-tags (1 microMolar concentration for 45 minutes of incubation), thereby allowing the oligonucleotides to hybridize to pendant oligonucleotides of array-bound nucleic acid nanoparticles.

Affinity reagent constructs were formed by attaching BirA biotin ligase enzymes to anti-HSP antibodies. Affinity reagent constructs were incubated with arrays of peptide targets for 45 minutes at 1 microMolar concentration. The affinity reagents were incubated with arrays in a buffer containing 10 millimolar (mM) HEPES buffer, 120 mM sodium chloride, 10 mM magnesium chloride, 5 mM potassium chloride, 1 mg/mL sheared salmon DNA, 1% Pluronic F-127, 0.1% Tween-20, 0.1% BSA plus 1× BirA buffer (50 mM tris pH 8.1, 5 mM magnesium chloride, 0.15 mM biotin, 2 mM ATP) for 45 minutes. After incubation with the affinity reagent constructs, arrays were incubated with fluorescently-labeled nucleic acid nanoparticles containing attached streptavidin molecules. The fluorescently-labeled nanoparticles were incubated with arrays for 45 minutes at a 20 nanoMolar concentration, thereby facilitating attachment of streptavidin molecules of fluorescently-labeled nanoparticles to biotinylated avi-tags. After fluorescent labeling, arrays were imaged by confocal fluorescence microscopy. Fluorescent images were analyzed to determine the fraction of peptide-containing array sites with a fluorescent signal, thereby indicating presence of the affinity reagent construct for a sufficient time to record the binding interaction via biotinylation of an avi-tag.

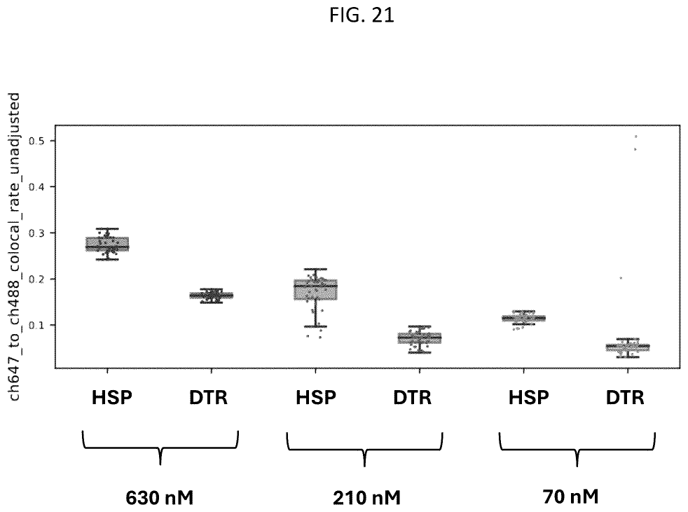

FIG. 21 displays titration of affinity reagent construct concentration against both HSP-containing arrays and DTR-containing arrays. Tested affinity reagent concentrations were 70 nanoMolar (nM), 210 nM, and 630 nM. The quantity of sites having a recorded binding interaction is observed to increase with increasing concentration. At each concentration, substantially more signal is recorded by the affinity reagent for its target peptide (HSP) than for the off-target peptide (DTR).

1. A method of detecting a first reaction, comprising:
(a) providing immobilized on a solid support:
(i) an analyte; and
(ii) a first reactant, the first reactant being immobilized on the support within a first distance from the analyte;
(b) contacting the immobilized analyte with a probe, the probe comprising an affinity reagent and a second reactant, the affinity reagent having binding specificity for the analyte, and the second reactant being capable of a second reaction with the first reactant when within a second distance from the first reactant;
(c) forming a first reaction between the analyte and the affinity reagent, thereby bringing the second reactant within the second distance of the first reactant;
(d) after forming the first reaction, forming the second reaction between the first reactant and the second reactant; and
(e) detecting the first reaction.

2. The method of claim 1, wherein the first distance is an optically non-resolvable distance between the analyte and the first reactant.

3. The method of claim 2, wherein the first distance is less than 300 nanometers (nm).

4. The method of claim 3, wherein the first distance is less than 50 nm.

5. The method of claim 1, wherein the second distance is less than the first distance.

6. The method of claim 5, wherein the second distance is no more than 10 nm.

7. The method of claim 1, wherein the probe further comprises a linker, wherein the linker couples the affinity reagent to the second reactant.

8. The method of claim 7, wherein a length of the linker is greater than the first distance.

9. The method of claim 7, wherein a length of the linker is less than the first distance.

10. The method of claim 1, wherein forming the second reaction comprises forming a non-covalent binding reaction.

11. The method of claim 10, wherein the first reactant comprises a first oligonucleotide, and wherein the second oligonucleotide comprises a second oligonucleotide.

12. The method of claim 11, wherein forming the non-covalent binding interaction comprises hybridizing the first oligonucleotide to the second oligonucleotide.

13. The method of claim 11, wherein forming the non-covalent binding interaction comprises hybridizing a third oligonucleotide to the first oligonucleotide and the second oligonucleotide, thereby coupling the first oligonucleotide to the second oligonucleotide.

14. The method of claim 1, wherein forming the second reaction comprises forming a covalent binding reaction.

15. The method of claim 14, wherein the second reactant comprises an enzyme, wherein forming the covalent binding interaction comprises attaching a detectable label to the first reactant with the enzyme.

16. The method of claim 14, wherein the first reactant comprises an enzyme, wherein forming the covalent binding interaction comprises attaching a detectable label to the second reactant with the enzyme.

17. The method of claim 1, wherein forming the second reaction comprises forming a photon transfer reaction.

18. The method of claim 17, wherein detecting the first reaction comprises detecting a photon from the photon transfer reaction.

19. The method of claim 17, wherein the first reactant comprises a first fluorescent label, wherein the second reactant comprises a second fluorescent label, wherein forming the photon transfer reaction comprises forming a Forster Resonance Energy Transfer (FRET) interaction between the first fluorescent label and the second fluorescent label.

20. The method of claim 1, wherein the probe further comprises a detectable label.

21. The method of claim 20, wherein detecting the first reaction comprises detecting a signal from the detectable label.

22. The method of claim 1, further comprising binding a bridging molecule to the first reactant and the second reactant.

23. The method of claim 22, wherein the bridging molecule brings the second reactant within the second distance of the first reactant.

24. The method of claim 22, wherein the bridging molecule further comprises a detectable label.

25. The method of claim 24, wherein detecting the first reaction comprises detecting a signal from the detectable label.

26. The method of claim 22, wherein the bridging molecule non-covalently binds to the first reactant, the second reactant, or to both the first reactant and the second reactant.

27. The method of claim 22, wherein the bridging molecule covalently binds to the first reactant, the second reactant, or to both the first reactant and the second reactant.

28.-97. (canceled)


SEQUENCE LISTING (1 sequence)

SEQ ID NO: 1 - protein, 15 aa, synthetic construct
GLNDIFEAQKIEWHE